SEQUENCE LISTING (67 sequences)

SEQ ID NO: 1 - DNA, 156021 bp, Homo sapiens
  source 1..156021: mol_type = genomic DNA
gctcccggcttagaggacagcggggaaggcgggcggtggggcagggggcctgaagcggcggtaccggtgctggcggcggcagctgaggccttggccgaagccgcgcggtgagtctagggcctggcacgacccctctagggcggcgtaatgtccagacccacggccagcctgcccgtgggggggccaggggaagccgcccgtctgggacgtggggtccccacattgccggccccacgacctgggcaccgtctgaggccttgactcccacccctcgaggcaacgcccacctccccgggacccccaagagacccctggacccttcggcccgcggtacgtccgcccgaaggccggcccatcagctctggtattgccctttgggtcttttgcctgtcacagccaccctttcttttgagccacgtataaaaattgtgtatctctgccccagggaacactagcgtccgtgtcagccccttgtcctccactgtggacacctctcagagatccagtccccgaaactgagcttggttacatcgttggggtacatcttctctccctctcccccagccccatccctgtttcccccatccaatcttaacttccttaagccctatcaacaccattaggatatttgacttcagatatcctaagtttaattgaattcagtctggagcagatggcctgtgggcctcagatcatataaagatacactggttctttcctgagaatagaaatcccttgccagccaccttcccccctttcggacgcacacacatacacacaaactggagtagttttcttagcagggattcttaagtttcttctcccctaaaggatgacatttctttctgcagtcttcttcttggcagtggagtatttgaaagctttacaaaaccaattattcccaggtttttcctctgtgccttacaaattctttcaaaaataagaattttgagaaattggttttgtagcataagctgaacatacttgggataggtgtatgttacacccatggcagtgtgggcataattgaggaatgagggaatgagctcaggaactaattggtgttttttgttttgttttgagacggagtttcgctctgtcgcccaggctggagtgcagtggctcgatctctgctcactgcaagctccgcctcccgggttcccgccattctcctgcctcagcctcccgagtagctgggactacaggcgcccgccaccatacggctaatttttttttttttttgtatttttagtagaggcgggatttcaccgtgttggccaggatggtctcgatttcctgacctcgtgatccgcccgcctcggcctcccaaacccggccaggaactaattactttcttttagcttacatttgaagagctagtcccctgtagaactgcgttgttcagtacaagaaccaccagccacatgtggctgtggagcacttgaaatgtgactagtccaggccgggcatggtggctcacacctgtaatcccagcactttgggaggccaaggcgggcagatcacctgaggtcaggaatttgagaccagcctggccaacataataaaaccccatctctactaaaaacacacaaaaattagccaggcgtggtgctgcacatctgtagtcccagctactcgggaagctgaggcaggagaatcatttgaacccgggaggtggatgttgcagtgagccgagatcatgccactgcactccagcctgggtgacagagcgagactctgtctcaaaaaaaaaaaaaaaaatgactagtccaaattgacattgttgtaagtgtaaaatgcacattagattttgaagactcgctaaaaaaaaagaatggacactatatcaattttttaaatattgataacatgttgaaatgatattttggatatattggtttaggtaattaatttcacctgtttctttttaccttttaaaatatggctacaagcagccttaaaattacatttgtggatcattgtatttcctcttttttttttttttaattgagatggagttttgctcttgttgcccaggctgtagtgcaatggcccaatctcagctcactgcaacctctgcctcctgggttcaagtgattctcctgcctcaacctcccaagtagctggggttacaggcacgcactgccacgcctggctaattttgtatttttagtagagacggggtttcaccatattgcccaggctggtctcaaactcctgacctcaggtgatctgcccaccttgggctcccaaagtgccgggattacaggcgtgagccactgtgcccggcctaaatttccattagatagtgctgctgtagaataataatctctctttttagactgaacttgaggaaaggggcagtcttagtcatttttaattctttccctacctcctgacatttgttacctggcattttgccatgtacatagtagtagtagtagtaataataatagctaacaattattaagaacttagtatatgccctgcactgttttaaacactttttgacacgtattaagtcatttagttctcacatcaacccatgaactaggtactattatcttcctattgtagttgaagaaacacattttgagtgtctttcttgaggtatataaccttgtttttgttttcgtttgtttttgttttgagacagtgcagtggcaggatctcggctcactgcagcctctacctcccaggttcaagggattctcctgcctcagcctcctgggtgacacatgccaccacgcccagctaatttttgtatttttcatagagatgggttttcaccatgttgggcaggctggtctcaaattcctgacctcaggtggtcctccaggctcagcctcccaaactgctcaaggtatataacccaaggattcaaacccaagtaggctaggccgatagcccacaatttaacctccctactacactgcctcttttaaacatcagtcattgtttaatgaatctaggctttctaaatgttcctcaatcagatctctcaacctttttagaatcccctgcctgacaagggctagggggagatctgtgttgggtgaccagggccataagtaaatttatcatcatactttcttatccagaatattaaaggaaacttctctctaaagaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagaggtgcttctgggatctggggccagtcattgtgggatgtgtgggctaaatgtaccatcactggtttacatttttttttgtacagctaagtgaggatttggaaaacagagattattttataaaggcacccaacctgtcacatccataaaacatgcaggttcttttgcagcagttgcacatttttgtagcatttttcttgtttgtggtcacattttaggggccagaatagacttagctgtttcttttaactttttattttttccttaccacataccacaccctcaaatgctttctgtctgataacagtgattgggatgggtttggagagttgagagctaggggctttctaaatagagaccagacttgaggtcagagacagtatcttgtttctccttttatccctggtactagaaagaacctaggacaggacattttctgccacagagtagatacctaataaatatttgttaaataatcagatgattgcaagttcaggtactctacagtgtaatcctatagaaatgggcagtaacttgtcttctttggtttgaactatacctcctgcctctcaggatctaatctttagatgccagcccactcccaccaccacagttagagaattattagaaattgttctgattatatcttcttttgcagatgagtcaagaggagtctactcgcttctatgaccaactgaaccatcacatttttgaattggtttccagctcagatgccaatgagaggaaaggtggcatcttggccataggtaaggacagagtctctggtcacaaaaacaacacactcacctttgtgcccactgcaccctgtgtctgccacttactggatactagttcatatttgcttaatgaaatttaagaagcccgtggcttttaattctagcagcagccctgggccacaggaaaaacgtatttttagatgtggaagtggagtctcggaaccatcgagtgacttttcagacagtttaccagagctgagactagaagccagctttctttccactccttcatattgccttttgtgggctctgcttcctcagtagagcagggtttcgcatagtggggctcacgaatggaaaagtggaatcggtatagttaataaaaggcaggtcttttttttttttttgagatggagtctcactcttgccggggctggagttcagtggtgcaatcttggcttgctgcaacctccgcctcctgggttcaagcgattctcgtgcctcagcctcccgagtagctgggattacaggcacctgccaccatgcccagctaatgtttgtattttcagtagagacagggtttcaccatgttggccaggctggtctcgaactcctgacctcaagtgatctgcccacctcagcctcccagagtgctgggattacaggtgtgagccaccacaactggccaggcaggtcatttttaacttctaaggactggtcatcagtcctgtgaattattatccagtcccagttgttttccctttttggggggtaggggagaaaattatactagcatgagattattttgctttatgtaaatatttctggcacaagtattaaatatctgggttaaaatgaaaattgtatctgtccttgccatctagaaatgtactcaaagtagaatggagggttcactcgccagatttttttttttttttttttttttttctctgagacagagtctcactctgtcgcccaggctggagtgcaatggtgcagtctcggcttactgcaacctctgcctcccaggtccaagcaattctcctgcctcagcctcccaagtagctgggattacaggcgtgtgccaccacgcctggctaatttttttgttttcttttttttttttttttttttgtatcttagtagagacggggtttcaccatgttgcccaggctggtctcaaactcctgagctcaggcaatccgcccgcctcagcctcccaaagtgctaggattacagacttgagccaccgtgcctggccggcactcaccagcttttagttgaagtctgcgtactttcctggccagcatacgccccatcatcaccttttgccttcctctcttttgcagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtgagtgtctacagggaggagcacagccaggcttggggaacgatggtgtcatgggggggtcttttgcttccttctgaggctcattccttaaaaaagctggttagaagagctaggattccaggctgtgagatggggtagagtccgtccttaagagggaatctagcatggtgtggtggggatcagaagcatcagctgcgtgctttccagtctgctgcccttcggaggaggcagtactgtgaggagggggcacggtaccaggaaaaggacacacacttggaccctggctcaagtcttggctttgcaacttcctacctaactctgtaacctggggcagcttctgaccttcagtttcttcctctgtcaaataaaaataacaacaggattaaacattaaacaggattatccattcagtaaatatttactacatacctactgtattccaggtagagttctaggctgttagtatagtggtaaagaaaacataaaacacatctgcacttccagaacttatgttcaaacattacaagcattctgtgtttgtaaacaacctaggataatgagcactgagatgtttaatcaattttgctttccttcctctgtcctcagaatttcctagctagaagagtgagcaagccttctctgttcctggtgtgtatggctctaagatgacaagagactgtactcctgagagaacaaaactctggtaccaatgagagttctcggtttgcttttttctccaggtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacagggtaaggagatggctccattcccaccactagcaaagcacatctgcgtagagaatcacctggcctaaagggcagccacaagcccttgccatggagcaagaatcacgttttggtttgctctcaaatgaatttttttttttttttttttgtgatggagtcttgctttgtcgcccaggctggagtgcagtggcgcaatctcggctcactgcaacctccgcctcttgggttcaagcgattttctcccaccttagcctcccgaatagctaggactacaggcgtgtgccaccacgcccagctaatttttgtaatagagacagggttttgccatgttggccaggctggtctcgaattcctgacctcaggtgatccacctgccttggcctctcaaagtgctgggattacagacatgagctaccgcgcctggcctccaatgaattttttatgctcagtatgaatcaatagttactttattgcattttattaaagcaaacatctattgatagccataccattattctatacattattttatgtatgactaagaatgcaaatatgccaattaaactgtgacttggtacctctttctttttttttttttttttttttttttttggagatggagtcttgttctgtcaccaagctggagtttagtggtgtgattttgtctcactgtagcctctgcctcctgggttcaagtgattctcctgcctcagcctcccgagtagctgggactacaggcatgtgccatcacgcccagccaatttttgtgtttttagtagagacagggtttcaccatattggccaggatggtcttgatctcttgacctcatgatccacctgccttgacctcccaaagtgctgggattacaggcatgagccactgtgcccggccaactttgtaccttcttcttattaataattaattataagacatctcagctggatgctcacgcctgtaatcccaacattttgggaggccaaggcaggaggattgcttgagctcaggagtttgagaccagcctgggcagcatgtgggatcccgtctctacaaaaaataaaaaaattagctgggcctggtgttacacgcctgtggtcccagctatttgggaggctgaggcgggagaattgcttgagcccaagagatcaaggctgcagtgagtcatgatcatgccactgcactccagacttgacaacagagtgagactctgtctcaaaaaaaaaaaaaagacatctcttttcagagatgttaaaatacggaaaaataggtgtctcaaaatcagtgagatacaaatgatactaatttagaaagtgccatttctctctgctctagtataatgaggcctgtgctgattactgctgcagagggaacttatcagtgagtcttatattagaaaaagagctctgaacatgggattgggagatgcgggttccagtcttgcctttgctgtgtgatcttggacaagtcacttaaattctttgagcctgtgtttttagctgttaaatagggatgatgatggtatcttcctcaaaggcttattatgagaattgaagctggggctgggcgtggtggcttacacctgtaatcacagcactttgggaggctgagatgggtggatcacttgaggtcaggagtttgagaccagcctggttaacatggtgaagccctgtctctactaaaaatacaaaaattagccaggcgtggtagtgtgtgcctgtaatctcagctactcagggggctgaggcaggagaatcacttgaacccaggaggtggaggttgtggagagccgagattgcgccactgcactccagcctgggcaatagagcaagactccatctcaaaaaaaaaaaattgaagctgaattaaataaaagagattgatcagttagttttgttgatgactggcactaggcactgagcaggcgtggtagcacctcagaacccccagtgtagatgggatgggcctgtattctttctgggtttgtactctgtctctaatgaaggcaccctgtctctctacatagcacacatttgaagaagcagagaagggatttgatgagaccttggccaaagagaagggcatgaatcgggatgatcggatccatggagccttgttgatccttaacgagctggtccgaatcagcagcatggagggagaggtgagaagccacggcagaggggagtccaggctccccgtgtaccattctgtgagcgaggcaaggcgagatccatgtcctcatggagctgacattttgtggcaggaaacataataaataagtaaataagataatttcagatagcagcaagttctatgaagaaagtcaaaccagaatagaatagagagtgatggggagggggtgagtggagggtcaaggaaggccctccctgaggtggtgttatttgacgggggtccacatgacacgaaggagctaccttgcagcaggccgggggactgtgttccaggctgaggctttgaggtggaagagtgtgagacattgatgaaacagaagaaagggaagtatggctggtgtaccgggagcaagggagagggaaggaggggccaaattaataggcccacggttcacagtaaacacgagctttcacaggggtaaacattggaagtatgcacacaggcgaataatgtgatgtcatgcgtttctaacaatcactgactgcttataggaaaaaatgtggggaaaggcaggaatgaaagcagggagaccaattaggagatgtgttggcttgagaaggtggatttaaaagtgcatggattttattagggaaacagccttcaagagaatatggggaggaagctgggagaagcatcggaccacaatgcacatctgaccctgagtaagcaagagggtaaaattcggatagaagtgttctagcctactctgcagggccaccgcggggtcctcaagccaaagttggctgtcagagaggtctgtgtctcccaagagcaggatagccttagcatccctactgtgttctgtcattggctgggagcaatgggtgacgttggcacaaacttagtgatgggtttcagagcagcagctggggcccttggttatgcgccttgtagttggaggttgcattctcacggctgccagacgaagctattacggtagtctgggtgagtgacagtggtggctttgactaggagtggtggcacagatactgagaagtagttgattcaggatatatttgggaggtaaagctccatctgtgagactggctgtagaaaatgagctgtgaaattttaccccggggattcctgcttataatagacacagtgcttgttagataatagccatgcccagatgtgggagcagcgcatgcagcagtgtgcagaagcaggaggagtctggcttcagatccccaccgctctgatgactgtgcgactcaggcaagttacctgtttagcctcatttttctccctttggacaggggctcttacctacctcattgggctcttgtgcggtttctatatgatgttggctctctatatccatgagttctacatctggattcaactagtcatggttcacaaatacccagggctgggtgcagtggcccacacctgcaatttcagcactttgggaggctgaagtgggaagatcacttgatcccaggagttcgtgaccagcctgggcaaaagacggtgagaccgtgtctctacaaaaaaatttttttaactttttaaaaagaaaaaaaaaatcaaggaaaaaacaaacaatttaaagataatacaaatttttaaaaatacagtgcaacaaatatttacatagcatttactttgtataaggtgttataactaacatagagatgatttaaagtatgtgggaagctatacataggttatatgcaaatactatgctactttatcggagggacttgagcatctgcagattttggtatcgcagtgggtcatggaaccaacccccatggatactaaggggatgactatgtgtgtgaagtgtccagcacagcacctaatacatagtgggcattctataaatggtaaccttcagattttacctcctgagtttaacataatgttttccactgtaatctaggcacctctgagatctttgtttcctcccattaaaacaggacagtatcacctagctcatagagtggttgtcagatttcattgaaatagttcttgtgcaggtggtttgtaaattataagaaactatccaaggctgggcatggtggctcatgcctgtaatccaatccaagcattctgggagaccgaggtgggagtttgagctcaggagtttgagaccagcctgggcaacataatgagaccttgtcactaaaaaaaaaaaaaaaaaaagaaactatctaaatataaggtattaaaatttcataatttgttcattcattcaacacatattaattgagcacctagtatatgccagctacactgtgtcctaagagctagaataatccagcagtgaacaaaatagattccattcttataagggaagatagataatatacaagtaaaaacctattatatatcatatggttataagagctatggagaaaagtagggcaaggaaggggaatagagtatgctcacctggattaaggattacattttaaaacacggtgctatttgagcacagccatgaaggaggttagaaaaaagccctacaggtatctggggaagagcattctaggcagtggtccctgaggcaggagcgtgctggtgtattagaggagcagcaaatactgcagtagcagctcccctaccatggctgagcagagtgagcaggagaagtgtgtggggatatggggtcagaggaacacctaagggaaggaggggcatatcacatagggcctttgtgtgggaagcgggaagctataagtgggctttgagcagtggagtgacatttaactcatatttcaaaaggatcactcttgccattgtgttgagaatggattgttagagggtcaaggactaaagccaggaatccagttaggagggaattgccatggtcttagtgggggatatttgtggctcagaccacagtgggagctatggagttgcaagaagcaactggattctgaatttgggatttgctgatggtttggaggtagatggagagaaagaagtgtcaaggtgtttggcctgagtattaggaggatgaagttgccattcactgcagtgggaagactgaaatgggagcagcaggcttgcgggagaagactgggaatttggttttgaacctgttaacttagagacctgttgggcacctgagtgaggatactgaatagttctggggagagagctgggcaagggggatggatttgggcattgtcagcgtatagatgttatttcatcatgagactgggtgagaagcacaagggaactagtacagttggagaagagatggagtctgcaaacctgaggtcaggagatgaagaggaccaaggcagatggggaaggaacagccagggagggaggaggagagccaggagagtttagagtctgagaagccaggtgaagaaagggcgtcaaggaggtggaagtatcagttggatgaaatgctgcagataagtcaaataagatgaggactaagaattgaccattggatttagcagcttaagactcactggttgaggggcgcctctgcccggccgcccctactgggaagtgaggagcccctctgcccggccagccgccccatctgggagggaggtgggggggtcagccccctgcccggccagccgccccgtccgggagggaggtggggggtcagccccccgcccggccagccgccccgtccgggagggaggtcggggggtcagccccccgcccggccagccgccctgtccgggaggtgaggggcgcctctgcccggccgcccctactgggaagtgaggagcccctctgcccggccagccgccccgtccgggagggaggtgtgggggtcagccccccgcccggccagccaccccgtccgggaggtgaggggcgcctctgcccggccgcccctgctgggaagtgaggagcccctctgcccggccaccaccccgtctgggaggtgtgcccagtggctcattgagaacgggccatgatgacagtggcggttttgtggaatagaaagggggcaaaggtggggaaaagattgagaaatcggatggttgccgtgtctgtgtagaaagaggtagacatgggagacttttcattttgttctgtactaagaaaaattcttctgccttgggatcctgttgatctgtgaccttacccccaaccctgtgctctctgaaacatgtgctgtgtccactcagggttaaatggattaagggcggtgcaagatgtgctttgttaaacagatgcttgaaggcagcatgctcattaagagtcatcaccactccctaatctcaagtacccagggacacaaacactgcggaaggccgcagggtcctctgcctaggaaaaccagagacctttgttcacttgtttatctgctgaccttccctccactattgtcccatgaccctgccaaatccccctctgcgagaaacacccaagaatgatcaataaaaaataaataaataaataaataaataaagatggagtgttttagtttcaaaaaaaaaaagactcactggtgactttgacgagaggacaagattttctgaaaagtgaaaatatgtgtgtcaagaaaagtttagaagtttggaaaacttgtatctccttctcaatttggagtcttggaggatgagtagtccccaacccccctcccctggcccccaaaaccaaggcttgctctgctgcccaggctggagtacagttggtgtgatctcggctcactgcaacctccgcctcctgggttcaagcaattctcctgcctcagcctcccgagtagctgggagtacaggcatgtgccaccacacccagctaatttttgtatttttagtagaaacgggttttcaccatgttggtcagactggtctcgaactcctgacctcatgatctgcccacctaggcctcccaaagtgctgggattacaggcgtgagcccttgcgcctgactcaggatgagtgattaagcagcccattgtaaaggtaatagacatgagttctcagtcagcttattccatactacctgtggaatcctaggattctgagaaaattatttggttaagtttttgttttttgtttttttgagacagatttccctcttgtcgcccaggctagagtgcagtggtgcaatcttggctcactgcaacctctgcctgctgggttcaagtgattctcctgcctcagcccccaagtagccaggactacaggtgcccaccaccacgcccagctaattttttgtatttttaatagggacggggtttcaccatgttggccaggctgggcttgaactcctgacctcaggtgatctgcccaccttggcctcccaaagtaccggaattacaggcatgagccaccacgcctggcctgcataagtttttacctgggtttctagggagaagaaagagatcagacagtccttaacaggatgctggcaggggagctcccatcacctttgcaagcctcattgcagtggcctaggtcagtagtcacagcagtatgcaagtcagcccttccaaaccaagtgaggagtcctgggaggtgagaacccttggcaaggcagccctctgctgctgctttattgttcaataggtggtgctcttctgcctaacttagaacaaaatcaggtcccttgcaaggttctggggaaattgtaaccctaaacatgaagagtggccacccttcactttggaagtagttgcgtttcgggattaggcaaatgtaattgtctgtagaatccacagtgcccagtttgaatgccagtcataagtaaagatgatttttttcctcctcatatatatagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtaagtggtgagaggccctttccctcttttcctaagctccacacctcacttttcccctgccaaccctttatccttcctctctcaggtgtgccagtgggtgctgaaatgcaggaatagcaagaactcgctgatccaaatgacaatccttaatttgttgccccgcttggctgcattccgaccttctgccttcacaggtgaggatgtccatggaagacatcattacccatctcagggtaaacagggaacaggtgattccagaaaagccaaatatcttttcctttgaagcaacgccaacaaatctctgatttctttgtctcatgtccaatttcaatgctatttagttcaggttttctaatgtcagaggcagcagacacaggaacaatcagaacagagaggcaattctatggccaagcaaattttcatgtatggatgccaacaagttcacaagtagacttaaaagagaccaaaggaagggcagttttgatgtagctactgtgcagcatctacgtcaaagctttgtgagtcagggagggtttggggatcgtcctgcttctgtccctgaggtggtcaccaagctgcctcaccacttgcaggcagcatcaatccttggctcctgaagctgccccaactcagagtccaacttctcaacgaagccttcctagattgctccagctgtaaatgtccttgccttcttccatcatagccctcagctgtggaaaggtttggcatcagacctgagatgaaccccacctccgcccttaaccatgaggttttggacagattatttgacctacctgggcctcagcttcctgacttgtaaaatcagaataacaatctctgcctcccaagatgagcagcattaatgtctcagcatctactatagtgcctggcacagagccctaataagtggaaaccattatatattattacagcctcctcaagctacttcaaatatgttttggaaatagggtagaggtaaaatttataatgctctagttgcctgttcatacttaatgtccttgtcttacctctctacttgctgagtgtaggcacccagtcttgaacatctcttcccactttaccgtggtgctcattgcctgtgcttagtgaagcattcaggacccattgagagccaggaacataagcaaaaggccagcctggccctcagcagaccctattccctagcacacagactcacttcacgccacccagccagacaaaccacaggagaaatgtagctggtcaggtgttaaacacgctgttcgaaagaagtagaacgtgttgggtgaggacatgacaattcgccacataaaaccactgaaccagagagaaataacagaaggaaaatcacccgtattttgttagtttctattgttctggcctggcacccagcagcttttctcttttttaatgaccagacaggctgctgttcagtagagctttaaatgatcttggattgaaagtctgtggttcaaaaggaactaataactaggccaaatttgatcaaagtctggcacattacccttgtaagttacccttgtaagttcagaaggctgcctagcagcaggatacaaaggctcagagggccctagatttcttcttttgatgtgggtgtttgttattgttgttgctctcttttttaaggttagtcaaggttgttttattaaatatgctaaaatatgccagtgacattcagcctgggcacaccgtataagtatttgcttcattgcataagtactatttgtttaaagcttgcagtttggaagttattgcttaaacttctcaatgttataagctttagaaatataaaaaaatactccaagactgaactagaatctctacagtcagatttctaatctcaggatgctaaatccaggaaagaaaattatactctcttggggttttttttgagacagggtcttgctgtgtcacccaggctgaagtgcagtaatgcgatctcagctcgctgcagccatgacctactgagctcaagcgatcctctcacctcagcctcccaagtaggtaggaccacaggcatgagtctccactcctggctaattttttaaatttattttttgtagagatgatttgggatctcactgtgttgcccaggctggtcttgaactcctgggctcaagcaattctcctgccttggcctcccaaagtgcttgggattacagacatgagctgtggcgccttgccaggaaattatgctcttgattttaccccgtttcctgccgaaatgctatccccttagtttttcttgttgacccactctaattttagggttgtatctccttggtcacctgtcttcaagccccagcatgcctcattgtcatttgaccacaagagccatgggagaaaacatttctcagagtaaatgcagtcatagcatgtgctattttagaagtctaattcattgtctaaatgtggttctggtagctttcagtatccctaacttcctaagggatgtgtggttttcccttctctcctattttatgtgtactactggtgcctggacttgggaccaacatcaatggaagagaagaaataaatagacaagaagcaagaatcaggaaattccagccttcagaggagaaataacactttagcttggattgtatgagaatcgcctacccatgcacactggcttatgtgaacctaatgaaaatatacatgtggctttccaaattatagactaaatcttcttctattaaatataactgtcataaaaatctctaggtaatagcaaaatgatttccaggcctatgctgaatggaatggcctgtttattgcaaacagtgctcagtattacagtcatatattgcttaacgatggggatacattctgagaaatgcgtcttaggagaatttgtccttgtgtgaacatcatagaatgtatttacataaacccagatggtatagcctactctacacctagctctgtggtgtagcctattgctcctgggctacaacttgtacagcatgttactgtaatgaatattctaggcaactgtaaaacagtggtaaatgtttgtgtatctaaacatagacatagaaaagatatgtaaaaatatactgtattataatcttatgggaccaccatcatatacatggtccattgttaacccaaacgttgttaagtggcacacgactgtactgtttttttgtttgtttgtttgtttttctgagacagagtcttgctctgttgcccaggctggagtgcagtggcacgatcttggctcactgcaacctctgcctcccgggttcaagtgattctcctgcgtcagccttccgagtagctgggattacaggcgcactaccatgcctggctaattttttgtatttcagtagagacggggtttcatcatgttgtgcaggctggtctcaaactcctgagctcaggcagtccacccgccttggcctcccaaagtgttgggattacaggcgtgagccaccgcgcccgaccctgtactggttttttattagtttgagatggggtcttttttttttttttttttttaaatgagatgtagttttgctcttgttgcccaggctggagtggaatggcgtgatctcggctcaccgcaacctccgcctcccgggttcaagcgattctcctgcctcagtctcctgagtagctaggattacaggcatgcacccttatgtccggctaattttgtatttttagtagagatggggtttctttatgttggttaggctggtttcaaactcctgacctcaggtgatcctcctgccttggtgtcccaaagtgctgggattacaggcatgagccactgcatctgagacagggtcttattctgttgcccaggctggagtgcatggctcttggctcagtgcggcctcaaactcctggactcaagtgatgttcctcagcctcccaaagtgctgggattacatgtgtgagacactgcacccggcccatgactgtacttcaaatttcattataatgtaaaattgtttagatgatcttctgtttttaaacaaggtttgacatatatttcttcagcaagagcatgtaattcttgtgtgaaaatgtaacagtaatggagacaattctgtgatattgttttggcaaccttttacgttttttgggtttttttttttttttgagacaggctctttgtcactcaggcttgagtgcagtgctgcacacgatcacacctttctgcagcctcaaccatctgggctcaagtgatcctcctgcctcagcctcccgagtagctgggattacaggcatgcaccaccacacctggctaatttttatttttgtagagacagggtctcactgtgttgcccaggctgtttcgaactcctggcctcaaacactcctctctccttggcctcccaaagtgttgggattacaggtgtgagccactgtgcctggccactttcttaatttattcttaaactgaattgctttataggacctttgactgttgatatgtagaccctaaccctgacctggagctatctactttgtcttttctgtgctcagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagtaagcacctctgttatgatttcaccttccactccatttggttggagaggaaggacgctctatttcagatccacggagcttacgcccattttttgcatcattaaactccagggtatagcaaatcatatgaagaacagcccttccttagttgaattctaagcttttccaaatgaaataacatatatgaaatatttggggaggttgaataccctgagaacatgtgagatgccaatgctattttcagtgttattatttactgggaatctgctagttttgcttttttctctggcttttaaaagtcttattttgatataagacctaaagagaggaatccttgactggaactgaagggacctaagggtcatctttttcctcatcgctgaagttgaataagttattccacctctttaaacctcctggcgaataaaatatgagaaaacccctcagcatcctgccagatggagagactggggtgtcttcgctaaggcgagggggtcagtgttcatcatcctttgtctcaggttgtctttatacttccctgtttattagcgaagattgctgtggtgaattaatacgccccactcattctttttttttttttttttttttttttgagatggagtcttactttggttgcccaggccggagtgcagtggcacggtcttgggtcactgcaacctcctcctctcaggttcaagcgattctcctgcctcagcctcctgagtagctgggattacaggcatgtgccactatgccaagctaataattgtatttttttgtagacacaggttttcaccattttggccgggctggtctcgaactcctggcctcaagtgatccacccaccttagcctcccaagtagctgggactacaggcgtgtgccagcacgcccagctaatttttgtatttttagtagaggcggggtttcaccatgttggccaggctggtctcgaactcctgacctcaggtgatccacccacctccgcctcccagagtgctgggattacaggcgtgagtcgctgcgcctggccccattctaaagcccagcattctcatcatagaagaatttctttccatatgatggctgtgtactgccttcttgtccctgatgaacacattgttcagctctcttcctacataattatcttggtgagcaccacaataacccatatggtatagctgtgtgaattcagaaaaaaggccccttccttaagacactttactgccatctgctggattgttgtagaaatgtacaaatctacagtaattgtgatgtgatgggtccctttgaactgtgctctggacaatttgcatacccatctgtggtagccctgcctgagtaacctgctctccttgccccaaggtgattttgaggtgggacattaaagaatgtcattatgtctcaactagccactctccatttctctacaggaggcagaaggcaatgcaggtggatgccacagtcttcacttgcatcagcatgctggctcgagcaatggggccaggcatccagcaggatatcaaggagctgctggagcccatgctggcagtgggactaaggtgggtgtcagaaacctgagcttgccctgtatcagcgtgaggcttggcccccagtgttgggactgttaaaagaatgaggcatggttgtttgcacattcaactggatggacaggatagcttagactaaacacacaggctagtactgaatcctagtttcatcactagctttctatatgatcttgactaagttatttaatttccccgagcctcatttttttttttttttttttttgagaaggggtctcgctctgtcgcccaggctggagtgcagtggcccagtcttggctcactgcaagctccgcctcccgggttcacgccattctcctgcctcagcctcctgagtagctgggactacaggcgcctgccaccacgcctggctaattctttgtatttttagtagagttggggtttcagtgtgttagccaggatggtctcgatctcctgaccttgtgatccgcccacctcggctcccaaagtgctgagattacaggtgtgagccaccgcgcctggcctcattttttttttttaatctgaaaagtgggtgcaatagtaattcttattgcctagggttgctgtgggttcatggtatagtcactcttagcaccatgcctggcatataggaagtactcaataaatatttgcaaatattgtgtatattattattgagcaagttgcttaagcttcctaaactaagttctcttcatctgtaaaataaggacagtgttcttaatctgcttcccagagtgtttgttgagacttggtcttctgtagtggctgaattatagtgagcaagtcttctgtggcaagaaccagaaactcatagattgtttggagacctgagactaagccaagagagtttacacttttattcttattttataatatcctttctactctgaagaacagaggcctgatccccttgctgggagagaaagctgtttcttcccaagaacagactgagtgtcatgtgcccagcccttatgtgacttgtttccttcccctcagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggtaagaaattacaacggctcttgccaagatagtgagagatgctggaaactttgggaaaggaatgaaggtatctaggattcctggatcttgctggggtgctaaagacagggtagctgattctgtagcagctcatccttcctagactctttccacgtattggtaattcctacttttgtttctttgtcccatactgagtttgggggcaaatggtttgagggaaagtatacgttgatccaaggaaataatgcttcagagacagatccccctcacatcctcacttttccacttctttgtagctgagatggtatgtaatgctttaaaatgtataagtaggggcttttgggtgtttccatgtctatggcagtatgatttcttttttctttcttttttttttttttttgagacaaagtctctctgttgcccaggctgatgtgcagtggcacaatctcagctcactgcaacctctgcctcctgggttcaagcgattctcctgccacagcctcccgagtagctgggactataggcgcgtgtcaccacacccggctaatttttgtatttttggtagagatggggtttcaccatgttggccaggttggtctggaactcctgacctcaagtgatctgcctgcctcgacctcccaaagtgctgggattatatccatgagccactgtgcccggctggcggtatgatttctaaaagtaccatactgtgtagttctaaactgcaaataaacccataataataccccacagagatatatttcagggggagaagttgatacttttgaaatttaagatcatataggtagtattggtcaagaatgtggggaaacaagcaagtacagaaagcctgaacatgctgtctggagacagccacccatggtttctctattgcagttcctccacttaacattgattcacacccatgataatttatttctagtgaatagaaagtagctatgtttaaacccagcctacacagactctttgtctggtatttaaatccgggaactaaaagaggcaaggtaaatttgatcaagaattgtattatcctctgagaatgtaaggggttgagttggaataaaacaaaataatttattatttaaagggaatataaaaaggctaggattgggggataccttttatctgtctctttgctccttcaccacccccttttacctttctgctgattgaaatcccccttgttaccctttaaaagttctgggtcaggcctggcgcggtggcttacgcctataatcccagcactttgggaggccgaggcgggcggatcacaaggtcaggagatcgagaccatcctagctaacatggtgaaacgctgtctctaataaaaataccaaaaaaaaaaaaaaaaattagccaggcgtggtggtaggcacctgtagtcccagctactcaggaggctgaggcaggagaatggtgtgaacccgggaggcggagcctgcagtgagtggagattgcgccactgcactccagcctgggcgacagagcaagactccgtctcaaaaaaaaaaaaaagaagagttccgggtcagtatcgtattgatcaatatctagttgaaagtagaattagtagcctcaaaatggaaaattcagctagcaagctgcagacctacctgtctacagcagcagtgtttattctgttttctctctgcctttctatccaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctggtaaggcttgccagacagaaggaatctaggggagttaaggagcttaggacaattgggagtagccagtttcattacatggcctctctagagttcttcggctcaaaaagctgatattcagctatttctctgccaaggcgtgttaggcatggctactgtagcctttggtgtaagataatctacctttaattcttttgtcttttactcttgtgagtagagaaaagaatgacagaggttagatgagaaaaagcatctggaatcttcccactacgctgatggcagtggaggtagaagctgggaagggctggagtgatgggatcatgaggaaatgttcaaaggctccattgctcttgcagaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccaggtaaccgaggtgatggccagacccacaggcagggaagacaggactctcgctgtgagaactgcttgaggaagcggagttgaggacaaggatgggggagaaagattgaggtacctggtaaaagccgcatttgtctgcaaaggtccaaatgtttccctggctttataatggcagttccagtgctcatcacctcatgagccctttgtcaatatgactctaaggtggttcagagtcctctcatgcagctctttctttttcctggcaagtcaccagtgatcccatttcttggccctctttgcttgctgggttgatctgctgcagatggtgtcagacttcccagcgcctccctatccttcagctctcctgcttttcttgctggtgccaggtcaagcacggaggccgttgttcttcacaaaaataaccagtgtgagaccgtttccactatttgttctaatagtcattgctccagtctgtgatcctggctttcgttcttaaataatatgagaatgccaagctcagctgcttggtctgtagcgtaaggtagaattgctctggagacatggctcctcagtttttgtcttcacctactgagtaagatctgtggcctcctggtgagcaatacatggagtcctgccaatacaccagtcctgcctggtgaagttatttccacctgcctcttgtgggtctctatgttgctcacccagtcatttcctgtccccattgctcactgtttgcttcctgatcctccttcatggtttgttttgaagtaaacactttttataacatataaggaacttaaacatagaaaatcattaatatatgtaactcactctagagaaatgtttgttcacaatttgttataaagtggctgtaaattatatcatcagtcaacaaattaggaagttgtacttatgttggcacttctatttttagcgtaatgatttatttaactgccggcctgcgttagaagtgcctgccattttaagtcttaaaaacattcctcagttataggaaggaatagaaatcattgttgaaggccgggcgtggtggctcatgcctgtaatcccagcactttgggaggccgaggtggccagatcacctggggtcaggagttcaagaccagcctggccaacatggtgaaaccccatctctactaaaaataaaaaaaaaatagccaggcatgttggcaggcgtctgtaataccagctactctggaggctggtgcagggagaattgcttgaacccaggaggtgaaggttgcagtgagccgagagcgcgccactgcactccagcctggcgacagagcgagactctgtctcaaaaaaaaaaaaaaaaacgtgaaatcactgttgagcctgccagctcaaatcatgctaaattaatttggcgtagaaatatctcctaaacggctgagtgcagtgtctcatgtctgttatcccagcacattgggaggccaaggcaggcggatcacaggagcccaagagtttgacaagaccagcctggggaacatgacaaaccacgtctctacaaaatatacgaaagttagctgggtgtagtggtgcatacctgtagtcctagctactcaggaggctgaggtgggaggatcacttgagcctggtaagttgtggatgcagtgagccgtgattgtgccactgcactccagcctgggtgacagagtgagaccctatctcaaaaaaaaaaaaaaaaaggaaaaaataagaaatacctcctaatgagaatatcaatatgttcattctgtcagcagactttttgaccacatgctatgttccaggtactgctgggcactgggaatatagcagtcaacaagccaggcgtggctgcaatcttctttcttttttttttttgagacacagtctcgctctgtcgccaggctggagtgcagtagtgtgatcttggctcactgcaacctccacctcccaggttcaagcgattctcctgccttggcctcctgagtagctgggactacaggcgcccgccaccacgcccagctaatttttgtatttttagtagagatggagtttctccatgttggccaggctggtctcaatctcttgacctcatggtctgcccgcttcggcctcccaaagtgctgggattacaggtgtgagccacctcgcccggctggctctaatcttcttaaagctgatattaagctatcaggccatttcattttttcaatattgcagagtgtgaagctcctttgaatgaatttatgtactgaatttagcccttgaaatgttaaaattactcttttttgtttttaaactttgatatagatttttccaaattgaggtgtacattttcatgccttggtaaaggctttggtgttttgttttgttttgagacagaatctcactctgtcaccaaggttagagtgcaatggtgggatctcgtctcactgcaacctctgcctcttgggctcgagcagtccacctgcctcagcctcctaaagtgctgggattacaggcatgagccaccgtgcctggccgactttggcctttcttgcggtcttggtcatcagggtgggcattggttattgttattaggatgcagtgaggctctcgggctctaagtgaacctggggttctttggattggccccgcactgctgcagatagttgggctctgctccgtgaggcacatttccttgcagtcagccatgtcttcttggtgctgcaggtctattctcagtatccccttcatttcttttccctgtggtctgctatgactggggaaaacaggctgccaggggaatccagaaaaatcgggttattaagtcagtaacctaagacaaaatgtaatgttaaattgtagactcctctgggtagctgagttaacctgagctctgtgacatctgcttttgtcactgctcatcgatttccttctggcagctttatccagaaatctgtctatattatgctgtctatgtcaccatgagcaagatatgctgatttgctggtgtcctaaggatgtcagctagaaccaatgtgttatctccagtcagtaaggcttcgggggatcctgccagtgtgatccccacctattgaggtttatctagatatgccccaaacagctacagagtatcagcacttctcttggcatcatagctgttctaactgttctttgattcctttgccattatttgtagtcttcagtaatttgctgtctggacaaagatttttctcccaaggggccctaccttctttcctcatttttatagaaccctactcacagctctccctgttggttttctcagtcttctctggctgtgctacatggttaaaagctctgctttagagagggccatgggacattggagtgctgtgataaaaggaaagggagcaggttttcgaatagtttttcttcctgtcacagctgtatctaaggctgttctgcctgttgcagaaaagtctgttgctcattgagtatctagcagctacttcatctgtcgtggaggtctgggtttttcccccagttccctgccactttttctgttggacacagtctctctctagaatgtcttctgccatagagaacatatgagcctttcttccctacagattttgacagagttggagcacagtgggattggaagaatcaaagagcagagtgcccgcatgctggggcacctggtctccaatgccccccgactcatccgcccctacatggagcctattctgaaggtaacacaaaccgagaggctggtgctttctctgtgcgttgtcatcagctcatatggggagatgggagtgtttcagtgttcactagtacactgaacaaagtaggtgatttctttgctctcacgcttggagaccaaagggccatatcaagcactttcagttaaagaataccaagaatcttatcagttgcaggaaagtgctgtccttacaagaggtaagagttaatgcttcaggcacatcatcgcagatgtttggaaaggagaagtaggtagatagtagcataaaatagctcgcccaaaatgaggaaaaaatgagcttgaaaatctgaaacaaccttcaaatctcaacacattagagtcacgtgaagtcctttataatgtatgttaatctggatgaacaatcactgagactcggtttagaggagacatgagaaatactttcacattatggtgcttcccagctctctcacatgtcagacttagacttcatatggagaaaagtttgagaatttacattccagagaaatcttggattcataaagcctagaaggaatttctttatgactgagatggtctcttgggtgaattttacttatataaaagaaaggtttctagtagagtctaatctgcattcattttctcataacttctttctctaggcattaattttgaaactgaaagatccagaccctgatccaaacccaggtgtgatcaataatgtcctggcaacaataggagaattggcacaggtaaaggggcagctactttttccatagcggagatagggtggaaaagaaactaacctctaaggaagtcaaaatactatttttttttttaaatgaaaggtctcacatatttattgcggaacccagccaacaatgcattcataacagattcagagagaaataatatattctcaataaaacatgtccaactctccagatagtggtgacattttcagcttgatatggtaatatcattgtgaccttcagacagcataaatgtgtgtgccatctcatgtgcaattccttatagaccctgcttggttcttctccagtgtctttttttggagttgtacctggttttattaccagttttcatccgaatccactggggaatgggacgattttacttttgtttcttggccaggaattgcttaattctgaaagtcttgtgagaagaatggcaagaatcagagtcaggcacacaccatgatggtggagaaaggaagagagggcttttttaaaaaaaattttttattttatttatttgtttttgagacagtttcactttgtccaggctggagtgcagtggtgtgatcttggctcactgcaacctccatctcctgggttcaagtgattctcgtgcctcaccctcccaagtagccgggattacaggtgtgtgccaccacacctggctaattcttgtatttttagtagagatggggttttaccatgttggctaggctggtctggagctcctgatgccaggtgatctgccctcctcagcctcccaaagtgctgggattacaggcatgagccaccgtgcccggcctccaaaatactttctaatgccagaatttctaaccctttctgccaccctctttttcattttgcaggttagtggcctggaaatgaggaaatgggttgatgaactttttattatcatcatggacatgctccaggattcctctttgttggccaaaaggcaggtgagtattactgcttcctgattgagcaagacagaccccatgagtccagggactttgcttctctttattcaccttagtgtcctcacccagtgcctagaacagagtgtgacacatagtaggtgctcaaaaatatttgttgaatgagtaaaagttagaaagatttgaaaccagaattacctgttgtcctgacccagagtcagtttcagtaattatcggggctgactagtgccattttgtctatacaaaagaaaatgcaggtcaattgtcagagccgtagctctcagagggaaaaagaaagccttggccactgcctatgaaggaacagaagtttagctcatccttgcccagccctggttctgctttcccaatttgaggctattagatgactagactaaaattaagaacagtcttaatttatttctgggcatggcagctcacacttgtaatcctagcactttgggaggctgaggcaggaagattgcttgagctccagagcgagagcaacctgggcatgatgagactttgtctctactaaaaaaaaaaaatagctgggcatggtggcatgtgcctgtagtcccagctacttggaggctaagatgggaagatcactcgattccaggagattaaggctgcagtgaactgttatcacaccactgcattccagtctccagtgagaccctgtctcaacaacaacaaaaaatcttaatttccttttgaatctcctgttaatcatttgggatatggaagaatgatgtgttttctttgagaaaggtgggaattttctttgtctcttgatccagtgattgttgcagggttgtgtgcttgatcaaccttggttttatgcccttttgggatttctgctcatggttagtgtctttactggaaacctcttttcttcttacagcctctaacacttcattataatcattatacaaagagttcaactatgctttcttttctctcgtactggctcattgaatcgtgttcttgtgcctatccaggtggctctgtggaccctgggacagttggtggccagcactggctatgtagtagagccctacaggaagtaccctactttgcttgaggtgctactgaattttctgaagactgagcagaaccagggtacacgcagagaggtaggggacgtggcaaacttggtctttaaagactttgctgggacgatggcaagagagagatggttgcagcattaacatgctctgacagtgtcagtcagacttcctgtccctctccaattttgttcatgtagctggtcatttggatatgaactggggattatgtaacctgagaggagtatccgtgatacttgttgtaatcatgttgtgatgaatgttttcttttttccctcccctttcccacgcaggccatccgtgtgttagggcttttaggggctttggatccttacaagcacaaagtgaacattggcatgatagaccagtcccgggatgcctctgctgtcagcctgtcagaatccaagtcaagtcaggattcctgtaagttgggggtagccatgaaagacttgccaagttctcactcacagagcccttgctggttgtgtttagaaagtctggagctggattactgagaaattataggatattggctcgtgtagcaacttttgaatgaaaagtcagtagtttcttcttgaatcccggaagggttgctgcttcccattgtggccagagtcctcatgtctgtctagattctttgtcaagggttggcatgccaagtctgtctgctttttgatgccaatgcaagagatacacattatcaccccatcacctctatctatctctcattctttcttagtgcctctttgatggttctcattcccatggtatctacagaagaagaggatagaattgattcttcctgttttaattgatagggaaatcagaggcgaggctcagggccatagggaaaactagcagcaatcagaaatttggggctcttttttccgtgaaatggtctggcccaagagctgcactgcctttttttgtttgtttgtttttgagatggagagtcttactctgttgcccaggctggaatacagcactgcaacctctgcctcccgagttcaagcaattcttctgtctcagcctcccaggtaactgggactacaggtgcacaccagcacacctggctaatttttgcatttttagtagggaggggttttgccatgttggcctggctgttctcaaactcctgacctcaagtgatctgcctgcttcagcttcccaaaatgctgggattacaggcatgagccactgtgcctggacgccttttttttttttttaaaggtttgttttatttttaattgactcagggagggggaatagggagaggttggtgaatgagtgcaaggttccagttagacaggtggaataatttctggtggtctgttgcccattgccttttattgtttttattttaacttaattttatttttttggagacagggtctcactttgtcacccaggctgaagtacagtggcatgatgtagcctcgacctcctgggctcaagtgatcctcctgcttcagcccctcaagtagctgggactacaggcgtgtgccaccacacctcgatactttttgtatagcccgttgccttttgacaagtagcctgagtgaatgggggtggggtatggggttgcaggagggttttctagacttaacccatattccctcagggcaggcctatttctgacattgaaagttaacattgattctcccaactattccaggaaaatattattaccccaaaataaattgtttctgaactttctagatgggatcatattgcttcacatttcttcctcttctggtattagagaggcatgcctgtgatgtcattctgtcagccccatagctgttgagagcagggcctgagccatgctgctcctggtgttcttccttattcatggcaccagtcagcccagctcttcatctgtttagttcttaccctcctcgctgcactcttcatgttagtatacctccctcccttgccactgcccccgctgccaccgtcagagtccttgaggtccttactgttcaagtcaggtaggtagtatttgcctaccaacttgtctgaattccctaggggaaccaaggagctgctgaacagatgtttgcctaatttaattccctttactttattttgggggagtaattatttttattgatggtttcagttatgaagttgtgaacggtaagaagctaaattaaacagagcaatttagagaaaaccaacccatcatgtactgttttaggcttttgtggtttgagtaatagaaaagccaaaaaccgtttactccttgatggtgagaggctattcattggttcctaatagaccttgaactcccaacagtgttagaacatactaggaagaaagatatgaatgctaatgtctgttaggaggttgtttgttactaaccatgctctgcctgaagtcacgtcatgccatgtgtagctctaacactctctgttgttttgtcctagctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaagtaagtacctcaaaccccaatccttctctttctcctctgcaccacacatgcacagatccatctgtgcttcagcttcttaattgtgcattctctgtgcccctgagcccaatgccttcctcccccttaactcccaacatataaagtgtacaatcagctcactccagtgtcctggctaaaagggccaggtgcagtggctcacacctataatcctagcactttgggaggccgaagtgggcagatcacctgaggtcaggagtttgagaccagcctggccaacatggtgaaaccccgtctctactaaaaatacaaaaattagccatgcgtgatggtgcacgcctataatcccagctactcgggaggctaaggcaggagaatcccttgaacctgggaggtagaggttgcagtgagctgagatcgagccaccgtgcttcagcttggacgacagagtgagactctgtctcaagaaaaagatggctaaaagatgaatagcctgggggaggaaaagcattgtcattctcagtaatctagagttcagtccggttgtagatgccccacaaatagaaaatcagtctggatttctggttgctccttcatgacgagatttaagtcgtaacatttttagtgaaaatactacacaggtgaagttgaatgcttcttactggatctcatactgccaccagttggtcccattactgttgatcctcagtgcgagcatgcggccgagtaggtgtttatccgatctctccattgtaagcttatctgttttcctttgccattagtgagtaatctgtggggtgacactttgagactccatggctatcctgttcctcagctaccttccatccggtgattttagcatccgttgataatcctcacttgagttgattatcacactggtggttgcaaagtgatgactttctaattttgctattccttctccattttattagctggcagcatttttctgtgaagaggaattttcccctcccccacctctcactcttgctgtttcttgtgtctttctttaaaccacagaatatgcaaaatggttctaagattatatgtccctactactaccaaccacagatctaagtaaagttcagagtagctttgtagttcctgttattcttagaatacactctactaagggtgtacagtaagagtactatgtttaaaagatacctcaattagttacttttccctctgatcatattgtcaacatgatgtacatttagaggagatagcatgtttttagaaattatgtttcctaactttgtgtctggagtatagacatactgtttatttgtttctattgtatttataactatcaacctaattgaatttttttttttttttttttttttttttttgagacaaagtctcactttgttgcccaggctggagtgcagtggtatgatctcagttcattgcagcctctgcctctcaggttcaagcaattctcctgcctcagcctcttgagtagctgggattacaggggtgcaccaccaagcccggctaatttttgtatttttagtagagacagggtttcgccatgtttgccaggctggtcttgagctcctaacctcaggtgatccaactgcctcagcctcccaaagtgctgggattacaggcatgagccaccacgcctggccggaattcttattaactctaataatttacctgtaaattctctcaggtttttactctatataattatatagtctggtgggcgggggcgggggcgggggtggggggaagaaaaattaaaaaaggaagttttgttttttagttttaatcttgtaaaccaccacttagtgacacagtaaggaaaagtaataagtactaaaaaagtcataagacctctactaccatgtttttggtttttgagatagggtctagctctgttgcccaagctggtgacacagaggttgcaataggtgatatgggctcactgcaacctccacctcctgggctcaaataatcctcccacctcagcctcccgagtagctgggactacaggcatttacaaccacacccagctgattttttgggtttcgccatgttgcccaggctggtatcaaactcctgggctcaagctatctgcccaccttggcctcccaaagcactggaattacaggagtgagccactgtacccggcctacttccatgttaattagatgcagtgatagtggaaatctataaccttgctcctgattttaatgggactgctgttaagatttcaccatgaagtataatatttgttgtagctttttggtagatatctttttattttatttttatttttaaaaaaatacggaatgcttcacaaatttgcctgttatccttgcatatgggccatgctaatcttctctagattgttccaatgctagtatacgtgctgccaaaatgagcactagatagctttataaaggtcaaatattctcatataaagagaatactaaagagttttattatgaatgtatattgaattctttagtctgtaatagtcatatggtttttctcttttaatgtgttaaatatgttgaatcacactgataacgtgttctaatgtaagaatgctggcattcttatattaaaaccaccttggttataatgtatttttaaatttcgatcttatgtcagttcagcttagaatttttggatctgtgttcttgattgaattggtctataattttctctttcttaactatgggctcaaagatctgtttagcttttctgtatctccttgaagcagttttggctacttttttttaggaagctgttcattttgtctaagtgataaaacttgttgacatgaaattgttcacactgttctcttacattttaatttttttaagatctttttttatgccccagaactgagtggattatgttttaattttctactagagatttgttaaagcatttttcaaagaagcaactatcggccaggtgcggtggctcatgcctataatcccagcactttgggaggccaaggcgggtggatcatctgaggttcggagttcaactccagcctggccaacatggagaaaccccatctctactaaaaatacaaaaattagccgggcatggtggcacatgcttgtaatcccagctactcaagaggctgaggtgggagaatcgcttgaatctgggaggtggaggttgcagtgagctgagattgtgccattgcactccagcctggatggcagagcaagattctgtctcaaaaaaaaaaaaaaaaagaaccaatttttgacttgttgatctattattattttactttttctgctttttaaaaaaaattgtgttagaatgttaagtcattgtcagtcttcttttctaatgtaatctcttaaggctatgaatttcactccaagtattaactgctttccaaaatttcttaaagcttacataccgtaagatatgtggggggaaaatttaaaataaaaaatttatttatttatttacaagtcagataaaatttacataccgtaaaattcattgatttttaatatatttacagagttatgcagttatcactaataatctaattttagaaaacttaaatcaccccagcaaaaaaacctcctgtccattttcagtcattttctgtttctgcctcatccctgggcaactactaatctgcttctctctctatagatttactttttctgggcatttcatgtaattggtgtcatagaatatgtggtcttttgcatctggcttctttcacttaaaacttttgaggttcatccatgtgtggcatgtatcagtaattcattcttcctttattactcagtagtgatgtagtattttcattattgtccagaaatattttccagtgttagaatttcttcttttacccttgagttatttagaagtgggtattttatttgtaaacttaaagggtttttaatttctcttttttatattgatttctaacttaattgcattgtgtttatgattctttcatgcttgctggaactgctttaatggtctagtggttgattgattttcataaatatttcctatatccttgggaatacttgtgtatttgctaacgttatggacagggttttacataagtcattagattgaatttattacttattgtgttcaaatcttttgtgtccttctaatttttgtttgtttgatctatcaatactgagagaagtgagacaaaaatctccctttacattggtgggcttttttctgatagtttagtcaatcttgttttatgtatctttgagattatattactagatgcatgcaaattttaaatcctgatatcttcctggtaagttgactcttttatcattatgcaataatccttcttatctttaataataccttaaaacgtattttacgtaacattaatatagctgtaccagcttttttttggaggtatatgtcttttccaattcttttacttttaactgtcttgtgactttttgttttggatgctgtcttgtaaatatagctagattatctttttaatccaatttaaaaatgtttgtcttttaaccagtgcattctaggccaattgcatttattgtgattgctgttatatttggcctatttattttattttattttattttattttattttattttattttattttgagacggagtatcgctcttgttgccagggtggagtgcaatggtgtgatctcggctcactgcaacctctgcctcttgggttcaagtgattctcctgcctcaccctcccaggtagctgggattataggtgcatgctaccatgcccagctaattttgtatttttagtagagataggatttcaccatattggccaggctggtctcaaactcctgaccacaagtgatccacccgtctcggcctcccaaagtgctgggattacaggtgtgaaccactgcacctggccatgtttgcgtttatttccaccatcttacttagtgcattctttttgtcctgctttgtctgtttatctttcttttttgtctttttctggatttgtttgagcctttattttttatttcatcttttgccttcactcattggccacctttaatttttcttaacattttgtaatagactatttcaatcatgaatatagtagacaacctattatattagattctcacatacccatcagtaattactagcattttgtcaatttggttttatctcttacctccatcgcctttccccccgacacatgtacatacatttctgcagtattttaaagcaaattacaggtatcatattatttcaccaatacatactttttcccatgtaaccacaaatatgacacctacaaagctaacaatcatttcttagtattatttaataccaacccttcttcagatgtccctgattgtcttaaaaattcattgtaatttggtttattcaaataaaaatctatataaggtctacatctggcatttggttgatgtatataattctttcactacttatttaccttttccctctacttaaaaaaaaaaaaaaagtcatgtatttgtagaagaaaccaggtcatttgtcctgtttctcatactggatttggctgattgtatcttcgaagtgtcttaatttcctataaactagttagatttagaggcttgattagattcaggttctgttcttttgacaagagatacttcatagattacagtactgtatacctcctatttcaacatttagtaaacagatattttccatatgacccactgctagtgatgtaaaggctgatcagtgggttcggatattgtcatctgatccatctgttataaagttccctatcagcctttttcccagtcattttagcattcattggtagttatttcctagagctgttatttgattagggactgcaaaatggtgattttcttacttatatatatattttttttattaaaaaaaatgtttttttccatgacatatctctcaggagctcctgagaacatgtgccccaaaatggtgattttctaatttgattattccttctggatttattagctagaaagttctttagaaataaagaacttggctgggcatggtggctgtaatatcagcactttgggaggctgagttgagaggattgcttgagcccaggaggagtgtaagaccaacctgggcagcatagcaaaaccccatctcattttatttaaaacatttttttaggctgggcgcagtggcccacgcctgtaatcccagcactttgggaggccgaggcgggtggatcacaaggtcaggagatcaagaccatcctggctaacacgatgaaaccctgtctctactaaaaatacaaaaaaattagccaggcatggtggcaggcacctgtagtcccagctacgtgggaggctgagacaggagaatggcatgaacccgggaggtagagcttgcagtgagccgagattgggccactgcactccagcctgggtgacagagcgagactccgtctcaaaaaaaaaaaaaattttttttaaataaattttaaaaaagaactttatataatcaactactgaggtaatttgaagtgtagatcatgtaggaaaagcagaatacatgcatgaatctttccctttatttatcaatttatcaattttttagtataatgagtcaagcctctaacagcttttcagaggtgagccaggcaccttcccacctcccaatcctaccctcattctttggagtatcagtatgaactcatgaagatacatatatatatataatttatctttatttatactgtaaaccaagttggttgcacagtcactcaaagaagactgtcaagtgaaagtcatttagtcatcttattgttaatactaatattggtcatttactttactacttgtgtgtttaagaaagatacatatatatatgtaatctcactaaaagaaacagagctctctatatagagtatttctatatatatagaatatatatatgtatatatacactatatatatatgtatatataatatttctgttcattgcaggcaccaatctttttcacaatcagattgtcctgatcaccctggctgttgtgtcctttttacatggcccggtagactttcttgctttctggcggaaaaaaattactccaagcgtatcaagtatatttcactcaaacctgggcatagccatttccaaggagctctgatttcttttagtgagattacatacatatatatatattttatagatgtaaatagaaaatgaccaatgttagtattaacaataagatgactaaatgtagtttaagactttcagttgatattcttcttgagtgattgtgcagccaacttggcttatagttatactcatttgtttctgtttattttcaatttttagaaatttccttgagtttacaaaaaatttaggctgagcgtggtggctcacatctgtaaccccaacactttgggaggccgaggcaggtggatcactcaaagtcaggagtttgagaccagcctggccaacatgacgaaaccccgtctctactaaaaatacaaagattagccgggcatggtggtgcatgcctgtagtctcagctactcaggagactgaggcaggagaatttcttgaacccgggagacagaggttgcagtgagccaagattggaccactgcacttcagcctgggcgacagaggaagactccatctccaaaaaaaaaaaaattaacttttgttttagttatataagacattaacatagttccagagctagaactataaataaaatcttatctagcaacgtctagcttctgtcaccatctttatacctcgttttcttccttcctttattatttcctattgctgctataacagattaccacaaatttagtggcttaacacagatttcttatcttacagctctctaggttatatttctgacatgaggtctcactgggctaaaatcaagatgttgacaggactgtgttcccatctggaagctctaggggagaattgttttccttgctttttctagcttctagagcacgaggagttggacaagcttgttttagagggtgcctgcgttcctaggcccccttcctccatcttattaaacattattgtcgttttatgtcgtgtttgtttatattcatccacacatatttcatgtctcatcaccctgtcttccatattagagctttctcctagaatcatattctttcttcataggacaaattcctcttaggagcattcattgaaataaactgctcttatctgaataagtctttatttcaccctctttttaaatcttcatttggagatttaaaaattaaattaaaaaatttatttttttagccaagaatattattctaggttctcctacaactgtagatacgatcccactttctgacttctttttgttgccgttgagaaatcgtgaatcccttttacatattttttctgtggctttacttctcattgttttgttatcctgaagtgtttactattatatatctagatgtgaatttcttttgattatcctgtttgagacttggagggcttcctgaatgtgaggattggtatcttatcagttcctgagaaattctcatctattatcttttttggaattttgtccttatacccttctattttttttttctttcttttcttttcttttcttttttttttttttgagatcaagtctcactctgctgccaggccggaatgcagtggtacgatctcggttaactgcaacctccaccttccatgttcgagtgatcctcatgcctcagcctcctgagtagctggaactacaggcgtgcaccaccatgctggctaattttttttgttttttgcattttcaatagagacagggttttaccatgttgcccaggctggtcttgaactcctgtcctcaagcaatccacccgcctcagccttccaaagtgctggaattataggcatgagccaccgtgcccagccttattcccactttttttaaggcattttaaacagactaaattttctgtcgaatacttctattatatataaaactcttagtgggtctgattgtgtatattgtttctgagaagtcatgcttgtagtggtctgtttcctcacgtgttttataatttcgaatcatatagcatattcaactgggctttccctggagcaatactagaaaatctgctctaaatttgcatctttccagagaggatgtgcatttacttctgccaggtggtccatgagaactttaatttcttggcttagagattcccaggccacacagttagtgaaaataaatccctaaacctgtacaatagatgcctgaaattatgaactcttaggggaggcatttttctgcctagaccccacatcacaactggcaaatttctttatctccttttaccagtggacagattttttttttttaactcaccatttctcttaatatgtagctctttgaaagtctggctttatgtgagaggctttagttctttttttttttttgaaacagggtctcactttgttgcccaggctggagcgcagtgacatgatctcagctcactgtaacctcacctctgaggctgaagtgatcctcccacctcaaccttccaagtaactgggactacagacacatgccactatgcccagctaatatatttttttgtacttctggtagagacaaggttttgccatgttgctcaggctggtcttaaacttctgagctcggggtgatctgcctaccttggcctcccaaagtgctgggattacaggcgtgagccactgcgcctggccacttcagttctaataatcttataatcatgtttatatttttgtacttaattttaggcttttcatagtgggggagtttggaaggagatctagtgcatcatattttcaggagcaaaactgtgttcttttaaaatactcatgttccgagcaaaaagagaacacatgtgaaaggctaaggaagaagaggcagctcctctagctctctactcagcattgaggtgcttgcctgtccatgtcccttgggcagctactccttatgaataagccacggaaatgctccctaatcatgcctaataaaatggcaccaaggaccaggaagcttgtttcctctttctgtctctcagttttctctgacctccccgccatcgctgtaggcctgttttcctccagcttaggttttctactttctttcaggctttgcataataaaaatttgcctgaagaacctttatatttagtctcattttccttgatctttgtaataacatttttttttttttgagatggagtctcgccctgtcgcccaggttggagtacaatggtgtgaccttagctcactgcaacctccgccttccggtttcaagcaattctcctgcctcagcctcccgagtagctgggattacaggcacatgccaccatgcccagctaaattttgtatttttagtagagatggggtttcaccatgttggccaggctgatctcaaactcttgaccttgtgatctgcccgcctcagcctcccaaagtgctgggattacaggcgtgaaccaccgcgcccggcctgtaataacatatttgtatagggaccactatacatatttgtatggggaatcactacaaaactgtagtgattcaatttggggaatggtggggaccaggagataggtagaaatgagcctactgagtcaaatgtgtgccacatggggaccaggctgtaatcaccttagaagtcaaagttatcattagggaatatgttcaaactcttctttaagcccaatcctgtatgtcctatcgatcttgcctctttgggtaatgagtactataaatccattactcactgtttgagatagcacttttttttttaagtaatgctttttatttattccaacttacccgacttgcatgtggataggagtgaaaatacatatgttcattttctaaattatccttgttaatggagagaaacaaaggcagagagagctgtaaaaatttggggaagggggccaggcgtggtggctcacgactgtaatcgcagcactttgggagggcaaggcgggcggatcatgaagtcaagagagcgagaccatcctggctaacacggtgaaaccctgtctctactaaaaaaatacaaaaaaaaaaaaaaattagccgggcatggtggcgggcgcctgtagtcccagctactcgggaggcagaggcaggagaatggcttgaacccgggaggcttgaacccgggagcttgcagtgagccgagatcatgccactgtactccagcctgggtgacagagtgagactctgtctcaaaaaaaaaaaaattgggggaaggggtgtcaaatattatttgctacttattactggcgtgtaatactatctgaagctcataacaaaataaaacctacatcttgaagacaaatctttaacctggaaagtagttgagattttctttacccacaccttccttattcccagctttatttagtgcatgttttttcttcctacacctaaaaggtactgacttcagtagaatctcctcatcctatccctaccccagtttgcttgcttattttggctcataatctctcagagacatagttaattaattaagacctaagtaatgagttaattactataaatgagctacaatacccataatcaagagttagctgtatttattttaggaaggaagtggtaattaattagagccaaataaacaaaaggtacatttacagttggtaaaaactttcaggtttatttttaagtttagtaattccaggaatccttcaatgatacagacattcattgagcactgaatagtattcacgtccctgttctagtgcttgacatgaattatcccatttaagccttagaagtctatgaggtaagtattattatctccattttcccatggaagagcctgggcacagggaggttaaatgagggttacaaagcccctgctgtgcagtaactggctcgccttgtgtgtaaaaacctctgacaattgttttgagtccccaccactatcaccatttaagtttgtgtcttttttttttttttttttaagagacagggtctcaccctgtcaccctgcactctgtagagtgcagtggcacgtgcatagctcactacagccttgaacttctgggctcaagcatttctcccaccttagcatccagtagctagaactacaggcaagtgcccagctaattttttgtattttttttgtagagacaggatctcgccatgttgcccaggctgttcttgaaatcttggcctcaagcagtcctcccgccttggcctcccaaagcactgagcctccaaacctagccaagcttgggtctttgaaatatattctcagtagatagtgtagtgtggaaacccattccatagttgcctttatttgttcacatgagcttaaggtaagcctgggggttcaatgtccttcatgataccagctggttgacctcttgtccatttcagtttttgttccagcagctgggaatgttggtgtcctttgtgaagagccacatcagaccttatatggatgaaatagtcaccctcatgagagtgagtagaagttaatgctttggcctcttccatgtttgggtcaaggaaggctcagaagcaagtttgaatgacatagactttttttcacggatctttgtagagctgttacagtcttagtacaggaagtggaacaaagcccactggattttgagggggaggaagggctgttgctccaggttcccaggtacaaaactacaaggcatgaaggctgaagagaaatcctgcagtaattgttctgccagaaatagacaattgggttatttgcctcacacacaacaaagcaattttactttaatatcacaagggtgcttttctattttcatgaaagccctctttgttactgctcataaaccatgaagggattgggttttcttagggctgttaaatatgatggacacgtgttgggcaccaaggaacaaggtgatgctttaccctgctacctccaaaaccatacatatcaccttttgactgcagtggcacatgagaaaaggctcaggttccaggattttataacatgcattaagtaggtgccagaagacaccccatgggcaatatcatttttccttgtgttctatataatttaaatgtctctacggggaagattgcttcacagggatggcatgtcctcaaagagttggaagatttctgttctttcacaccagacctttgtagttagtttggactaaaatcaaaatggctggactatttgattttagcaggactaaaatcaaaatggctggtttcaaatggaacttcccagcacctggatctcagaactgaaaacctccctacatgtataaataattaaaagctgctctattccctgactcttaccacagaagcctctgtacaaggaggatttaggcagaatgctaaagaggccgatggctagtcttagtgggaggaggggcaagggaagaagcagtgtcctcatgggaggaggatgaagtaatctgtgacttaatccccccaagaaggctaaacaaggatacaatatgaccatattatagtcataacaggttgatgaccaaaacacagggaattgaggcagggagcctcacacctgcaaaatacagctttcatttcatcccagatagagaaggtgcaacgggggctcttcctttgactgttgattctgggcattttaatgagctcccccgagcctgggaagaaaatagtctttgtgaacagtgatccagctgacatcccttggcaggctgcctgacatctttgaggtagagaagcccctgtgaggagctgatgataatgcagctgggaggtaggttggccttctctaaagaaggcagagtagggttcaagtggatgaacatcagacagactctggaggctgtgaaatctgtggtgtttctttggcaacacttttaacctagaaaacacagctttttcagagagaatacttctctccaagtagagagaaaggaagttttaccagcatcttctagtatagtttttgctttctaaaccattatgcttttaaagatacctttttaaataaaaatactatatctttttataaagcctttcatctgaggagttatccttgttgttatggacttctactttgaactatttagaggctccaagaatattaacattcatgtgttgccatcattagtagggtttctattatcattaaatttcctactccagaacttttaaagatggtggtaggcagctgtctttttgagttgatagaaatgatatcttttcctggagttagaggcttggactaatttgctttgcagcactgaggcaggatctgtggccagttggccaagtttttattttagtaacttgtgtggaaaataaccatgtgggtgtagaaaggggctcattttgtggcactgttcaggcctgatggatagatttttgggaagggacagatatcagatgctatctattaatagcaaaatctaaatccagaaaagccaggaaatatttgtaatactggagtcaccctctggtagagatcaggtgataccaaagtaaggtccaagtccaccctggaccacttcagagttttttgagtgacttagtcatctgttcattctagaatattccagatgctctgattgatagattctggggatacagagataggatacttgtccctaagtagcctacagtctagagggaggacagcaaagcaaaggaagtcttacaactgctatcgctatcaaagggagtgcaaaggacccaagtggatcatggaaggagaagaagaaagcctgcatgggagaagtgaggctagagccaggtctagaaagacagacagggcttagctgagcaagaagtgggattctaggcagaggaagcagcatgagcaaagcccagaggactgaggtagcctggtgcatctggggaaatgcaattgagtttgtccagtgtagctgcaacataggacacaagaagggcccagtgggaggaggggagtgaggagttgatctcggcgcagaccatgctcactacagttttgcttttctggccatcttgattcctttgttcatatatcatcagaaagggacctgactcagctcctctgacttttctctctttgtaggaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaaggtgagtagcctacgtcatcctccagagagattttctgatttcctctgagtccctgggtgatcagctaaaagctgagacctcattctgagtgacaggttgatgcccattccataagacagaatcccaagaatactaatacccaatgtgtgcagtttacagaatgtctgtaatcctctcttgattatccttatgttttgtatctgtttcaatggattaatcttgggaaatattttatcccagactaattttctttattttccagcaacggattccttatcaactcaaataagcacagagaaagcaaagtaatatgtaagcaaataaaatgaggggaagaaagtgctatcaaaaggatatagttcaaggccatttaataaagagttttcccagtccccagagaactttgaattgtctacaccaccacccgctgcgtgtccttagatacttgggacctgactattcatcaccatgcttttgctcccttttgcagttactggctgcaatccagctgtttggcgccaacctggatgactacctgcatttactgctgcctcctattgttaagttgtttgatgcccctgaagctccactgccatctcgaaagtgagcacctcctctttgggactagcaatctttaagctttaatgttttgttgaaaatgttcatttcttaagttcccaggccatctttctattttttttgttatttcaactcacattattttagtctagaaatatcccaggtttatggaaatcttaactgtccctatcccattggaataaatgttggatccatgtcatccaaaataccttagaagccgttcattcccatccctttggtgctgattgttaagctcaccctcagtcacgtctcttccttggagattgtatgttagcagttactgtatgttagtgattgttgtagggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaaggtaagatgggagtctggacagacaggtgctcttctgccttatttggaaagaagactttgtctgtgagccactcagactttgtctgatgccagccaccatcagagaacgtggaattacttgctttttttcttttcatgtgttcagcaaacatttattgagcaacctatgtgagctaagtgccgttctgggggtaacacggtaaacagaataaagtccttgctctcatagagcttccatcctgatgaagcaaagcaaataaacagtttgtttcttaagagtgatgggttgaagtggggagaagtgctctttatttagggtagacagagaagacctctgatcagttgacactcaagcagagacctagataaaataaaggcctggttgcaaagaattggatgcaaggagaagaaatagcaagtgctaaggttgaggtggcagcttgctcaaagagttcagggcacagcaggaaagcagtgtgacttcagaggagcaagcaatggggaagtgcttgtagttaaggtgatggagataaggcagaatagtagagactagattttgtaggcctggagctctcaaaatatcattcctggacaagcagtatcagtgtctacatacctgagaacttgttagaaatgcagattcttgagctaggcacagtgactcacacccgtaattccagcaacttgggaggctgaggcaagaggattgcttgagatcaggagttggagaccaactccagacatagcaagaccctgtctccacaaaaaaatttaaaacttagccaggcgtaggaacacgtgcctgtggtcccagctactcaggaggctaaggcagagggatcgcttgagcccaggagttccagactgcagtgacccccataattgtgccattgcactccagcctgggcaacagagcaagacctcatctcacctaagacctactgaatcagatcctctagctgtggagcccagcagtctctggtttcatgagctgtccaggtgattctgacgcccactaaaatatgcaaatcactattgtgggccattgtaagtactttggcttttactctgagcaagactggaagtttttggtggactttgaacacaggaaaaataagatctgacttaatatttttaaatgataactcaatgccatgtgggaaaataggctgcgggggcaagggcagaagcaaagatctgttaagaggccaccatcataaaccagatgatggggacttgaactaggattataacgggagatgggaagaaggattcttttgaatatcacagagctagccacaaacccagaagaaatggtcataatgtagttgtataatattttttcctctactttttctccactctccaaaatgatagtttctcatctcttccccctcctgttttagtaccaaattttcattccaatggtgaataaagttctggtgcgacaccgaatcaatcatcagcgctatgatgtgctcatctgcagaattgtcaaggtgaactatacttcttttctgttctgaagtccctgttgtctttactacttgatattcttgtaatcttggctatgggagtttgtgtagctttttggctgtaaattagggattggcaaacttttttccagttttgtagactatgtggtctcttttgcagctcctcagctctgccactacagcctgagagcagccatacacaatgtgtaaatgatgagcctggctgcattccactaaaactttatttatggacacctaaatttgattctctcaattttcatgtgttgcaaaatatcaatcttcttttgattttttttccccccaaccatttaaaagtataaaaacccttcttagctcttgggctatataaaaacaggcagagggctgggtgcggtggctcccgccttgggaggccaaggcaggtggattgcttgagcccaggagtttgagaccagcctgggcaacatggtaaaaccccatctgtacaaaaaatacaaaaaattagctggtcatgatggcgggtacctgtggtcccagttaccgaggaggctgaagtgggaggatcacctgaggctgcagtgagctgtgattgcaccagtgcactccagcctgggtggcagagtgagaccctatctcaaagtagattaaaggctaggcacaggggctcatgcctgtaatcccacaactttgagaggctgaggcgggaggatcacttgaggtcaggagttcaagaccagcctggccaacatggcgaaacctcgtctccattaaaaatacaaaaattagccgggcatggtggtgtgcacctgtagtcccaactattcaggaggctcaggcaagagaattgcttgaacccaggaggcagaggttgcagtgagctgagattgcgctactgcactccagcctgagagacagagctagactctgtctcaaaaaaataagtaaataaataggttcgattagataggcaggtagggagtggcacaaataaataagtgaatagatgattctttttaaaaagttttaaaaagccaggggatgggggctgtttgccacctttgctccaaacactttatcacttttcccattgccgacaattgtttatctttgctagttgcatggttgcttgatcttaatactaagtcagtgcttgtcagtaactaggagtgactagcaaagactgtctacagttggggagtcagcccatctgtggttcattttgtccatatgtgcaactttttacttcttactgaaatcctggctctaccaggtctgcacctcccaaaatttaaggatatttcctaaaccagaagcatctagtagagttataggtgtttcctataaatctttggttgtagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcaagtccattgtttcaggtgactgggaaaggagagctctgctcttctcacttactcaaaacttttaactgtttaaactgggcaaccgaaaaccagcccaggtgttttttccagaaatctagataccatgaacttagatcattttgtctttttacaggtgcctaaattgtaattttgttttgaatagagaaaaagtagagaacaaaatgtccttttgtgctcccttggaatatggatggtagaaccttttgttgcaaatacattctcattttttacatgaagggaacaaagctagtgagccccctagttggcattgagactgatgaaggctctttcaaacagtgcttttcaaacttctcaaactcattgggcttaccagttgctgggagtcttgtaaaaatgcttattctgttgttggcagtctgatttggggcctgatattctgcatttctgacaagctgccagctaatgtgatgctgctggtccatggaccatactttgagtagtgaggttttagatgtttccaagtgattactgaattatctctgctggccaaagcctaagatatgaccagcttttcaccatgtatgccatcagctatccacctaaaaacaggcagtttcaaagcatctctagtgaatcagagcacaataatcataattacttcaccaactttaaataaatataccacatcgagtattttccattttgcattagtctttcatgtttctggtgactgtaagcaacttccagtttagatttcagtgttatttttcatctattagccatctcactccatgtgcagcagcagatagaggtgaagaactgggcaagactgtgtcctagctgactcgttcacttcttttggaaaaacccataaagatttgaacacagtcctttgtgcatgtgtacgggctcacgcccagctttgcggagcactgggggtttattactgttgtttcagagtctttactcacacaaacagcttcaagtgaccactgctaagaaacgtttgctgggttaagatggaggtagtaaaatattcctgggtcaggagtgatgctgctttccctctggggattttgtaactgctactctgagactttaggaggaagaaaaaggttatcagtcacttggcattcccaggtaatcattgaggccaccctgctgacacctcacactggagggaggggccaggcaggaggccagatgtacatttccacccagatgttttcatcctgactaggttcagaatgtccttccaaccaggcagccgtccaccctgctttagagctttgttctctctattttaggaaattcctcagtaatctagtttgcttccaggaagttttgctttctacccagacttggaaaaaataagcataaaccttgccagatagacaaaagaaagactcttagtttatctcacctgagaaagtagatcaagttcaaaactctagacagaatcctccacagctcactctcagatctatttcattgggaagtaaattgtgggagctgggcatggtggctcatgcctgtaatctcagtaatttggcaggccaaaatggaagaatcacttgaagcaggagtttgagaccagcctgggcaacatagtgaaacaccatctgtataataaaaatgaaaaatgaaaaaaattaatttcagcctgggcaacagagtaagaccctgtctcaaaaaaaaaaaaaaaaaaggaattgaaaaaattttaaaaattagctgagcagagtggcacctgcctgtagtcccagctactcaggagactgaggcaggaggttcacttgagcccatgagttccagggtgcagtgagctatgattgcgccactgcactccagcccaggtgacagagcaagattctcttcaaaaaaaaaaaaaaaaaagaggtagggggccaggtgcagtggctcatgcctgtaattccaacactttgggaagccaggcgggaggattactcgagcccaggagttcaagaccagctggggcaacatggcaaaattccccctctacaaaaaatacaaaaattagctggacatggtggtgcatgcctgtagtcccagctactcgagaggctaaggtgggaggatctcttgagcctgggaggtcaaggctgcagtaagcagtgatcatcccacttcaccactccagcacaggcaacagagcaagaccctgtctcaccaacaacaacgacaaaaaaaggcagcagacctcattgttcagaagtaaattgtgcctccttataatcatgtgatgataattagggattggtgcatttatcagtcttaaccaactttcagagtaacaatttgccacctcagtcaccattttatttgttatgataatattagatttatgtgatacagaaaacataaaaaccatttatgtatacttctagaatgccttccagaattttcagcagctttactgtctgtctgaatgataatctgtttgggtttaccttcaccatgagtaagctggcattccatgaacaggcaccagcatcaccagatgaggcgatcagcagtgggtggatggcaagaggaagcagctcgtgtttacacagttgttatagaacccagagaattgagagttgataatgttttacagagcagggggaaagggccacatgggggacttggcctagtcaataaattaagctatgattttttgaaaacctatttactgccatttatcaagtaatagtaacaactgacatttattgagtgctttccgtgtgtcaagcactgtggtggaacactatattaactcatttaatcttcatgataaccccatgagttaggtgctgtcgttgctgtcccactttacagatgtggaccgtgaggcactgacgggttaagtaatttgcccagtgtcaactagcatcaatggtggagctgggatttgacctagatagcatgactccagagcccactctcttaaccactgtgttgaattgtctctgccttacccaggcccaacctcatttcattagagggaaaaagaaactagtaatacttccctgtttatgtgcagcttttaacaattgctgtataatataatacagctacacacaaacccatcttgtagggaagaaaccaggtatctgtgggcaggaaaggactggatgttcccattgtgctcaacagagcaaggtctaaaaatcagtctttggtatctagtctccctcttccttctcattcagaggttctcgttgtatggtcagtggtattatttagtttgtctcttgtaacatttctgcttcggtttttgacataaagtgataggttgtctaattctattcatccgtaactctttagaagcaacagcacatctccagtgtatcccagggaggtgatggttgagtccttgatggtatagcacctgggggtcttttcactcactggaccacttcactagtccaaagtttgagaccttctgctctgtcttacatgatttcccccatttagggaggattgatgtgtcctgctggaaaaatctatgaagaacatattttaaaagttagggatttagtgtagttaggcctaggccagttcaaataggtgttgttttgacaataataacaatcattttttgctcctttattccggctaagccatttagacacattatccttagatgaggcactctgaggctcagctgcaaaatacaagtaatttgcccagggtgttacagctgctgagtggaagagccaggatcccaggcaggtgtttctaactttagagaactaggcaaatagtactaactttgtgccaagcactgttataaattcattacatttattaacttaaaacaccccaggaagcagaaactataatcctcattttacagatgaggaaactaaggcacaggggagttaagtgacttgccaaagatcagagagccaattatgttatataactgggatttgaactgaagccattgagtcaagtgtagctgttgcccactacactatactgttctccaaaacctcagcctttccacccacctgaataagctgcccctggtagtaactcttgatgtctggagagttcagagttttgagagaagttggaggggatagagtagaaattaaatctgttcaacccctttttcttgttttaccacagtgccatgtgctttgggctcactgctcacctggttgcaacatgattcactatggtgtaaacctgagccttttccctcttcttgcgtgactcttcactgtcagatgagctggtgttctaatgcagaggacacagctcccttcatgccctgctggagcccattaggctttgtcgaatgcacccgagtgtggcaatcccatgtattctggttctgtagtgctcctttgctgcagtcagagtccagcacaaaggagttgtgaaacaggctttttccacagtgtggtatgttccagagttgctgttcttaattaaatattattcttttgtatttgtactctaggggaattttatcgtgaaagatggattttataaatctaaaagtagaataatcattaaatagatgaggacttttttcaattggcccttgaaactgattgttttccattgtcacctgtgggatcacaggcctggggcgctgccaggagggtctccaaagatgactggctggaatggctgagacggctgagcctggagctgctgaaggactcatcatcgccctccctgcgctcctgctgggccctggcacaggcctacaacccgatggccaggtatagtgtgtcagggcgggtggagaagatcagcacatagaaaacgatatatacaaattagtttttattagaagtaataatcaaggggctggtatttttaatttttctaattttgtgggatacaaagaaagagaaagacaaattcaacaagctcatactgtagcatctatcttaaccttgctagtagaccacaaattcctttttctagtagctttaggtctaacattttaattacaaagttatttataactctaaaagcaactgtgtaatagttgaatgtaacctatttccaataacaatggtattctaattattgctgtttgttacaaataccacaggaaaaaaaaggtctgaaacaataacagttatttattttactcctgaatctgcaatttgggaggacttttgggacagctcgcctctgctccacatggtgtcagtggagatggtttgactggggcaggatggaggatccgcattcaaggctggcttgtgtgtctggcaaagtgcaggtgactctcgactaggaacccagctctggctgggggccttggttcctctccatgggatcactccatgagtttgcttgggccttctcacagatggtgactggcttcaagagtgagtgtcccaagagaacgaggtagtagaagagcatggtgtttttatgatctaagtcataaagagtcttggaagtcatgaagagtcacttctgccttgccctgttgctcaaggcagccacaaaggcgcagtcaggctcaaaggaagaagacatgggcacccacctcttgagagaggagaagtaatattctaacagactatatgggacaggagccactgttgcagacatctttggaaaatagaatttgccagaatttattttttctttttttgagagtcttgttctgttgctcaggctggactgcagtggcacagttgtggctcactgcaaccctgcctcctggttcaagagattctcctgcctcagcctcccgtgtagctgggattacaggtgtgtaccaccacacccagctaattttgtatttttagtaaagacggggtttcgccatgttggccaggctggtcttcaactcctggcctcaagtgatctgctcaccttggcctttcaaagtgctgggattacagacatgagccacttggccaaaattttttttattgatacataatagatgtacatatattttcaaagtacatgtgatatttcgatacattcatataatgtgtaataaaacaaatcaggataattgggatattcatcactttagacatctttttttgttgttgttgtttggttggtttttttgttttcatttttgtttttgtttttgagatggagtcttgctctgtcacccaggctggagtgcagtggcgcaattgtggctcactgcaacctccgtctcctgggttcaagcgattctcctgcctcagcctcccgagtagctgggagtacaggcacacgccaccacgcccggctattttttgtaattttagtagacgtggggtttcaccatgttggccaggctggtcttgaactcctgacctcaaatgatccacctgccttggcctcccagagtgctgggattacaggcgtgagccaccacgccccgcctggttttttgtttttgtttttgttttggaaacagggtctctgctgcccgggctggagtgcagtgggacgatctcggctcactgcagcctcaacctcctgggctcaagtgatcctcccacgtcagcctcctgagtagctgggactgcagacacgcaccaggcccagctaattttattttttgtagagacgaggtctcactatgttgcccaggctgatctcaaactcctggactcaagcagtcatcctgcctcagcctccaaaagtgccgtgatttataggcatgagtcatcaccattttttttttaatttttaaaaatttttatagattttggagtacaggtgcaggtttgttacatggatatgttgcatagtggtgaagtctgggcttttcgtagaaccatcacctgaataatagtgtacatcgtacctatcaggtaatttctcatccctcaaccccgccaaaaaaaatattgttttaaatagtaaatctttttttttttttttttttttgagacggagttttgctctgtttcccaggctggagtgcagtggcgcgatcttggctcaccgcaacctctacttcccaggttcaagccattctcctgcctcagcctcccaatgagctgggactacaggtgcacacctggccaattttttgtacttttagtagagatgggatttcaccatgttagccaggatggtctcgatctcctgacctcgcaatctgcccacctcggcctcccagagtgctgggattacaggcatgagccactgtgcccggcctaaatagtaaatcttaaacagccatttaaaatttggtttacaggctgggcattgtggctcctgcctataatctcagcactctaggaggctgagacaggaggatcacttgaggccaagagttcaagaccagcctgggcaacctagcgagactctatctctacaaaaatgaaaaattagccaagcgtggtggcatgaagctgtaatcctagctactcaggaggctaaggcggggggattacttgagtccaggagtttagaggctgcagagagccacaatcgtgcccctgcattccagcctagatggcagagcaagactgcatctcaaaaaaatttttttttggtttataaaacttctaatgcatgtgaaagatttataatataatgtaagtaaataaaatggaatacaaaattgaaaattaaaataaatgtgaataggataaagattagaaagtaatatagcaatggttatctttagggaaggcttaaggagtgattattttcttttttattctcttctttatttcttaaatttttctgcattggatattaattatttataatgaggcataaaatggtgatttaattaataagcagcccttcgcattggcaagaagcagaaagcaaggcatttcacttcatttttgtcagcagctggtttgctgttatgatatatatttagcagtagtgatggcccctgagactgttggtttgatgccatgatttgcatcctctaatttctttcaagtattttttaatgctttaaaatagtttctaggacttccctgtttccgtagtgtggagatatcatgtactcagctgatgtatttattattcctcttttctctccagtcacctctgagcacagtgttatccatgagaggcttgtgatatacacaatgtttttatccccttagtcccaaatacgttcattcaagatccaatgtggcctctgacctagatcaggaaaactttttttatgattttagagatgaaggtcttggttattgccctggctggtcttgaactgctaatctcaagagatcctcctgcctcagcctcccaagtcaccaggatgaccagcgactgtaattacctggctccgaaaacttacttttaattttttttttttttttgaaacagagtctcgctctgttgcccaggctggagtgcagtggtgtgatcttggctcactgcaaactctgcctcccaggttcaagccattctcctgcctcagcctcctgagaagctgggactacaggccaccaccaccgcgcccggctaattttttgtatttttagtagagacggggtttcaccatgttagccaggatggtctcgatctcctgaccttgtgatccgcccgcctcagccccccaaagtactgggattacaggcgtgagccaccacgcctggccttatttttaaatttataacagactttttttggtcttcatttgatcctttggatttcaaattttgttttttctatcaaattccaaaattatacacattggtctaacttaacctgttaatatgtaacagaaaataggtatagaggcatcaaatgctaaatctatccattgtaggtatatcctgttttttcccatttccttccccgcacagtcaccagaacagtggggaggacagaattaatgttagatgtaagggatggcaaatgtgtccatcatgtggtactgtggtaatgtaaagtggtcatagtaatctttgaaatggaacctctggttccatgtctttgtgaggccccaactgttggcttagaaaatcttttagaacttaaggatttggatcctatttgctaatgcttctagtcctgttgtcattttttatgaccatccaactaaggattctggttatattttgaaagcaattgcagatgctgcctttgacatttctgaccttaagaatttagacattgtcccagaaacatcccaagaatcatttaatgattattattttctgcatttgttaatgccaacttctatgttagaagtccctatactgttcatttcccatttcctccagtgaagagaaataatgcaaaaactgccaagaaatattcctttggagaaaagtcactttgctctgctcttctccccgccctgccactcgccccccaagtccctgaatatcacaatgtctgtacctgcagtgtaaaattcagtagacactagccatatgtggttaattaaaaataaataaaatgtaaaattcagttccttggccacacttgccacagttcaagtgctttagtgatcactggctaccatattggacaggaaaattctcttggacagcactggtctgtatagcactggtttgtataaccaaaatcagtgatgccaaaagcgaattgataggtgtgactttgaggcagcaggcagaggcacgggaggtctctccattttccaccttctctgccttcagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaaggtgagacttttccccattgtccagatggaggctgacagggaaccagctggtgggaggagagaagcatccacacagccttctcttgtttgtgccccatctgccattttcctgtgctgtgaggttttgctcttctccagggccccctgccactgagagatgacaatggcattgttctgctgggtgagagagctgccaagtgccgagcatatgccaaagcactacactacaaagaactggagttccagaaaggccccacccctgccattctagaatctctcatcaggtaggccataaaccctttttaattgctgccttcatgcaaaatgacctctcttctcccactgcctggtgccacttcactggagtcacttgaagacttctgctctgtgaaatctgcctctgggtctggccagcatgtttaaatgacatgttgttgcagatggctctccccaggaatctagccatgattgtcatttctgttgacccttatcacctgggctcctgttgttttccaacctgtcctttcttgttccagtgaaatttagttgagaagcaattctgtggccatggcagcctaggaagataggttttatgtctgggttagatagcgctgtctagagctggagtagaatgtgctggttcctgggcttcagagggtactcaagggcacagtggggttttatcaactaaatctattgatgcctgcatcttcctcctctggtgttttgtgaatataaaaggcagagcataatctgaaatctaatggtcttttctctgatgtggttatgttcgctcctctgttaagacagttttcagtcttctgggtatggaaaaaatagttcgtttaataaattttctgctgttaggaaattacaagcagcatgaaacattagctccagctcctaaaaaaccattcacttcccctaatgattatcagagaatatcaacttcgggatctcattatcttcatgtgaatgctaaatggtcggtcatttcactgcagatagtttagaaaaaatcatctggctagtaagttccttcaccgtggctgatagagcctgctgtgtggctctatcaaaggaggatagagcctcctttgtttgagagggtaatcaaatagccaagtcctttcctagctgtgcttttgagaagatatttaaaagagttcctttaacactttaaaaagttagcgaaaagatagaaatagaatcctgtttttgcccaaataaattggacttggagaaaatgttgatttctgaaataccatgtaggaataattaaaaacatgccgtcctctgaggcatgcttcagtttcacatcaggcacagtaaatgtgaaaatccactttcttccttccatctctggcttaggtaatgtgctgtcacttgatagctcatagaaaatacggtaactaagtatgatcactttaaggtcagataactcattttgcatattaatttccttgctcacctgtttgagacctatttgaaaccttcccaattccacccttaatttttccgcatatttgatacaatcataaaaacttaaagttatgcagatttgtagcagaaaaatatagtcagaggcctggaaaagtgctctagaaggttatttagctttgtaagcaaatataattgaaagtaaatcctcagaattccctggagattgatcttgtcctttgtttaaaacgctgttccaggccgggcggggtagttcacacctgtaatcccagcactctgggaggccaaggtgggtggatcacttgatgtcaggagttcgagaccagcctggccaacatggtgaaacacccatctctactaaaaatacaaaaattagctgggtgtggtggcgcacatctgtattcccagctccttgagaggctaaggcaggagaattgcttgaacctgggaggtggaggttgcaatgagccaagattgcgtcactgtactctagcctggatgacagcgagattctgtctcaataaaaaagtgaatgaatgagtgaatgaatgaatgctgttactggggaaagccagtgtgctcggctctttgggtgtcctcagctaagtcagctcagcttgcatttctgcctcccttctgctggtctcctgtgtcttcccatcacctttctttttttgcttccaattgcagtttttacttctgattttgtaaagctgcttttcctaagcccggttgtggtggtatactgtcgtctttactgaaggaagttttaagaaacagccttagacaaagttgctcatagcactacccctagttctgtgggtgagcctcatggaaattcttctcaggggtcttttcccactgttggttttatgtccctgagccactacttggatctggccttggagacatcgctttgcagtaagggattgaattccttcctcacagtggctgctagagagcttgaagctaaattcctggtctacctccaacaaattccagacatcttacctgtttttactactaaccctcccccaacccccacaggctttgttttatttttcctactcttattttcccttcaccaagttgtctgctttttcagagcttattttagtatatacatttgagtcaacttccctaaattctttttgaaacaattagggtagccctacacaaaaacatctttgttcagctatttcttttcttttcttttcttttttttttttttagatggagtcgcgctctgtctccaggctggagtgcagtggcacaatcttggctcactgcaacctccacctcctagatgcaagcgattctcctgcctcagcttcctaagtagctgggactacaggcgcatgccaccatgcccagctaatttttgtatttttagtagaaaggggtttcaccatgttggccaggatggtctcactctcttgacctcgtgatccacccgcctcggcctcctaaagtgctgggattacaggcgtgagccaccgcgtgcagctatttcttatttttttgtctttgctttaaaaaactgtgttctgttgacagtattgttctcttgctactagttcaatccgtcttgcaacctattgacttttttaacctatctgctacagggcaaattaaatccgagatgtaatcccaaatcagggctaacactgagatagtataaactacccccatctccttaaagctcacatatcagtaaatatatttgctttcttggggacagaggattgagtggttatttgttagaaagcgcacacacaccacttttacttggttagccccgagcattagggtaaggtctgctggcctgtttttgcttctctatcctaagtagaatagagggtctgataaggctggatgccttcactgcccagccccagacgtgtaatcccctttatgaagaattgtcctggttgctgcccttttgtcccttacctgttcccttttggcttctgctccccgcttggttgccctgaacaccaaaacaaaccccgggcctctgtcactgctcaccctctgatttaactttaactttttatcctgctgtaagtggtaatggtaagaatttaaaaagaagatactcacattttgaaagtgtttattgtagaaggaaaagaaacgttgaatgtttaaaacaaaaccacaatattttatttttacaaagtgagatgcttatttacagcatttattattaactataacaccatctaccttcagttttcaaccatttcctacagacaaaatttaaaaggaagctccttgagcttgtttcttacaaagtaaataaaaatagatatacctgtattaactatgaatattttaaggcagttttgtgccggcatccatgagagactctgtccacctcctgctggtgcttcattcctttcagtgctgtacatgaccaaggcgagcaggggcagtgctcagagtctgatcgccagatgcagtgccctggagtttcctccccgcaagtccagcctgggtcagttccagccgaacaatttttgtgttttaaaaataatgtgtagactcaagataatttcactggatttaaaaatagaaataccatttcccacctgcctccttcactcctttctctaacactcaaaggcctatcctttgagaatcaaaataatatggttctttttagcaaagggatttgtgttttgccctcacatagcctaagccgtagagaaaatagataaaacgatctcaaaattattttctttcacagacaaatcatgtggaaaagacaagctactgctatgtcagggatcttgggcagagagaccccactcagtctaccaccttgagaaggatgtggcaggacccagtgggtttaaatagttcatcctgtttactgtaacatccttggtacatatgatttttttgtgctccttggagacttattctttctttggagattataggctgtttctcagtcctaccctttctaacactctcttcctgtcctcatctgccctcatccagtctccacgtgtctcagtttctgtatccgtgctccacggcagcctccttttaaggcttgaagtcttctgggcggattttcatctccacccgtttgagggagtaggtagatccatgccagccataccaggtgatgccatccaggtgcttattgtgctcacccaggcggtagtacactccattgaggttggagtctgtgcagcagttgtaccagtagccacctggcatgagaaacagacccagtgctagtaagttgactgtgatagaccagacatcaggtaaggagttagagccagggtcagaaagaataccaaaaccggaatatcaactgtacagttataattcactctttctcctcaagtgggattatgagcttgtacctgaacttctcctttccggtccccccaaatctcacctttgcggagctgtgcacacttgtccaagcagttgtcattgtccttgtccttggtgctgaaggctgtgttgttatgatactggagggcgtcgttccccacattgccagtgtagttccccaggaagaggcgatagctgttgagttcattgcccaaaacaaagtggctatactcagcgtagcgcaggttgccctcccagtcctgtcaggaggagcagagccttaaagaaagtgcagcaggctccatttcatgctgtctctataccccttctctcctgacagtcccatagccaaacatctttttataggtgtgaatctaataagactttattacttgtttacctccagtttactgtgttaacctgggaatcagaaaacaaggctcgaacttctttcctgattcaccatatggccttgattagtctctctttctatattcctcactttcctgcttataaggtgcagctatgtttttgctgaatcccacacagagctgctgtaagagaaacatgaggatatagtgagtgctttcagaccttcagcaagggtccccaaacagtatcagtaagatttgcacctaaaggacctcatgctcggccagagcagagactagtgttcggcacagctgactgttgaatttgctctactctggcctgggtctggcctcttcctgtcccacctgcagccccacttttgtcggggggttgggggggttgcagaaaggaaaaatacctatttttcctcaactccattacaacttgcaggaaaaccatcacaatgaatatacagatttgtcttgaatcagaataggaatggcacccccggagttgtacgcggtcatgtgtggtggcactggtccagtcatggggcccctggccttgtgcttacctccatctctacacgcagccgggttggctgtctggagagccggtggatgtgttcgttccccagccagaagtccccacggatgctgccaaagccctgcttgtactgcttccagtcccggtagaaggagacaaggccactttttcgtctctggatgatggtccagcctccgcctgaagtctccatgtcacagaacacctgaagcagaggggatactctggtgagggacttgcaggggcagaaggcaggagcaagagggcaggcttcccaacctgttcctgtccctgcagagccagtagatgtgtgcactcccgcagtcacactggctcactcgggcagtgatgctgccccactgctggtgagctggggtgggtggtgcattgctctggaagaacgatgcaaattgggcccgggggtgagtgcacatgaagcttggagggaaaaatccagatactttttttttttttgaagcagaatttcactcttgttgcccaggctagagtacagtggtgcaatctcggctcactgcaacctccacctcctgagttccagtgattctcctgccttcctagtagctgggaccagagtgcctgtcaccacactgggctaatttttgtatttttagtagagactgggtttcagcatgttggccaggctggtctcaaagtcctgacctcaggtgattgttccgcccacctcacctcccaaaaaacttgggattacaggtgtgagccaccatgcctgggtgaaaatccagataatttgatgctgccttcgtgttttcttttctcctcccccaaggaatagctgttctaggacatggcaagagatttaaactaagttatgtggtggttataagaatgatctgatctatgttagaaggcttgttgtttgactgataatgatttgaaggggccaaacagatgcctgggtttggagtgcagggcataaactgagtgtttgcatagctctgtggtgtttggtgtgtttgcttgtgtttgagttttccttttttttttttttttttttaattgaaatggagtctcactttgtctcccagattatagtgcagtggcgtgatctcggctcactgcaacctctgcctcctgagttcaagcaatcctcctgtgtagcagggattacaggtgcccaccaccatgcctggctaatttttttgtatttttagtagagacagggtttcaccatgttggccaggctggtcttaaactcctgacctgaagtgatccacctgcctcggcctcccatagtgctgggattacaggtgtgagccactgcgcccagccgaattttcctttctttaaactgtgaatggccctgttgttgtagtccccttgagggggtgaaggacaggtattagggcatggccagtgactgtaatgacctcacctccagttcagggctgcccaggaagtcatcaggaggaagcttatacactccagagatgcggtagttcttctggtagagggaagagcagtcgtagatggcatctacaagagaacaaaccacagggtgagcatttagacccagtggctccatctgagttgagtgtttagagactgagcctttatttctaccctgggattctcagtggtgttaattacccatcagtgtctgcatttgggagaggtggatatggggagatccagagggggtgtattttggctttactagcggtggagcagaggggtagggaagcccttcacctggatagcattagtgggacgttccaacaggtggtttcaggttaactctgaaataaattattctgctatagctgggccatgaattctagaaatgttacagtgtatgagggccacgtaacagatactcctgtcagatagattcagacttcttagacagccgccactataacaaatcagtcgtgaatatctcatagagtctggttttgttgatgtttcctggttggttgtttttgttgttgttcagagatggtggctactgccacctgagaccataattaagggagatagattttattatcacatattttccccttccatccagctagcacctcaaaacactcagaagaacctttgaggccttcctcccagaagttttgttaaaacaatgaaatgagagcatcagactggccagtttttttactctagggccatggagctgctactttatgcaggatttaagctctgtattctaaatatcaaagagcatagtggaagagacagccttttctgataggattcagagactgcggcccttactatccaagagaagcttcctgcctgctatgtctcggggtgcttttcttggtgaccactgcactgggaccttagagaggagcaccaagatggcagatgggagttgtctgaggcacataggttctcttgcagagcaccttgggaaagggaggtagagtacgtgtgccttcgccatcactattcttcatttcgtcttagtggtgaaagcaccaacttactgcttcctctaagggactacctacttatagaatataagggtaactgagcacttggtcatagagaagaaaaataaggaatgatgtcctgggcaagaagagcaagagacctggtgggagagctgcacttgaccccaagaacctccttgcaggatggtgagaaagcctcctgtttggatgagcaggaagtggcacggtcagattcctctgtctggcctggtcttccgcatgtctggtttcatctcacagaccaaatcaggactcacttgtctggaaaatggcctgagaatacctttctaatcccaaactgagggcccggaagtccatgcttagaaagtgattgatacatgttggttcaggtaacagaattttcaagcttggtttcatgttaaaactttttcttaatttgagctgaggttaaccattgacttagtcagtagaagctgatcatgtctttgttcttcatgtagtttataatgggaccaatggtttttatatcagcaagaaatgacatgtctataggatatgctccttacaactaatattttcctgataatttcagaaaaccaaatttttatctctaaaccgtaaattcatgttggcccacctgagctgagctaaccagaaataaaagttattttgcaaaagaaagattaaatttatcttatttttggcctattaagtctatatttcagcattgcatttaaaaaatcctgtattcggagaaattcatttaagaccatggaaaaaaatagacgtatcacaattgaggaaccttaaggagccgcatccttcctgaacacaaagggactcttcacatggttatgtgtctctacacttaactctgagccaatgagaagagttacgtttacagagagaggacacaaaattccattaatgctcattcctggtaaatcttaggaacaccagagaactgtttctggcaaaactttgcttccagaggaacagctgttacagtttggtgcttccttggaatttcttgagatatagcctgtcagctcttggtgaggaagtagcagagctacaccaatcaggaagacttgaaccctgcttctaagacttgctgggtgactttgggcaaaccatgtagtatctcagcactccaagatggtaggctgaaacactgtgtggataaaagagacccgtgaaggtgttaggtaaacttacatagcattagaagtcaaaggaaatactgcataagcctttactaaagggaaagtgtacccacaaaagaggccccagtagtggttctgatgaccaggatatgtagaagcagtgctgtagatggggaggcactccacgctccctcaggggactggtctccttacctgcggaggtctgagtgaccgtctgtgctgcctgcagctgcatgatgtcaatttggttgttcatctcggagtacttgctctcagcatctgtgagccgcgactccatgcgcttgctgttgctctccagctccatcacctgcatgaccacgctgacccagtccctctcctgcttcttgttcagttcactcagcaggctgctaaggttggcaacttgggccttgagctccttcacctcctcacagcagttggccgctttgagctgtggctgtgctggtgtcttgtgcttagagagcttctgcagccacgctgggtggctgacaaaggccacgatgaaaatgcagagccaggtcacagctgagagaggctttttcagcatcttttgtgggtttgggtgaggaaggctcttccttcaggggagtctggagatgcccagctgaggtctgcaaagctgcagcaggctgactgaaccttacgctttcactctgagggcctcgccactttgttgttttctctctttccttgctctttgctagcctttctcaaagcttgagtctctgacaggggagctgaatgggtagcctatagctttccttccaagcccgaatgctccacaagtcagcatcttaaatattgtttgctgcctgtacctccacccctaccatggctgcaccaggctttagcccctcccagctcatctgggagggtcaggtgcctgttgggtgaagtcagcggcaacttaggccagaggaggggaggagggagatggggcgagtctttctttccttgcctgtacagtctctgggctaagacaagaggactgtgaatttgtgtgtttccagtttcgcctattagattatgcccctgccagccactggctcaactctatcagcaagcaggacagggtcagaagaatttccaaattccgttcaaaaggttatataaccatggggtgagcgtaaggtaggcgaaagagacgctactcacccagggaggcctgttaatgtaatccatgctgtttatatgaggatctggtatattttgacatcctgctgaatatgcttttatctaggctgattattcccatctctgatcattatttagtattttataaaatccttactcaacctggatctaacactatctcacatttgaaatcttccaacttagaaaagttctagaaatagaagaatagagtaaatctcttaggaagccaaaaaaagactttgtagccacagggtttggacagttgtctttttacatgctttctccttcaacaaagagagggccagtattagcatataacacttgatattttgcaaggaggtgtcagaagattacaacagagatgaaaactcagcagtgccaagcatgtctgggattggtcacccatgcttctgtcagtttctaggcaggacagagaacaggcccatccttagcccgtggttcaaaacttggaaaatatattaaaatatagagaaaagaatccctgtcacctaataaatccatatctagagtctcatttccctctttctttgctggtatggtttgcaatttgaaattattcctcctctatttgtatatgactttgtatctaaaaaacatgaatattgtaatttttcatgtactatatatgaaggacaatgctcttttgaaaagaaatgtgaaaaacatggagtttcagcaaacatccttaaaataagcttttcactaagaatctatacaaaggttcctctgtccttaaaacagtaactctgtctttggaacatacgattgctgtttccatgttatggagccactcaggcagttacttgtactgttgtagtcagcgtgggtccttcccacataagcctgatgcctgacagctctcctcccatgccataatcccccagagctcttttccagtcagggcttctagctcctcagaaagtaatttctgttcaggcagctggagttactgaacaatttttttttttctcagaatcaaaaagcagtggaggatcaggtggatcagagtttgttttctcacatacccagcctcctttgcctgctaaagggtgaggatgttactgatacccttatagcaacttggcattctaatcaaaagattctgttgagaattggcactgtctgccagtctgctctctttttgcaaatgtctagcccagctgggcaccacagccttagctccatagcattcattttagtaacgagactctggccccaaatattggcatcctacaaggtctgctaaaaatagggattttcaagtggatctcctggccatcaaatgacactgagcttaaaccacatgccacagaagtgctatgtaccatacagtcatgtatagcaggggtccctaatccccaggctgcagacctgtacttgtccatggcctgttaggaatagggccatacagcaggaggtgagtggcaggtgagtgagcattaccgcctgagctctgcctcctgtcagatcggtggcagcattagatcctcacaggagtgtgaaccctgttgtgaactgcacatgtgagggatctaggttgcgtgctccttgtgagaatctaagtactgcctgaagatctgaggtggaacagttttacctcaaaaccatcccccatctccccacccccaccctggtccatagaaaaattgtcttccacgaaaccagtccctagtgccaaaaagattggggaccactgatgtatagcatcccacgtgaccctgctgccttggaactctcatgtaacagtgcagcagagaaagcacaaaagaacagtggaactttgtggagttctagccagcttttgtttattcttcccagtagctcacccaaacaggaaagtgttatgaattgattcttatatgtgggcagaaaataaattaatcattcatatttgtaccccatgtaacagtcccttttaggaaaggtaggaggaggttcgtaagtttgtattggaagcatctagcctttagcatgagtgtctggtctttgacatttattttctggaaacttatttctaacataaggcaatacttgaagacagaagacagaagctcttgaattgcataagctacccaaatgtcaatactcaccccccctccagttttgaggaaaaatagctaattaaggatatttctcatgccagaagattatctattttctaaaacatccacaaccttatgatgtcctagattctgaatatctaaatgaaggaaaaagaagtccttgttttgagcacagctaagccagtagagaaaaccatgaaggatgtggaggactaaaatggtattgccttctctgaaagaggcttttaaatccttcttctgcctttcagtgagcagagtgaccttaaaaatgggaattaactgacaaaatctggaaaacctaggttggatactgtttagattttccttttctccattttgctggtgttgaaagagggctagcttggtgctcctggaaaaggtgtttatgaaaattaagcttttgttcttaatgaaaaaagtcagtgaggttgtactcaacctggaggttctcaaaagacttcacagtgtttgggccgcactgccttgggctggcaccaaggtagtctgagggtcacagggcaggggagcacctaggggtggtctctccttctatccagagaggagcatcagatcccatttgcttgaaaatgccccgttggagagactgctgcccactctcagcttgtggaatgaatacatctcagaagaagctgccttgctgttagttttaattgtgtatctcaaaggaatatttcacttcttctgaagtctttgaaaaaaccacagcctgccatactctatacagccgttgaatatctgcctttttttttttttttttttttttgagacggagtcttgctctgtcgtccaggctggagtgcagtggcacgatcttggctcactggaagctctgcctcccgggttcacgccaattctcctgcctcagcctcccaagtagctgggactacaggcacctgccaccatgcccagctaattttttgtattttttagtacaggcagggtttcaccatgttagccaggatggtctcgatctcctgatctgttgatccacccgcctcggcctcccaaagtgctgggattacaggtgtgagtcatcatgcccggccacatatctgcctttctagcactccatcccattcctgtctcaactgaaagtccaagtccatcacagaagatatttccatcttttccctgagctttctaatttgccacttctatcaaagaatgtgtcctgaacctattagaagtcttttctctcaggccttctcttagagtcctgttttgttttgttctgttttgttttgtgacagggtttcactctgttcaggctagagtgcaatggggcaaccatggctcattgcagcctcaacctcccagtctcaagtgatccatccacctcagcctcccgagtagctaggactgcaggtatgcaccaccacacccaactatctttttttctttctttctttctttcttttctttttttttttttgtagtgatgggggtctcactgtgttgccagggctggtctcgaactcctgggctcaagtgatcctccagcctcagcctctcaaagtgctaggattacaggcatgagccaccacacctggccagagtcagttttaaattatggaatatagtaaaaaaaaaaaaaaagtctctctgatttctgtcaggactttatagctctgttagcctccactcaagttgcacaatccagcgattattctggcccagtgtctggcagtctagttagacagctgaactcttggacagtgtgacatgctaggatgggagacacagaggacaaggagggccttctagagaagtgatacctaggttttaaagaaaaaggaggaaatagccaggaatgggagaggatgttgtaagcaaaggtgcagctatgtgaaagacttggactagtgggagaatgtaagtagtcgagtgtggcctaagagtcaaagggaagaagaatggcagcagaagctaaagctggagaggtggaggggggccgtgcccggggagcccaggtgaggagcgggactgatcctctgtagacatgggaaggaatcctagaggtttttccctcttgttccattgttttcctgagttccaccaggaaagtggccttggtaattgccaaacaatttttttttattttttattttttttagagacatagtctcattctgttacccaccctggagtgctgtagtgcaatcatatagcttactgaaacctggaactcgtgagctgaagcagtcctcctgcctgagcctccaaagtagctgggaatacaagcgcacaccaacacgcctgcctgtttttaaatttttttgtagagacggagtctcactatgttgaggctggtgtggaactcctagcctcaagagatcttcccatctcaacctcccgaagtgctaggattataggcctgagccaccacacacagcccaaacaagattttttcaccgcaaatgaatgatttagaacgtgagaatttcttaaaattgtaatgcattcatattaaataccctgacggggtcatttggcattaggattaaggaggtagtacatatagcacttgagttcttaggcctagaagcagacaaatctggattctaatccctccctagcacttggttgctgtgtggccatggacatattacttctctgagtttgccaacctgtgaaatggagataaaaatcaaaactatctcataccacaggcttttcatgagaattaaataaagcaaagggttaaataaccaaggtatttttggataaggaaaataaatttggaagagttacactactacatatcaagatttatcataaagctgcagtaagtaaggcagtacagtagtgacatggatgaacaaatggaacagaattaaatgtccagaaacagacccacatatgtagagatacttaattttgtgtatttatatttatgtatttattcctttcctctcaagccatgtgatatagacacttgatttataacaaggtagcaccacagagtggtgaggaaaggactgctaagtcagctcagtagccttatgggggaagaaaatttttttttttccctcagtgatcctttttctgaaagaaaagactgttgatcgttacctcacaccacacacaaaagctcaagtccaggagatttaaactcttaagtgtgaacagtaaaacaataaaatttatagaggataacgtaggagattaagtggcttcctaagataagcaaagatttcttaagatagaaaaaacaccaatcataaaggaaaatattaagaaatgcaacaaaagtagtcattctgttcattggaagacacaatgaagagaatgaaaggcaagaaatatttctattatcttcaatattgtatatgtattatacccagagtgtataaagaactcttatcaaacaataaggaggaaaaaggtagcacaataaaaagattggtaaaagaagccaggtacagtggtgtgtacctgtagtctgagctactcgggaggttgaggtggaaggattgcttgagcccaagagttggaaaccagcctaggcaacatagagagactctgtctcaaaaagaaaaaaataaaatattgacaaaagatgtgaacaggcacttagttcacaaagatatccaaaaggcttataaatacatgaaaagctatcccatctcattagtcattatggaaataaaaatttaaatcataataaaatgccactgcacaccaaccagaagggctgaaatggaaaaggctgaaaatagcaaatgttgacagagattgtgaagcaattgggcttttcatgtcctattggtagaaatgtaaattggtaaagccactttggaaaacaatctgtcagtatcttataaagctaaacatatccttatatgtaactcaacaattctgctatagatattttccagcagaagtgcaagcacacatgctccagaagacatggacaagagtgtccgcgtactacccctcgtaatagccaaacactgaaaacaatccaagtgttcatctgcagtaagataggtaaatggtggtatattcacaccgtgaaatactgttcagcaatagaaaggaaaagctgttgcgacgtgcagcagcctgggtgcatgtcacaaacatagtgttgaatgtttatataattaagctcaaaaactgagcaaacaaccccattgtgttagaagtcagagtagggtttacagacctctggggacacagaggggagcagtgattgggagggggctcagtgcctctgagctgctggcagctggttgtgttctgtttctggtctgggtggtaattacaaagatgtcttcactttatgataattcatcaaactgtacacttaggtgttatattctcctatatttatgtatacttgaattaggatgtttgtgtaaaaatagataatgcacataaaacacttgaactagtatctgacacatagtcaacgtctagcaaatatcaagtagtagtattgtaccaagtagtagtattgtattatttagtacacaattgttgtctcgtcataaaagtatttgctgactactctagtctaacataaatccccataaatccccatgtttttctctaagcactctgttctcttcatggtatttgccacaaaagcacttatttcagtttgtacttttttttttttttttaagatggagtttcgctcttattgcccaggctggagcgcagtggcgcaatctcagctcactgcaacctccgcctcccaggttcaagcaattctcttgcctcagcctcctgagtagctgggattacagtcatgcactgccatgccttgctaattttgtatttttagtagagagggggtttctccatgtcggtcaggctggtctcgaactcctgacctcaggtgatccgcctgccttggcctcccaaagtgctgggattacaggtgtgagccactgcacctggcctcggtttgtacttttttgtgtatctacttctctaacctgtaagctctgcgaaagcaaagaacatgtttctcttattttccacagtacctctagcatctagcacttagcataaagcctaagacaaaatgcatactaaattagaacttgtcaaatgaaataatgaatttcaaagagaaggttctcttccccctaaatatctattatggagacttacagtctctctccatggacgtttccagtgttttactgtggtgaggtcttcgctgtagggcatcacattctgtcctgcatggggtcagttttccttcttcaggtcttagtcatagaatcaaagaaagtgaaaactgggagagttgagatcctttgtttcaaccctctttttaatttttattttatttgtttatttattttgagatggagtctggctctgtcgcccaggcttgagtgtagtggtggggtctcagctcagtgcaacctccgcctcctggattcaagcagttctcttgcctcagcctcccgagtagctggaattacaggtgcccaccactacatccggctttgaccccattttttaaatcaataacttgaggtagcttccttattgtataatatattgcatacatattcctagacatcaccaggctgtgtctcatcacttttttttttacttaggaagatgatcagcaagaggaccacagagtatctgagatcaggttttctccctctcctgtttattccagaagccagtgtaaatgacatgtctgattacttcaacctgcactggccctaggggctgcatgcaacagctaatgttgacaaaatgaagaaagtaacatgtccccataaaaactgccttatttcgggcgggcaaggtggctcatgcctgtaatcccagcactttgggaggccaagacgggcagattacaaagtcaagatatcaagaccatcctggccaatgtggtgaaaccccgtctctattaaaaatacaaaaattagctggatgtggtggcacactcctgtagtcccagctacttgggaggctgaggccagagaattgcttgaactcgggaggtagaggttgcagtgagccgagatcgcaccgctgcactccagcctggcgacagagcaagactccgtcaaaaaaaaaaaaaaaacctgccttatttctgggcagctgtagtccccatattttatccccagtaacctaacaaagctcatctgtccccaaagggaaatcaaatgtccattatattgcaatttcagtaaatcacccttagctagagataagaccttgtcaggggttgcaggacaagatcacatgtccaccccaccaagctgttctctgaactacagagcttctctttgggttagacatctccaccctgacttaggcaccacaaactggaaaacaggcctggtcattctttaagaatgttagaggatttgtgccagttatgcctgagcaaccaattaatggcaagggtgactgggttttacagtctgcaggcaaggggctaaatgggcaaacaaaacccactctttgtcttaattctaaacacatgtttccagggtgccaatattgtgctgcttgaaagtatgattcctgcttttacagttgtttcctggcttccagaaaagatatgcggtccttttctttttctgtacctctgctggaagaaaaagcagtcagtccacagaggtgaggttttgctcacagcaaaacacattccaggctgggtgcagtggctcactcctataatcccagcactttgggaggccaagacaggaggattgcttgaagccaggagttcaagaccagcctgggtaacatagcaagaccttgtctctgtaaaaagtttagtggggcatgatggcatgcgcttgtggtctcagctactcaggaggccaaggcaggaggatcacttgagtccaggagtttgaggctgcagtgagctatgattgcaccactgcactccagcgtgggcaacagaacaagacactatctcaaaacaaacaaaaaaccacattccaaacttctatgttcgtgctgacttcagaagggccagtgtgcctgtaagaatctgtgtcctttgtagaaaagctccatgctgcaaagagtaggagaaaagagcaaaagcagaagcagtttgtatctttgctgtagccccattttttttttcaaatgcacttttagattcagcccaagaggccacatctctcaccagttatcagtagagcatgcatttacttctagctgctgtcaagacttcccccagggtcactgtaacagcttgctcttgcagagcgtcttatgaaccccagggaaggctcttcagtggagaaacctcgccatcagcgaggaattgtaagaagaggagagttctggggtttctcctaaagctggccaaaaggacagaggggctattgttacaaaacaagacccttctaaggggagaaacttgctgtatttcagtggtatccctaaactcttctacaaaagctagcttatccttcagactacctggagatttaatgggactgctcttatgctgacttagagaaactgcttctgatttttatataacactgggggagggaaagaaagtgctagcagttacactgtgtcccagtgctctgaaaccttgaattgtatgctgcatcccaatttcagagatgttaaggtgtgaaaaaaatgtgtgtcttagaactgatgaaatctagtaggtgctgggctctgcctcatggccacctctgactgttcatgcagtgatgatatcagtaggggcaccactggtctctccaaacatttctggacttttcactggctgtgaatcctggtaatccagttatagagtgccacagttctcttccatgaggcatccattaccaagaaggtgggcatggttttttcttttccctgaacccagaatgggcatcattattcctacactgtggcccaactccttaatagagtggaagaccagctcacatctgcttctacaattaaatgtgaagagttacaagctgccagatcactaggtgccagcttgtttaagttttgtttcccaactttatccacataatggactgagtcagagttcttggacattcagaagccaagaaatcaggactttacccaaaatatcacctcagttttgagtcaagagagatgaacattagctttggagtcactctatcagcccaccccttttgctccttttccccatccttttcctgactgctgcttctttctagtagtctctctcagagctctttgtttctctctggatgtgaagaagcagaaagggggagaagaacctgcaggcatcacaagaaaaggagcagtgctcattgttgtagaataaaaagataatggccaatttgtcaagaagtgttgacaagtaagataaagttggaagtaattaactggctgacatgctgggagcggtgtgcctgccaatagctccctaaatcagccagagccttggaaatgtgacagccattcatcatttttacaacacgcctctgacaggctcctcagttgcaaacctggccagcaaaagtcgtgtgctttgacattctggggttgtgcacctctctctgccttattctgggttgtgctaaggtacctgggccctatatccaaccaggagatgcctggagacggtgagtcaaatgggccagcttgccagctcatcgctgggcagtattcctctccgaggctgggcactaggattcccagagctgactttgtaaacttctgcaggacagttcacttagcatagagagtgctgaatgggagccatcagcagcactcctggtggcctggtatctaacaagtgctgctgctaaccagccatgggatctatcagtgggaggaaacgggtatgcagccagtaaaagcaattatgaagactgaatggagtggaaaatacctgtgttagtgaaaaggcagcatacaaaattgtatctatactttcatcaccagtatgtataaaatatgcatttatgcagaatatgcgcaggtaaaagtatcttggaaaggtgattatattacaatcaacttccctcctccatttttttctttttaaaaagtggtacttccactcaccaatcttgaaaagatgagtttttttgcacacgcgcccccattcccccacgcacttcctaatctcaaaaactgttaacattctaccctctttattctaagggctcacactttctcacatacagatttagcgtgtcctctaggcattgtgacagacaaaccatttaaactgtgaccagacagaaacaaggatgaagacatagtaagaattctttttatttttaggccactctgagcctgagacatcaggaatcttttgttttgtttttggagacagagtctcactctgtcacccagtctggagtgcagtggtgcgattgtagcccactgcagcctcacactcgtgggctcaagtgttcctcccgccttagtctcccaaatagctgggactgcagacacagatcacctcacccagctaatttttaaattttttgtagattcgaagtcttgctatgttgctcaggctggtcttgaattcctgtcctcaagcagtcatcctgtctcggcctcccaaggtggtgggattacaggcatgagccaccgcgcctagccaaagaaacctattttgactgtctaaagctggccacctctcttgcaccaaacttttttttatcttggtgccttaggtccccaagagctccaagtacaccaaatgccccagctttgaaaccttgtaatggggccagccttctcttaatatataaagttctttttcctaagttccagccctgccagacccagaggtactgtcttcatcatctgattctggctttgtttcccttcacttcccctcctgtctgcattccggcagccctcccaaccttctgccccgtcacttttcagatgttttcattttgcttctgtctgccctgctgtcctttctcttctacattagtctgttcattcttgagttccccagaccattctcctgttcccagattcccaaggtagcttcctgataaagacatcaaggcctagccaaccccagatcttcctggccctagaaggaatcagactatttggttgttaatcctgcgttgggcacagagctaagcctcagtgctgggagaagtgacaggagaatctcaagcagttaccactccatgagagaaactgcttgatacagtggttaggacccaaagcagtgtccagggtggcctcagtgtggctggtcaggccacttcagtcagacctgccacagccccatcagctgtgatgcatgtgtcaccagcctggcgccaggggaattcagagaattctcacttgacagggaaccaaatgaaactcagactgtggtctgcactagaaatagacaggagaaagaaggtccacagccatttgggaagccagtgggtgtgttctcatacattcctgatgggagcgctggggaagcctgagcgttgccccagctgtgcctgcagatgctccttgctgcctgaggactttccgacaggcgccgccttgtgtggaagaggcttgcatcactttgctctgaaccccagaggtcacgcggcaccaagactgagggatgagggcgataaccaagtgggccactcaaggaggcactgccatgtcaagttactgtggactcagcatctgtccagaagcctgtggagccgtgactgagggcacttccagagacaatgttctccaggtttctcttcaggtggcaggaaaggcgtttagactgctttgctgtgggtcagcattaagtccttgagcagttaatgacccactatttcagtcaatctcatcctcctccaataaaaggcagtgagggggctgggcacagtggctcatgcctgtaatcccagcactttgggaggctgaggcggttagatcatttgaggtcaggagttcaagaccagcctggccaacatggtgaaaacacatctctactactaaaaatagaaaaattagctgggcagtagtggcgcgcacctgtaatcccagctactggagagactgaggcatgagaatccttgagcctgggaggcagaggttgcggtgagccgagattgcgccactgcactccagtctgggtgacagagtgagaccctgtctcaaaaaaaaaaaaaaaacctgctagggagagcctgagaggaaagggctatggggaggcagcaggcgccactgcccacgtgagctgctctaccgtcactcctgacagggcctttgccgggggaagtgtctgcacagctgaaccacccgctgctcagctcgcatgcttgccctgccttgggaacaaggtttctggatagtggggataagatgttcagcgtaatggtgtgatgtttgctataggaaggcagcaaggcaatttcttagtattagttctgctcagctctagaaaatgttttccttgattgccaagaactttcccagctcaattcaaaactcgaatttgatgaaaggaggctctgcaattcagcctcctttaatcagaattcaatcttgggctggggtggggagagtggagaatgaatgagaatgaatatgtctgtacagcggcctccttctgcccctgatacccagattcaccatgttctcctttccgcctgtcttcgttgtcttcctaacgccaggttcagtttgtgggctctggattctattatgcggataggcagtttctgtttgttctacagattcacaactgtatgatagcaactggatgttataactggaaactggagtagaataccaacataccccacacccatgttagggaggcagtgggaatcactagtttagaaaattcttgggactcagttctcttttccttttggcctcagtaggcgtcaagaggagatgggaaatctgattgacctgtcatttctgcacactgttataggcacgttgctgacaagcctcagtttctttaaacttgtttgtgttcagatcttttccaccagaccagcatcagcagggtctggtttgtggtgttacgtactcctcttctgttgggctggagctttgaacagtttgccctttataagcctgcagcattaatagggaaagactgaggggcacccaagggggtgtttatagaatgggctttataaaactcccagctgccccgtaaaacaagagttcctcgctaagggggcttttgagcaaaggtgatttcttcacaggagccgaacatacagtctctgccttcagtctcataaaaaccacatctcattagaaaagggacataattcaaaggacggctttgtatcacccaggtaggcaggagatctgggacttagtggctgtgaactcctgcattttcacagagacttgcttgtcctgcactttgcagcctgaactataagcctggctgtctttgaaaagcctgctgtgatggcatgctgacttgtcacgtggaaggctggaggcatattagtagcttctggaaggaaataaaaaatgtgagatgggagcttgggagtaagagaggaatgttagtgaggaagttaaaatgaaggcacaggagggctcccttctcctgcacaatgagttctagagagagagttaatggaaacatcccacccattgctcacactcgttcattttacagctaattcctgggtgcctactgatgccaagaggagaaagctaagacaccgcctgtccctcccattgcagagcttgcagtatacccttcattattacttaagatacttttttctctttacctcaacatggcactaaagatacttttccgtggtaacatttttaagagaaagtcaccttttaagggaaggcaatagcctgtgacctactgggctgtgttttcttcatatataaaatgagaatattaataatagtttcacagggtcgaaataatacatagaaacaataagcgcagtgcctggcacatatggtgagctctcttcttattttggtatcttcttcttgtatcttcttcttgttgcctcccgtgatggagtgccagaaggcatggaggaagaagaaaaaattaagatcccaaacaagataacagactctcttgatcaccaggattaataagcctgctgacttcactgtccagagcatccacatccataattagtattctccagcagctttagaggcgtgcatgggaactcctcagaaggaaataagagaaatgtagcttagggagaaagtcctgtaggaaattaaagtgaaaaagggggaagaggaaataaatacacaggctgatagcagctggttcactaaaaatctctttattctttgaacaatcaagagagtaggtgtggctgggcgtggtggctcatgcctgtaatcctaacaatttgggaggctgagacaggcagatcacttgagctcaggagttcgagaccagcctgggcaacacggcaaaacttcatttctactaaaaatacaaaaaaaaaataataatgtgtgtggtggtgtaggcctttgagaggctgaggtgggaggattgcgtgagtccagggggtggaggttgcagtggcctgagatcgcaccattgcaccccaccttggataacagaaccagaccttgttgtgaaaaaaagagagtgggctaggcgaggtggctcacatctgtaatcccagcactttgggagaccgaggcggacagatcacctgaggtcaggagttctagaccagcctggccaacgtggcaaaaccccatctctactaaaaatacaaaaatgagccaggtatgctggtgggcaccagtaatcccggctactcaggaggctaagacaggagaattgcttgaacccaagaagcggagggtgcagtgagctgagatcgtgccattgcactccagcctgggtgacagagtgagactcggtctcaaaaaaaaaaaaagaaaagagagtggatatggacagtgaccttcttaccttcaggttctcaaatcagagacaaagaaattaatttctcagagcgagagtgttcatttttcatgagtttagtcaaagttgtaatttttttttttttttttgagacagagtcttgcttttttcgctcaggctggagtgcagtggtgcgatctcagcaacctccacctcctgggttcaagtgatcctcctgcctcagcctcccgagtagctgcaattacaggcgttcgccaccacgctccactaacttttgtatttttagtagagatggggtttcaccatgttggccaggctggtctcgaactcctgacctcgtgatccgcccacctcggcctcccacagtgctggggttataggcgtgagccaccgcgcctggcccaaagttgtatttttttttttttttttttttgagacagagtctcgcggtcacccaggttggaatgcagtggtgtgatctcggctcacagcaagctccacctctcgggttcatgccattctcctgcctcagtctcctgtgtagctaggactatacaggcgcccaccaccatgcctggctaacttttttgtatttttagtagagacggggtttcaccatgttagccaggatggtctcgatctcctgacctcgtgatctgcccacctcagcctcccaaagtgctgggattacaggcgtgagccaccgcgcccagccaaagttgtaattttttaatgattatgttcttactgctcaataagagcttatcatggatcccaaggtgcacccccaagtttaatggccactggtaaaggcctggtcccccagtgagcccagcctgttgccctctttcctgcccactgagctctgtctgcagcctgacctgcctccacacattcagatgggaaatattatacagataagtcataggactctccctctctctttttttttttttttttgagatggagttttgctcttgttgcccaggctggagtgcagtggcacaatcttggctcactgcaaccttcccaggttcaagtgattctcctgcctcagccttcccagtagctgggattacaagcatgtgccacaacacctggctaatttttgtatttttagtagagatggggtttcaccatgttggccagcctggtctcaaactactgacttcaggttatcctcctaccttggcctcccaaagctctggaattacaggcatgagccaccgtgcctggcctggactctctcttaaaggagttaaggccatatgtttttacatttttaatttgcaaagctatgctggtcagtccttagaggtggtatatcaaggctgtagctgtattaaggtaaaatttgtttctctttcgaggtccctaatcactacttcctaattttgatcagtttgttgtaaaactcattttttatgcactgtgataattgggagagtggattaggaaaagacttgtatttgtttttgtttttgttgttttctcctcggaatgaatcgagcttcggtaatgttaaaatttaaaaataggctggaggccaggtgcagtgcctcacacctgtcatcccagcagtttgggaggccgaggccagaggattgcttgagccaggagattgaggctgcagtgagccatgattgtgccactgaacttcagcctggataacagagcaagaccctgtctctttaaaaaagaaagaaagaagaaaagaaaatagaatgttttccagagcacaagattgaagacagttgttagaactcctggattttctttttcttttttttttttttgagatagagtctagctctgtcgcccaggctggagtgcagtggcataatcttggctcactgcaacctccacctcctgggttcaagcaattctcctgcctcagcctcccgagtagctgggattacaggtgcccgccaccacgcccagctaatttttgtatttttagtagagacggggtttcactggattggccagactggtctcgaactcctgacctcatgatccacccacctcggcctcagttttttatgtcgtttcttagagttagtttcttctggttcaggtgaagaacactttatcttccttatcagaacaattttttactcctaccaaggccgcccggcgtgtgtggtgtggtggtgtgcgcctgtaatcccagctactccagaggctgaggcaggagaatcacttaaacctgggaggcaggggttgcagtgaactgagatcacaccactgcactccagcctgggcaacaagagtgaaactccatctctcaaaaaaaagaaaaaaaaaaaagaaaaggataggattgaaataaagatggccttcatcccaagaatccattgtaataatacagggaccatatcattaaaatatatagactatatactggctttctttttttttattattatttttctttaatagagatggggtctcactgtgttgcccaggctggtttaaaactcctgagctcaagcaatcctcctgcctcaacctcccaaagtgctgggattacaggcatgaaccactgtgcccagccactggctttcttttttcctgttttcttatgcttccaaaatctttatttggcttcaaggaactagataatcccaaatctatacctagtaatattattagtacagtttacccagccccatgtgattgggcacaaagcccagtacctgatttatggggccaagtggcattgggaactgcttcccaatgaacctacagtggagtaggtccctttagacttgcttttcattcttagcttagaaaacctcatatagggcttcctaagggaaaccatcatgccgtggtttcactctgaaattaaaaacatctccgaattttgtagcaggctaacttatggagcatattctggttcattgccttgcttagtaccagctcaagaagcaccagccgcatagcttccccaacgaatctggagcctagaaactggatactggacactcgaacagttctactcaggaaacattgggaattcgcagtgatgacttcctagtcctaacttaagtctccaccgtaggtgtatgttatttctgcagacaagcatatcactgtgttttagcagcttttatgcttggacaaacacaggtgaaggacctgcttagcaattgcatgttcattgtttaattccccgaagaaccctctgaaggaggaggtgatattagccctgttatatagataaggaatttacattcaaaaaagatgagttaaatatgtcctactatgtacctaaaaaaattaaaaattaaaatttttgaaacgacttatttatgatcacacagcttataagtggcagaattagacctccagaaagttaaacccaagtttcaatccttttgtatacatttatgcatgatgattgggtttttaccatcacatgtggtgagatgtgtctccctcaaacttgttatcacgtcagcatgtgatcagtctgacatggggaaaaaccccatcttctgactccaaatccaaggctgtttcacccccaacttttccttaacaaagaggcataaggtgcttgatttactgtggggactttcctgattcctgtgggccttggggaaaatccctgcctttcctgggctttagcttccccgttagtgaaacaggtctgataacacctctcccttcccactcttagggatgcatggggtcaaagaagtgatggattggaaagattatttgaaaacgctttgtaaacatgaggcattagattattactttcacaaatgttagcttctttagatttctgggcaatttcctttggttttattggaagaaatgtggtggaatttggatttttaaaaataatatttataacagcaatttcttgccatattaccacattgaactcaaatcctggaatttcccaaaaccacttaaaaattaattctggtcaagcagacagtttgtactcaaagcagtaaactggatccttgctcattaaaccagtctagactcccaggttgatgtgtcacattttgctccctacctttaaacagatataatcattgtggtcttgtggatttctcaagcgagctccttcaccctcccctctttcccttccttccataactcaggcttatatttagtattgagcttagttgcataagttcctacaggaataaggatctaagcccccatggctactctgttttcaaggcctcctagcagcctctgatgaaccaactgcaggcaccaggcctcccagtggcactgcccagttgtcccagtgtggtaacagtcctccagcctaaagatggcatcagcagagcagggtgtcattagtgaaaagcttctctacagtagaggccagttcaagaaactctagatatggtcccttggcagccttctgcaaagggtggaagggaacagcccctgaagagggtctagggaggcatctgaatttgaagggggtgggctctgtcagaacggtcagcagggctgcattgccacagctcccagggttctgtcattgacgtgcatgccccaaccttggtgccacctgtgtcactcacagagtctgatggttttgattaattttgttattcctactactgaaggcaactgatgagaagttaagacttagaagttcagaggagccaggtgctgtggtgtgtacctgtaatcccagctactcaggaggctgaggcaggaggatcacttgagcccaggagtccgagaccagcctggacaacatagcaagacccggtctcaaaaaaaaaaaaaaaagttcagaggagtctcatatgattgtgtactacaacagggataatttcgtatccaaggtttctggtgtttcagaaacagacacatgttgcctgcctgccggcgtctgagaggaaaccagtctatgctctttttttttttttttgagacagtgtctcactctgtcccccaggctggagtgcagtggcacggtctcggctcactgcaagctctgcctcccaggttcacgccattctcccgcgtcagcctcctgagtagctgggactacaggtgcctgccaccacgcccggctaatttttttgtatttttagtagagacagggtttcaccttgttagccaggatggtcttgatctcctgacctcgtgatccaaccgcctcagcctcccaaagtgctgggattacaggcgtgagccaccacacccagcctagtctctgctcttaaaggaagggccacagagcagcaagatgagtgttcccattactgacccccagaatcatgggaggagagcacaaacctctttcagatgaaacgatttaaactgagctttctggatggtaatagataattgatactctttcagcatctgcaagacattgtcctcaaagtgctggtgaaccatctgcccacaaatgacccccacatctcctaccctcagacctgttgagtgtaactaccttttccctttgttttctcagcattaataataagctacagcagccggaggcagcggccggagtgttagaatatgccatgaaacactttggagagctggtaagcacctcattctttttggaaaagcaggaggtagagacttggctagggagttattggccctgaccttcagaaagttactttccttttctgttttttctccaattcataacagtctgatgctgacttaccagtcttcaggatgaaagagataaactgtgtctgtagggactctatttctttcttttttttttttaattatactttaagttctagggtacatgtgcacaacgtgcaggtttgttacatatgtacacatgtgccatgttggtgtgctgcacccattaactcgtcatttacattaggaatatctcctaatgctatccctccccgctccccacactccacgacaggccccggtgtgtcatgttccccatcctgtgtccaggtgttctcattgttcagttcccacctatgagtgagaacatgcagtgtttggttttctgtctttgcgatagttcactgagaatgatggtttccagcttcatccatgtccctacaatggacatgaactcatccttttttatggctgcatagtattccatggtgtatatgtgccacattttcttaatccagtctatcattgatggacatttgggttggttccaagtctttgctattgtgaatagtgccgcaataaacctcatgtgtgcatgtgtctttatagcagcatgatttataatcctttgggtatatatccagtaatggtatggctgggttaaatggtatttctagttctagatccttgaggaatcgccacactgtcttccacaatggttgaactagtttacagtcccaccaacagtgtaacagtgttcctatttctccacatcctctccagcacctgttgtttcctgactttttaatgatcgtcattctaactggtgtgagatggtatctcattgtggttttgatttgcatttctctgatggccagtgatgagcattttttcatgtgtcttttggctgcataaatgtcttcttttgagaagtgtctgttcatctcctttgcccactttttgatggggttgtttgattttttcttgtaaatttgtttaagttctttgtagattctggatattagccctttgttagatgggtagattgcaaaaattttctcccattctgtaggttgcctgttcactctgatggtggtttcttttgctgtgcagaagctctttagtttaattagatcccatttgtcaattttggcttttgttgccattgcttttggtgttttagacgtgaagtccttgcccatgcctatgtcctgaatggtattgcctaggttttcttctagggtttttatggttttaggtctaacgttgttaagtctttaatccatcttgaattgatttttgtataagatgtaaggaagggatccagtttcagatttctacatatggctagccagttttcccagcaccatttattaaatagggaatccttcccccattgcttgtttttgtcaggtttgtcaaagatcagatgattgtagatgtgtggtattatttctgagggctctgttctgttccattggtctatatctctgttttggtaccagtaccatgctgtttggttactgtagccttgtagtataatttgaagtcaggtagcgtgatgcctccagctttgttcttttggcttaggattgtcttggcgatgcgggctcttttttggttccatatgaactttaaagtagttttttccaattctgtgaagaaagtcattggtagcttgatggggatggcattgaatctataaattaccttgggcagtatggccattttcacgatattgattcttcctatccatgagcatggaatgttcttctatttgtttgtgtcctcttttatttcgttgagcagtggtttgtagttctccttgaagaggtccttcacatcccttgtaagttggattcctaggtattttattctctttgaagcaattgtgaatgggagttcactcatgatttggctctctgtttgtctgttattggtgtataagaatgcttgtgatttttgcacattgattttgtattctgagactttgctgaagttgcttatcagcttaaggagattttgggctgagatgacggggttttctaaatatacaatcatgtcacctgcaaacagggacaatttgacttcctcttttcctaattgccttttatttctttctcctgcctgattgtcctggccagaacttccaacactttgttgaataggagtggtgagagagggcatccctgtcttgtgccagttttcaaagggaatgcttccagtttttgcccattcagtatgatattggctgtgggtttgtcataaatatctcttattattttgagatacgccccatcaatacctaatttattgagagtttttagcatgaagcgctgttgaattttcttgaaggccttttctgcatctgttgaggtagtcatggtttttgtctttggttctgtttatatgatggattacgtttattgatttgcatatgttgaaccagccttgcatcccagggatgaagcccacttgatcatggttgataagctttttgatgtgctgctggattcagtttgccagtattttattgaggatttttgcatcaatgttcatcagggatattggtctaaaattctcttgttttgttgtgtctctgccaggctttggtatcaggatgatgctggcctcataaaatgagttagggaagagtccctctttttctgtttattggaatagtttcagagggaaggaatggtaccagctcctctttgtaccactggtagaattcggctgtgaacctgtctagtcctggactttttttgtttggtaggctattaattattgcctcaatttcagagccagttattggtctattcagggattcaacttcctactggtttagtcttgggagggtgtatgtgtccaggaatttatccatttcttctagattttctagtttatttgtgttgaggtgtttatagtattctctgatggtagtttgtatttctgtgggatcggtggtgatatcccctttatcattttttatgtgcctttttgatatttgattcttctctctctctctcttttttttttttttttttttcaggcagagtcttactctgtcgcccaggctggagtgcagtggcatgatctcggctcactacaagctccgcctcccaggttcatgcaattctcctgcctcagcctcctgagtagctgggactacaggcgcccgccaccaagcccggctaattttttatagagatggggtttcaccgtgttagccaggatggtcttgatctcctgacctcgtgatccgcccacctcggcctcccaaagtgctgggattacaggcatgagccaccgcgcctagcctctcttttcttctttattagtcttgctagtggtctatcaattttgttgatctttttcaaaaaaccagctcctggattcattgattttttgaagggttttttgtgtctctgtctccttcagttctgctctgatcttagttatttcttgccttctgctagcttttgaatgtgtttgctcttgcttctctagttcttttaattgtgatgttagggtgtcaattttagatctttcctgctttctcttgtgggcatttagtgctataaatttccctccacaccctgctttaaatgtatcccatagattctggtatgtggtgtctatgttctcattggtttcaaagaacatctttatttctgccttcatttcattatgtacccagtagtcattcaggagcaggttgttcagtttccatgtagttgagcggttttgaatgagtttcttaatcctgagttctagtttgattgcactgtggtctgagagacagtttgttataatttctgttcttttacatttgctgaggagtgctttacttccaactatgtggtcaattttggaataagtgcgatgtggtgctgagaagaatgtatattctgttgatttggggtggagagttctgtagatgtgtattaggtccacttggcgcagagctgagttcaattcctggatatccttgttaactttctgtcttgatctgtctaatgttgacagtggggtgttaaagtctcccattattactgtttgggagtctaagtctctttgtaggtctctaaggacttgctttatgaatctgggtgctcctgtactgggcgcatatatatttaggatagttagctcttcttgttgaattgatccctttatcattatatagttgccttctttgtctcttttgatctttgttggtttaaagtctgttttatcagagactaggattgcaacccctgctttttttttgttttccatttgcttggtagatcttcctccatccctttattttgagcctatgtgtgtgtctgcacgtgagatgggtctcctgaatacagcacactgatgggtcttgactctttatccaatttgccagtctgtgtcttttaattggagcatttagcccatttacatttaaagttaatattgttatgtgtgaatttgatcctgtcattatgatgttagctggttattttgctcgttagttgatgcggtttcttcctagcattgatggtctttacaatttggcatgtttttggagtggctggtaccggttgttcctttccatgtttagtgcttccttcaggagctcttgtaaggcaggcctggtggtgacaaaatctctcagcatttgcttgtctgtaaagtattttattcctccttcacttatgaagcttagtttggctggatatgaaattctgggttgaaaattcttctctttaagaatgttgaatattggcccccactctcttctggcttgtggagtttctgccaagagatccgctgttagtctgatgggcttccctttgtgggtaacccaacctttctctctggctgcccttaacattttttccttcatttcacctttggtgaatctgacaattatgtgccttggagttgctcttctcgagaagtatctttgtggctttctctgtatttcctgaatttgaatgttggcctgccttgctaggttggggaagttctcctggataatatcctgaagagtgttttccaacttggttctatttcactccccgtcactttcaggtacaccagtcagacgtagatttggtcttttcacatagtcccatatttcttggaggctttgtttgtttctttttattttttctctaaacttctcttctcgcttcatttcattcatttgatcttcagtcactgataccctttcttccagttgatcaaatcggctactgaagcttgtgcatgcatcacgtagttctcatgccatggttttcatctccatcaggtcatttaatgtcttctctacaccagttattctagttagccattcgtctcatcttttttcaaggtttttagcttctttgcgatgggtttgaacatcctcctttagcttggaaaagtttgatcatctgaagccttcttctttcaactcatcaaagttgttctccgtccagctttgttccgttgctggcaaagagctgcgttcctttggaggggaagaggcgctcttattttaagaatgttcagcttttctgctctggtttctccccatctttgtggttttatctgcctttggtctttgatgatggtgatgtccagatggggttttggtgtggatgtcctttctgtttgttagttttacttctaacagtcaggaccctcagctgcagttttgttggagtttgctggaggtccactccagaccccgtttgcctgggtatcaccagtggaggctgcagaacagcgaatattgcagagcagccaatgttgctgcctgatcgttcctctggaagcttcgtctcagaggggcacccagccgtgtgaggtgtcagtcggcccctactgggaggtgcctcccagttaggctactggggggtcagagacccacctgaggaggcagtctgtcgcctctcagatctcaaactctgttctgggagaaccactattgtcttcaaaggtgtcagacggggacatttaagtctgcagcagtttctgctgccttttgttcagctatgccctgcccctagaggtggagtctacagaggcaggcaggcctccttgagctgtggtgggctccacccagatctagcttcccggccactttgtttacctactcaagcctcagcaatggtgggcacccctccgccagcctcgttgcctacttgcagtttgatctcagactgctgtgctagcaatgagcaaggctccgtgggcgtgggaccctctgagccaggcacgggttataatctcctgatgtgccatttgctaagaccgttggaaaagcgcagttttagggtgggagtgacccgattttccaggtgccatctgtcacaacttcccttggctaggaaagggaattccctgaccccttgtgcttccggggtgaggtgatgcctcaccctgctttggcttacattccatgggctgcacccactgtcctgcacccactgtccgacaagccccagtgagatgaacccagtacctcaggtgaaatgcagaaatcacctgtcttctgttttgctcacactgggagctgtagactggagctgttcctattcggccatcttggaacctccgggactctatttcaactgagataaatcatgagggtagattcgaatatgcagtaagcactgattttatcaaactcttagcatgtgcctggccctttgctaagtgtttatatacttcatgtaatttggtcctcacaacaatgctatgaagtggttactgctgttatcacattttacagatgaggagactgtggtcccagcaggttaagaactggtctgagctgacagagtgaataaatggcaaaaccaggacttgaaccagggcagtctgacattagagccaaggcatttgcccctgatctgagttatctttgtgggagcttgcattgtgtctaggccacagttaatcctcattagatacttaggaagacaccatggccagttctttagaaaatatatgctatacagccgggcgcagtggctcacgcctacaatcccagcactttgggaggccgagatgggcagatcacctgagatcaggagttcaagaccagcctgaccaacatggagaaaccccatctctactaaaaatacaaaattagcctggcatggtggtgcatacctgtaatcacagctactcgggaggctgaaacaggagaatcgcttgaacctgggaggcagaggttgcagtgagctgagattgttccattgcactccagcctgagcaacaagagcgaaactctgcctcaaaattaaataaataaataaataaataaataaataaataaatgctataattgatatctgacattgaggcatttttgtgttcgttattgcttacatagctacttccctactgccatctttggcctaccaatatttaaaatgtttctgttattctttgtggtcttggcagggcattttgataacttctaaaatacataaagcatcaacttaatcaacttagtattgaattcccggaaggtcaagaagctctaggtccatatatttgatgaggaaccttttgttagtcatgtaggttgcttactgtggtttgccttgatcaggagcccaggatgttaaagtgagaaaaattgggaatatttaacaggcttttgtttgatcatctagcattttgcctcacagaagggctagtaaattagaatgaaacgtttaagggaatcttttgccatctttagcacatattatcttttttttttttttgagacggagttttgctcttgttgcccaggctggagtgcaatggcacgatcttggttcactgcaacctctgtctcctgggttcaagcgattctcctgcctcagcctcccaagaagctgggattacaggcatgcaccaccacgctcagctaattttgtgtttttattagagacagggtttctccatgttgatcaggctagtctcaaactcccaacttcaggtgatcctcccacctcggcctcccaaagtgctgggattacaggcgtgagtcaccatgcccggccttagcacatattatctgtaactagactaagacagcatttctccagatgatggcaaagtgaaagaactgtatctcatccatccttgccacaatccttgtttgcagagagaagaaagtagggcgagagggcacttcatgtactccacctagatcaagatgttctctcctttgatatccttagagcttagatcccagaaagtgctgattgagacaaggactccgcagacatctggtattcaggtgatctgccagcagatggtttataccccatactcagaatgccagccttcaatgaaactttctccatcttcaggttccaaaggtggccattagctccaggattattgctaaacccgtggtggattttctgttatatttaggtagctgaatcatattaagcttgacaatggtttgctttttcttggcagagcctgtgctttctcagcaaaatttatgtaacagcaatctcattagaattttgtgtgattcattgcttaaaccttatagtttctgcaatctattgcaattcaaaatatcctaccctctaactgacccactccatatgctgccccctctcccctctgcttgttcatgctgagtgtatgccacacattagtcaccagctcttttttttttcttttccacccagtatagaaatttgaaatgagaaatctaccagtgcttttaaattttacagtttttgtagtcactcttttctaagtttcctattttgtatttaggcataatagatattattttggacaggcttgaaaaatgaagttgagatagtttgccttacgtggagccagatacgctcattttgacaaactccactgatgttgggcaaagactttatggcagcagaattagtctaggggcagcagagggcaccggctctgccagaaagaaccagaccagggaaagtaggtacaagggaacaatctccagcataaatgacctcttttcagaggttcaagaagaaaagattattttagacagcttagcagagtagttgttagtctacatactgcacaaacatcagttttatttagaaaggggaagagggtaatgggagaaactagtgtgggccctgcattatatcatacaaatttcactgcagaatcatattgttgttaaagaaatccctagagagccactacccttgcctgtgcaaaaatagtcatttattgagctgatgatgaagtagcccaggagagaaactgagatggagggcagacagaaagcatataaaacagtctcctgatcatttggagagtcatagtctgtaactttcagactgtagcacctcctactcacgtggacaacaccctctttcagcgtggcacaccatctgctagggtaatgcaatccctcctttggcccctgacactggtaaacctgccaagcactgagcaggagggaacattggatgggagactctagaagaataggggaaagactttccaaaaaattttaagtggtggtgttatattttaaattattatttcctgaaagtttaaaaatgtgttaagaaatgcagagaatcaagaggttttaaaaagtgagttgttttcctccagcaaacatgcagatgaggcctggtctttctggccccatcaggaaagcagctgccatccctgtcaagtagcccagtgtcagcactccagcagatcggagcgatggcacctttgaaacagctctggtgctctggtcgacgttttcttctatccctaaatgatttcccctgtcctcgtgtccgcacatcctggtgtgttctgatgacccatgtgtctgggaaggaaataatgggggaggagtatggcctgacgtctgctctcagataatcccaggatcattccatccatgtgccaaggaaccagtatttccaccttccccaagcagcactcctccagattgtctgaagttgtttttaatccaagcccagacgtaactgtgataggtgaagtgtcatgcgtacagcagcacattaggaaagagagtatgtggattaaacaatgtgatcccttcttctgtgtacctcacagcagtctttctttcccatgtaacaggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggtgagcttcctagaatgatggcatgtgtggctgcaagagacttcgtctctttgatctttggatttgaaaggaaggcttcccccacacggagttctcacttcagactcattttgttccacttgatacttctcaggaatcatctttcacttttggtgtttttctctctccattctatctcagctgctaagtaggaaggccttcacttaaactttataatgctctaggacaggacagccttgggggtgctcctgggatctacttctcctgtctactctcctgatctttactgacccagtgaagagctcatattcttacttggcaaccttgatttttaaaattagctcttgcagatgtttttaggaaagaagaacagtgactgccctcttcaggtgcttcacctatatcggatgcttactgtgtgtccagcactatgccaggctccaaggaaacaaaatacacaagaaaacaggcctttcctgtgaggttgatgagacagccacgtaaacagagggttattatagcctgagaggatgttgataatgtgtgagccatgctaccttgaaagcccaaaggaggaagcagggtgattttgcagtacttcctggaggaaggactgagagttaggagataggtaaaggagaaggtaggggggaaacaggtactccaggtgcaagcaacagtaatgcgtaactgagaacatttggctaggttttggtttggagagttgcttgggaatgttgaagggaaaaaatggcctccattctgtttccaacaacaagtgcaacttttaaaaaatagttaaaataaaatgctatactatcattgttaaactgtttaattgcagttttaaatgactaaaacaggccgggcacagtggctcacacctgtaatcccaaccctttgggaggccaaggcaggcagatcatttgaagtcaggagtttgagaccagcctggccaacatggcgaaacctcgtctccactaaaaataaacaattagttggacgtggtgttgcaggcctatgatcccagctactcgggaggctgaggcaggaggatcacttgaacccgggaggcggaggttgcagtgagctgagattgcaccactgcactccagcctgagtgacagagcgagactctatctcaaaaaaataaataaaaataaataaaaaataaatgactaaaacaaagttccatgctacctcccgactagtaggttatccctactttctctggaacatgatactgagggtccactgttccttcattcagccagtaactatcacgtgtgtattcattgctagtgtctgtgccaggccctagggacacagccgtgagcaagacagttacagcccctgccttcccagagcttacaggacagacaaccaggctgtcgtaatacattgtgataccatcaacaaaggagcccagggggctgtgggtaataggaagcaccctaatcagccttggaagataccgggaggaaacgatatctaacttggacctgtacaatgaggagcacagtatgggggattttagtaggcctttttgttcagattcttctctgtgaccccctgtcttcagaaataaggatgtacctttcctccaggtatagggaaggcatctctcacatgagggccttatgacctccttcaggggaaggtcaaaatatttttcctagatgttatgacctgcttcagggcataagggcaggggagagtcagagtaattttttttttgctgccattttcttaaaatattcagtatgccaaggtgtgctatatttcagggtagtatatcctgacccccatcacagggatgtacattgacggcaatgtgctgtttttaccattttgcactctgctgttttcacttaacaagtctaggccactgttccatatcagcacctgtagagctactgcctcatttttttaatagctgcatgatatttcatcatatcaataaatctagtctatttaatataggcaaatttttatttgtttagacatttcctgccttggaaagtttaagaatattggacaaaatcaaagcaagtcttctaaaagaatgatctttcactacatgtagtctttctgtactaccttagcctcccttgtagctgagactacaggcacacaccaccacagtgggctaattttttttattttttgtagagacggagtctcacagtgcagtctaggctggttttgaactcctggggttcaagcgatcctccctctttggccttccaaagtgctaggattacaggtgtgagccactgtgcacagccttcttttttttttttaagaggcaggactttaatctgtctctcagtggagtacagtggtgtgatcacagctcactgtggtcttgaactcctggactcaagcaatcctcccacctcagcctcctgagtagaaggaactacagaaatgcaccaccatgcctggctaatttttttaaattattttttgtagggatagggtctcattatgttgccaagtttggctgttatttcttaatataaagttattactacatccccaagatagaagatgttataatgttttgcatgaaaatttaatgttagtgaacttagccttaggcattggatcatatatatatatatagagagagagataatattccacaattatttgaaagtggcttgtctgatttaaccatcattaggaagatttttatttatttttatttttttatttataggaagtgttatttatttttataaattaacattaaaaggctgagtgtggtggctcacacctgtaatcccaaaactttgggaggctgaggcaggcggattgcttgagcccagaagtttgacactagcctgggcaacaacaacaacaacaaaaattccatgttcatttacctcaataaatttaggaagcatgtcttttaaaatccgctttatttatttatataactaaagctgttgttgttgttgttgttgttgttgttgtttttctcagtccaacaagtatctaaagctgtattgtccaatatggtagcctctagccacatgtggctttttttttttttttttttttttttttttttttttgagacagagtctcactctgtcacccaggctggagtgcagtgctgtgatttcggctcactgcaaccttcacctcccagggtcaagtgattttcctgccccagcctcctgagtagctgggattacaggcatgcaccaccactcccggctgatttttgtatttttaatggagatggggtttcgccatgttggccaggctggtcttgaactcccaacctcaagaaatccacctgcctcagcctctcaaaggtctaggattacgggtgtgagccactgcatccggccttatggctatttttaaattaaataaaatgtgaaattcagttcctcagtcataccacattttaggtgttcagtagccacatgtggccagtggctgctgtattggacagtgcacatagagaacttttccatcatcacagaaagctctattggacaacactgatctaaggcaaaagaaaagtatgttaataagaattattcacattatttcaaaaacaatttttctagtttcatatcagctgtcatacaatcatatcttgttatgagaaagataaaacgagatggaagtgttcttaaaattcaactctaatcaattcctaaaatactgcaatgtttcactgttgccattggatttacatataaatatcacagcctggcattcaaagtctttcctattatggcaccagcccgcttcttcagcacatttctctgtgaacccttctctcataatctgagctctggccaaattattcactcttctccaagtcctacattcattcgttccattctttgaattgccctcttcttctgcctctttttctacttttgaaatcctctccactgtttcttccctcttgaaacctctcctggccccttccccttccactcctctcctttcacattaaagatgaccacttttcctagctgggttagtaggagcagattggatacattggaagagacttgagatgtggtttcctccaattcctttgtgttatagttgaagaaactgaggctcacagaagttgagaaaaccgagctgagtgatatcgatctcctgatgttgccgttctcagtcccaaatacctctgacctcttgggttctttcccgttttatagttacccataaagcgtaagtggctagtaatgctgaccaaaggatttaagaatgtgggagactaaaagaggttttaaaacgagtcgaaagaagcagaatatttttcctgatcatatgcaggtttctgatacggtttattcagtcaattctgtttagagtgcaatttctaaatttcttttgtgtgcgtatatcctgaatatgcctgaggcagatgttcatcagaggtgacatgaagaatgatctggagttaacagtgagctgagacccttcccatttccccattcttatctgcctccaggaggctgtgagagctaggcaacccgcaccccctgccctgcccagccctatggtgcaggcagccacgcctgcagccaaagtgaactaaagtgaaccccctcctcaaactgttccccacacagctaggtattgagaacagattcctcgggagtgagatcataggccctcaaccctaagaggagccatggagccattccaaatgaaagaatacttcctaagctttttatttgttttaaatcttcagggaccatgctatcttctctgtatcgttccaattttagtatatgtgctgccaaagcaagcactttctgcgttttttgccagaacgctaggtgagaaaatcaaagtctcagagggtgcaccttttacccagtcctccctatgtgtagtgaaaggtatcagaaaaacatatcctagttgttgccccataaacaatctcatgttttttatttatttggttttgcccctcacccaaccgcccttgagaggtaggactcggtatattctgcattattttttgtaacaaatgtttgtggaagagcaatgctcttgtctgtgtactgaccaagctctgacagctgttcccacacagctgcatggtgaaggccgtgcgaagttcactgctagggcttgcccttctgagttgctgttcctacattgctccctgctttgtctcttggggtaacaatttgtgggatttcagcactgaacttgcttcctgctctagatgaaaagaaaaactacatccttggtcctgtggcacagaatcagctgatcctgaaaaaataatagagggaggctgagttaggagggacagcttgcatgaaggatgcattcatccaagttgcttgtgttcaccagctgtaaattttgaactatagggttggaaggaccatttggagatcatgcagctttctcttctacctctagtcaggatccctacaaaaatatgctgtgcagatgagctgtgcctctgctttaagccagtggttctcaaactgtgacccctggtccagcagttttggcgtcacatagaaacttgttacaaatgcacattctcagcccctatcccagcactactgagtcagaaactcagagggtggagcccagccatctgaaggagccttccaggggattctgatgcacttagtagtttgagaactactgctctaagggaacccctagcagggcccaggagtgaagatttctaagtctcggtggcagaaaaccaacacagaggtttatcctgttcccttaatggagcttcagtaaatgcatagggcatcatggccaccagttcctctttttggtctcagctctgtattgagccagagtgcttgtgtccctggttttcttgatcatgtgccagagagacaacaaagttaatatctgctgtactgcgatgctggataatggccctgagtacctctcctagacgtggtccacagtcagcttcttggtgagctcagcctagattccttctgcagggattgacttcagcatctttatccccgccctcctacaccaccttagagcgcagagcagctcatgttccatgaggcttctgacctgggccacttagaacttgtgcccttcctcatgactccacctccagtgccaagttaaatcatcctgcagcatgtctttcagaaagcaggaaaaattaaggagtctcctagctgcatcttgaagctcacatgaaatcagggactcggatttgcataatagccgagtttattttttttcactgagaagcgaaacatgagagggagtttcaacagtcaatagcaagaccctattctagggcttccatgttctcttttgtttattggggtgtcttaagcaccatttatatctcaatatctcatagtcctccttcacattcagcaattgtgcaaaattctgagaataagcagcatctagccttctggtagatgtgaccattgtggagcctttcttctgtacagattgatttcatctgggtttcatttcagactgaaaccattggtataatacttttaggagccagttctccctatagctttgagcccaaaatgaatgtccagatgcgtctcttcagattgctttctctgtaggcctgccaaagggaaagagcccagaattctttggcataggagagaagttatttaatatttatgaagggagccagatgttcaggacatttcatatcacagagttaggcatggttcctatccttgataaactcacccagaaaaattacgagcaccgggtcatagaaacaacaaatagcccattgattctctattatgtgacctagtagaatgtgcttttttattaaaggtgtccagttcctgtgtagtcctcacctaacccaaggggcaggagaggaagattggtagtttaaggagatttggattaatctgactatataattcattttcttcaggggtcaactccaccagcagtgctgtgaaaagtggaccctggttaatgatgagacccaagccaagatggcccggatggctgctgcagctgcatggggtttaggtaaagcttccctcacaggcaccctgtgaaggatggggtgagtgaggctaggggctcgcatcagaagagtaaggtgctggctctaggtggagatccaggtctatggaagggtaaaaatggtctgtatccacctacttctacaccttccatctgtcctttctacctgatgatttttattaacttgttagacatggggaaaattggatcttcgtggggaggccaaggaaaacggtgcacaaaatcaaaattgagatttagtgggtgagaaatacacacttagcacactcacacagagtactgatgccagatgcttttccccatacaccaagccattgtcagcagacaccagtggggtgtcaagtagttcatttctgaccctatctacttggaaatagcatcagatcccacaggttaagggctcgtcccaaaatatcgccccacttcagatgccaatcacaagttccaggttgtgatttgtgtttctcactgaccagctgtaaatcagggttcccataaccctcctcctcaggtttgataatttgccaggacagcgcacagaacaaaaggagacactttacttacatttacttgtttattataaagggtattataaaagatagaggtgaacaggtacaatgagggaaggtctagaagggtctggagtgcagaagcttctgtcctgtggtactggggtttaccttcaccctcccagcaccttcatcaacctggaagctcattgttcaagagtttttattgcctttcattttcaacctcttcacctttcctggatgtcagtggacagggctgaaagttcaaacccgataattacttggtctttctggtaaccagccccatcctggaaggggagaaggagggagagacaacataaggcaaagtttaagtcacatgctactcaaagaaccaatctctttggcaataggtttggatattctgtaaggttagtggggtcatacacctacacattcagcagggtactagggtatgcatctctgtgtctccatttacctcttcagttgaaagcagaaataagagatgagaaggctgggtgtggtgactcacacctgtaatcccagcactctgtgaggcttgggcaggaggatcacttgagcctgggagtttgaaatcagcctgggcaacatagtgacactccgtctctaccaattatatatatatatacacacacacacacacataattttttttttttgacatggagtctcgctctgtcacccaggctggagtgcagtggcacgatctcggctcactgcaacctccgcctcccgggttcaagcaattctctgcctcagccctccgagtagctgggattccaggcactgccaccacgcccggctaatttttctatttttagtagagacggggtttcaccatgttggccaggctggtcttgaactcctgatctcgtgatccatcctcctcggcctcccaaagtgctggaattacaggtgtgagccacagcgcctggccctaatttttttgttttaaagagagatgaggagaagtctctttaggttcctgcatccagctatgcctaaagtcataaagtcatctctataattggccttctcaataaataaattttttcttaaaccattctaccttatcagcataatcaataggggctcattatgaataataaggaacactcctatcactcaggaaatgcagagggttttcgatagctctataccaggcactagggacaaagaccaaacatatttcttcttataccacagtaggatttggctttggttgcctaaagcaagttactgatttattttcctctcttataaatgaaatctagaggtaggcgtttccagactagtattttggctctattatcattagcctttttccagatttctgctttgcatgctcagcctgtgcctccattttaaagttcacctcatgatctagcatggttgctggagctccccatgtcatgtttttatcccaagcaggagtaaagggagaggacagttgtctatctccttttgttaatatttcttaacttatattttagaagtttcccaatatatataacagtgaagattatagtataatgaacccccatctgcccttcaccttttccagcaattacaaacattttcaccatttattgtctctattctcctctttgctgcccaattcttacccacctcactcctactcccagagttaaagcagagatttcttttcaatccctgcagacatgtcaatttctgacactgggccccagctaagaggattatcaatatgtgttccagagatggtcttgataagaagggagaggatatgtagagtgtccctggtgaaaagcttcacctgtggagcccagaggaaagttacataaccctctacctccattctcttcttctgtccctcttgagtcacagagcttataccttttcctttcatgagctctgggctagatccggaacatttttcaaaaacaaattgcttcctgaggagtcattgttagctatttgcttgtgcccagaggcatagagaatggatgagccgatggtctggagtgagaggcaggaaaatgccttgtaaaattggcagccctgtagtttgcagctaatgaagctgatatcttttctgcagatggtcttggaaatcctggcatttgatacatactcaccccaccaggagaagtgaatacctcttcccggatgcagctatttcccctgtctaatatgacacagagagcactgtgctgtgacctagcccatctttttgttataatggagtcgcttaaaatatttatctcctccctcctgaggcaaagatattgccagaatgaaatgggcaagctatttgtctaatcctgtcccctatcatctgtgtaatggctattttcaattgtttggaggaaaatgtaatttcccaacataccattgaactgtgagctgtctcagtagtaaggaagaaagcaattttgttgcacctggagaaacctgagagagttctctaggataatgaggtgctgcattcagcccacttgatgtgcagacttcacactcatgcacttcccctcatctgtcaagatgctccctgggcagccatggagtcctgtgtttggaagacagatgtggctttttttgttacgtcaacctgcatagtgtgtataggggctctgcaggattttggtacatgaggctcaagtttatttgtcttgtttatatcccaagctaaaggaaaataacagtggcaagacctatttgagatgctgcctttagcccaaccagccaacccccttgatgctttctgtctatgtgtgtgtgtataggtcagtgggacagcatggaagaatacacctgtatgatccctcgggacacccatgatggggcattttatagagctgtgctggcactgcatcaggacctcttctccttggcacaacaggtaaaaacctacatgcacccagatctctcagtgaggaaaagattgtagtgcttctaagcccacggtgctttggtaaagaagctgttgtacttcaggtctgatgtttactcggctctgtgagtacatttgaatggttttgattcattgtactcagaagactgccagcaggtcagctggggctacttattgttctgagggggacaaatgtgttctggaagctactgtctcggttgcttttggagttagcttgttgggtcatcattgtatcccttgttgtagagagaagctgtatttcaccaaaaggtaggattcagagagccagcagtctcactgtgcagcagaaatcagtcacaagggtcttctggccatgcaggcatgtcacacattgattaccttccgaggaagctagtacttacttgcaaatagtatgctgacttgcagaatgaaataatgaagtaatcccatccaccctgtcagacacgagacttagatgaggtgcccgggtaaatgatctgagcacttaaggcattgggacactataattaaccttaaacattcaaaaacgggaaaagctctgcatgaggggtttttaattgatggcacagaggaaactcaaactgcagtgctgtggcgtcccccaggctgtcatttgtgtgatgagaaccaagatcttggttgtctctgtcagagacacaagagttcccgttcatgtggacaggattgggcatctctgccaattttagagacactccctcataaggacagagggtgcttggaagccagagctaccacctggagccattgaagtgtgtgcagaatctattttcataaacatgcccagcaagaaaaagttaaaagagggtagatccccaggctgggcacagtggctcacacctgtaatcgcagcactttgggaggccgaggcagacggatcacttgagaccaggagttcaagaccagcctgcccaacatggtgaaaccctatctctactaaaaatacaaaaattagctgggcgtggtggcgggcgcctgtaaccccagcatcccagctacttgggaggctgaggcaagagaatcgcttgaaccaacatggagaaaccctatctctactaaaaatacaaaaattagctgggcgtggtggcgggcgcctgtaaccccagcatcccagctacttgggaggctgaggcaagagaatcacttgaacccgggaggtggagactgcagtgagccaagatcacaccactgcactccagcctgggtgacagagtgagactgtgtctcagaagaaaaaaaaaaggtagatccccagttatgtcctcttgagatgcaagaacccatgattcggttttacagcccacattcccatgcccttgccacctaagattttgtgtgttaccttagatagatgttaaagttttggatgccatacatttctgtcttcgatgagaaattggataatacctcctctcaagccattcagttctgggaaaatgcccttcttcagcgaatttctcttttcatccctggaatttaagtgagacagataacttctgtagctagttggggtgccctaaaataaccctaagctcccaagtccagagctcagtcccaagggttgtttctcttctagtgcattgacaaggccagggacctgctggatgctgaattaactgcgatggcaggagagagttacagtcgggcatatggggtaagtgtcaactattctgcacattgtcatttttgaggcttatttccatacttttttcagaatgggctattgagaacttacaggctgctggaaacatgcctgttttctctgatcggcagataccctgacaccagaaggaatgatttaaattaatcctgtcttttggattgttcgtgcagtaattagtttggaatgcgatttgcttccatcaggccatggtttcttgccacatgctgtccgagctggaggaggttatccagtacaaacttgtccccgagcgacgagagatcatccgccagatctggtgggagagactgcaggtgagcttggtagaaagccccagttcacccacctacccctaccccttccaggacctgggtgagtgcccccctccaccctggctcttactcaacttgcttttcctgcctcctccaccagtgtattgcttggcttgcatacaaagcagaatactcaaaggctcctcacctaaacttaagctgtgagagaatgaccaattttccagtctcttcttattctcttctctaggaattaaaaaaaatgttttttagtaagtgacacctgtgaccagtcagttaggaatcataacctcaaaatcagtggttctcaaccttagcagcacagtagaatcacctgcagagctttttaaaatcctgatgcccaggctattccccaggccaattaaatttctggggagagactcaggatcagtatcttttcaggttcccccagatgattccaccagccaaagctaagaaccactgctctaactacacttggaggccacctgcaccagcttttcagatggggttgccttaggttctcattggccaactcctgctatttatagctacttaggtcagagtcatcctgtcgcctctattgagactttttttccactgcagtttccgcttgtagagaagaagctttcccaagtcctccatcagtgttttcattctcacattctgatcattttaacatgagaaactgtcactccaggtctgggagtcaggagtctaaatgttctgggaaggatggccagttatcccattccttgagtgggtaggataggaagttcatcagcaaaagaaaaaagagagaaagcctttaccactccagaaaccatttgtctccctacctatcataatccatccccgcaattataaaccatttcctctcaactagatccccacctaattgctaattccaaattctcgcttcagaccaatcgaagtttgtgcttccaggcgtctcagtaggctgcgacaaaaccttttgactccaaacacattcctgcctaggaagctcagggcacattcccctggacaggaaagagcaatgggcaaaggaacatctatgaatacagtcatttatggctctgaaatgagcacatacagttcttttacatgttttattagtaattatgaaatgcccagtgaatgttttgatggaaaattgtctcctgggcaccagaaagaattccacaaataagcatttcccacttgatttgtacacaccccaattctttgaaatgcctataataaagagtttctacagcagagatctgatgaaccttctgacagtctgacaagtgggacttgatccaaatgtagacttagaggagaggggaggcatggaatgagtcctgggaagagagatgcctgtctgactttactgggtccaggacactgccaccaccttaagccttcgtgactttcctatcttgaggcagcaagatgctttttggcttacaggaggccaaattatttcagcatctgggagaacctaactattggtgagaagtcacaaaaaacaatcactcagtgtacttttttctgttattttaactttcgaaatcattgttgtcacacttcatgggatggaaaaatgtggtccaaggcctgaaagtaaagcattacatttatagtggaaatttggctcttgtggatggcttgctgtcaaatgaactgctggggctagaaaactgccattttgtggtcaaatcatttaaattaaaatccccaaccagtattatagtagtggaaaaatacatgtttgttacttttctttggagttgcaccaatgcttttggttcctaagaccctcccatagggcagttagagaggtcagccttactggcgctgggtctcttctagccttgggttctccagtcaggctcagactgcatcttatcatagctaaaagcctgagatcattgtcgctagacttttaagtttatctccatggaggacagctccatgggttcatggaatttaccatgaagcagtagatctcaggaactccccttctgagtctgacttcttatcaacaaggatgaaaaaggagttcacttggccaggcatggtggctcacacctgtaatcccagcactttgggaggctgaggcaggtggattgcttgaggtcaggagtttgagacttagcctggccaacatagagaaaccccctctctgctaaaagaaaaaaatacaaaaattagccaggcgtggtggcgcacacctgtaatcccagctccttgggaggctgaggcaggagaatcgcttggacctgggaggcagaggttgcagtgagccaagattgcgccactgcactccagcctgagggacagaatgatactccatctccaaaaaaaaaggagttctgcctgcttctgctcccaaagttctgggattacaggcatgagccaccatgcctggccttctgatacattttaacttccctgcaagccacagccaaatccactgtgaaacacaacggagatttgctgatagaatctcaggttaagggtactgtgagtgaaatacccttaggcagccgtccccaacctgactctctatagttgttagatgtaaaattagcttaagcccctttgataattaattcacttttttattattttattttatttatttatttatttatttatttttgagacggagtctcgctgagtcccccagtctggagtgcagtggcacgatctcggcttactgcaagctccgcctcccaagttcacgccattcttctgcctcagcctcctgagtagctgggactacaggcgcccaccaccacgcctggctaattttttttttattattttttttattttttagtagagacggggtttcaccgtattagccaggatgatctcgatctcctgaccttgtgatccccccacctcagcctcccaaagtgctgggattacaggcatgagccaccgtgccccgccttttttcttttttttttttaattataaagaatttgtggccaggcacagtggctcatgcctgtaatcccagccctttaggaggccgaggtgggtggattgcttgaggtcaggagttcaagaccaggctggccaacatagcaaaaccccgtatctactaaaaatacaaaaattagccaggcatggtggcacacacctgtagttccagctactcaggaggctgaggcagaagaatcgcttgaaccttggaggcagaggttgcagtgagccgagaccgagccactgcactccagcctgggagacaagaacgatcccacctgtaatcccagcacattgggaggctgaggcaggcagatagcttaagcccaggagtttgagaccggccttggcaatgtggcaaaaccccatctcttcaaaaaatacaaaaattagccagatgtgatgtgcgcccatagtcccagctactcaggaggttgaggcgggaggatcgcttgaacctgggaggttgaggctgcagtgagccatgattgcaccactttactccagcctgggtgacagagtgagaccctatcaaaaaaaaagaaagaagaatttctaagtgtaactaaatgaagcatttccccttctgtgtatactttttgatctgagactagtagaaatgaagaagaaagaatctaagatgccaacatacatggcatctcaacagtcttttttttttttttttcagaagtcttaatggaggtttaggctttaataacttcagaaatataaatgctaccaacttaaaaacaatgtcacttcaaaaatttttaaacttctttacatttaattcatttttcaactttgaatgatacattttaaatttaatatctttatcagtgatagtcatcttcaaccagaaagactgaaacaaaattttaaagttgaagatgtattatgtgttattcaccattcacaaaataaattgaaatcaatcatttaatttatttaatcctcaccgtatccctatgaaactggtactatttttattccaccctacagatctgtcagaggttaagtgatttgcccaaagtcacatagggtagcaagtgacagaggcaggattcaagctccaaacccatgctataccatctctcaaactgctgagacgcccctaagggcaggggtccccagacccggggccggggaccggtgagtagcctgttaggaaccgggaggcacagcaggaggtgagggatgggcaagctccacctcctgtcagatcggcagccacattggattctcacaggagcatgaaccgtattatgaactgcgcatgcaagggatccaggttgcatgctccttatgagaatctaatgcctgatgatcatctgaaaaccatccccccgccccccacccaatctgtggaataattgtcttccacaaaactagtccctggtgccaaaaaggttggggactgctgcctaagggtatttcactcacagtacccttaacctgagaaagctgagattctatcagcaaatctctgttgtgtttcacagtggaattggctgtggcttgcagggaagttaaaatgtatcagaaggccaggcatggtggctcgtgcctgtaatcccagaactttgggagccgaagcaggcagatcagttaaggtcaggagttcgagagcagccttgccatcatggtgaaacaccatctctattaaaaatacaagaaaaggccgggcgtgctggctcacgcctgtaatcccagcactttgggaggccaagacggttggatcacgaggtcaggaattcaagaccagcctggccaagatgctgaaaccccgtctctactaaaaatacaaaacttagccaggcatgatggcacacgcctgtaatccccgctactcgggaggctgaggcaggagaattgcttgaacccgggaggcagaggttgcagtgagccaagattgcaccactgcactccagcctgggcgacagagcaagactctgccacaaaaacaatgtatcagaagacatatctaatcttcacttctgaatgttccagggctgccagcgtatcgtagaggactggcagaaaatccttatggtgcggtcccttgtggtcagccctcatgaagacatgagaacctggctcaagtatgcaagcctgtgcggcaagagtggcaggctggtgagtgacaaccacccacccacatgggcaggtagggccccatggtgtccctgggctgtccagttctgtctataacccagtttctcttatttttaaggctcttgctcataaaactttagtgttgctcctgggagttgatccgtctcggcaacttgaccatcctctgccaacagttcaccctcaggtgacctatgcctacatgaaaaacatgtggaagagtgcccgcaaggtctgtatcccaatgcatccagaacagaccaccttctccagaatgctcgcctaatctgcttcgctctgcctctttaaggccagaaaagaaacagtgtttaaaccacctactcacaatgtcagggagtctcttcctgttacaaagctatttggcctgatttcatagagtgaatcatagttgaggagatggtgtgtataccattgatcaaaccaatgaatgtgatcacagttatatgtatttgtatctagaaagtgctgggacatagaaagggcacactttctacccctaagagaagatggactgtttggtacaaaaaagagcagatttgtaaatacacagactagaactttcacaagaccaatttgggtttttcagacctctgtctcctctcctgacatcagtttggattcaaaaggaataacatgcagggtataatgctgaaaccaggagggaaagttatcttgaagccaatgtagaattcttctgactttatgtacagacaagcccttgggaaatagatgtggctcctggtttgggggtttttttctttaaacaatctgcctggatgatgctgaggactaataaacagtaggccctggaggcaaagtcatcacctgctttggcttccccttcctccttcttaaaaggaagagggagtctcactagccttctctccccattgtgacccccacagtgaagtacctgagatgagagaggaataaaacccagccactggagcctctgctgtgggaagatctcataaaatagaagttgtgtttctgcagaaggcacacttaggagatctggggactgggtctagaaattgactaattttatacatgactgaatcagagtggctgtgaataccacccagagtgggatcagccagcctgtacccaggcacctggtctttgggatttactgcagtaccaatgacctaagtttccttcttcaaatatgcagcttggagattttatttgtttttttgttgttttgttttatttttcagtctctgtgagcctcccaggccagagcatttctgattcaaagcctgacctcctccaggcaaatgagaaaaacagaaggtgctggatctgagtatagtctggtttccctcctatgtatagcctccaagtctttgactgtcttgcctaaggcaggccgaagaagtcttttgggtaatatctcaagcaaggagtggcttctttcaacataagaccccagcccaacttctccatggaatagtctttcttagtagcccaaattctgtggaattaaaaatcaggaacctatttaaagtgctgggacttcaagcccagaactagagatgtcatatcttcatagttaaggagctacccaaggaaaactgactacttaataattgtgctactgagcaagacactcactgagccttactttcctccactgtaaacaagaatcatgctctgcctcgcctgaggcaaaggaaagtatttgtgggaaaagcagaccctgttatcctggaaggcatcaggctatgggtatatgtatcatgttttcctagacaagacacttcacagagtccagagacaggctggtccttctgtaaaatttcactgagccaaggccattgttcttgcccacaaacccacccatgcaatctagaaccagaatattgtgcctagctaaaatcctgtgggtctatgagaagccccttcttccaagtgacaggtccttcatttataagagggaaagaatcacttttcccttttttcatttcctggggatggagaaaacaaattttaaatagaaaatttccaattccttgaaaataagttgccttgtactatacagtagaaagatactggattctcttatgctggataaacgaaatattgggcttcaaatccaaacttaatacccagcctccccagctttggcagtttatattttgttcagctccttattaagtccaccaaagagtttaaacccagtgtctggattgttgatgacaggtagcttatgggctctgaaaagcacctaccatcagagttcctagtttagctcctacctgcgtgacgtatgcaatgatcacactgagttatcacctttttggcgattgagtttttttactaccgcctcccaattctttttttttttttttttttttttaatcagatttaagccaggtgtggtggctcacgcctatgatcccagcactttgggaggctgaggcaggcgaatcacttgaggtcaggagtttgagaccagcctggacaacatggtgaagcaccgtctctactaaaaatgcaaaaattagccgggtgtggtgatgcatacctgtactcccagctactcaggaagttgaggcgggagaatcgcttgaacctgggaggcagaggttgcagtgagccaagatcacgccactgcactccatcctgggtgacaagagcgaaactctgtttcaaaaaaaaaaaaaatcagatttaagactggtcaatgtagttctcctagaaaaaaaaaaaactaaagggtggttcacacctgtaatcccagcactttgggaggccgtggtgggaggattgcttgagcccaggaatttgagaccaacctgggcaacatagcaagacctcatctctactaaaaattaaaattaaaattaactgggcatggtggtgtgtgcctgtagtttcagctactgggaggctgaggtaggaggatcacttgagcaaaagtgttcaagtctgcagggagctatcattgcaccactgcactccagcctgggtaacagagtgagagaccctgtctctaaaggaataaaaatttaagttaaaaaaattaaaattaaaggaacatttatcacttcccaagtgtgggatgctattgtagttcatttttcaaagttggtggtgagtaatggatccctgtgaagacagatgtctgagggagacaggtgtcttgttaagtgttggaacatctgtgtgcctgccctgtcctcatgcatggattagctgcttgatcaattgtttgtcatgtcattctggctctgggttaacccagtctcttcatgccaattaattccttatattgaaaagcatctgaggttggtcctggcccccaagctagggagtttcattttcttcaaggccatcatcactaacatgttttttgtttgtttgtttgtttttttgagacagggtttcgctcttgtcacccaaactggagtgcagtggtgtgatctcggctcactgcaacctctgcctccaggttcaagcaattctcctgcctcagcctcctgagtagctgggattacaggcagccaccaccacgcccagctaatttttgtatttttagtagagacggggtttcaccatattggccaggctggtctcgaactcctgacctcaggtgatccacccaccttggcctcccaaagtgctgagattacaggcgtgagccaccacacccagccttttttcttttcttttttttttgagacagtctcactctgttgcccaggttggaatgcagtggcacgatcttggcccactacaacctccgcctcccgggttcaaacaattctcctgcctcagcctcccaaatagctgggattacaggcgcgccccaccacgcccggctaatttttgtatttttggtagagacaggtttcaccatgttggccaggctggtctggaactcctgacctcaggtgatccacccgcctcggcctcctaaagtgctgggattacaggtgtgagccactgcacctggctaacatgtttttatttaaaacaaacaaaaatacctcttatatgatttaccataaggagaactgttatttatctgaagtcttgattttttccttcttctgcagcacagaacacattgcctctttttcacattctctcatataatgtttcctttccctgtgttaatgcatttagattttgccatttttccatttgagcttttgatgtttgtacattctatctccatccttgaagccctttattattatttttttttctatttgtgggttttcccccctctagaaaaaccagaaaagatttgtttttccctttaaaagtgatatggaccgcacttatcaagatgctggttcaactggatgtggcgggcttgtacagtagtcccagatctgtgattctgtctctgcccttcctcagttcagctgtctgttcctgactcacatgccctctgctcactttgtatacaagccggtagtataaatagatctagctgctttaggatgctctggagattttgcccctaagtcaccctgttcaaccagatgtctgctgagctaatatcttctccctattccctgtgaatgcatgcgggttttcactccaaatctgtgttcttatttgggtgctatatgatttttaaatccatagtttttgctccgctggactgtccaaggtaccctggtgttgtaaacaaataccaaagttttattagcctgaaacaattcccaaaagcctaatatcccccagggtaacatattgatagtgaagtaagcagttgtttcaaattactttgtaggaatttttatttggggtagccaggcatgaaactttgccaaggcttataaaactggtaattatagttggcaaaggagagccaagacttgagttgaataagtcatgaatctttttgaaaagcccagtctctcccagtatcacgccctggtcctctgtcctatggggtcatcattctgtgctctagggtattctgtttggagaggggttgactcatgcctatcagatgaacggtagctcccttcctctgaagctgggccacaagccaagtggctctctgtgcctttgtcctgttacagatcgatgccttccagcacatgcagcattttgtccagaccatgcagcaacaggcccagcatgccatcgctactgaggaccagcagcataagcaggaactgcacaagctcatggcccggtgaggtccccactgacctggggcaagtcatattccagccctgtcatcccagcagcttctgcagcaaggaggagttcaaggcttcttgggtggctgattgctgacatctctctttccttttccagtagggatcccaggcaaccaccaaagcatactgctctcaaagtctctgccacttgctttcctaggttcagcagcccaagagcacccatgtgctgttctcctggcgtctggttttagagtccttgttttcaaatgtcagatatgctgcccctcccttttttttttttaagatggtcttgctgtgtcacccaggctagagtatagccttgatctcctgggcttaagcaatcctcccacctcagtttcccaagtagctataggcacgtgccaccacactggctagttttttgtagaaatgaggtctctacaaaagaacaggcatagagcctgcatttcctcctacaagtttaatgttgcccaggctggtctcaaactcctgtgctccagccagcctccctccacagccttccaaaatgctgggattataggtgtgggccactgtgccactctgtgctgcagatttgatatttgacttcagagaagtgacttaacctctctatgcctcagttaccaatataaaagggatagtaacagtttctatttcctagggtggtataaagtagtgtgaggattaatgcaaagagtacaatgccaagcatttcaagcagcacttggaatacagtagcagtcagtaaatgtgggctaatctcattacaaagaatggaggcaaagggaaaagtaagggagaagccatatagcaatcaataaagaagtaactctcaactgtcacccacaaaaaattgggtttggggtaggtaagtccatacagacctatctgtaccaggtacatggtttatgctgtcattgagagacaaaggtcaagatggaaagatggtatcttaaaaatgtattccaaaagcccatctcctttcttgcgtgtggctgaaaattttgctagctccttcacattgcctcttgagaataagaggaaataccagaagggcttcagtgtcccctaacaatttgtggtcctcttaggagggctgttttgaggagggaatgtcatgggggcttgtttctctgcttccagatgcttcctgaaacttggagagtggcagctgaatctacagggcatcaatgagagcacaatccccaaagtgctgcagtactacagcgccgccacagagcacgaccgcagctggtacaaggtgatcagaagcaggccacctgtgagacccagcaccctggctggctcgtgtcctcctgtgtgctcgtggttacagctgagcttctgcggagctaggctgatgctgtgaacttactccttgagtccctggagcctatctataagaggcccagggagcctgcatttccccctacaagtttaattaatacagaaagattattggagaaatacttgttacaccattattatgtgtatgtggtgttggggattgctgctgtcttacagactctgagctgcttgttttcctgggagcatctggacacatggttaatctctggagtgacatcttcccttaggtcatctgttcttgttcagcgtttttctgatttgcatccttgcctgctcctcagctaagaacttaactttctttgtttttattggaaagatccaagtaacatcctgttatgttacaggaaacttttgaaagagataaacagaattagaaaatatataaaagatgtgattgatggaggggtgatttttggaagtgtatatctttggtgagaatatactttcttatttctttttgagaggctttctggtaactacttggtgtggcagatgtcattgacatctttccagtatttccttggcttgccaatactttgtgaattaatctctctgaaatctaaacttgtatctgaggactgcagaagcacttataatacagatcttacctttgtttaggcatgcttcattctggtttgaaaaatgtccattttgtcataagagtaatactctctctaggacagcttgcatgtccaaagctggaaaatacatccggtaaacactattaactggaagcttattaatcagagttgtattctaaccagagttactttcattcattaagcttttaggaagtaagcaaatcacaagaaaaaagttggtttcaggaactttagttttagtttttaaaagcttttcctaatgtgggtcccttttgttttatgatgtattctaccatcaacgtcatggccctgtatgcatgccaggactgtttgtcttgttcaccttgaccccaggtcattgccaggcacatagcagatacacaagagatattcagtgaataaatgaaggtatgggaaaactgaaacctgggtcattgtagaattcttactcatcaagtaaaggttagttgtgatcaatctattctccttagttgagaaaacggatacacctgtgttcaaggtttttggcaaggcatacaaacgaatttcccaattattcagaaagttcagctattaatgggtcacgtagtgtcctactatcactgcaaagttaccatgcctggaagcaaactctgcctcacactcgctaaaccaagtggtggtacgctgtagagtcattgccgctcagaaccccagagagagtagtattcctttatccaaataagcaccagcggttcttccttcaagcattgagcactggttctctgtcaggccgtgggctaggtgttgggaccaggcagatgggtgagattctttctctgtcaggctcctatgctcttcatgtcttcctttgtccactatctggtgacctgtcaaaactttgctcccttgaaatattgtgatgactgctgcttgcccccacttctgcttgccatttctgctctcccctaaatgtacactttatacccagtgcagctctgctttctgggtgaaagtgccagccctgtgcctgctccaggcgtgctttgctggcctcgctctcactcctcgcctgcatgtatcccctgcacggtcacattcatgggcttgtttcccgtgaaccaatctgcacatctctggccttattcttcccccttgccaagtagtccccaccgctgccttcttgtttccctccttgcccttctgcagctctcttccagacctaccccaaatccctcttcttgacaaagctttccaaatgcctgtccttgctactcccattgtgccttgcagagtacctaggtcttcaggtttgtgcgtgagctcccctaggaggccgggagatcccttgaggcaagagctctctcctgctcctcttgctgtgcccttctcgcagtgcactcatagtacgttatatagttagtgctccacaaagtgagtggatcatgttgaatttagtaaaacagccatttctgagtgtctccttgaagtgtttgctttatggacacacagaacaggcaggagcagctgttcctcagcatcgaccttgcgctcctctgagccctgctgttggtcacagctccagtgtctgtccttgcctttctccccaaccaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggtacccttcccttcttccttattaacaaccaaccaaggtgccaggctctctgagaaatggaatcgttgttaagcttaaaaggaacctgaggctgtatctgaagtgccagatggagctggaaaatactgcctatctaccatccagcactgcctgagatagggctttgtgtgtgaatatttgtgtttggggagctgacgcagcctctatgctgaagagcaagctccttgtcaagcttccctgtggacgagctcattctctacgtgctgcacctgtgtgtgggtggctgtacacagctgcgggtccactctgaggatggggcttgggaggtccatcctctgcctttttctctccacttccacttcttccccttccccttgggctgtcagttcagaatgcgccagggccgctggcaggcccagagatccgggtgttcagagcctcagctccacctcctccctccatcctgttcaggaagccactgtcagcccaagataataacagttatgatacagttgccatttattgagtctttgctatgtgccaggtaatgattaaagatttttcatgcattaaccgatttaattgttataaccaccctttgaagtaggtacagttagtatgctcttatgcccattttacagatgagaaagcagaggcaagagtgtcgctaacacgtgtgtgttccaacaggatctgtccaaaaccctcctgatgtacacggtgcctgccgtccagggcttcttccgttccatctccttgtcacgaggcaacaacctccaggatacactcaggtatcagagaagggaaggatgcaactctggtaggccatgttaatggcaagacctagaagtcacacgtaaaagtcctctttcccttcctgaccctgatcaggacacccaagatcgatgtatgtgccttttttctgcactgcatttaaggagtaacagctgactgggccccacctgtgtgggtgaaacctacagagagtgaggaggagaagtggagtgagaactctccatgtgtggtttattatggagggattattaacccatctgttaatcacaatatttctttttttctgaccaccctgttgcctattgaagtcacagtatttctaaaatccctcagcaacttggagaggtctaaacaaatgactcaaatcagttaacccagagggtaaaggtgacaaggtccacatcctccttcatttgaggatctccagccagctaagagccaggtgtccctgttgcctctgccgtacagcgactcttacatgtgcagacttgtggctttctaaaggttatcctggctcgtctgtttgatgtttgcatacatgccatggtgacccctcggtgacctgggatgaaatgctcatttgctcagggtgactcttacggtgttcagtcaagagcactgccacccatcacagacacctgctcccatgggaggccaaacacatttgagagttactggttggagtccctgccagctctcttctcaggagccttagagatgaaaaacaggcaggcaaaaactgggaaactaaattttctgtgactttctcatttctttttcagagttctcaccttatggtttgattatggtcactggccagatgtcaatgaggccttagtggagggggtgaaagccatccagattgatacctggctacaggtgaggatacttggttacaggtgaaggtggctccaagtctctcttctgtgtgtctcctcttacaagcatgctgtgttcagtgtcacaaggcagtgtgtgttaatacagtgctaaggaagtagggctcggtcccagtgtcaagtgtccatattttctttattttcttttagaaactaaagggaagggaatgaaccatttcagagcatcaaccgaaacaagttaaaagaattgatctttgcctagaagaataactagttgtttcaagtctcatatgctacatactgtgtctttttgaataccaggttatacctcagctcattgcaagaattgatacgcccagacccttggtgggacgtctcattcaccagcttctcacagacattggtcggtaccacccccaggtatgtggagactctgggcagtttctcatctaaccactggtttggtggtcagcctttccaaaagctgccctggcgacacaagcgcaggaactgggagccatgtgttccatcctggcccaggagggtctgtccagccagccgtcccagagggcccgggagccttgctaaaaagaaagcgttccctcgaagactgagccccttcctggtagtctcaagcagatgttaatgtcacactcaccacttgttttgttattctccttaaaattcataccagctcttccccaacccacatttcctttatagatgtttcttctgtttttccctcaggccctcatctacccactgacagtggcttctaagtctaccacgacagcccggcacaatgcagccaacaagattctgaagaacatgtgtgagcacagcaacaccctggtccagcaggccatgatggtgggtcggagccactgtggggcttcatagaccctggtcccttagctggtgggcgcaagtgggattgtttttcttcctcgcattggtgctgtgcacttcctctgactgctggaaatagtacctcctgtgtctccaaaacagacctttctgaggttagctgattcttgatgcttctctccttcttctcttggcttcaggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcaggtaggatcttcaggctcctggcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctagctcctctgaaacgggggcttctggctcttgaaatttttttctttttttttttaccatcatagtctctgtggtaaatgattgtgtaaaatgaacaagtggattctcacagtgagatttgttaagcaagtgggctctggaatttcccttggggtagagtgttacttatgggggatgttttagtgaggcctgagtgctgtggtggcactttgaggcctctcgctttttttttttttttttttttttttgagccagagttttgctcttgttgcccaggctggagtataatggcgcaatcttggctcactgcaacctccacctcccaggttcaagcaattctcctgtctcagcctcccgcgtagctgggattacaggcacgcaccaccacacctagctaatttttgtatttttaatagagttggggtttcaccatgttggccaggctggtctcgaactctgacctcaagtgatcagcctgtctcagccttccaaagtgctgggattacaggtgtgaaccactgcgcccagccacctcttgctcttttttttttttttttccccaagacagagtctcactctgttgcccaggctggagtgaggtggtgtgctctcggctcactgcaaccccacctcctgagttcaagcgattctcgtgcctcagcctccctaatagctgagattacaggcgcatgccacacaatcagctaatttttatatttttagtagagatgggatttcaccacgttggccaggctggtctcaaactcccgacctgaggtgatccgcccagcttggcctcccacaatgttaggattacaggtgtgagccaccgcgcccagcctcacctctcgctcttaataatgctcaatgtttcttaaatgcagtgattattcaccaacaccaattaatcatggtccacaggtgattatctctgcatgagctgttgtcttctgatacaaaatgtgggtcaggctggttcttggggtgtttaattaaagagagtttaagaaaggaattgtttccagaggtgtaagcaaagctaagagaatcaacaaggagcgctcatgaacaagcacaagtgagaaattgttactcagccctcacccacccccacactccagaactgaaagagcaaggagcccagctagaaccgtcaccatgccagagaggccatctagcagagtctacagtccagagagagtggtagcttctagaactggggccaggctggtgatgggggacacataccctgacctctccctctacccatcctctgacctcctgccagtacccttccaccattggccacgcccaagtgggatcccaggtgatgcggcttgtaggagtcaggcttctcagaacagaggaggacagagtgggtcggaaaacaggaatgatcatttccatgcctaaccgttcctggtcctcaaaaacagattttgttttgttttaatttgaacaacactggtcattgagaaccataaaaggcaaagccactcttttcattaattctaaagattgttttttctttttgtgaggaacgtgtgattaggtaaggcatatgtagtctgtgggatctcttgaatgcagtggtgctctcagctgaagagctgaggacctctgatgtacactcaccgctcccactgttccttacaggcagcaacggacatgagtttgttttccttctaaaaggccatgaagatctgcgccaggatgagcgtgtgatgcagctcttcggcctggttaacacccttctggccaatgacccaacatctcttcggaaaaacctcaggtattcagaaaccctgaaatggcaacaatttgagaagtcttctggttttttcctacatttgtctcgtgtagtcatgtattatatcttaacaaaaaactataaaattagggagccaggcatggtaactcatgcctgtagtcccagaaactccagaggttgaggcagggggatggcttgagcccaggagttcaaggctgcagtgagctgtgattgtgccactgccctccagcctgggtaatagagcaagactgtctctttagaagaaaaaggctgggcatggtggctcgcacctataatcctaatattttgtaatgccaaggtaggaggatcacttgagcccaggaattcaagaccagcctaaacaacatagagagaccccatcactatttatttaaaaaaattaaaaaggtgaccgggcacggtggcccatgcctataatcccagcactttgggaggcccaggcaggtggatcacttgaggtcaggagttcgagaccagcctggccaacatggtgaaaccctgtgtctactaaaaatacaaaaattagctgggcgtggtggcggccacctgtaatcccggctactcaggaggctgaggcacgagaattgcttgaacctgggaggcggaggttgcagtgagccaagattgcaccattgtactccaggctgggcaacaagagtgaaactccatctcaaaaataaataactaaagtaaggaaaaaaaaattaagtggccaggcatggtggctcacatgtaatcccgacactttggaaggctacagcaggaggattgcttgagaccaggagttcaagaccatcctggacaaaaaagtgagaccctgtctctacaaaaaataaaaaaatcagctgggcatggtggctcatgcctgttgtcccagctccttgggagatggaggcagaagaatcatcttagcccagttggttgaggctgcagtgagacaaggttgtgcctctacactctagcctgggcagcagagcaagaccctttctctcaaaaaataaagataggctgggcacagtgactcacgcctgtaatccctgtctctacaaaaaaaaaaaaaaaaaaaagctgggcacagtgtagtcccagctactcagggggctgaggcaggagaatcacttgagcccaggaggtcgaggctacagtgagctgtgatggggcaactgtgctcgagcctaggtaacagcgagaccctatctcaaaaagtaaataatattttttaaatgttttaaaaatgaaaaattaagttataaaattttataatttataaccaagttcttccagagtttttagggtattttcattgggaaatgcctttaacaacctccaaagaatccagcagggagcaatttgtggcgggtagactggagagtctacagatgggatgggtctgaccccttgtgggatgaaagccaaacctttcccatcgtccgtattctggcactcctcttcctgttaaaaccatgagaattcacttgagaggtattttagtcacttgattgttcccccaaagccatgagtttcttgggcaggttgaggagacctgtgttgctttcactgcaccatcagtgcactgtctttctgtgatgccttattcttcacaaagcatccctcaacacacacttcacaccagcctgctgcggcgggggctcattaaaaatagtcagggccgccgggcgcggtggctcacgcctgtaatcccagcactttgggaggccaaggagggtggatcacgaggtcaggagttcgagaccagtctggccaacatagtgaaaccccgtctctactaaaaatacaaaaaattagccgggtgtggtgttgtgcatctgtaatcccagctactcgggaggctgacgcaggagaatcgcgtgaacccaggaggcagaggttgcagtgaaccaaaatagtgacactgcattccagcctgggcaacagaacaagactccatctaaaaaaaaaaaaaaaaaaaaaaaatagggctgggtgcagtggcttaggcctgcaatccagcactttgggaggccgaggcaggcggatcatgaggtcaagagattgagaccatcctggccaacatggtgaaatccgtctctaccaaaaataaaaagattagttgggcatggtggcacacgcctgtagtcccagctactcgggaggctgaggcaggaggatcacttgaacccaggaggcagaggttgcagtgagccaagattgtgccactgcactccagcctgggcaacagagcaagactctgtctcaaaaaagaaaaaaaaaacagtctgcctgtgttctgagctgctcaagggtatactctctttacggtacactaaccctgcttttctgtgggcgccagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcgggtatgtgatgcggccacgagagctagtgccccttccgtggcagggaatctccgtagcgctgcttttctcattcctctctgtatgtctgtcactgaaaaatctctttgagagaatcagctttgaagaaggtttaataaagtgagtcctaaagcagataagcttgtaagaaactgaaggacccttaagctgagaaattgttttcaccatcctagggaagactagacagataaatattcggcaccatatatttactaatgtatacaaagcactggccatatggagaaataatgtttagtagatgtgtaagtaataagaaagagaattgcttgttttccaaaaccttgaaacttccagtactctcatctgcacagaaggcctataactgaggtgaagtctcgtgtttttcccttatgccttggggttccagtggtcatcaggttagagtggtcacttcgtttctccttttgccatgttcttatttcccttctcctttggctatgatggggaaattagaactcagctctgcctggaccccaagccttgtttcttcctggacccaaactacatgtcttaggctgctctcttactgaacagttgtgtcctgatgcagatggctccggactatgaccacttgactctgatgcagaaggtggaggtgtttgagcatgccgtcaataatacagctggggacgacctggccaagctgctgtggctgaaaagccccagctccgaggtaggccactgggccctgcagacctgcgctctcctggaacccccactcgcaataggatggccctcccatctgttcatgtgcacacagcggctcttctttgctgtgattggctgtagagcgtgtccagacttttctaacaaaaatgaaatgggtgagcaagacaagttcacagagttagaatgagtagtttaatatttcctccagaactctttatatattaaagaattaaagatagttgtcttttttatatatgttacaaatttcctattaacttagtttttaaattttgctaatggtgtgttctttggtttggagtttttgtttggtttggtttggtttttgatacacagtcagccctccatatccgtagcttctgcatctgtggattcagccaactgtggatagaatattttttaaaaatatatataacaataagaaatgtaaatttttaaaaatatagtagaatgactgtttacattgtataacattcacattgtattaggtattatatgtacccaagatgatttgaaatataagggaggttgtgcataggttacatacaatactatgccattttattaaaaagttgtttttggctgggtgcagtggctcacacttgtaatcccagcactttgagaggccaaggcaggaggatcacttgagcccaggagttcaataccagcctgggcaacatagtgagactccatctctttttttttttttttccccgcccaggctggagtgcagtggcaagatctcggctcactgcaacctctgccccctgggttcaagcgattctcctgcctcagcctccccagtagctgggattacaggcacgtaccaccatgtgggttaatttttgtattcttagtagagacagggtttcaccatgttgtccaggctagtctcaaactcctgacctcaagtgatccacccaccttggcctcccaaagtgctgggattacaggtatgagccaccacacctagcctgtgagaccccatttctacaaaaaaattagccgggcatggtggtacacacctgtaagcccagctactcggaaggatcatttgaatccaggagaatgaggctgcagtgagccataccatgccactgcactccgtcctgggtgacagagcgagaccttgtctcaaagcaattttatatatatatatataaatttttttttttttttttagagacaaagtctcactgtgtcacccagcctggtcttaagctatcctcctgccttggcctcctaaagtgctgggattagagacatgagccaccacgcccggcctacaccattttatataaggcacttgagtattgcagattttgggatccgatgggggccctaacttagagatatgactgtacaggcattttttaaagttagaagttaccaaatttttaatccttttctttaaagtttcttcctttggtttgtttttgttttttttgtttgtttgtttgtgtgttttgttttgttttttttttttgagacaaggtcttactctgttacccaggctggagtgcagcagcacgatctcggctcactacatactccgtctcccaggttgaagtgattctcctgcctcagcctccagagtacctggaattacaggcgcgtgccaccatgcccggcttttttttttttttttttttttttttctcgagacggaatctcagtctgtcacccaggctggagtgcagtggcgcgatctcggctcactgcaagctccgcctcccgggttcacaccattcttctgcctcagcctcccaagtagttgggactacaggcacccgcctccatgcccggctaatttttttttttgtatttttaatagagacggggtttcaccatgttagccaggatggtctcaatctcctgacctcatgatccgcccgcctcagcctctcaaagtgctgggattacaggcgggagccaccgttcccggccactcttttgtatttttagtagagacagggtttcactgtgttggccaggctggtctcaaactcctagcctcgagttatccgcccttctcagcctcccaaagtgctgggattacaggcatgagccacggtgcctggcctcttcctttggttttatatttagaaaggccctctccaccttgaggttatttttttacctatattttcttatactagttacagggttttttttacatttaagtcttaacccaagtcagacactatggtacacacctgtagtctcagctacttgggaggctgaggcaggaggattgcttgagcccaggagttcaaggctgttgcacattaagaccacacctatgggcggggtgcagtggctcacgtctataatcccaacacttggggaggccaaggcaggtggaatgcttgaagctcaggcgtttgagaccagcctgggcagcgtcgcaaaaccccatctctacaaaaaataaaaaattagccgggcatgatggcacttgcccattgtcccagctacttaggaggatgtggcgggaggatcacttgagccaggaggcaagggttgcagttagctgagattgtgccactgcacaatctgtaactcagcctgagtgacagagccagaccctgtctcaaaaaaaaaaaaaaaaaagaagaagaaaaagaccacacctatgaaaagccattacactccagcctgggtaatatagtgagatcgtctcttaaaaataaacaagaccaggcacggtggttcacgcctgtaatcccagcactttaggaggccgaggcgggcggatcacgaggtcaggagttcaagaccagcctgaccaacgtggtgaaaccctgtctctactaaaaatacaaaaattagctgggcatggtggcacgtgcctgttatcctagctactcaggaggctgaggcaggagaatcgcttgaaccagggaggcagaggttacagtgagccaagattgtgccattgcactccagcctgggcaatagagcaagactctgtccaaaaaaaaaaaaaaaaaaaaaaaaaaattaactaagtttggaatttaaggcgtaactttatttttttaaacatgactagccatgaccacactgttggccaaataattcatcctttccctgctatttagaagattttgtttttattgaacactgaattttttttaattttattttttgagagagagtttcgctcttgttgcccaggctggagtgcagtggcatgattttggctcacttcaacctctgcctcctgggttcagtgattctcctgcctcagcctcctgagtagctgggattacaggcacgcaccaccacacccggctaatttttgcatttttagtagaaacgggatttcaccatgttggtcaggctggtctcaaactcctgatctcaggtgatccacccacctcggcctcccaaagtgctgggattacaggcatgagctaccgtgcccagcctaaacactaaattctacgtgtccttggatttctttctagggctcctcttctgtttcattgctctttcggcctgtttctcaagttccatggtgttaatacaagtcccttctcaggggctatcgtcatctattttttttcttgaggctgttaacatctttatactcataaggggatatccttaaactgatatttttcatttactcagatctttaagtctaacacatttctaattaagtttaattcaaggcatagttcattgttagaattgtgaatgaggtctttataatgttatacattctaaccagttattgatggactgtaagaaaacaactgattaggccaggcacagtggctcaggcctgtgatcccagcattttgggaggccgaggcaagcggatcacttgaggttaggagctcaagaccagtctggccaacatagtgaaaccccgtctctactaaaactacaaaaataagctgggcatggtggcgggtgcctgtaatctcagctactcaggaggctgaggcatgagaaccgcttgaacccgggaggtggaggctgcagtgagccaagatagcaccactgccttccagcctgggcgacagagcgagactcggtctcaaaagtacaaaccaaagacagcttattagagtataattatgttgaaattaaagtttccagaaccgtagttcacatgcactcctgtgttctaggtgtggtttgaccgaagaaccaattatacccgttctttagcggtcatgtcaatggttgggtatattttaggcctgggagataggtgagtaatttaatttggtttttttagtcttctttctccattgtagttttcctgaatggtttcatttggttttccaattaaatatatggctttttggtaaataaatattggtattgtatatgtaaggtctcttttacaacattagccttagttccctattgattgtgatgccatcagaagaatacatcaaactaaagaaaaggaaaaatagttcaagctgggtgcagtggctcatgcctgtcatcctgggaggccagggtgagaggatcacttgagcccaggagtatgagaccaacttgggcaatagcaagaccccatctcaataaagccccacatagttcaaggatactactttctccttagtctggcttaatgtattaaaagctgtattgcagggcaggctcattggctcatgcctataattccagcactttgggaggctgaggtgggaggatcacttgagcctaggaatttgagaccagcctgggcagcatagcaagatcccgtctctacaaaaaataattagccaagcatggtggcatacacctgtagtcccagctctttgggaggctgaggtaggaggatcacttaagtccagaaggtcaaagctgtagtgagccgtgattgcaccactgtgttccagcctggtcaacagagtaagactctgtctcaaagaaaaaaacaaaaatactaaatcatattgattttattgattcaagaaacatttgtcagtcaccactgtgctaagataaatcattttatttctgtaattgaaaccaaccaaagtagatccttctgatatttttgcataataagttttgtccaagtgtactgactgaacatgtaatttacgaatgttcagaaacggactcacaagaatgtaaaaaagactggttctctgatgtctactgtttttctcctggaatcatgtctcatgcaactatttgagagtaaaaataattttaccccacaaaaatagtagatttttgtttttaaaagtctagcagaatatctgttgataaaagccaggtggttgccgggattggctcagggacttaaggagatgagaccctgttacggctgctcagtaaaaatgatagagccttgtcaccagaaggtgccagtgctgccagaaatgacttattaggtcaccaaagacctcagaaaagcacagtatctcagtacagtgggacaacctgtgattcacacataagcccacatttcagcaggagttggcaaactgtttctgtaaaggaccagttaataggtcgttttaggctttgtgggccctttggtctctgtcacaactagtcagctctgctgtcgtagcatggaagcagcctggaacagtacttaaacaagggagcgtgctgtgtgttccagtaaaactggacttgcagaagtggaggtggaggggctggattggctgagggctatcgtttgccaactcctagcttacagtatcacgtacttattgattttttcatccgttatctctgttgatccctccctgatctgtcttctgtttctcaaagacacccatccaacctgatgctggaccgtctgagtgggaagatcctgcacattgactttggggactgctttgaggtgaacacatgttctagaaccccaacctcatcttcccgggtgatcacttttccccctcctctgacactgtaggcagacttaagcccaaactgccttactcttaacccctgctggccctaccaaggctgtagttaagtcagtctaagaaatgaaatctgggctcttagttggactctaacccccttggagcaagggctgctgttcccatgccctctgagtagagtaggcccctaccctccataccacctgttgcccaagagcctggacacgatgcacatttgtcttttttcctttttgttttctcttcttttctgttgttcgtttctttttttttttaagttgtctttcaaatccctgtgacaagtcctctcactaaacccaagctgcttcctttccctgctataattaaaatcagtgcaggtgatgcagaggaaagccacctgctccacctggctccaggtaaagaaaataaacattgatttggcttttcccatttattattctgtaggttgctatgacccgagagaagtttccagagaagattccatttagactaacaagaatgttgaccaatgctatggaggtgagtggatatcgggaacgagctgcttccaaatgggatcaggacagttacagccttctgagaacacgcttgttagagcccatcagttctccaaagtgcctttgtgatttttgtagcagttttctgttatttcctattgtgttagaaatgacctatctatgaaaggcttaccacttcacttctgacctggtttcctgtggatcacagtcctgccctgattccttctcctaagacattctcaccctgatacgtcaacatggcctgtgtctgcttcctaagtgagggtaacagaccccctcggcttgctttgggtggagagttagtaactcctgtggtgagtggctctgtcccatttctaggttacaggcctggatggcaactacagaatcacatgccacacagtgatggaggtgctgcgagagcacaaggacagtgtcatggccgtgctggaagcctttgtctatgaccccttgctgaactggaggctgatggacagtgagtatcatcaagtgcctgggagctgagctcagtggctcacacctgtaatcctaacattttgggaggccaaggcagaggagttgcttgaggccaggagttcgagaccagcctgggcaacatagtaagacccctttctgcaaaaactgaaaaaattaggcatgtattagtctgttctcacgctgttataaagaactgcctgagactgggtaatttataaaggaaagaggtttaattgactcacagttctgcttggctggggagaactcaggaaacttacaatcatggtgggaggggaagcaaacacttccttcttcacttgggggcagaagagagaagtacagagtgaaggggagaaaagccccttataaaaccaccagatctcatgagaactcagtcactatcacgagaacagcatggaggaaccatccccatgatctaatcacctcccatgaggtccctcccacaacacatggggattacaattcgaattgcagttcaagatgagattttgggtggggacacagccaaaccatattaaggtgggtacagtggcacatgccggtggtcctacctgctcaggaggcagaggcaggaggatcacctgagcctgggaggttgaggcacagtgagctatgatcatgccactgctgcactctagcctaggtgacagcgtgagaccttgtctcaaaaaaataaaaaaataaaaaatttaattttaaaaataaaagaaaacaaataaaaaataaaatcaagtgtgtggaagctggggttggcagcctcccaaattagcaatatccagcacataacattttacaaacctatgccccttcaagcctgcagcctgggatacattgcagagctattgcattacatttgcacagagtaactctgacacctggaaaagaacttcaagtatttttattaactaaaacagaaaatgccattatcaatttagatactaaaatattcttcagtattaaccaagtatcctagtgatttttctctgggctttatatagagtagtaagctgaaattttaataagaaacgcaaacagttatgatttgggacagcagtatcttcatctaccctttgttctgccaccttaggggatgttcaccaaaagagcttctaatccattgaaattaaatcatactttgaccttatattatctgtattacattgtctaagtccaaatcaacagtatagagtgtccccctgggtttcacatcccatgctaatacccacccacttattcctgagtaatatgtctgtcatctctttttttggggctatagctttacgaacaattatatttttatgactggaagaaaataaccaagtttctttcaagtcaaaatttgaagcatgtttcaatactctttatttctctccctagcaaataccaaaggcaacaagcgatcccgaacgaggacggattcctactctgctggccagtcagtcggtaggtgcatccacgggaggtcgcaaaggctctcccccttgtgcagagtgtttgtagagtggggtgggcttcagtcggtgggtgcatcccgcaggacgttacagaggctctcccccttgtgcagagtgtttctagagtggggtgggcttcagtcagtgggtgcatcccgtgggaggctgcagaggctctcccccttgtccagagtgtttctagaatgggatgggctttgttattgacagtaggaggcataacttaaagtggtaatggctactgaacatgaaggaagtaaatctagttttctcgtggttcttgacagtgatagttttgcaaaggtgctattactgctagcttatcacctgataagcatttaattgaacttcaaatacttggtctagaaatatttactgagcaccaagcagctaatatgttttttatttactgtttcacacatggagtgggtacctgaagctctgagatttttatcaaactatctgattttaatctatctgtaaaaacacagtttaaagtatgtctgaatatcagctcactaatggtgtctgggttggaataattattaacagtattaccttggtcctcaagcattgataatagctcagaaatgggaagatggaagcagcagaaatttatttgcgttgctgggaggaaccctggaaccacatggaacagtgtttctaggagcgcgactcacagagcaggggtctagaagacttgtgtccctctgagagccagagtcctttttgtgtgtggaaacctctcaggagtgtttgagtcaagctgtttaacttttattttatttgtattagtttccagcacacacagcattgaagttactggttttggggggtttttttgtttgtttgtttgtttgtttttgagtaaaattgtttaactttgtttagccctcccctcttccccgcactccccatcccaccccttttttccttcgtgacatttagcaacaccctacatagttcatactttgggaattactgttagggtgtaaaaaagtatatcttttctacaaatgtttacagagcacttacaactagttaataaactaagccaaataatgtctaggggtgcaaaaagaaaatgtgaggtacagtggtcgcctgaagaagcacagaaagcattacagaattgaccataacctgcgtgaaatagtcagagagcaggagactctgccgtggagcctgcgggtgctttcgtgtggagtagggctgtggagattcagaaaaggggacttcactgtgaaccgtagctgcgaagagagcacttgggttggtggcatctaaccatcacttcacctgacaaagggtaatttttctttcttttttttttttttttcttgagacagagtctcgctttgtcacccaggctggagtatggtggcgcagtctcggctcgctgcaacctctgcctcccgagttcaagcaattctcctgcctcagtctcccaagtagctgggattacagacgcatgccaccatgctcagctaatttttttgaatttttagtagagatagggtttcaccacgttggccaggctggtttcgaactcctgacctcaagtgatctgcccgcctcggcctcccaaagtgctgggattacaggcatgagccaccccaccttgccaaagggtaatttatccttattggatgggtggaatctgatgggtagaaaggaaaagggtgtttcaggcaagaaattgggatactggcttataatgtcatacatggacactgtgacaggatttgtgaatgcctgaatcaccaggctcataggacacacagtctctgggggagcttatactggtggctggtctcaaacgaagtgtgctgagacttactggcttcattctcagggcccacctccctaacaattctcaagtggctactatatgctgtctgtagcatacagaagggaactaggacagccagttctgctggcaccagatggtggtggatttggttgtttagcttaaaagttaaattccagctgggtgttgtggtgcacacctgtaagcccaggaggagcacttgagcccaggagtttgagactagcctgggcaacacagcaagaccctgtctcaaaaaaaagaacttccaatggatcattaatagaagtataactatcacccgggtgcggtggttcatgcctataatcccagtactttgggaggcccaggcaggcggatcacctgaggtcaagagtttgagaccagcttgaccgacatggagaaaccccatctgtactaaaaatacaaaataagccaggcatggtagcgcatgcctgtaatcccagctactcgggaggcaggagaatctcttgaacctgagaggcagaggttgcagtgagccgatatcatgccactgcactccagcctgggcaacaagagcgaaactccgtctcaaaaaaaaaagaagtataattatcactaccttcattcagaaggtctgtgatgtacaaggacagcattcaaaaaacttcataattctgatttcagactcttctctgctaaaagccataatcagtggagggtgaacctaacacctgaggactttttctacccagagctgatatgaaaattcctccaaggaataacatttcaaccttgtgtgtgacacattccgaagttaattttctttatcagtttttgtccaaacacattttgtcacgaggcttttgacctgttcttattcctatgagtttattttgttaatgcaatttgcctgaaaatccatgtttcaattctgataagcaccaaattttttcaataattattaactcacatttgaaaatggctttacagatttaaaaatttttttttttttttttgcaaatggagtctcaggctggtcttgaactcagctactcaagtgatccttccacctcagcctcttgagtagctgagattacagctatgtccagcttaaaactttgaaatacataaagagtaacataaaactttccttctcttttcccctttagggtaggtagggcaggcgttaaaggaatagagtgcgtagagaattagatgctaacaccaacagtggctttttggtgtttgaattttctgttaattgatttcttttcccatagaaattttggacggtgtggaacttggagagccagcccataagaaaacggggaccacagtgccagaatctattcattctttcagtaagttcagcctctgaggaaccgcaccagtgactagctcattaagaaaatagcagctgtgttataccctgagaggggtcaacaagatgaagcaacaaaactgcctttgagcagaaaaggttccatgaatttctcattatattctgaggacaaagacggacatagaggaaggattgcttaacacttacaaaagtgaatatttgagaaaaacagggcacccattgaacctgttgtattgctcccattcttacagttatttctacttttgtgtattccagttggagacggtttggtgaaaccagaggccctaaataagaaagctatccagattattaacagggttcgagataagctcactggtgagtgtgtcatgtgtttacttaaaagggacataaaatctgagcacactttcctctctgagtggtggtcagccccatgtccagtggtgttagtgacgagctttggcaccatgcagctgacaaagagtgtctttgaggtcatcagttaaacattgtctctgattcttctttctgatgactgataacagtacttaatgttagtttaatctcatttaactcaggaggtgaataaatttcttttgggtccaaaatcagccaaattcagagaagtgttttttaatcatgtcttttttttgtttgtttgagacagttttgctcttattgcccagtctggagttcagtggctccatcttggctcactgcaacctctgcctcccaggttcaaacaattctcctgcctcagcctcccgagtagctgggattacaggtgcacgcccagctaattttttgtgtttttagtagagatggggtttcaccatgttgggcaggctggtctcaaactcctgacctcaagtgattcacccaccgcacccggccgaatttttctttttttcttttcttttttttttttttttttgagacagggtcttgctctgtcacccaggctggagtgcagtggtgcgatcttggcccactgcaacctccaccttccaggttcaagtgattctcctgcctcaggctcccaagtagctgggactacaggcatgtgccaccacactcggctaattttgtatttttagtaaagacagggtttcaccatgttggccaggctggtgttgaactcctgacctcaggtgatctgcctgcctcagcctcctaaagtgctgggattacaggcgtgagccaccgcacctggccaaacttttcaaatccaactatgatggtatccttcatcttcatgtctgacgataagctgttggcatagacagttaagcccacagggaacaagaagtgcatcgattgccaggaagagagtgaaatgtttttgttcctcctgtgctaggtcgggacttctctcatgatgacactttggatgttccaacgcaagttgagctgctcatcaaacaagcgacatcccatgaaaacctctgccagtgctatattggctggtgagtggggctctcaaagtgactttgttaaaataagcaatcaggtcccaagcctaggccccaaagccagagtagcaaaaagagatttggtgaggtatctgtaaagggcagttagagctcagatctctttaaaaattcatatttactgcatgtgcccattgttataaaaaaagaaactgagaaatatggatgcatacttggaaggcaaacactaaaattaaaattacattaagataagtggttcaggagaataggcaaggcttgtttgtttgtgaggggttccttttgggttttgctgtcagtccagtgcagagatacaggaagggcgaaggaaagaaaaagctagagcagtgacagagagaaggagataaaaagacagatttatgatctgggagtctaacctcaagggccactctggggtgtgctttgattaattgcccaaggcatgcttgaaggcagaaacttgagagagcgggcgctggggaagtgtccttgcgggacctcggtgggcattagccttgactggaaatgatgagtgtgtcccatgcaattagctgtgggagagctcagggaataccctagggttgtcagaccttggccttttcattttcaagttatctgtgtaaacctttgaagaagctcaaattattaaaatattccttttctttctgccttcttaggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgta

SEQ ID NO: 2 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - DNA, 20 bp, Homo sapiens
  source 1..20: mol_type = other DNA
cacatgataatagaggtccc

SEQ ID NO: 4 - DNA, 20 bp, Homo sapiens
  source 1..20: mol_type = other DNA
gtcggaacacatgataatag

SEQ ID NO: 5 - DNA, 20 bp, Homo sapiens
  source 1..20: mol_type = other DNA
gcagctgctttgagattcgt

SEQ ID NO: 6 - DNA, 20 bp, Homo sapiens
  source 1..20: mol_type = other DNA
aggtaggatcttcaggctcc

SEQ ID NO: 7 - DNA, 20 bp, Homo sapiens
  source 1..20: mol_type = other DNA
ggatcttcaggctcctggca

SEQ ID NO: 8 - DNA, 25 bp, synthetic construct
  source 1..25: mol_type = other DNA
tgtgttgcgacgaatctcaaagcag

SEQ ID NO: 9 - DNA, 28 bp, synthetic construct
  source 1..28: mol_type = other DNA
tcatgtgnnkagacgaatctcaaagcag

SEQ ID NO: 10 - DNA, 28 bp, synthetic construct
  source 1..28: mol_type = other DNA
tcatgtgttgagacgaatctcaaagcag

SEQ ID NO: 11 - DNA, 28 bp, synthetic construct
  source 1..28: mol_type = other DNA
tcatgtgtttagacgaatctcaaagcag

SEQ ID NO: 12 - DNA, 28 bp, synthetic construct
  source 1..28: mol_type = other DNA
tcatgtgatcagacgaatctcaaagcag

SEQ ID NO: 13 - DNA, 28 bp, synthetic construct
  source 1..28: mol_type = other DNA
tcatgtgatgagacgaatctcaaagcag

SEQ ID NO: 14 - DNA, 28 bp, synthetic construct
  source 1..28: mol_type = other DNA
tcatgtggtgagacgaatctcaaagcag

SEQ ID NO: 15 - DNA, 28 bp, synthetic construct
  source 1..28: mol_type = other DNA
tcatgtgaagagacgaatctcaaagcag

SEQ ID NO: 16 - DNA, 28 bp, synthetic construct
  source 1..28: mol_type = other DNA
tcatgtgggcagacgaatctcaaagcag

SEQ ID NO: 17 - DNA, 23 bp, synthetic construct
  source 1..23: mol_type = other DNA
gctcctgaggaatatctgcagtc

SEQ ID NO: 18 - DNA, 24 bp, synthetic construct
  source 1..24: mol_type = other DNA
cctctgcttggatgtgatgacttg

SEQ ID NO: 19 - DNA, 23 bp, synthetic construct
  source 1..23: mol_type = other DNA
ccatcctctggcatgagatgtgg

SEQ ID NO: 20 - DNA, 22 bp, synthetic construct
  source 1..22: mol_type = other DNA
agaagcccccgtttcagaggag

SEQ ID NO: 21 - DNA, 22 bp, synthetic construct
  source 1..22: mol_type = other DNA
cctggaagaggcatctcgtttg

SEQ ID NO: 22 - DNA, 20 bp, synthetic construct
  source 1..20: mol_type = other DNA
ctagcaccttccctgcttgc

SEQ ID NO: 23 - DNA, 56 bp, synthetic construct
  source 1..56: mol_type = other DNA
acactctttccctacacgacgctcttccgatctgctcctgaggaatatctgcagtc

SEQ ID NO: 24 - DNA, 59 bp, synthetic construct
  source 1..59: mol_type = other DNA
gtgactggagttcagacgtgtgctcttccgatctgactataatgacagttaaccctgcc

SEQ ID NO: 25 - DNA, 4553 bp, synthetic construct
  source 1..4553: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaagatctttatcagacgtcgacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctgcatcattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacgggaccggggttttcttccacgtcgcctgcttgctttaacagagagaagttcgtggcgctagcgcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctctgcggctttccccccatctcagggtcccggccacgtctcttgtccaaaacatcgtactcctctcttcgtcctagattgagctcgttatagagctggttctggccctgctggtacgcgggggcgtctgcgctcctgctgaacttcactctcaggctagcggagcgataggctgcgaagtcgcgtggtggggcatagggctggtaatgcttgcgggtgggtccgggccgcctgggagtcatgttcatgtagtcactgtgcaggagcctgctcctcttactcctcacccagaaaataataaaggccactgttactagcaagctatagcaagccaggactccaccaaccaccaccagcacccaaaagggcttagaaggaggtcttctatcacaggcgaagtccagccccctcgtgtgcactgcgcctccggctgctggtcgacaagcctcaggtcgcagactgagaggctgactcgcaatagttggcgctggcgtcggtggccgaggagcgggcgttgtggtggtacctgcggccgctgaggagacggtgactgaggttccttgaccccagtagtccatagcatagctaccaccgtagtaataatgtttggcacagtagtaaatggctgtgtcatcagtttgcagactgttcatttttaagaaaacttggctcttggagttgtccttgatgatggtcagtctggatttgagagctgaattatagtatgtggtttcactaccccatattactcccagccactccagaccctttcgtggaggctggcgaatccagcttacaccatagtcgggtaatgagacccctgagacagtgcatgtgacggacaggctctgtgagggcgccaccaggccaggtcctgactcctgcagtttcacctcgcccttggtggatccctcgccagatccgggcttgccggatccagaggtggagcctgttatttccaacttagtcccccctccgaacgtgtacggaagcgtattaccctgttggcaaaagtaagtggcaatatcttcttgctccaggttgctaatggtgagagaataatctgttccagacccactgccactgaaccttgatgggactcctgagtgtaatcttgatgtatggtagatcaggagtttaacagttccatctggtttctgctgataccaattcaaatacttagagatgtcctgacttgccctgcaactgatggtgactctgtctcccagagaggcagacagggaggatgtagtctgtgtcatctggatgtctcctgttacttgaattgaagggaataagttgagagccaagagcagcctgagcatggtggccttaaggctgagggtacccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggatgcattaatgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 26 - DNA, 2775 bp, synthetic construct
  source 1..2775: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaagatctttatcagacgtcgacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctgcaggttatcagaccaccgggcgcgggcacttacagacacggcggcggttgcgatggttacagtaaagagtaatcaccagagacaagagcagaaccccgcaggtgccggccaggggagcccagatgtagatgtcacatgcgaaatccaaaccgcgagtgtgcacggcgccgccagccgcgggtcggcaagcctcgggccgtagcgaaagcggctgggaagcgatggtgggggcgggggtaggaggcctcggggccggggagcctcctcccccagagcacagactggggttgctataggggcacgctgtggtcgtaggcttggcagggctcacgttggtggacacgtttgaaaacgtgccctgggttggcagctcggagccacccccaccagaacagagggatgggttggagtatgggcaggcgtcggcgtggtccgcgcccagcaggcacagcgccatccagcacagcaggctggtgcccatggtggccttaaggctgagggtacccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggatgcattaaaaaacctcccacacctccccctgaacctgaaacaagcttgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 27 - DNA, 2487 bp, synthetic construct
  source 1..2487: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaagatctttatcagacgtcgacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctgcaggttatcagctgaagcagaacaggtggatgatggcgttggccacgaaaaacaggaaaattccgccggagatattagaagaagcgctagggctgctggtctgggaggtgtcgttctggccgctcaggccggtttggatctgcaccatcacgagcaggctgattgtcagcagcagaaacaggaatctcttcatggtggccttaaggctgagggtacccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggatgcattaaaaaacctcccacacctccccctgaacctgaaacaagcttgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 28 - DNA, 1464 bp, synthetic construct
  source 1..1464: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaggctccttgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 29 - DNA, 3096 bp, synthetic construct
  source 1..3096: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaagatctttatcagacgtcgacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctgcagggtttaaacgcggccgctcgagttatcactttttcttttttgcctggccggcctttttcgtggccgccggccttttcttgtacagctcgtccatgccgccggtggagtggcggccctcggagcgttcgtactgttccaccacggtgtagtcctcgttgtgggaggtgatgtccaacttgcggtcgacgttgtaggcgccgggcatctgcacgggcttcttggccttgtaggtggtcttgaagtccgccaggtagcggccgccgtccttcaggcgcagggccatcttaatgtcgcccttcagcacgccgtcctcggggtacaaccgctcggtggatgcttcccagcccattgtcttcttctgcattacggggccgtcaggagggaagttggtgccgcggagcttcaccttgtagatcagggtgccgtcctccagggaggtgtcctgggtcacggtcacggcgccgccgtcctcgaagttcatcacgcgctcccacttgaagccctcggggaaggactgcttatagtagtcggggatgtcggcggggtgcttgatgaaggccctggagccgtacatgaactgaggggacaggatgtcccaggagaagggcagggggccacccttggtcaccttcagcttggcggtctgggtgccctcgtaggggcggccctcgccctcgccctcgatctcgaactcgtggccgttcatggagccctccatgtgcaccttgaaccgcatgaactccttgatcactgcctcgcccttgctcaccatggctccctgaaaatacagattctcggcggccgccttaaggctgagggtacccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggatgcattaaaaaacctcccacacctccccctgaacctgaaacaagcttgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 30 - DNA, 4463 bp, synthetic construct
  source 1..4463: mol_type = other DNA
cctggaagaggcatctcgtttgtactttggggaaaggaacgtgaaaggcatgtttgaggtgctggagcccttgcatgctatgatggaacggggcccccagactctgaaggaaacatcctttaatcaggtatggggtaaaaaacacgaaaggacataaccctgtagaaatattctgccactaaaaaaaaatgaaatgagaaaagtttttatagaatgaaaggacgtgacccaaggcctttgtcagatttcttttcctctggcctttccactgactttaataaaacgagctctgatggaggtcacagagcataaaacatcagaaattagttacagggacccttctgaattttaactggaaaagcccaagatttctcaagaggtcatctcgtctctcctctcacccttccttccctataagccatgctgctggtttggaagctccagggttggaggcctgcacctatgctcttgctttctccagccccctgccagtgttttcaggccacagcgggctcctgaggaatatctgcagtcagcctggggattttgcctcctgggttggttgagactttgataacttgaaaacgtgcctgctttctctctcaggcctatggtcgagatttaatggaggcccaagagtggtgcaggaagtacatgaaatcagggaatgtcaaggacctcacccaagcctgggacctctattatcatgtgttgcgacgaatctcaaagcagctgcctcaggtaggatcttcaagatctccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattattggcgccttcggtgccggcacctgacacagaagaagatgcctagcccaatgaaaagcaggaggccggcgacgccccccagcacaatcagggccatgcggccgccttccagttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcaaatttctttccatcttcaagcatcccggtgtagtgcaccacgcaggtctggccgcgcttggggaaggtgcgcccgtctcctggggagatggtttccacctgcactccgctcccacctccgccgctagcgctggacacggtcacgcttgtgccctggccccagtagtccatggcgtagctgccgccgtagtagtagtgcttggcgcagtagtagatggcggtgtcgtcggtctgcaggctgttcatcttcaggaacacctggctcttggagttgtccttgatgatggtcagccgggacttcagggcgctgttgtagtaggttgtctcgctgccccagatcactcccagccattccaggccctttctgggtggctgtctgatccaggacacgccatagtcaggcagggacacgccggacacggtacaggtcacgctcagagactggcttggggccaccagtccagggccgctttcctgcagtttcacttcgcccttggtgcttccctcgccagatccaggcttgccgctgccgcttgtgctgccggtgatttccagcttggtgcctccgccgaaggtgtagggcagggtgttgccttgctgacagaagtaggtagcgatatcttcctgttccaggttggagatggtcaggctgtagtcggtgccggagccgctgccagaaaatctgctgggcacgccgctgtgcagtctgctggtgtggtagatcagcagcttcacggtgccgtcgggtttctgctgataccagttcaggtacttgctgatgtcctggctggctctgcagctgatggtcactctatcgcccaggctggcgctcaggctgctggtggtctgggtcatctggatatcggaaccagtggaacctggaacccagagcagcagtacccatagcaggagtgtgtctgtctccattgaaggcccggggttttcttcaacatctcctgcttgctttaacagagagaagttcgtggcgccagatcctgaccttgggggcagggcctgcatgtgcagggcgtcgtaggtatccttggtggcggtggacaggccctgatacaggccgtcgtggcccttgccccgcctccgctcgcccttcatgccgatctcgctgtaggcctcggccatcttgtctttctgcagttcgttatacaggccttcctgggggttcttccgccgaggcttgccgcccatctcagggtcccggcctctccgcttatccaggacgtcgtactcttcccttctgcccaggttcagctcgttgtacagctgattctggccctgctggtaggcaggggcgtcggcgcttctgctgaacttcacccgcagttcacatcctccttcttcttcttctggaaatcggcagctacagccatcttcctcttgagtagtttgtactggtctcataaatggttgtttgaatatatacaggagtttctttctgccccgtttatgcatggtgatcacgaggctcagcagcagcacgccacatgtgccggccagaggggcccagatgtagatgtcggaaccagtgccggcgctagccttgctgatgcgccggaacacgtggtagtacaggtcccaggcctgcagcaggtccttcacattgccggacttcatgtactttctgcaccactcctgggcttccatcaggtctctgccgtaggcctggttgaagctggtctctttcagggtctgaggtcctctttccatcatggcgtgcaggggttccagcacctcgaacatgcccttgacgttccgctcgccgaaatagagtctgctggcctcttccaggccctcatgccacatctcgtgccacaggatggatccgggtctggcggcatggagcagcagggccagaggcagcagcagagctgtcacaggcagagccatggtggccttaaggctgagggtacccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggatgcattaatgcagggttaactgtcattatagtcctttctgttttactgtctatgaatgttctaacactcattccaaagcatctggttttacttctgctttgggacaagtaattgttattagcaatccactgaaaaatacttaaattgcgtttaaattcttccctggaggcagaacactaaatcctttggtaattatttacagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggtaaggaccattgtgcacttctgtcacccacttctccctcctacactggctgagaccccttccctcttctgaaattactgcttgccattcccactactcacctatcatagctacatctctcccatgctcattgagaggaaaatagagggcaagcaaatttctcattatgtcctcaggacttagttcattcatgtagttgggcttatgtgaagcatgatcacacataaagggaatgacaaaggcatgcaaggcagagaagtaacagtggaaggcaggtgttggagactgatgaggccagggaggaggcagctgggtgcaagcagggaaggtgctag

SEQ ID NO: 31 - DNA, 55 bp, Homo sapiens
  source 1..55: mol_type = genomic DNA
agcctgggacctctattatcatgtgttccgacgaatctcaaagcagctgcctcag

SEQ ID NO: 32 - DNA, 28 bp, Homo sapiens
  source 1..28: mol_type = genomic DNA
gtaggatcttcaggctcctggcagggtt

SEQ ID NO: 33 - DNA, 83 bp, Homo sapiens
  source 1..83: mol_type = genomic DNA
agcctgggacctctattatcatgtgttccgacgaatctcaaagcagctgcctcaggtaggatcttcaggctcctggcagggtt

SEQ ID NO: 34 - DNA, 10 bp, Homo sapiens
  source 1..10: mol_type = other DNA
gtgttccgac

SEQ ID NO: 35 - DNA, 36 bp, Homo sapiens
  source 1..36: mol_type = other DNA
tcaggtaggatcttcaggctcctggcagggttaact

SEQ ID NO: 36 - DNA, 10 bp, synthetic construct
  source 1..10: mol_type = other DNA
gtgttgcgac

SEQ ID NO: 37 - DNA, 36 bp, synthetic construct
  source 1..36: mol_type = other DNA
tcaggtaggatcttcaggctccttgcagggttaact

SEQ ID NO: 38 - DNA, 10 bp, synthetic construct
  source 1..10: mol_type = other DNA
gtgttgcgac

SEQ ID NO: 39 - DNA, 36 bp, synthetic construct
  source 1..36: mol_type = other DNA
tcaggtaggatcttcaggctcctggcagtgttaact

SEQ ID NO: 40 - DNA, 25 bp, synthetic construct
  source 1..25: mol_type = other DNA
ttccgacgaatctcaaagcagctgc

SEQ ID NO: 41 - DNA, 25 bp, synthetic construct
  source 1..25: mol_type = other DNA
ttccgacgaatctcaaagcagctgc

SEQ ID NO: 42 - DNA, 23 bp, synthetic construct
  source 1..23: mol_type = other DNA
cctctattatcatgtgttccgac

SEQ ID NO: 43 - DNA, 23 bp, synthetic construct
  source 1..23: mol_type = other DNA
cctctattatcatgtgttccgac

SEQ ID NO: 44 - DNA, 23 bp, synthetic construct
  source 1..23: mol_type = other DNA
cctctattatcatgtgttccgac

SEQ ID NO: 45 - DNA, 10 bp, synthetic construct
  source 1..10: mol_type = other DNA
gtgttccgac

SEQ ID NO: 46 - DNA, 28 bp, synthetic construct
  source 1..28: mol_type = other DNA
tcaggtaggatcttcaggctcctggcag

SEQ ID NO: 47 - DNA, 10 bp, synthetic construct
  source 1..10: mol_type = other DNA
gtgttgcgac

SEQ ID NO: 48 - DNA, 28 bp, synthetic construct
  source 1..28: mol_type = other DNA
tcaggtaggatcttcaggctccttgcag

SEQ ID NO: 49 - protein, 11 aa, synthetic construct
YGRKKRRQRRR

SEQ ID NO: 50 - protein, 12 aa, synthetic construct
RRQRRTSKLMKR

SEQ ID NO: 51 - protein, 27 aa, synthetic construct
GWTLNSAGYLLGKINLKALAALAKKIL

SEQ ID NO: 52 - protein, 33 aa, synthetic construct
KALAWEAKLAKALAKALAKHLAKALAKALKCEA

SEQ ID NO: 53 - protein, 16 aa, synthetic construct
RQIKIWFQNRRMKWKK

SEQ ID NO: 54 - protein, 8 aa, synthetic construct
KKRRQRRR

SEQ ID NO: 55 - protein, 9 aa, synthetic construct
RKKRRQRRR

SEQ ID NO: 56 - protein, 14 aa, synthetic construct
SGYGRKKRRQRRRC

SEQ ID NO: 57 - protein, 11 aa, synthetic construct
YGRKKRRQRRR

SEQ ID NO: 58 - protein, 8 aa, synthetic construct
KKRRQRRR

SEQ ID NO: 59 - protein, 21 aa, synthetic construct
KETWWETWWTEWSQPKKKRKV

SEQ ID NO: 60 - RNA, 20 nt, synthetic construct
  source 1..20: mol_type = other RNA
taatttctactcttgtagat

SEQ ID NO: 61 - protein, 7 aa, synthetic construct
PKKKRKV

SEQ ID NO: 62 - protein, 16 aa, synthetic construct
KRPAATKKAGQAKKKK

SEQ ID NO: 63 - protein, 11 aa, synthetic construct
SPKKKRKVEAS

SEQ ID NO: 64 - protein, 6 aa, synthetic construct
KKKRKV

SEQ ID NO: 65 - protein, 18 aa, synthetic construct
SPKKKRKVEASPKKKRKV

SEQ ID NO: 66 - protein, 5 aa, synthetic construct
KKKRK

SEQ ID NO: 67 - protein, 7 aa, synthetic construct
PKKKRKV

(19) United States
(12) Patent Application Publication
(10) Pub. No.: US 2024/0352487 A1
(43) Pub. Date: Oct. 24, 2024

(54) MTOR -TARGETED MODIFICATION AND USES THEREOF

(71) Applicant: UNIVERSITÉ LAVAL, Quebec (CA)

(72) Inventors: Sébastien LEVESQUE, Quebec (CA); Yannick DOYON, Quebec (CA)

(21) Appl. No.: 18/682,277

(22) PCT Filed: Aug. 5, 2022
(86) PCT No.: PCT/CA2022/051201

§ 371 Date: Feb. 8, 2024

Related U.S. Application Data
(60) Provisional application No. 63/231,847, filed on Aug. 11, 2021.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12Y 207/07049; C07K 2319/00; C12N 2310/20

(57) ABSTRACT

Described herein are reagents, products, methods, and uses thereof for modifying the genome of a cell at a MTOR locus, for example by nuclease-based (e.g. CRISPR/Cas9) modification. The modifications include a MTOR modification which confers resistance to an mTOR inhibitor. The modifications may further include a modification to introduce a nucleic acid or gene of interest. Introducing both modifications at the MTOR locus is an efficient and advantageous strategy which allows for targeted modification of both aspects (resistance and gene of interest) at the same locus, and further allows for the use of an mTOR inhibitor to select and enrich for such modified cells. Such modified cells may be used for a variety of applications, including therapeutic applications.

### CROSS REFERENCE TO RELATED APPLICATIONS

The present application claims the benefit of U.S. Provisional application No. 63/231,847 filed Aug. 11, 2021, which is incorporated herein by reference.

### REFERENCE TO SEQUENCE LISTING

This application contains a Sequence Listing in computer readable form entitled “11229_454_SeqList.xml”, created Aug. 5, 2022 and having a size of about 232 KB. The computer readable form is incorporated herein by reference.

### FIELD OF THE DISCLOSURE

The present disclosure relates to genome editing and uses thereof. More specifically, the present disclosure is concerned with reagents, products, and methods for modifying the genome of a cell at an MTOR locus, and for selecting and isolating and various uses of cells bearing the targeted modification.

### BACKGROUND OF THE DISCLOSURE

A number of genetic engineering-based strategies have been and are being developed for various therapeutic applications. For example, adoptive cell therapy for cancer with chimeric antigen receptor T (CAR-T) cells is a promising tool for cancer therapy. CAR-T cells are genetically engineered T cells that are able to recognize and target cancer cells expressing a specific antigen on their surface^{4,5}. Randomly integrating vectors based on gamma retroviruses or transposons are routinely used for cell therapy applications, leading to safety concerns regarding clonal expansion, oncogenic transformation, transcriptional silencing, and variegated transgene expression⁶⁻¹⁰. A correlation between genomic modification by CD19-CAR vector integration and clonal T cell proliferation in 39 patients has also been observed¹¹, suggesting that randomly integrating vectors could lead to inconsistent treatment outcomes.

There is thus a need for novel therapeutic approaches, such as in the area of CAR immune cell therapy.

The present description refers to a number of documents, the content of which is herein incorporated by reference in their entirety.

### SUMMARY OF THE DISCLOSURE

The present disclosure relates to genome editing and uses thereof. More specifically, the present disclosure is concerned with reagents, products, and methods for modifying the genome of a cell, e.g., at an MTOR locus (and optionally at other loci), and for selecting and isolating and various uses of cells bearing the targeted modification(s).

In various aspects and embodiments, the present disclosure provides the following items:

1. A method for nuclease-based modification of a target polynucleotide at an MTOR locus in a target cell, the method comprising:
(i) introducing a first modification at the MTOR locus, wherein the first modification confers resistance to an mTOR inhibitor;
(ii) introducing a second modification at the MTOR locus, wherein the second modification comprises introduction of a nucleic acid of interest at the MTOR locus.

2. The method of item 1, wherein said nuclease-based modification in the target polynucleotide is introduced by homology-directed repair (HDR) and the method comprises introducing into the cell one or more donor nucleic acids comprising the first and second modifications.

3. The method of item 2, wherein a single donor nucleic acid comprises the first and second modifications.

4. The method of any one of items 1-3, further comprising:
(iii) contacting the target cell with the mTOR inhibitor;
(iv) selecting a cell comprising the first and second modifications by virtue of increased tolerance to the mTOR inhibitor, thereby enriching for the cell comprising the first and second modifications.

5. The method of any one of items 1-4, wherein the mTOR inhibitor is rapamycin or a rapalog.

6. The method of item 5, wherein the rapalog is temsirolimus, everolimus, deforolimus, umirolimus, zotarolimus, ridaforolimus, or any combination thereof.

7. The method of any one of items 1-6, wherein the method comprises providing the target cell with a CRISPR nuclease and one or more gRNAs comprising one or more guide sequences having one or more target sequences in the target polynucleotide, such that the one or more gRNAs direct the cleavage of the target polynucleotide at the MTOR locus to introduce the first and second modifications, wherein the one or more target sequences are each contiguous to a protospacer adjacent motif recognized by the CRISPR nuclease.

8. The method of any one of items 1-6, wherein the method comprises providing said cell with:
a. a1) at least one guide RNA comprising one or more guide sequences having one or more target sequences in the target polynucleotide, such that the one or more gRNAs direct the cleavage of the target polynucleotide at the MTOR locus to introduce the first and second modifications, wherein the one or more target sequences are each contiguous to a protospacer adjacent motif recognized by a Cas9 nickase; or
   a2) a vector comprising a nucleic acid sequence corresponding to the guide RNA for expressing the guide RNA; and
b. b1) a Cas nickase-deaminase fusion protein comprising (i) a Cas nickase polypeptide domain and (ii) a cytidine deaminase polypeptide domain or an adenosine deaminase polypeptide domain; or
   b2) a vector comprising a nucleic acid sequence encoding the Cas nickase-deaminase fusion protein, for expression of the Cas nickase-deaminase fusion protein; and
wherein at least one modification is introduced into the target polynucleotide, wherein the at least one modification comprises modification of a cytidine into a thymidine or an adenosine into an inosine, and wherein the at least one modification results in the introduction of the first modification.

9. The method of item 8, wherein said Cas nickase polypeptide domain comprises a SpCas9 nickase, a SaCas9 nickase, a CjCas9 nickase, a ScCas9 nickase, a CasX nickase, a CasY nickase, a Cpf1 nickase, a eSaCas9 nickase, a eSpCas9 nickase, a HiFi Cas9 nickase, a Cas9 H840A nickase, a St1Cas9 nickase, or a derivative of any thereof having nickase activity.

10. The method of item 8 or 9, wherein said Cas nickase-deaminase fusion protein is SpCas9n VQR BE3, SpCas9n EQR BE3, SaCas9n-KKH BE3, YE1-SpCas9n VQR BE3, YE1-SpCas9n EQR BE3, YE1-SaCas9n-KKH BE3, SpCas9n VQR BE4, SpCas9n EQR BE4, SaCas9n-KKH BE4, Target-AID SpCas9n VQR, Target-AID SpCas9n EQR, Target-AID SaCas9n-KKH, Target-AID-BE3, a fusion protein comprising an St1Cas9-derived nickase, a fusion protein comprising a St3Cas9-derived nickase, or a derivative of any thereof comprising nickase and deaminase activity.

11. The method of any one of items 8 to 10, wherein the nucleic acid sequence corresponding to the guide RNA and the nucleic acid sequence encoding the Cas nickase-deaminase fusion protein are comprised in the same vector.

12. The method of any one of items 1-6, wherein the method comprises providing said cell with:
a. a1) at least one pegRNA comprising one or more guide sequences having one or more target sequences in the target polynucleotide, such that the one or more pegRNAs direct the cleavage of the target polynucleotide at the MTOR locus to introduce the first and second modifications, wherein the one or more target sequences are each contiguous to a protospacer adjacent motif recognized by a Cas9 nickase; or
   a2) a vector comprising a nucleic acid sequence corresponding to the pegRNA for expressing the pegRNA; and
b. b1) a Cas nickase-reverse transcriptase fusion protein comprising (i) a Cas nickase polypeptide domain, and
   (ii) a reverse transcriptase polypeptide domain; or
b2) a vector comprising a nucleic acid sequence encoding the Cas nickase-reverse transcriptase fusion protein, for expression of the Cas nickase-reverse transcriptase fusion protein; and wherein at least one modification is introduced into the target polynucleotide, wherein the at least one modification results in the introduction of the first modification.

13. The method of item 12, wherein said Cas nickase polypeptide domain comprises a SpCas9 nickase, a SaCas9 nickase, a CjCas9 nickase, a ScCas9 nickase, a CasX nickase, a CasY nickase, a Cpf1 nickase, a eSaCas9 nickase, a eSpCas9 nickase, a HiFi Cas9 nickase, a Cas9 H840A nickase, a St1Cas9 nickase, or a derivative of any thereof having nickase activity.

14. The method of item 12 or 13, wherein the reverse transcriptase domain comprises an M-MLV reverse transcriptase.

15. The method of any one of items 12 to 14, wherein the nucleic acid sequence corresponding to pegRNA and the nucleic acid sequence encoding the Cas nickase-reverse transcriptase fusion protein are comprised in the same vector.

16. The method of any one of items 1-15, wherein the target polynucleotide is within an exon of an MTOR gene, within an intron of the MTOR gene, or spanning an exon-intron boundary of an MTOR gene.

17. The method of any one of items 7-16, wherein the one or more target sequences are within an exon of an MTOR gene, within an intron of the MTOR gene, or spanning an exon-intron boundary of an MTOR gene.

18. The method of item 16 or 17, wherein the exon is exon 45 of the MTOR gene.

19. The method of item 16 or 17, wherein the intron is intron 45 of the MTOR gene.

20. The method of item 16 or 17, wherein the exon-intron boundary is the exon 45-intron 45 boundary of the MTOR gene.

21. The method of item 16 or 17, wherein the target polynucleotide or the one or more target sequences are within a nucleotide region set forth in any of FIG. 1, FIG. 2 or FIG. 12, in the sense or antisense sequence.

22. The method of any one of items 7-21, wherein the one or more target sequences comprise one or more of 5′-CACATGATAATAGAGGTCCC-3′ (antisense strand, SEQ ID NO: 3), 5′-GTCGGAACACATGATAATAG-3′ (antisense strand, SEQ ID NO: 4), 5′-GCAGCTGCTTTGAGATTCGT-3′ (antisense strand, SEQ ID NO: 5); 5′-AGGTAGGATCTTCAGGCTCC-3′ (sense strand, SEQ ID NO: 6); and 5′-GGATCTTCAGGCTCCTGGCA-3′ (sense strand, SEQ ID NO: 7).

23. The method of any one of items 1-22, wherein the first modification results in an amino acid substitution at position 2108 of an mTOR polypeptide.

24. The method of item 23, wherein the substitution is a F2108L, F21081, F2108M, F2108V, F2108K or F2108G substitution.

25. The method of item 23 or 24, wherein the substitution is a F2108L, F21081, F2108M or F2108G substitution.

26. The method of any one of items 23 to 25, wherein the substitution is a F2108L substitution.

27. The method according to any one of items 1-26, wherein the method does not result in a modification at a locus other than the MTOR locus.

28. The method according to any one of items 1-27, wherein the MTOR locus comprising the first and second modifications encodes an mTOR polypeptide that retains mTOR signaling activity.

29. The method of any one of items 1-28, wherein the first and second modifications are introduced in a single step.

30. The method of any one of items 3-29, wherein the first and second modifications are introduced in a single step via the use of the single donor nucleic acid comprising the first and second modifications.

31. The method of any one of items 1-30, wherein the nucleic acid of interest is not present at an MTOR locus in a corresponding cell lacking the second modification.

32. The method of any one of items 7-31, wherein the CRISPR nuclease is a Cas9 nuclease.

33. The method of any one of items 7-32, wherein the CRISPR nuclease further comprises one or more nuclear localization signals (NLS).

34. The method of any one of items 1-33, wherein the cell is a stem cell.

35. The method of item 34, wherein the stem cell is a hematopoietic stem cell (HSC), an embryonic stem cell, a totipotent stem cell, a pluripotent stem cell, a multipotent stem cell, or an induced pluripotent stem cell (iPSC).

36. The method of any one of items 1-35, wherein the cell is an immune cell.

37. The method of item 36, wherein the immune cell is a T cell, a natural killer (NK) cell, or a B cell.

38. The method of any one of items 1-37, wherein the nucleic acid of interest encodes a therapeutic protein.

39. The method of any one of items 1-38, wherein the nucleic acid of interest encodes a chimeric antigen receptor (CAR).

40. The method of any one of items 1-39, which is an in vitro method.

41. The method of any one of items 1-40, which is an in vivo method and the cell is in a subject.

42. The method of any one of items 1-41, further comprising modification of one or more further target polynucleotides at one or more further loci other than the MTOR locus in the target cell, the method further comprising introducing one or more further modifications at the one or more further loci, wherein the one or more further modifications comprise introduction of one or more further nucleic acids of interest at the one or more further loci.

43. The method of item 42, wherein the method comprises providing the target cell with a CRISPR nuclease and one or more gRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by the CRISPR nuclease.

44. The method of item 42, wherein the method comprises providing said cell with:
a. a1) one or more gRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by the CRISPR nuclease; or
   a2) one or more vectors comprising one or more nucleic acid sequences corresponding to the one or more gRNAs for expressing the one or more gRNAs; and
b. b1) a Cas nickase-deaminase fusion protein comprising (i) a Cas nickase polypeptide domain and (ii) a cytidine deaminase polypeptide domain or an adenosine deaminase polypeptide domain; or
   b2) a vector comprising a nucleic acid sequence encoding the Cas nickase-deaminase fusion protein, for expression of the Cas nickase-deaminase fusion protein; and wherein at least one of the one or more further modifications comprises modification of a cytidine into a thymidine or an adenosine into an inosine.

45. The method of item 42, wherein the method comprises providing said cell with:
a. a1) one or more pegRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by a Cas9 nickase; or
   a2) one or more vectors comprising one or more nucleic acid sequences corresponding to the one or more pegRNAs for expressing the one or more pegRNAs; and
b. b1) a Cas nickase-reverse transcriptase fusion protein comprising (i) a Cas nickase polypeptide domain and
   (ii) a reverse transcriptase polypeptide domain; or b2) a vector comprising a nucleic acid sequence encoding the Cas nickase-reverse transcriptase fusion protein, for expression of the Cas nickase-reverse transcriptase fusion protein.

46. One or more gRNAs or pegRNAs as defined in any one of items 7-45.

47. One or more donor nucleic acids as defined in any one of items 2-46.

48. An expression cassette comprising (a) a modified MTOR nucleic acid comprising the first modification as defined in any one of items 1-45 and encoding a modified mTOR polypeptide that is resistant to an mTOR inhibitor as defined in any one of items 1-45; and (b) the nucleic acid of interest as defined in any one of items 1-45.

49. One or more vectors comprising nucleic acid sequence(s) encoding the one or more gRNAs or pegRNAs of item 46, the one or more donor nucleic acids of item 47; the expression cassette of item 44; and/or the CRISPR nuclease as defined in any one of items 7-45.

50. The one or more vectors of item 49, wherein the one or more vectors are viral vectors.

51. A cell comprising the modified polynucleotide comprising the first and second modifications as defined in any one of items 1-45.

52. A cell comprising nucleic acid sequence(s) encoding the one or more gRNAs or pegRNAs of item 46, the one or more donor nucleic acids of item 47, the expression cassette of item 48, and/or the CRISPR nuclease as defined in any one of items 7-45.

53. A cell comprising the one or more vectors of item 49 or 50.

54. The cell of any one of items 51-53, wherein the cell is a stem cell.

55. The cell of item 54, wherein the stem cell is a hematopoietic stem cell (HSC), an embryonic stem cell, a totipotent stem cell, a pluripotent stem cell, a multipotent stem cell, or an induced pluripotent stem cell (iPSC).

56. The cell of any one of items 51-53, wherein the cell is an immune cell.

57. The cell of item 56, wherein the immune cell is a T cell, a natural killer (NK) cell, or a B cell.

58. A composition comprising nucleic acid sequence(s) encoding the one or more gRNAs or pegRNAs of item 46, the one or more donor nucleic acids of item 47, the expression cassette of item 48, the CRISPR nuclease as defined in any one of items 7-45, the one or more vectors of items 49 or 50, and/or the cell of any one of items 51-57.

59. The composition of item 58, further comprising a pharmaceutically acceptable carrier.

60. Use of the one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59, for modifying one or more target polynucleotides in a cell.

61. Use of the one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for the preparation of a medicament for modifying one or more target polynucleotides in a cell.

62. The one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for use in modifying one or more target polynucleotides in a cell.

63. The one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for use as a medicament.

64. A method for treating a disease, condition or disorder in a subject, the method comprising administering an effective amount of the one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, to said subject.

65. The method of item 64, wherein the disease, condition, or disorder is associated with expression of an antigen, and wherein the nucleic acid of interest encodes a recombinant receptor that specifically binds to the antigen.

66. The method of item 65, wherein the recombinant receptor is a chimeric antigen receptor (CAR).

67. The method of item 65 or 66, wherein the disease, condition, or disorder is a cancer, an autoimmune or inflammatory disease, or an infectious disease.

68. The method of item 67, wherein the disease, condition, or disorder is a cancer, and wherein the method further comprises administering an effective amount of an anticancer agent to the subject.

69. The method of item 68, wherein the anticancer agent is rapamycin or a rapalog.

70. The method of any one of items 64-69, wherein the cells are autologous cells.

71. The method of any one of items 64-69, wherein the cells are allogeneic cells.

72. Use of the one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for treating a disease, condition, or disorder in a subject.

73. Use of the one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for the preparation of a medicament for treating a disease, condition, or disorder in a subject.

74. The use of item 72 or 73, wherein the disease, condition, or disorder is associated with expression of an antigen, and wherein the nucleic acid of interest encodes a recombinant receptor that specifically binds to the antigen.

75. The use of item 74, wherein the recombinant receptor is a chimeric antigen receptor (CAR).

76. The use of item 74 and 75, wherein the disease, condition, or disorder is a cancer, an autoimmune or inflammatory disease, or an infectious disease.

77. The use of item 76, wherein the disease, condition, or disorder is a cancer, and wherein the method further comprises administering an effective amount of an anticancer agent to the subject.

78. The use of item 77, wherein the anticancer agent is rapamycin or a rapalog.

79. The use of any one of items 72-78, wherein the cells are autologous cells.

80. The use of any one of items 72-78, wherein the cells are allogeneic cells.

81. The one or more gRNAs or pegRNAs of item 46, the one or more donor nucleic acids of item 47, the expression cassette of item 45, the CRISPR nuclease as defined in any one of items 7-45, the one or more vectors of items 49 or 50, the cell of any one of items 51-57, and/or the composition of item 58 or 59itemitemitemitemitemitem, for use in treating a disease, condition, or disorder in a subject.

82. The one or more gRNAs or pegRNAs, one or more donor nucleic acids, expression cassette, CRISPR nuclease, one or more vectors, cell, and/or composition for use of item 81, wherein the disease, condition, or disorder is associated with expression of an antigen, and wherein the nucleic acid of interest encodes a recombinant receptor that specifically binds to the antigen.

83. The one or more gRNAs or pegRNAs, one or more donor nucleic acids, expression cassette, CRISPR nuclease, one or more vectors, cell, and/or composition for use of item 82, wherein the recombinant receptor is a chimeric antigen receptor (CAR).

84. The one or more gRNAs or pegRNAs, one or more donor nucleic acids, expression cassette, CRISPR nuclease, one or more vectors, cell, and/or composition for use of item 81 or 82, wherein the disease, condition, or disorder is a cancer, an autoimmune or inflammatory disease, or an infectious disease.

85. The one or more gRNAs or pegRNAs, one or more donor nucleic acids, expression cassette, CRISPR nuclease, one or more vectors, cell, and/or composition for use of item 84, wherein the disease, condition, or disorder is a cancer, and wherein the method further comprises administering an effective amount of an anticancer agent to the subject.

86. The one or more gRNAs or pegRNAs, one or more donor nucleic acids, expression cassette, CRISPR nuclease, one or more vectors, cell, and/or composition for use of item 85, wherein the anticancer agent is rapamycin or a rapalog.

87. The one or more gRNAs or pegRNAs, one or more donor nucleic acids, expression cassette, CRISPR nuclease, one or more vectors, cell, and/or composition for use of any one of items 81-86, wherein the cells are autologous cells.

88. The one or more gRNAs or pegRNAs, one or more donor nucleic acids, expression cassette, CRISPR nuclease, one or more vectors, cell, and/or composition for use of any one of items 81-86, wherein the cells are allogeneic cells.

Other objects, advantages, and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows studies of the identification of highly active guide RNAs (gRNAs) targeting the MTOR locus. (A) Schematic representation of SpCas9 target sites surrounding the F2108 codon at MTOR exon 45 (SEQ ID NO: 31) and intron 45 (SEQ ID NO: 32). Full sequence in figure corresponds to SEQ ID NO: 33. (B) K562 cells were transfected with 0.75 μg of SpCas9-sgRNA-expressing plasmid and genomic DNA was harvested 3 days post-transfection. The percentage of alleles harboring insertions and deletions (indels) was quantified by TIDE.

FIG. 2 shows studies of the introduction of the F2108L mutation to the MTOR locus, which allows the selection of rapamycin-resistant K562 cells. (A) Strategy for the CRISPR-driven insertion of the F2108L mutation to the MTOR locus using a plasmid donor. (B) Schematic representation of the SpCas9 target sites and the plasmid donor sequences harboring the F2108L and PAM mutations (WT sequence: 5′ portion corresponds to SEQ ID NO: 34 and 3′ portion corresponds to SEQ ID NO: 35; G49 donor: 5′ portion corresponds to SEQ ID NO: 36 and 3′ portion corresponds to SEQ ID NO: 37; G54 donor: 5′ portion corresponds to SEQ ID NO: 38 and 3′ portion corresponds to SEQ ID NO: 39). (C) K562 cells were transfected with 0.5 μg of SpCas9-sgRNA-expressing plasmid and 1 μg of donor plasmid harboring the PAM and F2108L mutations. Cells were treated with 0.5 μM rapamycin 3 days post-transfection until all non-resistant cells were eliminated. The percentage of edited alleles was quantified by TIDER after selection. HA; homology arm.

FIG. 3 shows studies of targeting a CAR-2A-EGFP gene cassette to the MTOR locus, which allows the selection of rapamycin-resistant K562 cells. (A) Strategy for the CRISPR-driven CAR-2A-EGFP gene knock-in to the MTOR locus using a plasmid donor. (B) K562 cells were transfected with 0.5 μg of SpCas9-sgRNA-expressing plasmid and 1 μg of donor plasmid. Cells were treated with 0.5 μM rapamycin 3 days post-transfection until all non-resistant cells were eliminated. The percentage of cells expressing the EGFP reporter was quantified by FACS after selection. HA; homology arm. PA; polyadenylation signal. EGFP; enhanced green fluorescent protein. 2A self-cleaving peptide. CAR; chimeric antigen receptor. hPGK1; human phosphoglycerate kinase 1 promoter.

FIG. 4 shows studies of targeting suicide gene cassettes to the MTOR locus, which allows the selection of rapamycin-resistant K562 cells. (A) Strategy for the CRISPR-driven RQR8 gene knock-in to the MTOR locus using a plasmid donor. (B) K562 cells were transfected with 0.5 μg of SpCas9-sgRNA-expressing plasmid and 1 μg of donor plasmid harboring. Cells were treated with 0.5 μM rapamycin 3 days post-transfection until all non-resistant cells were eliminated. The percentage of cells expressing the RQR8 epitope marker was quantified by FACS after selection using an Alexa Fluor 488-conjugated anti-human CD34 antibody. (C) Same as in A, but for CRISPR-driven CD52 gene knock-in. (D) Same as in B, but the percentage of cells expressing CD52 was quantified using a FITC-conjugated anti-human CD52 antibody. HA; homology arm. PA; polyadenylation signal. EGFP; enhanced green fluorescent protein. 2A self-cleaving peptide. CAR; chimeric antigen receptor. hPGK1; human phosphoglycerate kinase 1 promoter.

FIG. 5 shows that mTORC1 signaling remains functional after targeted transgene integration at the MTOR locus. (A) Schematic representation of the mScarlet-I mTOR Signaling Indicator (mSc-TOSI) degradation under mTORC1 signaling. (B) mSc-TOSI fluorescence intensity in bulk populations of K562 cells stably expressing the mTORC1 signaling reporter. Where indicated, cells were treated with the indicated concentration of mTOR inhibitor for 24 hours before FACS analysis. Representative FACS plots are from one out of two independent biological replicates performed at different times with equivalent results.

FIG. 6 shows that mTORC1 signaling remains functional after targeted CAR integration at the MTOR locus in homozygous single cell-derived clones. (A) Schematic representation of the mScarlet-I mTOR Signaling Indicator (mSc-TOSI) degradation under mTORC1 signaling. (B) mSc-TOSI fluorescence intensity in homozygous single cell-derived K562 clones harboring the MTOR-F2108L mutation with or without targeted CAR integration. Single cell-derived K562 clones were isolated in methylcellulose-based semi-solid RPMI media supplemented with 100 μM ouabain. Where indicated, cells were treated with the indicated concentration of mTOR inhibitor for 24 hours before FACS analysis. Representative FACS plots are from one out of two independent biological replicates performed at different times with equivalent results.

FIG. 7 shows that prime editing at the MTOR locus confers resistance to rapamycin. (A) Prime editing and small indels quantification at MTOR as determined by BEAT and TIDE analysis from Sanger sequencing. K562 cells stably expressing the mSc-TOSI reporter were transfected with 0.75 μg of pCMV-PE2 vector, 0.25 μg of pegRNA vector, and 0.1 μg of nick sgRNA vector. Genomic DNA was harvested 3 days post-transfection (before selection) and cells were treated (rapamycin) or not (vehicle) with 0.5 μM rapamycin until all non-resistant cells were eliminated (sequence at left corresponds to SEQ ID NO: 40; sequence at right corresponds to SEQ ID NO: 41). (B) mSc-TOSI fluorescence intensity in bulk populations of K562 cells after rapamycin selection. Where indicated, cells were treated with the indicated concentration of mTOR inhibitor for 24 hours before FACS analysis.

FIG. 8 shows studies of adenine base editing at the MTOR locus. (A) Adenine base editing quantification at MTOR as determined by BEAT analysis from Sanger sequencing. K562 cells stably expressing the mSc-TOSI reporter were transfected with 0.75 μg of pCMV-ABEmax or ABE8e vector and 0.25 μg of sgRNA vector. Genomic DNA was harvested 3 days post-transfection (before selection) and cells were treated (rapamycin) or not (vehicle) with 0.5 μM rapamycin until all non-resistant cells were eliminated (sequence at left corresponds to SEQ ID NO: 42; sequence at right corresponds to SEQ ID NO: 43). (B) mSc-TOSI fluorescence intensity in bulk populations of K562 cells after rapamycin selection. Where indicated, cells were treated with the indicated concentration of mTOR inhibitor for 24 hours before FACS analysis (sequence shown corresponds to SEQ ID NO: 44).

FIG. 9 shows the identification of rapamycin resistance mutation via saturation prime editing. (A) High throughput sequencing quantification of the MTOR-F2108 mutations introduced via saturation prime editing following rapamycin selection and FACS sorting. K562 cells stably expressing the mSc-TOSI reporter were transfected with 0.75 μg of pCMV-PEmax vector, 0.25 μg of epegRNA (tevopreQ1) vector, and 0.1 μg of nick sgRNA vector. Genomic DNA was harvested 3 days post-transfection (before selection) and cells were treated (rapamycin) or not (vehicle) with 0.5 μM rapamycin until all non-resistant cells were eliminated. Following rapamycin selection, resistant cells were treated for 24 hours with 0.5 μM rapamycin and FACS-sorted for low mSc-TOSI fluorescence intensity (functional mTORC1 signaling). The fold enrichment of each MTOR-F2108 mutation was calculated from the before selection sample.

FIG. 10 shows marker-free selection of rapamycin-resistant K562 cells with different MTOR-F2108 mutations. (A) Prime editing and small indels quantification at MTOR as determined by BEAT and TIDE analysis from Sanger sequencing. K562 cells stably expressing the mSc-TOSI reporter were transfected with 0.75 μg of pCMV-PEmax vector, 0.25 μg of epegRNA (tevopreQ1) vector, and 0.1 μg of nick sgRNA vector. Genomic DNA was harvested 3 days post-transfection (before selection) and cells were treated (rapamycin) or not (vehicle) with 0.5 μM rapamycin until all non-resistant cells were eliminated.

FIG. 11 shows that MTOR-F2108 mutations display different levels of mTORC1 signaling upon rapamycin treatment. (A) Schematic representation of the mScarlet-I mTOR Signaling Indicator (mSc-TOSI) degradation under mTORC1 signaling. (B) mSc-TOSI fluorescence intensity in bulk populations of K562 cells after rapamycin selection (See FIG. 10). Where indicated, cells were treated with the indicated concentration of mTOR inhibitor for 24 hours before FACS analysis.

FIG. 12 shows studies of the introduction of the F2108L mutation to the MTOR locus, which allows the selection of rapamycin-resistant CD3⁺ T cells. (A) Strategy for the CRISPR-driven insertion of the F2108L mutation to the MTOR locus using a linear double-stranded DNA (dsDNA) donor. (B) Schematic representation of the SpCas9 target site and the dsDNA donor sequence harboring the F2108L and PAM mutations (WT sequence: 5′ portion corresponds to SEQ ID NO: 45 and 3′ portion corresponds to SEQ ID NO: 46; G49 donor: 5′ portion corresponds to SEQ ID NO: 47 and 3′ portion corresponds to SEQ ID NO: 48). (C) CD3⁺ T cells were transfected with 50 pmol of SpCas9 RNP and the percentage of edited alleles was quantified by TIDE 18 days post-transfection. (D) CD3⁺ T cells were transfected with 50 pmol of SpCas9 RNP and the indicated concentration of dsDNA donor. Cells were treated with 25 nM rapamycin 3 days post-transfection and cultured for an additional 15 days. The percentage of edited alleles was quantified by TIDER after selection.

FIG. 13 shows that non-viral targeting of mScarlet-I to the MTOR locus allows the selection of rapamycin-resistant CD3+ T cells. (A) Strategy for CRISPR-driven mScarlet-I gene knock-in to the MTOR locus using a linear double-stranded DNA (dsDNA) donor. (B) CD3+ T cells were transfected with 50 pmol of SpCas9 RNP and the indicated amount of dsDNA donor. Cells were treated with 25 nM rapamycin 3 days post-transfection and cultured for an additional 8 days. The percentage of cells expressing the mScarlet-I reporter was quantified by FACS after selection. HA, homology arm. PA, polyadenylation signal. EGFP, enhanced green fluorescent protein. 2A self-cleaving peptide. CAR, chimeric antigen receptor. hPGK1, human phosphoglycerate kinase 1 promoter.

FIG. 14 shows studies of non-viral targeting a CAR to the MTOR locus, which allows the selection of rapamycin-resistant CD3⁺ CAR-T cells. (A) Strategy for the CRISPR-driven CAR-2A-EGFP gene knock-in to the MTOR locus using a linear double-stranded DNA (dsDNA) donor. (B) CD3⁺ T cells were transfected with 50 pmol of SpCas9 RNP and 1 μg of dsDNA donor. Cells were treated with 25 nM rapamycin 3 days post-transfection and cultured for an additional 8 days. The percentage of cells expressing the EGFP reporter was quantified by FACS after selection. HA; homology arm. PA; polyadenylation signal. EGFP; enhanced green fluorescent protein. 2A self-cleaving peptide. CAR; chimeric antigen receptor. hPGK1; human phosphoglycerate kinase 1 promoter.

FIG. 15 shows studies of rapamycin-resistant CD19-CAR-T cells, which maintain potent anti-tumor activity in the presence of rapamycin against NALM6 acute lymphoblastic leukemia cells. (A) CD3⁺ CD19-CAR-T cells (FIG. 14) were incubated at an effector to target (E:T) ratio of 10 with NALM6 cells stably expressing firefly luciferase (FLUC) and in the presence of 25 nM of rapamycin. Luminescence was measured after 18 hours of incubation.

FIG. 16 shows highly efficient CAR targeting at MTOR with AAV6 delivery. (A) Strategy for CRISPR-driven CAR-2A-EGFP gene knock-in to the MTOR locus using an AAV6 vector. (B) CD3+ T cells were transfected with 50 pmol of SpCas9 RNP and transduced with the indicated multiplicity of infection (MOI) with the AAV6 donor. Cells were treated with 25 nM rapamycin 3 days post-transfection and cultured for an additional 8 days. The percentage of cells expressing the EGFP reporter was quantified by FACS after selection. HA, homology arm. PA, polyadenylation signal. EGFP, enhanced green fluorescent protein. 2A self-cleaving peptide. CAR, chimeric antigen receptor. hPGK1, human phosphoglycerate kinase 1 promoter.

FIG. 17 shows rapamycin-resistant CD19-CAR-T cells maintain potent anti-tumor activity in the presence of rapamycin with lower effector to target ratios. (A) CD3+CD19-CAR-T cells were incubated with the indicated effector to target (E:T) ratio with NALM6 cells stably expressing firefly luciferase (FLUC) and 25 nM of rapamycin. Rapamycin-resistant NALM6 cells harboring the MTOR-F2108L mutation (NALM6-RapaR) were used to analyze the combinatorial impact of rapamycin and CAR-T cells' cytotoxicity. Luminescence was measured after 18 hours of incubation. n=2 independent biological replicates performed at different times with CD3+ T cells from two different healthy donors.

FIG. 18 shows the combinatorial impact of rapamycin-resistant CAR-T cells cytotoxicity and growth inhibition mediated by rapamycin using a FACS-based coculture assay. (A) CD3+CD19-CAR-T cells were incubated in the presence or absence of 25 nM rapamycin with a ratio of 0.5:1:1 with target (NALM6-RFP) cells and control (K562-EBFP-RapaR-CD19-) cells harboring the MTOR-F2108L mutation. The percentage of cytotoxicity was measured by FACS after 72 hours of incubation.

FIG. 19 shows highly efficient dimerization agent-regulated immunoreceptor complex (DARIC) targeting at MTOR with AAV6 delivery. (A) Strategy for CRISPR-driven CD19-DARIC gene knock-in to the MTOR locus using an AAV6 vector. Receptor dimerization is induced with rapamycin. (B) CD3⁺ T cells were transfected with 50 pmol of SpCas9 RNP and transduced with a multiplicity of infection (MOI) of 5×10³ with the AAV6 donor. Cells were treated with 25 nM rapamycin 3 days post-transfection and cultured for an additional 8 days. The percentage of edited alleles was quantified by TIDER from Sanger sequencing after selection. n=1 experiment performed in triplicate. (C) Vehicle-treated CD3⁺ CD19-DARIC-T cells were incubated in the presence or absence of 25 nM rapamycin with a ratio of 5:1 with target (NALM6-RFP) cells and control (K562-EBFP-RapaR-CD19-) cells harboring the MTOR-F2108L mutation. The percentage of cytotoxicity was measured by FACS after 72 hours of incubation. HA, homology arm. PA, polyadenylation signal. EGFP, enhanced green fluorescent protein. 2A self-cleaving peptide. CAR, chimeric antigen receptor. hPGK1, human phosphoglycerate kinase 1 promoter.

FIGS. 20A-20B: MTOR-F2108L_hPGK1_CD19-CAR-2A-EGFP_Donor sequence (SEQ ID NO: 25).

FIG. 21: MTOR-F2108L_hPGK1_RQR8_Donor sequence (SEQ ID NO: 26).

FIG. 22: MTOR-F2108L_hPGK1_CD52_Donor sequence (SEQ ID NO: 27).

FIG. 23: MTOR-F2108L_v2_Donor sequence (SEQ ID NO: 28).

FIG. 24: MTOR-F2108L_hPGK1_mScarlet-I_Donor sequence (SEQ ID NO: 29).

FIG. 25: MTOR-F2108L_hPGK1_CD19-DARIC_Donor sequence (SEQ ID NO: 30)

FIGS. 26A-26C show details of human MTOR in GenBank accession NG_033239, in respect of the human MTOR gene (SEQ ID NO: 1) and protein (SEQ ID NO: 2) sequences.

### DETAILED DESCRIPTION OF ILLUSTRATIVE EMBODIMENTS

In the studies described herein, a novel strategy is shown to both confer dominant cellular resistance to rapamycin and at the same time introduce a transgene of interest, such as a therapeutic transgene, for example for the preparation and use of CRISPR-engineered CAR-T cells. Thus, the strategy described herein may for example be used to simultaneously select CRISPR-engineered cells in vivo to increase HDR yields without using an exogenous selection marker while increasing activity of a transgene, e.g., anti-tumor activity by targeting cancer cell metabolism. For example, in the examples described below, we demonstrate that targeting a CD19-CAR to the MTOR locus allows the selection of rapamycin-resistant CAR-T cells to perform combinatorial immunotherapy with rapamycin.

While CRISPR has been typically used for gene inactivation and loss-of-function screens, achieving efficient precise modifications via homology-directed repair (HDR) remains challenging. Described herein is a novel strategy to expand the therapeutic applications of genome editing and to take advantage of its potential to generate therapeutically enhanced cells for cell therapy applications.

Therefore, in an aspect, the present disclosure provides a method for nuclease-based modification of a target polynucleotide at an MTOR locus in a target cell, the method comprising:
(i) introducing a first modification at the MTOR locus, wherein the first modification confers resistance to an mTOR inhibitor, and;
(ii) introducing a second modification at the MTOR locus, wherein the second modification comprises introduction of a nucleic acid of interest at the MTOR locus.

Thus in an embodiment, the method results in a modified MTOR locus comprising both a nucleic acid encoding a modified mTOR polypeptide that is resistant to an mTOR inhibitor and a nucleic acid of interest encoding for example a gene of interest as described herein.

In embodiments, the method may further comprise modification of one or more further target polynucleotides at one or more further loci other than the MTOR locus in the target cell, the method further comprising introducing one or more further modifications at the one or more further loci, wherein the one or more further modifications comprise introduction of one or more further nucleic acids of interest at the one or more further loci.

In embodiments, the nuclease-based modification in the target polynucleotide is introduced by non-homologous end joining (NHEJ) or homology-directed repair (HDR). In an embodiment, the nuclease-based modification in the target polynucleotide is introduced by homology-directed repair (HDR) and the method comprises introducing into the cell one or more donor nucleic acids comprising the first and second modifications (and optionally the one or more further modifications). In an embodiment, a single donor nucleic acid may be used which comprises both the first and second modifications. In such a case, the introduction of both a nucleic acid encoding a modified mTOR polypeptide that is resistant to an mTOR inhibitor and a nucleic acid of interest encoding for example a gene of interest can be completed together in a single round or step of modification. Further, in such a case, both the nucleic acid encoding a modified mTOR polypeptide that is resistant to an mTOR inhibitor and a nucleic acid of interest encoding for example a gene of interest are inserted at an MTOR locus. Thus, in an embodiment, the method does not comprise introduction of the first and second modifications at another locus in the cell, i.e. at a locus other than an MTOR locus.

In an embodiment, the method further comprises:
(iii) contacting the target cell with the mTOR inhibitor, and;
(iv) selecting a cell comprising the first and second modifications by virtue of increased tolerance to the mTOR inhibitor, thereby enriching for the cell comprising the first and second modifications.

In such a case, the introduction of the first and second modifications is facilitated by virtue of the creation of the resistant form of mTOR (as a result of the first modification), which in turn may be used for selection using an mTOR inhibitor as a selection agent. Such an approach is advantageous because the mTOR resistance can be used to enrich for cells comprising both the first and second modifications and, at the same time, avoids the use of external selection markers or drugs. Thus, in an embodiment, the method does not comprise the use of a selection marker other than that based on mTOR inhibition.

Further, in embodiments, the method may further comprise introducing one or more further modifications at other, non-MTOR loci, in addition to the first and second modifications at an MTOR locus. In such cases, the mTOR resistance may also be used to also enrich for target cells harboring the one or more further modifications. In such a case selection may be similarly achieved without the use of a selection marker other than that based on mTOR inhibition.

As used herein, the terms “resistance” or “resistant” refers to the resistance conferred via modification of an MTOR gene (i.e. relative to an MTOR gene lacking the modification) as well as cellular resistance by virtue of the presence of such a modified MTOR gene (as compared to normal cells, e.g., cells lacking an MTOR gene containing a modification conferring the resistance) to an mTOR inhibitor. Such resistant cells are also referred to herein as being tolerant to an mTOR inhibitor. “Tolerance” or “tolerant” as used herein refers to the capacity of a first cell containing an MTOR gene containing a modification conferring the resistance to be less affected by an mTOR inhibitor than a second cell that does not have a modified MTOR gene that confers resistance to the mTOR inhibitor. Tolerant cells grow and develop better in the presence of an mTOR inhibitor when compared to intolerant cells, i.e. have increased growth and viability relative to intolerant cells. Such tolerance can be used as a basis of selection of tolerant cells over intolerant cells, e.g. based on increased growth of tolerant cells relative to intolerant cells when treated with an mTOR inhibitor, in an embodiment the viability of tolerant cells relative to the viability of intolerant cells when treated with an mTOR inhibitor. In an embodiment, the resistant, modified MTOR gene is dominant for resistance to an mTOR inhibitor.

In embodiments, the mTOR inhibitor is rapamycin or a rapalog (e.g., temsirolimus, everolimus, deforolimus, umirolimus, zotarolimus, ridaforolimus, or any combination thereof).

In an embodiment, the MTOR locus comprising the first and second modifications encodes a mTOR polypeptide that retains mTOR signaling activity. mTOR signaling may be assessed by various methods, for example using and mTOR signaling indicator such as mSc-TOSI as described in the Examples herein. Active mTORC1 signaling results in rapid phosphorylation of the mSc-TOSI phosphodegron by S6K, ubiquitination, and degradation by the proteasome while its inhibition stabilizes the reporter.

In a preferred embodiment, the CRISPR/Cas9 system is used to introduce the first and second modifications (and optionally the one or more further modifications). In such a case, the method comprises providing the target cell with a CRISPR nuclease and one or more gRNAs comprising one or more guide sequences having one or more target sequences in the target polynucleotide, such that the one or more gRNAs direct the cleavage of the target polynucleotide at the MTOR locus to introduce the first and second modifications (and optionally the one or more further modifications). In embodiments, the first modification may also be introduced using methods such as base editing and prime editing, described herein. Each target sequence is contiguous to a protospacer adjacent motif (PAM) recognized by the CRISPR nuclease.

In embodiments, the target polynucleotide or the one or more target sequences for modification at the MTOR locus is i) within an exon of an MTOR gene, ii) within an intron of the MTOR gene, or iii) spanning an exon-intron boundary of a MTOR gene. In embodiments, at exon 45, intron 45, or spanning the exon 45-intron 45 boundary of the MTOR gene.

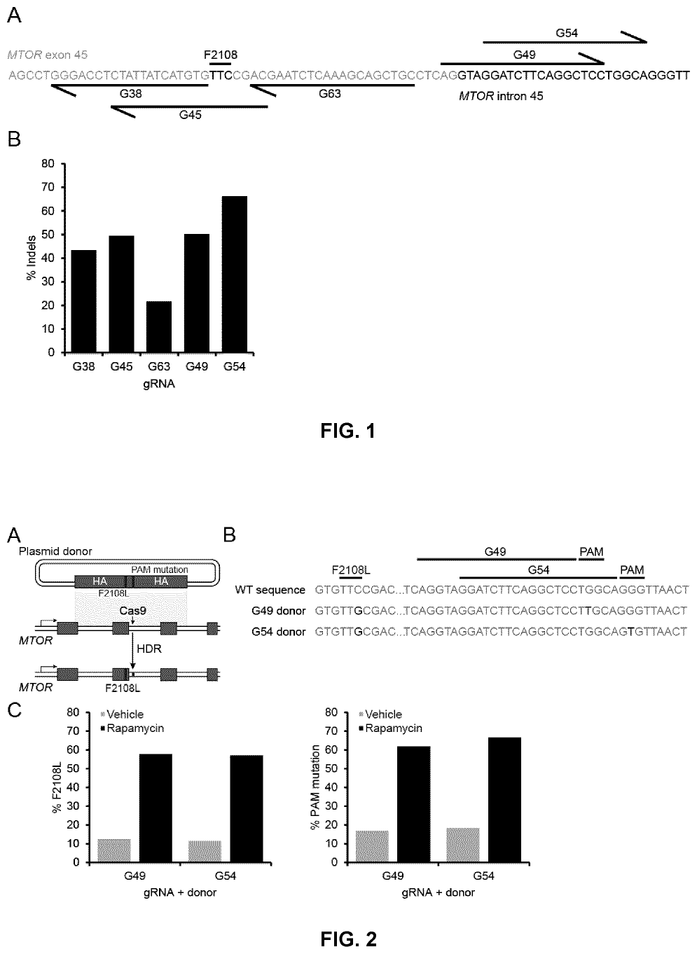

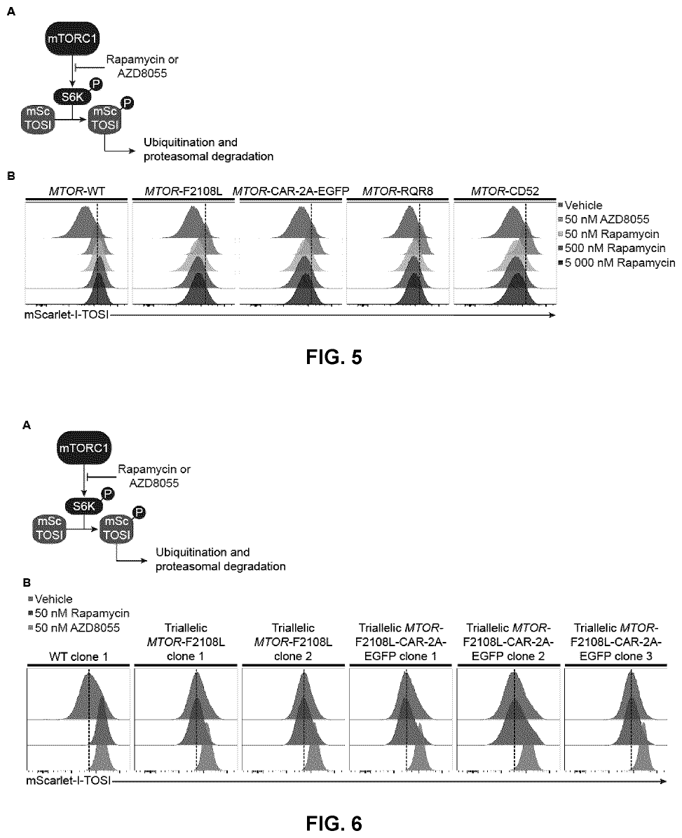

In embodiments, the target polynucleotide or the one or more target sequences are within a nucleotide region set forth in any of FIG. 1, FIG. 2, or FIG. 5, in the sense or antisense sequence.

In embodiments, the one or more target sequences comprise one or more of 5′-CACATGATAATAGAGGTCCC-3′ (antisense strand, SEQ ID NO: 3), 5′-GTCGGAACACATGATAATAG-3′ (antisense strand, SEQ ID NO: 4), 5′-GCAGCTGCTTTGAGATTCGT-3′ (antisense strand, SEQ ID NO: 5); 5′-AGGTAGGATCTTCAGGCTCC-3′ (sense strand, SEQ ID NO: 6); and 5′-GGATCTTCAGGCTCCTGGCA-3′ (sense strand, SEQ ID NO: 7).

In embodiments, the first modification results in an amino acid substitution, insertion, or deletion in a mTOR FRB domain, i.e., within the region defined approximately by residues 2015-2114 of SEQ ID NO: 2. In an embodiment, the first modification results in an amino acid substitution at position 2108 of a mTOR polypeptide, such as a F2108L substitution.

In embodiments, the cell is a stem cell, such as a hematopoietic stem cell (HSC), an embryonic stem cell, a totipotent stem cell, a pluripotent stem cell, a multipotent stem cell, or an induced pluripotent stem cell (iPSC). In embodiments, the cell is an immune cell, such as a T cell (e.g., a CD3⁺ T cell, a CD8⁺ T cell, a CD4*T cell, a Regulatory T cell (Tregs, e.g., CD4⁺/FOXP3⁺)), a natural killer (NK) cell, or a B cell.

In embodiments, a nucleic acid of interest encodes a therapeutic protein, such as a chimeric antigen receptor (CAR).

In embodiments, the methods described herein may be performed in vitro, ex vivo, in vivo (in a subject), or a combination thereof.

The present disclosure also relates to one or more gRNAs or pegRNAs and one or more donor nucleic acids described herein.

The present disclosure also relates to an expression cassette comprising (a) a modified MTOR nucleic acid comprising a first modification as defined herein and (b) a nucleic acid of interest as defined herein.

The present disclosure also relates to one or more vectors comprising nucleic acid sequence(s) corresponding to one or more components described herein, such as nucleic acid sequences encoding one or more gRNAs or pegRNAs as described herein, one or more donor nucleic acids as described herein, an expression cassette as described herein, and/or a nucleic acid sequence encoding a CRISPR nuclease. In embodiments, such components may be present on separate vectors or any combination of two or more of such components may be present on the same vector.

The present disclosure also relates to cells prepared by a method described herein, comprising a modified MTORlocus comprising a first modification to confer resistance to an mTOR inhibitor and a second modification resulting in the introduction of a nucleic acid of interest (and optionally one or more further modifications resulting in the introduction of one or more further nucleic acids of interest at one or more further loci).

The present disclosure also relates to cells comprising one or more components described herein, such as nucleic acid sequences encoding one or more gRNAs or pegRNAs as described herein, one or more gRNAs or pegRNAs as described herein, one or more donor nucleic acids as described herein, an expression cassette as described herein, a nucleic acid sequence encoding a CRISPR nuclease, a CRISPR nuclease, one or more vectors described herein, and/or one or more compositions as described herein.

The present disclosure also relates to a composition comprising one or more components described herein, such as nucleic acid sequences encoding one or more gRNAs or pegRNAs as described herein, one or more gRNAs or pegRNAs as described herein, one or more donor nucleic acids as described herein, an expression cassette as described herein, a nucleic acid sequence encoding a CRISPR nuclease, a CRISPR nuclease, one or more vectors described herein, and/or one or more cells as described herein.

The present disclosure also relates to various methods and uses of one or more components described herein, such as nucleic acid sequences encoding one or more gRNAs or pegRNAs as described herein, one or more gRNAs or pegRNAs as described herein, one or more donor nucleic acids as described herein, an expression cassette as described herein, a nucleic acid sequence encoding a CRISPR nuclease, a CRISPR nuclease, one or more vectors described herein, and/or one or more compositions as described herein. In embodiments, such methods or uses of the one or more components, include modifying a target polynucleotide in a cell, the preparation of a medicament for modifying a target polynucleotide in a cell, treating a disease, condition, or disorder in a subject, or the preparation of a medicament for treating a disease, condition, or disorder in a subject.

The present disclosure also relates to one or more components described herein, such as nucleic acid sequences encoding one or more gRNAs or pegRNAs as described herein, one or more gRNAs or pegRNAs as described herein, one or more donor nucleic acids as described herein, an expression cassette as described herein, a nucleic acid sequence encoding a CRISPR nuclease, a CRISPR nuclease, one or more vectors described herein, and/or one or more compositions as described herein, for various uses, such as for use in modifying a target polynucleotide in a cell, the preparation of a medicament for modifying a target polynucleotide in a cell, treating a disease, condition, or disorder in a subject, or the preparation of a medicament for treating a disease, condition, or disorder in a subject.

In embodiments, the disease, condition, or disorder is associated with expression of an antigen, and wherein a nucleic acid of interest encodes a recombinant receptor that specifically binds to the antigen, such as a chimeric antigen receptor (CAR).

In an embodiment, the disease, condition, or disorder is a cancer, an autoimmune or inflammatory disease, or an infectious disease.

In a further embodiment, the disease, condition, or disorder is cancer. In such a case, in an embodiment, an anticancer agent may be administered to the subject being treated. In an embodiment, the anticancer agent comprises rapamycin or a rapalog, in which case the anticancer agent can also be used for selection and the maintenance thereof of the modifications conferring resistance to an mTOR inhibitor and the presence of the nucleic acid of interest.

### Definitions

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics, and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the specification unless otherwise indicated. See, e.g.: Sambrook J. and Russell D. Molecular Cloning: A Laboratory Manual, 3^{rd} ed., Cold Spring Harbor Laboratory Press, Cold Spring Harbor, N.Y. (2000); Ausubel F. M. et al., Short Protocols in Molecular Biology: A Compendium of Methods from Current Protocols in Molecular Biology, Wiley, John & Sons, Inc. (2002); Harlow E. and D. Lane, Using Antibodies: A Laboratory Manual; Cold Spring Harbor Laboratory Press, Cold Spring Harbor, N.Y. (1998); and Coligan J. E. et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The use of the terms “a” and “an” and “the” and similar referents in the context of describing the technology (especially in the context of the claims) are to be construed to cover both the singular and the plural, unless otherwise indicated herein or clearly contradicted by context.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein. For example, for the range of 18-20, the numbers 18, 19, and 20 are explicitly contemplated, and for the range 6.0-7.0, the numbers 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

The use of any and all examples, or exemplary language (“e.g.”, “such as”) provided herein, is intended merely to better illustrate the technology and does not pose a limitation on the scope of the claimed invention unless otherwise claimed.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide”, and “protein” are used interchangeably to refer to a polymer of amino acid residues. The term also applies to amino acid polymers in which one or more amino acids are chemical analogues or modified derivatives of corresponding naturally-occurring amino acids.

As used herein, the term “non-conservative mutation” or “non-conservative substitution” in the context of polypeptides refers to a mutation in a polypeptide that changes an amino acid to a different amino acid with different biochemical properties (i.e., charge, hydrophobicity, and/or size). Although there are many ways to classify amino acids, they are often sorted into six main groups, on the basis of their structure and the general chemical characteristics of their R groups. (i) aliphatic (glycine, alanine, valine, leucine, and isoleucine); (ii) hydroxyl- or sulfur/selenium-containing (also known as polar amino acids; serine, cysteine, selenocysteine, threonine, and methionine); (iii) cyclic (proline); (iv) aromatic (phenylalanine, tyrosine, and tryptophan); (v) basic (histidine, lysine, and arginine), and (vi) acidic and their amide (aspartate, glutamate, asparagine, and glutamine). Thus, a non-conservative substitution includes one that changes an amino acid of one group with another amino acid of another group (e.g., an aliphatic amino acid for a basic, a cyclic, an aromatic, or a polar amino acid; a basic amino acid for an acidic amino acid; a negatively charged amino acid [aspartic acid or glutamic acid] for a positively charged amino acid [lysine, arginine, or histidine], etc.)

Conversely, a “conservative substitution” or “conservative mutation” in the context of polypeptides are mutations that change an amino acid to a different amino acid with similar biochemical properties (e.g. charge, hydrophobicity, and size). For example, a leucine and isoleucine are both aliphatic branched hydrophobic amino acids. Similarly, aspartic acid and glutamic acid are both small, negatively charged amino acids. Therefore, changing a leucine for an isoleucine (or vice versa) or changing an aspartic acid for a glutamic acid (or vice versa) are examples of conservative substitutions. “Coding sequence” or “encoding nucleic acid” as used herein means the nucleic acids (RNA or DNA molecule) that comprise a nucleotide sequence which encodes a protein, gRNA or pegRNA. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

### Sequence Similarity

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “substantially homologous” to another sequence if the two sequences are substantially identical and the functional activity of the sequences is conserved (as used herein, the term “homologous” does not infer evolutionary relatedness, but rather refers to substantial sequence identity, and thus is interchangeable with the terms “identity”/“identical”). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90%, or 95%. For the sake of brevity, the units (e.g., 66, 67, . . . 81, 82, . . . 91, 92%, . . . ) have not systematically been recited but are considered, nevertheless, within the scope of the present invention.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 96%, at least 98%, or at least 99%. Optimal alignment of sequences for comparisons of identity may be conducted using a variety of algorithms, such as the local homology algorithm of Smith and Waterman (Adv. Appl. Math. 2: 482-489, 1981), the homology alignment algorithm of Needleman and Wunsch (J. Mol. Biol. 48: 443-453, 1970), the search for similarity method of Pearson and Lipman (Proc. Natl. Acad. Sci. USA 85: 2444-2448, 1988), and the computerized implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described by Altschul et al. (J. Mol. Biol. 215: 403-410, 1990 using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably, stringent conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (Ausubel, F. M. et al., Current Protocols in Molecular Biology, John Wiley & Sons, New York, 2010). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (Ausubel, 2010, supra). Hybridization conditions may be modified in accordance with known methods depending on the sequence of interest (Tijssen P, Hybridization with nucleic acid probes, Part II, Volume 24, 1st Edition, Part II. Probe labeling and hybridization techniques, Elsevier Science, 1993, 344 pages). Generally, stringent conditions are selected to be about 5° C. lower than the thermal melting point for the specific sequence at a defined ionic strength and pH.

“Binding” refers to a sequence-specific, non-covalent interaction between macromolecules (e.g., between a protein and a nucleic acid or between a sgRNA and a target polynucleotide or between a sgRNA and a CRISPR nuclease (e.g., Cas9, Cpf1). Not all components of a binding interaction need be sequence-specific (e.g., contacts with phosphate residues in a DNA backbone), as long as the interaction as a whole is sequence-specific. “Affinity” refers to the strength of binding: increased binding affinity being correlated with a lower Kd.

A “binding protein” is a protein that is able to bind non-covalently to another molecule. A binding protein can bind to, for example, a DNA molecule (a DNA-binding protein), an RNA molecule (an RNA-binding protein), and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

As used herein, “a nuclease-based modification” refers to a modification in a polynucleotide e.g., an endogenous gene locus or genomic sequence) which involves the introduction of a cut (e.g., a double-stranded break in the polynucleotide) which ultimately will trigger a repair mechanism by the cell involving non-homologous-end-joining (NHEJ) or homologous recombination (HDR). The nuclease-based modification is made by site-specific nucleases targeting the polynucleotide of interest (i.e., an endogenous gene locus or genomic sequence). Site-specific nucleases (engineered) are well known and include (but are not limited to) zinc finger nucleases, meganucleases, Mega-Tals, CRISPR nucleases, TALENs, etc.

“Recombination” refers to a process of exchange of genetic information between two polynucleotides. For the purposes of this disclosure, “homologous recombination” (HR) refers to the specialized form of such exchange that takes place, for example, during repair of double-strand breaks in cells via homology-directed repair (HDR) mechanisms. This process requires nucleotide sequence homology, uses a “donor” or “patch” molecule as a template for repair of a “target” molecule (i.e., the one that experienced the double-strand break), and is variously known as “non-crossover gene conversion” or “short tract gene conversion” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing”, in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods described herein, one or more targeted (site-specific) nucleases (e.g., sgRNA/CRISPR nuclease) create a double-stranded break in the target sequence (e.g., cellular chromatin) at a predetermined site. A “donor” polynucleotide, having homology to the nucleotide sequence in the region of the break, may be introduced into the cell if desired. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another. In any of the methods described herein, additional sgRNA/CRISPR nucleases, pair zinc-finger, meganucleases, Mega-Tals, and/or additional TALEN proteins can be used for additional double-stranded cleavage of additional target sites within the cell.

As used herein, the terms “donor” or “patch” nucleic acid are used interchangeably and refers to a nucleic acid that includes a fragment of the endogenous targeted gene of a cell (in some embodiments the entire targeted gene), but which includes desired modification(s) at specific nucleotides. The donor (patch) nucleic acid must be of sufficient size and similarity (e.g., in the right and left homology arms) to permit homologous recombination with the targeted gene. Preferably, the donor/patch nucleic acid is (or is flanked at the 5′ end and at the 3′ end by sequences) at least 80%, 85%, 90%, 91%, 92%, 93%, 94%, 95%, 96%, 97%, or 98% identical to the endogenous targeted polynucleotide gene sequence. The patch nucleic acid may be provided for example as a ssODN, as a PCR product (amplicon) or within a vector. Preferably, the patch/donor nucleic acid will include modifications with respect to the endogenous gene which precludes it from being cut by a sgRNA once integrated in the genome of a cell and/or which facilitate the detection of the introduction of the patch nucleic acid by homologous recombination.

As used herein, a “target gene”, “gene of interest” or “target polynucleotide” corresponds to the polynucleotide within a cell that will be modified by the introduction of the patch nucleic acid. It corresponds to an endogenous gene naturally present within a cell. The target gene may comprise one or more mutations associated with a risk of developing a disease or disorder which may be corrected by the introduction of the patch/donor nucleic acid (e.g., will be modified to correspond to the WT gene or to a form which is no longer associated with increased risk of developing a disease or condition). One or both allele(s) of a target gene may be corrected or modified within a cell, in accordance with the present disclosure. Examples of target genes are described in Tables 3-6.

A “target polynucleotide” as used herein refers to any endogenous polynucleotide or nucleic acid present in the genome of a cell and encoding or not a known gene product. “Target gene” as used herein refers to any endogenous polynucleotide or nucleic acid present in the genome of a cell and encoding a known or putative gene product. The target gene or target polynucleotide further corresponds to the polynucleotide within a cell that will be modified by a nuclease of the present disclosure, alone or in combination with the introduction of one or more donor nucleic acid or patch nucleic acids. The target gene or target polynucleotide may be a mutated gene involved in a genetic disease.

“Promoter” as used herein means a synthetic or naturally-derived nucleic acid molecule which is capable of conferring, modulating, or controlling (e.g., activating, enhancing, and/or repressing) expression of a nucleic acid in a cell. A promoter may comprise one or more specific transcriptional regulatory sequences to further enhance or repress expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, CMV IE promoter, U6 promoter, a liver-specific promoter (e.g., LP1b; combining the human apolipoprotein E/C-I gene locus control region [ApoE-HCR] and a modified human α1 antitrypsin promoter [hAAT] coupled to an SV40 intron), human thyroxine binding globulin (TBG) promoter, CMV promoter, CAG promoter, CBH promoter, UbiC promoter, Ef1a promoter, H1 promoter, and 7SK promoter, any of which may be used to express one or more gRNAs or pegRNAs and/or a CRISPR nuclease in a cell. Sequences for the LP1b and TBG promoters are provided in Table 8.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may comprise nucleic acid sequence(s) that/which encode(s) a sgRNA, a donor (or patch) nucleic acid, and/or a CRISPR nuclease (e.g., Cas9 or Cpf1) of the present disclosure. A vector for expressing one or more sgRNA will comprise a “DNA” sequence of the sgRNA.

In embodiments, a vector may comprise “regulatory” or “control” sequences, which refer to nucleic acid sequences necessary for the transcription and possibly translation of an operably linked coding sequence in a particular host cell. In addition to control sequences that govern transcription and translation, expression vectors may contain nucleic acid sequences that serve other functions as well.

In an embodiment, the vector further comprises a nucleic acid encoding a selectable marker or reporter protein. A selectable marker or reporter is defined herein to refer to a nucleic acid encoding a polypeptide that, when expressed, confers an identifiable characteristic (e.g., a detectable signal, resistance to a selective agent) to the cell permitting easy identification, isolation and/or selection of cells containing the selectable marker from cells without the selectable marker or reporter. Any selectable marker or reporter known to those of ordinary skill in the art is contemplated for inclusion as a selectable marker in the vector of the present disclosure. For example, the selectable marker may be a drug selection marker, an enzyme, or an immunologic marker. Examples of selectable markers or reporters include, but are not limited to, polypeptides conferring drug resistance (e.g., kanamycin/geneticin resistance), enzymes such as alkaline phosphatase and thymidine kinase, bioluminescent and fluorescent proteins such as luciferase, green fluorescent protein (GFP), yellow fluorescent protein (YFP), cyan fluorescent protein (CFP), blue fluorescent protein (BFP), citrine and red fluorescent protein from Discosoma sp. (dsRED), membrane bound proteins to which high affinity antibodies or ligands directed thereto exist or can be produced by conventional means, and fusion proteins comprising a membrane-bound protein appropriately fused to an antigen tag domain from, among others, hemagglutinin (HA) or Myc. The nucleic acid encoding the selectable marker or reporter protein may be under the control of the same promoter/enhancer as the nucleic acid of interest, or may be under the control of a distinct promoter/enhancer.

In embodiments, the vector may comprise additional elements, such as one or more origins of replication sites (often termed “on”), restriction endonuclease recognition sites (multiple cloning sites, MCS), and/or internal ribosome entry site (IRES) elements.

Nucleic acids encoding gRNAs, pegRNAs and CRISPR nucleases (e.g., Cas9) of the present disclosure may be delivered into cells using one or more various vectors such as viral vectors. Accordingly, preferably, the above-mentioned vector is a viral vector for introducing the sgRNA and/or nuclease of the present disclosure in a target cell. Non-limiting examples of viral vectors include retrovirus, lentivirus, herpesvirus, adenovirus, or adeno-associated virus (AAV), as well known in the art.

“Adeno-associated virus” or “AAV” as used interchangeably herein refers to a small virus belonging to the genus Dependoparvovirus of the Parvoviridae family that infects humans and some other primate species. AAV is not currently known to cause disease and, consequently, the virus may cause a very mild immune response.

In embodiments, the AAV vector preferably targets one or more cell types. Accordingly, the AAV vector may have enhanced cardiac, skeletal muscle, neuronal, liver, and/or pancreatic tissue (Langerhans cells) tropism. The AAV vector may be capable of delivering and expressing the at least one sgRNA and nuclease of the present disclosure in the cell of a mammal. For example, the AAV vector may be an AAV-SASTG vector (Piacentino et al., Hum. Gene Ther. 23: 635-646, 2012). The AAV vector may deliver gRNAs, pegRNAs and nucleases to neurons, skeletal and cardiac muscle, and/or pancreas (Langerhans cells) in vivo. The AAV vector may be based on one or more of several capsid types, including AAV1, AAV2, AAV5, AAV6, AAV8, and AAV9. The AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery. In an embodiment, the AAV vector is a AAV-DJ vector. In an embodiment, the AAV vector is a AAV-DJ8 vector. In an embodiment, the AAV vector is a AAV2-DJ8 vector. In an embodiment, the AAV vector is a AAV-PHP.B vector. In an embodiment, the AAV vector is a AAV-PHP.B, AAV-9, or AAV-DJ8 (PHP.B: Deverman D. E. et al., Nat. Biotechnol. 34: 204-209, 2016; Jackson K. L. et al., Front. Mol. Neurosci. 9: 116, 2016; AAV DJ-8: www.cellbiolabs.com/news/aav-helper-free-expression-systems-aav-dj-aav-dj8, http://www.cellbiolabs.com/aav-expression-and-packaging; www.cellbiolabs.com/scaav-dj8-helper-free-complete-expression-systems; and AAV9: Saraiva J. et al., J. Control. Release 241: 94-109, 2016; Inagaki K. et al., Mol. Ther. 14: 45-53, 2006).

### Cells

In another aspect, the present disclosure provides a cell (host cell, engineered cell) comprising a nucleic acid, gRNA, pegRNA, expression cassette, or vector/plasmid described herein. In an embodiment, the cell is a primary cell, for example a brain/neuronal cell, a peripheral blood cell (e.g., a B or T lymphocyte, a monocyte, or a NK cell), a cord blood cell, a bone marrow cell, a cardiac cell, an endothelial cell, an epidermal cell, an epithelial cell, a fibroblast, hepatic cell, or a lung/pulmonary cell. In an embodiment, the cell is a bone marrow cell, peripheral blood cell or cord blood cell. In a further embodiment, the cell is an immune cell, such as a T cell (e.g., a CD3⁺ T cell, a CD8⁺ T cell, a CD4⁺ T cell, a Regulatory T cell (Tregs, e.g., CD4⁺/FOXP3⁺)), a B cell, or a NK cell.

In an embodiment, the cell is a stem cell. The term “stem cell” as used herein refers to a cell that has pluripotency which allows it to differentiate into a functional mature cell. It includes primitive hematopoietic cells, progenitor cells, as well as adult stem cells that are undifferentiated cells found in various tissue within the human body, which can renew themselves and give rise to specialized cell types and tissue from which the cells came (e.g., muscle stem cells, skin stem cells, brain or neural stem cells, mesenchymal stem cell, lung stem cells, liver stem cells).

In an embodiment, the cell is a primitive hematopoietic cell. As used herein, the term “primitive hematopoietic cell” is used to refers to cells having pluripotency which allows them to differentiate into functional mature blood cells of the myeloid and lymphoid lineages such as T cells, B cells, NK cells, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages), and that may or may not the ability to regenerate while maintaining their pluripotency (self-renewal). It encompasses “hematopoietic stem cells” or “HSCs”, which are cells having both pluripotency which allows them to differentiate into functional mature cells such as granulocytes, erythrocytes, thrombocytes, and monocytes, and the ability to regenerate while maintaining their pluripotency (self-renewal), as well as pluripotent hematopoietic cells that do not have self-renewal capacity. It also encompasses embryonic stem cells (ESCs), which are pluripotent stem cells derived from the inner cell mass of a blastocyst, an early-stage pre-implantation embryo. In an embodiment, the population of cells comprises ESCs. In another embodiment, the population of cells comprises HSCs. HSCs may be obtained from the body or an organ of the body containing cells of hematopoietic origin. Such sources include unfractionated bone marrow (from femurs, hips, ribs, sternum, and other bones), umbilical cord blood, peripheral blood, liver, thymus, lymph, and spleen. All of the aforementioned crude or unfractionated blood products can be enriched for cells having HSC characteristics in ways known to those of skill in the art. HSCs are phenotypically identified by their small size, lack of lineage (lin) markers, low staining (side population) with vital dyes such as rhodamine 123 (rhodamineDULL, also called rho 0) or Hoechst 33342, and presence/absence of various antigenic markers on their surface many of which belongs to the cluster of differentiation series, such as CD34, CD38, CD90, CD133, CD105, CD45, and c-kit.

In an embodiment, the stem cell is an induced pluripotent stem cell (iPSC). The term iPSC refers to a pluripotent stem cell that can be generated directly from adult cells using appropriate factors to “reprogram” the cells.

In an embodiment, the cell is a mammalian cell, for example a human cell.

A nucleic acid, expression cassette, or vector/plasmid described herein may be introduced into the cell using standard techniques for introducing nucleic acids into a cell, e.g., transfection, transduction, or transformation. In an embodiment, the vector is a viral vector, and the cell is transduced with the vector. As used herein, the term “transduction” refers to the stable transfer of genetic material from a viral particle (e.g., lentiviral) to a cell genome (e.g., hematopoietic cell genome). It also encompasses the introduction of non-integrating viral vectors into cells, which leads to the transient or episomal expression of the gene of interest present in the viral vector.

Viruses may be used to infect cells in vivo, ex vivo, or in vitro using techniques well known in the art. For example, when cells, for instance CD34+ cells or stem cells are transduced ex vivo, the vector particles may be incubated with the cells using a dose generally in the order of between 1 to 100 or 1 to 50 multiplicities of infection (MOI) which also corresponds to 1×10⁵ to 100 or 50×10⁵ transducing units of the viral vector per 10⁵ cells. This, of course, includes amount of vector corresponding to 1, 2, 3, 4, 5, 6, 7, 8, 9, 10, 15, 20, 25, 30, 35, 40, 45, and 50 MOI.

Prior to, during, and/or following transduction, the cells may be cultured in media suitable for the maintenance, growth, or proliferation of the cells. The culture conditions of the population of cells will vary depending on different factors, notably, the starting cell population. Suitable culture media and conditions are well known in the art. The culture may be carried out in natural medium, a semi-synthetic medium, or a synthetic medium in terms of composition, and may be a solid medium, a semisolid medium, or a liquid medium in terms of shape, and any nutrient medium used for cell culture, such as stem cell culture, which may be supplemented with one or more of growth factors. Such medium typically comprises sodium, potassium, calcium, magnesium, phosphorus, chlorine, amino acids, vitamins, cytokines, hormones, antibiotics, serum, fatty acids, saccharides, or the like. In the culture, other chemical components or biological components may be incorporated singly or in combination, as the case requires. Such components to be incorporated in the medium may be fetal calf serum, human serum, horse serum, insulin, transferrin, lactoferrin, cholesterol, ethanolamine, sodium selenite, monothioglycerol, 2-mercaptoethanol, bovine serum albumin, sodium pyruvate, polyethylene glycol, various vitamins, various amino acids, agar, agarose, collagen, methylcellulose, various cytokines, various growth factors, or the like. Examples of such basal medium appropriate for a method of expanding stem cells include, without limitation, StemSpan™ Serum-Free Expansion Medium (SFEM; StemCell Technologies®, Vancouver, Canada), StemSpan™ H3000-Defined Medium (StemCell Technologies®, Vancouver, Canada), CellGro™, SCGM (CellGenix™, Freiburg Germany), StemPro™-34 SFM (Invitrogen®), Dulbecco's Modified Eagle's Medium (DMEM), Ham's Nutrient Mixture H12 Mixture F12, McCoy's 5A medium, Eagle's Minimum Essential Medium (EMEM), MEM medium (alpha Modified Eagle's Minimum Essential Medium), RPMI 1640 medium, Isocove's Modified Dulbecco's Medium (IMDM), StemPro34™ (Invitrogen®), X-VIVO™ 10 (Cambrex®), X-VIVO™ 15 (Cambrex®), and Stemline™ ∥ (Sigma-Aldrich®).

Following transduction, the transduced cells may be cultured under conditions suitable for their maintenance, growth and/or proliferation. In particular aspects, the transduced cells are cultured for about 1, 2, 3, 4, 5, 6, 7, 8, 9, 10, 11, 12, 13, or 14 days before transplantation.

Culture conditions for maintaining and/or expanding stem cells are well known in the art. Typically, the culturing conditions comprise the use of factors like cytokines and growth factors, generally known in the art for stem cell expansion. Such cytokines and growth factors can be biologics or small molecules and they include without limitation IL-1, IL-3, IL-6, IL-11, G-CSF, GM-CSF, SCF, FIT3-L, thrombopoietin (TPO), erythropoietin, and analogs thereof. As used herein, “analogs” include any structural variants of the cytokines and growth factors having the biological activity as the naturally occurring forms, including without limitation, variants with enhanced or decreased biological activity when compared to the naturally occurring forms or cytokine receptor agonists such as an agonist antibody against the TPO receptor (for example, VB22B scFv2 as detailed in patent publication WO 2007/145227, and the like). Cytokine and growth factor combinations are chosen to maintain/expand stem cells while limiting the production of terminally differentiated cells. In one specific embodiment, one or more cytokines and growth factors are selected from the group consisting of SCF, Flt3-L, and TPO.

Human IL-6 or interleukin-6, also known as B-cell stimulatory factor 2 has been described by (Kishimoto T., Ann. Rev. Immunol. 23: 1-21, 2005) and is commercially available. Human SCF or stem cell factor, also known as c-kit ligand, mast cell growth factor, or Steel factor has been described (Smith, M. A. et al., Acta Haematol., 105: 143-150, 2001) and is commercially available. Flt3-L or FLT-3 Ligand, also referred as FL is a factor that binds to flt3-receptor. It has been described (Hannum C., Nature 368: 643-648, 1994) and is commercially available. TPO or thrombopoietin, also known as megakarayocyte growth factor (MGDF) or c-Mpl ligand has been described (Kaushansky K., N. Engl. J. Med. 354: 2034-2045, 2006) and is commercially available.

The chemical components and biological components mentioned above may be used, not only by adding them to the medium, but also by immobilizing them onto the surface of the substrate or support used for the culture, specifically speaking, by dissolving a component to be used in an appropriate solvent, coating the substrate or support with the resulting solution and then washing away an excess of the component. Such a component to be used may be added to the substrate or support preliminarily coated with a substance which binds to the component.

Stem cells may be cultured in a culture vessel generally used for animal cell culture such as a Petri dish, a flask, a plastic bag, a Teflon™ bag, optionally after preliminary coating with an extracellular matrix or a cell adhesion molecule. The material for such a coating may be collagens I to XIX, fibronectin, vitronectin, laminins 1 to 12, nitrogen, tenascin, thrombospondin, von Willebrand factor, osteoponin, fibrinogen, various elastins, various proteoglycans, various cadherins, desmocolin, desmoglein, various integrins, E-selectin, P-selectin, L-selectin, immunoglobulin superfamily, Matrigel®, poly-D-lysine, poly-L-lysine, chitin, chitosan, Sepharose®, alginic acid gel, hydrogel, or a fragment thereof. Such a coating material may be a recombinant material having an artificially modified amino acid sequence. The stem cells may be cultured by using a bioreactor which can mechanically control the medium composition, pH and the like and obtain high density culture (Schwartz R. M. et al., Proc. Natl. Acad. Sci. U.S.A., 88: 6760-6764, 1991; Koller M. R. et al., Bone Marrow Transplant. 21: 653-663, 1998; Koller M. R. et al., Blood 82: 378-384, 1993; Astori G. et al., Bone Marrow Transplant. 35: 1101-1106, 2005).

The cell population may then be washed to remove any component of the cell culture and resuspended in an appropriate cell suspension medium for short term use or in a long-term storage medium, for example a medium suitable for cryopreservation, for example DMEM with 40% FCS and 10% DMSO. Other methods for preparing frozen stocks for cultured cells also are available to those skilled in the art.

### Compositions

In another aspect, the present disclosure provides a composition (e.g., a pharmaceutical composition) comprising a gRNA, pegRNA and/or CRISPR nuclease (e.g., Cas9), or nucleic acid(s) encoding same or vector(s) comprising such nucleic acid(s), or a cell, as described herein. In an embodiment, the composition further comprises one or more biologically or pharmaceutically acceptable carriers, excipients, and/or diluents.

As used herein, “pharmaceutically acceptable” (or “biologically acceptable”) carriers, excipients, and/or diluents includes any and all solvents, dispersion media, coatings, antibacterial and antifungal agents, isotonic and absorption delaying agents, and the like that are physiologically compatible, and which can be used pharmaceutically or in biological systems. Such materials are characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

When the excipient serves as a diluent, it can be a solid, semisolid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders (see Remington: The Science and Practice of Pharmacy by Alfonso R. Gennaro, 2003, 21^{th} edition, Mack Publishing Company). In embodiments, the carrier may be suitable for intra-neural, parenteral, intravenous, intraperitoneal, intramuscular, subcutaneous, sublingual, or oral administration.

Some examples of suitable excipients include lactose, dextrose, sucrose, sorbitol, mannitol, starches, lecithin, phosphatidylcholine, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propyl-hydroxybenzoates; sweetening agents; and flavoring agents. The compositions of the disclosure can be formulated to provide quick sustained or delayed release of the active ingredient after administration to the patient by employing procedures known in the art.

Pharmaceutical compositions suitable for use in the disclosure include compositions wherein the active ingredients are contained in an effective amount to achieve the intended purpose (e.g., preventing, treating, ameliorating, and/or inhibiting a disease or condition). The determination of an effective dose is well within the capability of those skilled in the art. For any compounds, the therapeutically effective dose can be estimated initially either in cell culture assays (e.g., cell lines) or in animal models, usually mice, rabbits, dogs, or pigs. The animal model may also be used to determine the appropriate concentration range and route of administration. Such information can then be used to determine useful doses and routes for administration in humans. An effective dose or amount refers to that amount of one or more active ingredient(s), which is sufficient for treating a specific disease or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time, and frequency of administration, drug combination(s), reaction sensitivities, and tolerance/response to therapy. Guidance as to particular dosages and methods of delivery is provided in the literature and generally available to practitioners in the art. In embodiments, dosages of an active ingredient of between about 0.01 and about 100 mg/kg body weight (in an embodiment, per day) may be used. In further embodiments, dosages of between about 0.5 and about 75 mg/kg body weight may be used. In further embodiments, dosages of between about 1 and about 50 mg/kg body weight may be used. In further embodiments, dosages of between about 10 and about 50 mg/kg body weight in further embodiments about 10, about 25 or about 50 mg/kg body weight, may be used.

The present disclosure further provides a kit or package comprising at least one container means having disposed therein at least one of an gRNA, pegRNA, nuclease, nucleic acids, vector, cell, systems, combination, or composition as described herein. In an embodiment, the kit or package further comprises with instructions for use, such as for modification of a nucleotide sequence in a cell, or for the treatment of a disease, disorder, or condition.

### Methods/Uses

The present disclosure also relates to a method for inducing the expression of a nucleic acid of interest or gene of interest by a cell, the method comprising introducing the nucleic acid, expression cassette, or vector described herein in the cell. The present disclosure also relates to a use of a nucleic acid, expression cassette, or vector described herein for inducing the expression of a gene of interest by a cell. In an embodiment, the cell is a primary cell, for example a brain/neuronal cell, a peripheral blood cell (e.g., a B or T lymphocyte, a monocyte, or a NK cell), a cord blood cell, a bone marrow cell, a cardiac cell, an endothelial cell, an epidermal cell, an epithelial cell, a fibroblast, hepatic cell, or a lung/pulmonary cell. In an embodiment, the cell is a bone marrow cell, peripheral blood cell, or cord blood cell. In a further embodiment, the cell is an immune cell, such as a T cell (e.g., a CD3+ T cell, a CD8+ T cell, a CD4⁺ T cell, a Regulatory T cell (Tregs, e.g., CD4⁺/FOXP3⁺)), a B cell, or a NK cell.

In an embodiment, the gene of interest encodes a protein that is defective or absent in the cell. In an embodiment, the gene of interest encodes a recombinant receptor, such as a chimeric antigen receptor (CAR). In an embodiment, the gene of interest encodes a differentiation factor (for cell reprogramming).

The present disclosure also relates to a method for treating a disease, condition, or disorder in a subject, the method comprising administering a cell comprising a nucleic acid, expression cassette, or vector described herein. The present disclosure also relates to the use of a cell comprising a nucleic acid, expression cassette, or vector described herein method for treating a disease, condition, or disorder in a subject. The present disclosure also relates to the use of a cell comprising a nucleic acid, expression cassette, or vector described herein method for the manufacture of a medicament for treating a disease, condition, or disorder in a subject. In an embodiment, the disease, condition, or disorder is associated with the absence of expression of a protein or the expression of a defective (e.g., mutated) protein, and the synthetic expression cassette or vector comprises a nucleic acid encoding a functional (e.g., native) protein (e.g., gene therapy).

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g., cancer), autoimmune or inflammatory disease (e.g., arthritis, rheumatoid arthritis [RA], type I diabetes, systemic lupus erythematosus [SLE], inflammatory bowel disease, psoriasis, scleroderma, autoimmune thyroid disease, Grave's disease, Crohn's disease, multiple sclerosis, asthma, and/or a disease or condition associated with transplant), or an infectious disease, e.g. caused by a bacterial, viral, or other pathogen. In particular embodiments, the recombinant receptor, e.g., the CAR, specifically binds to the antigen associated with the disease or condition. In an embodiment, the disease, condition, or disorder is cancer or an infectious disease, and the nucleic of interest present in the synthetic expression cassette or vector encodes a recombinant receptor, such as a chimeric antigen receptor (CAR), that recognizes an antigen expressed by the tumor cell or infected cell. The tumor may be a solid tumor or a hematologic (blood) tumor. In an embodiment, the cancer is a hematologic cancer, such as a lymphoma, a leukemia, and/or a myeloma (e.g., B-cell, T-cell, and myeloid leukemias, lymphomas, and multiple myelomas). The infectious disease may be a disease caused by any pathogenic infection, such as a viral, bacterial, parasitic (e.g., protozoal), or fungal infection, for example human immunodeficiency virus (HIV) or cytomegalovirus (CMV) infection.

The cells (engineered cells comprising a nucleic acid, expression cassette, or vector described herein) or compositions comprising same may administered to a subject or patient having the particular disease or condition to be treated, e.g., via adoptive cell therapy such as adoptive T cell therapy or stem cell therapy. Methods for administration of engineered cells for adoptive cell therapy are known and may be used in connection with the provided methods and compositions. For example, adoptive T cell therapy methods are described, e.g., in U.S. Patent Application Publication No. 2003/0170238 to Gruenberg et al; U.S. Pat. No. 4,690,915 to Rosenberg; Rosenberg S. A., Nat. Rev. Clin. Oncol. 8: 577-85, 2011). See, e.g., Themeli M. et al., Nat. Biotechnol. 31: 928-933, 2013; Tsukahara T. et al., Biochem. Biophys. Res. Commun. 438: 84-89, 2013; Davila M. L. et al., PLoS ONE 8: e61338, 2013.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

In some embodiments, the cell therapy, e.g., adoptive T cell therapy or stem cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy or stem cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or super type as the first subject. The cells can be administered by any suitable means. Dosing and administration may depend in part on whether the administration is brief or chronic. Various dosing schedules include, but are not limited to, single or multiple administrations over various time-points, bolus administration, and pulse infusion.

In certain embodiments, the cells, or individual populations of subtypes of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose of recombinant receptor (e.g., CAR)-expressing cells, stem cells, T cells, or peripheral blood mononuclear cells (PBMCs), is at least 1×10², 1×10³, 1×10⁴ or 1×10⁵ cells, for example in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

The cells may be used in combination with other therapy such as other chemotherapy, immunotherapy, radiotherapy, or surgery, according to the disease to be treated.

In some embodiments, the synthetic expression cassette is used as a research tool, for example as reporter tool or in a commercial detection method (assay development). For example, the synthetic expression cassette may be operably linked to a nucleic acid encoding a reporter protein, which may be used for the detection of the expression of a gene of interest in a specific cell type, e.g., to confirm that the gene of interest has been taken up by and is expressed by the cell. The term “reporter protein” refers to a protein that may be easily identified and measured such as fluorescent and luminescent proteins (e.g., GFP, YFP), as well as enzymes that are able to generate a detectable product from a substrate (e.g., luciferase). The synthetic expression cassette may also be used for the cell-specific expression of a gene of interest in vitro, e.g., to assess the effect of the expression of the gene of interest in the targeted cells.

### CRISPR System

The CRISPR technology is a system for genome editing, e.g., for modification of a nucleic acid sequence, and may also be used for example to modify the expression of a specific gene.

This system stems from findings in bacterial and archaea which have developed adaptive immune defenses termed clustered regularly interspaced short palindromic repeats (CRISPR) systems, which use crRNAs and Cas proteins to degrade complementary sequences present in invading viral and plasmid DNA. The original CRISPR systems comprised a CRISPR RNA (crRNA) and a trans-activating crRNA (tracrRNA), which form a hybrid (which guides a CRISPR nuclease, e.g. a Cas9).

Engineered CRISPR systems use for example a synthetically reconstituted “guide RNA” (“gRNA”), corresponding to a crRNA-tracrRNA fusion that obviates the need for RNase Ill and crRNA processing in general. The gRNA comprises a “gRNA guide sequence” or “gRNA target sequence” and an RNA sequence (Cas recognition sequence)”, which is necessary for CRISPR nuclease (e.g., Cas9) binding to the targeted gene. The gRNA guide sequence is the sequence that confers specificity. It hybridizes with (i.e., it is complementary to) the opposite strand of a target sequence (i.e., it corresponds to the RNA sequence of a DNA target sequence). Other CRISPR systems using different CRISPR nucleases have been developed and are known in the art (e.g., using the Cpf1 nuclease instead of a Cas9 nuclease).

Because the original Cas9 nuclease combined with a gRNA may produce off-target mutagenesis, one may alternatively use in accordance with the present disclosure a pair of specifically designed gRNAs or pegRNAs in combination with a Cas9 nickase or in combination with a dCas9-Folkl nuclease to cut both strands of DNA.

Base editing: CRISPR/Cas technology has also been developed to permit base editing without inducing a DSB. For example, base editing may be performed using a guide RNA and a Cas9 nickase (cutting only one DNA strand) fused with a cytidine deaminase to chemically modify a cytidine into a thymine (Komor, A. C., et al., Programmable editing of a target base in genomic DNA without double-stranded DNA cleavage, Nature 533: 420-424, 2016). Base editing may also be performed using a guide RNA and a Cas9 nickase fused with an adenosine deaminase to chemically modify an adenosine into an inosine, the equivalent of a guanine (Gaudelli, N. M., et al., Programmable base editing of A*T to G*C in genomic DNA without DNA cleavage. Nature 551: 464-471, 2017).

Prime editing: Another use of CRISPR/Cas technology is prime editing, which uses an extended guide RNA (called a pegRNA) and a Cas9 nickase fused with a reverse transcriptase (the fusion protein is e.g., PE2) to replace any nucleotide by any other nucleotide³⁸.

Therefore, in embodiments, the methods described herein may utilize for example a Cas9 nickase fused either with a deaminase (e.g., a cytidine deaminase or an adenosine deaminase) or reverse transcriptase to efficiently modify a target gene (e.g., MTOR) in cells via base editing or prime editing, respectively. Thus in embodiments, the methods described herein comprise providing a cell with a Cas9 nickase-cytidine deaminase, Cas9 nickase-adenosine deaminase or a Cas9-nickase-reverse transcriptase specifically targeting a nucleic acid sequence in the endogenous MTOR polynucleotide gene or mRNA sequence of the cell.

Therefore, in embodiments, a method described herein comprises providing a cell with:
a. a1) at least one guide RNA comprising one or more guide sequences having one or more target sequences in the target polynucleotide, such that the one or more gRNAs direct the cleavage of the target polynucleotide at the MTOR locus to introduce the first and second modifications (and optionally, the one or more further modifications), wherein the one or more target sequences are each contiguous to a protospacer adjacent motif recognized by a Cas9 nickase; or
   a2) a vector comprising a nucleic acid sequence corresponding to the guide RNA for expressing the guide RNA; and
b. b1) a Cas nickase-deaminase fusion protein comprising (i) a Cas nickase polypeptide domain and (ii) a cytidine deaminase polypeptide domain or an adenosine deaminase polypeptide domain; or
   b2) a vector comprising a nucleic acid sequence encoding the Cas nickase-deaminase fusion protein, for expression of the Cas nickase-deaminase fusion protein; and
      wherein at least one modification is introduced into the target polynucleotide, wherein the at least one modification comprises modification of a cytidine into a thymidine or an adenosine into an inosine, and wherein the at least one modification results in the introduction of the first modification.

In further embodiments, a method described herein comprises providing a cell with:
a. a1) at least one pegRNA comprising one or more guide sequences having one or more target sequences in the target polynucleotide, such that the one or more pegRNAs direct the cleavage of the target polynucleotide at the MTOR locus to introduce the first and second modifications (and optionally, the one or more further modifications), wherein the one or more target sequences are each contiguous to a protospacer adjacent motif recognized by a Cas9 nickase; or
   a2) a vector comprising a nucleic acid sequence corresponding to the pegRNA for expressing the pegRNA; and
b. b1) a Cas nickase-reverse transcriptase fusion protein comprising (i) a Cas nickase polypeptide domain and (ii) a reverse transcriptase polypeptide domain; or
   b2) a vector comprising a nucleic acid sequence encoding the Cas nickase-reverse transcriptase fusion protein, for expression of the Cas nickase-reverse transcriptase fusion protein; and
      wherein at least one modification is introduced into the target polynucleotide, wherein the at least one modification results in the introduction of the first modification.

In embodiments, the Cas nickase polypeptide domain comprises a SpCas9 nickase, a SaCas9 nickase, a CjCas9 nickase, a ScCas9 nickase, a CasX nickase, a CasY nickase, a Cpf1 nickase, a eSaCas9 nickase, a eSpCas9 nickase, a HiFi Cas9 nickase, a Cas9 H840A nickase, a St1Cas9 nickase or a derivative of any thereof having nickase activity.

In embodiments, the deaminase is a REPAIRv2 deaminase (Cox, D. B. T., et al., RNA editing with CRISPR-Cas13. Science 358: 1019-1027, 2017) or an ADAR2 deaminase (Wettengel, J., et al., Harnessing human ADAR2 for RNA repair-Recoding a PINK1 mutation rescues mitophagy. Nucleic Acids Res 45: 2797-2808, 2017).

In embodiments, the Cas nickase-deaminase fusion protein is SpCas9n VQR BE3, SpCas9n EQR BE3, SaCas9n-KKH BE3, YE1-SpCas9n VQR BE3, YE1-SpCas9n EQR BE3, YE1-SaCas9n-KKH BE3, SpCas9n VQR BE4, SpCas9n EQR BE4, SaCas9n-KKH BE4, Target-AID SpCas9n VQR, Target-AID SpCas9n EQR, Target-AID SaCas9n-KKH, Target-AID-BE3, a fusion protein comprising an St1Cas9-derived nickase, a fusion protein comprising an St3Cas9-derived nickase, or a derivative of any thereof comprising nickase and deaminase activity.

In embodiments, the reverse transcriptase domain comprises an M-MLV reverse transcriptase.

In embodiments, provided herein are CRISPR/nuclease-based engineered systems for use in modifying a target nucleic acid in cells. Introduction of DSBs can knockout a specific gene or allow modifying it by Homology Directed Repair (HDR), where one or more donor or patch nucleic acids comprising the desired modification(s) are provided to introduce the modification(s) by HDR. CRISPR/Cas9-induced DNA cleavage followed by Non-Homologous End Joining (NHEJ) repair has been used to generate loss-of-function alleles in protein-coding genes or to delete a very large DNA fragment. The CRISPR-based engineered systems of the present disclosure are designed to (i) target and cleave a gene of interest) to generate gene variants (e.g., creating insertion[s] and/or deletions, also referred to as INDELS).

Accordingly, in an aspect, the present disclosure involves the design and preparation of one or more gRNAs or pegRNAs for inducing a DSB (or two single stranded breaks [SSB] in the case of a nickase) in a target gene of interest. In embodiments, the present disclosure also involves the design and preparation of one or more gRNAs or pegRNAs for inducing a DSB (or two SSBs in the case of a nickase) in a target polynucleotide located at a different locus within the genome of target cells. The gRNAs or pegRNAs and the nuclease are then used together to introduce the desired modification(s) (i.e., gene-editing events) by NHEJ or HDR within the genome of one or more target cells. When the desired modification(s) include specific point mutation(s) or insertions/deletion(s), one or more donor or patch nucleic acids comprising the desired modification(s) are provided to introduce the modification(s) by HDR.

gRNAs

In order to cut DNA at a specific site, CRISPR nucleases require the presence of a gRNA and a protospacer adjacent motif (PAM) on the targeted gene. The PAM immediately follows (i.e., is adjacent to) the gRNA target sequence in the targeted polynucleotide gene sequence. The PAM is located at the 3′ end or 5′ end of the gRNA target sequence (depending on the CRISPR nuclease used) but is not included in the gRNA guide sequence. For example, the PAM for Cas9 CRISPR nucleases is located at the 3′ end of the gRNA target sequence on the target gene while the PAM for Cpf1 nucleases is located at the 5′ end of the gRNA target sequence on the target gene. Different CRISPR nucleases also require a different PAM. Accordingly, selection of a specific polynucleotide gRNA target sequence is generally based on the CRISPR nuclease used. The PAM for the Streptococcus pyogenes Cas9 CRISPR system is 5′-NRG-3′, where R is either A or G, and characterizes the specificity of this system in human cells. The PAM of Staphylococcus aureus Cas9 is NNGRR. The S. pyogenes Type II system naturally prefers to use an “NGG” sequence, where “N” can be any nucleotide, but also accepts other PAM sequences, such as “NAG” in engineered systems. Similarly, the Cas9 derived from Neisseria meningitidis (NmCas9) normally has a native PAM of NNNNGATT, but has activity across a variety of PAMs, including a highly degenerate NNNNGNNN PAM. The PAM for AsCpf1 or LbCpf1 CRISPR nuclease is TTTN. In an embodiment, the PAM for a Cas9 protein used in accordance with the present disclosure is a NGG trinucleotide-sequence (Cas9). In another embodiment, the PAM for a Cpf1 CRISPR nuclease used in accordance with the present disclosure is a TTTN nucleotide sequence. In a preferred embodiment, the St1Cas9 may be used, which corresponds to the PAM sequences NNAGAA and NNGGAA. In embodiments, different St1Cas9 PAM sequences may be used, for example, inferred consensus PAM sequences for St1Cas9 from strains CNRZ1066 and LMG13811 are NNACAA(W) and NNGCAA(A), respectively^{24, 26}. Table 1 below provides a list of non-limiting examples of CRISPR/nuclease systems with their respective PAM sequences.

TABLE 1
Non-exhaustive list of CRISPR-nuclease systems from different species
(see Mohanraju P. et al., Science 353: aad5147, 2016; Shmakov S. et al.,
Mol. Cell 60: 385-397, 2015; and Zetsche B. et al., Cell 163: 759-771,
2015). Also included are engineered variants recognizing alternative
PAM sequences (see Kleinstiver B.P. et al., Nat. Biotechnol. 33:
1293-1298, 2015 and Kleinstiver B.P. et al., Nature 523: 481-485, 2015).

| CRISPR nuclease | PAM Sequence |
|---|---|
| Streptococcus pyogenes (Sp); SpCas9 | NGG + NAG |
| SpCas9 D1135E variant | NGG (reduced NAG binding) |
| SpCas9 VRER variant | NGCG |
| CRISPR nuclease | PAM Sequence |
| SpCas9 EQR variant | NGAG |
| SpCas9 VQR variant | NGAN or NGNG |
| Staphylococcus aureus (Sa); SaCas9 | NNGRRT or NNGRR(N) |
| SaCas9 KKH variant | NNNRRT |
| Neisseria meningitidis (Nm) | NNNNGATT |
| Streptococcus thermophilus (St1) | NNAGAA and NNGGAA |
| Treponema denticola (Td) | NAAAAC |
| AsCpf1(Acidominococcus) | TTTN |
| AsCpf1 S542R/K607R | TYCV |
| AsCpf1 S542R/K548V/N552R | TATV |
| LbCpf1 (Lachnospiraceae) | TTTN |
| LbCpf1 G532R/K595R | TYCV |

As used herein, the expressions “guide RNA”, “gRNA”, “single guide RNA”, “sgRNA” are used interchangeably and refer to a polynucleotide sequence which works in combination with a CRISPR nuclease to hybridize with a target nucleic acid sequence and direct sequence-specific binding of a nucleic acid-targeting complex to the target nucleic acid sequence, which in turn can introduce a cut into DNA. The gRNA comprises a gRNA guide sequence and a “CRISPR nuclease recognition sequence”.

As used herein, the expression “gRNA guide sequence” refers to the corresponding RNA sequence of the “gRNA target sequence”. Therefore, it is the RNA sequence equivalent of the protospacer on the target polynucleotide gene sequence. It does not include the corresponding PAM sequence in the genomic DNA. It is the sequence that confers target specificity. The gRNA guide sequence is linked to a CRISPR nuclease recognition sequence which binds to the nuclease (e.g., Cas9/Cpf1). The gRNA guide sequence recognizes and binds to the targeted gene of interest. It hybridizes with (i.e., is complementary to) the opposite strand of a target gene sequence, which comprises the PAM (i.e., it hybridizes with the DNA strand opposite to the PAM). As noted above, the “PAM” is the nucleic acid sequence, that immediately follows (is contiguous to) the target sequence or target polynucleotide but is not in the gRNA. Further, the embodiments and features described herein in respect of gRNAs also apply to pegRNAs, a type of gRNA used in prime editing. Thus in an embodiment, the gRNA is a pegRNA, which is for use in prime editing.

A “CRISPR nuclease recognition sequence” as used herein refers broadly to one or more RNA sequences (or RNA motifs) required for the binding and/or activity (including activation) of the CRISPR nuclease on the target gene. Some CRISPR nucleases require longer RNA sequences than other to function. Also, some CRISPR nucleases require multiple RNA sequences (motifs) to function while others only require a single short RNA sequence/motif. For example, Cas9 proteins require a tracrRNA sequence in addition to a crRNA sequence to function while Cpf1 only requires a crRNA sequence. Thus, unlike Cas9, which requires both crRNA sequence and a tracrRNA sequence (or a fusion or both crRNA and tracrRNA) to mediate interference, Cpf1 processes crRNA arrays independent of tracrRNA, and Cpf1-crRNA complexes alone cleave target DNA molecules, without the requirement for any additional RNA species (see Zetsche B. et al., Cell 163: 759-771, 2015).

The “CRISPR nuclease recognition sequence” included in the gRNA or pegRNA described herein is thus selected based on the specific CRISPR nuclease used. It includes direct repeat sequences and any other RNA sequence known to be necessary for the selected CRISPR nuclease binding and/or activity. Various RNA sequences which can be fused to an RNA guide sequence to enable proper functioning of CRISPR nucleases (referred to herein as CRISPR nuclease recognition sequence) are well known in the art and can be used in accordance with the present disclosure. The “CRISPR nuclease recognition sequence” may thus include a crRNA sequence only (e.g., for AsCpf1 activity, such as the CRISPR nuclease recognition sequence UAAUUUCUAC UCUUGUAGAU (SEQ ID NO: 60)) or may include additional sequences (e.g., tracrRNA sequence necessary for Cas9 activity). Furthermore, in accordance with the present disclosure and as well known in the art, RNA motifs necessary for CRISPR nuclease binding and activity may be provided separately (e.g., [i] RNA guide sequence-crRNA CRISPR recognition sequence” [also known as crRNA] in one RNA molecule and [ii] a tracrRNA CRISPR recognition sequence on another, separate RNA molecule). Alternatively, all necessary RNA sequences (motifs) may be fused together in a single RNA guide. The CRISPR recognition sequence is preferably fused directly to the gRNA guide sequence (in 3′ [e.g., Cas9] or 5′ [Cpf1] depending on the CRISPR nuclease used) but may include a spacer sequence separating two RNA motifs. In embodiments, the CRISPR nuclease recognition sequence is a Cas9 recognition sequence having at least 65 nucleotides. In embodiments, the CRISPR nuclease recognition sequence is a Cas9 CRISPR nuclease recognition sequence having at least 85 nucleotides. In embodiments, the CRISPR nuclease recognition sequence is a Cpf1 recognition sequence (5′ direct repeat) having about 19 nucleotides. In an embodiment, the CRISPR nuclease recognition sequence is a St1Cas9 recognition sequence. The gRNA or pegRNA of the present disclosure may comprise any variant of the above noted sequences, provided that it allows for the proper functioning of the selected CRISPR nuclease (e.g., binding of the CRISPR nuclease protein to the gene of interest and/or target polynucleotide sequence[s]).

Together, the RNA guide sequence and CRISPR nuclease recognition sequence(s) provide both targeting specificity and scaffolding/binding ability for the CRISPR nuclease of the present disclosure. gRNAs and pegRNAs of the present disclosure do not exist in nature, i.e., is a non-naturally occurring nucleic acid(s).

A “target region”, “target sequence” or “protospacer” in the context of gRNAs/pegRNAs and CRISPR system of the present disclosure are used herein interchangeably and refers to the region of the target gene, which is targeted by the CRISPR/nuclease-based system, without the PAM. It refers to the sequence corresponding to the nucleotides that precede the PAM (i.e., in 5′ or 3′ of the PAM, depending on the CRISPR nuclease) in the genomic DNA. It is the sequence that is included into a gRNA/pegRNA expression construct (e.g., vector/plasmid/AAV). The CRISPR/nuclease-based system may include at least one (i.e., one or more) gRNAs, wherein each gRNA/pegRNA target different DNA sequences on the target gene. The target DNA sequences may be overlapping. The target sequence or protospacer is followed or preceded by a PAM sequence at an (3′ or 5′ depending on the CRISPR nuclease used) end of the protospacer. Generally, the target sequence is immediately adjacent (i.e., is contiguous) to the PAM sequence (it is located on the 5′ end of the PAM for SpCas9-like nuclease and at the 3′ end for Cpf1-like nuclease).

In embodiments, the gRNA of the present disclosure comprises a “gRNA guide sequence” or has a “gRNA target sequence” which corresponds to the target sequence on the gene of interest or target polynucleotide sequence that is followed or preceded by a PAM sequence (is adjacent to a PAM). The gRNAmay comprise a “G” at the 5′ end of its polynucleotide sequence. The presence of a “G” in 5′ is preferred when the gRNA is expressed under the control of the U6 promoter (Koo T. et al., Mol. Cells 38: 475-481, 2015). The CRISPR/nuclease system of the present disclosure may use gRNAs of varying lengths. The gRNA may comprise a gRNA guide sequence of at least 10 nucleotides (nts), at least 12 nts, at least 13 nts, at least 14 nts, at least 15 nts, at least 16 nts, at least 17 nts, at least 18 nts, at least 19 nts, at least 20 nts, at least 21 nts, at least 22 nts, at least 23 nts, at least 24 nts, at least 25 nts, at least 30 nts, or at least 35 nts of a target sequence of a gene of interest or target polynucleotide (such target sequence is followed or preceded by a PAM in the gene of interest or target polynucleotide but is not part of the gRNA). The length of the gRNA is selected based on the specific CRISPR nuclease used. In embodiments, the “gRNA guide sequence” or “gRNA target sequence” may be at least 17 nucleotides (17, 18, 19, 20, 21, 22, or 23) long, preferably between 17 and 30 nts long, more preferably between 17-22 nucleotides long. In embodiments, the gRNA guide sequence is between 10-40, 10-30, 12-30, 15-30, 18-30, or 10-22 nucleotides long. In embodiments, the PAM sequence is “NGG”, where “N” can be any nucleotide. In embodiments, the PAM sequence is “TTTN”, where “N” can be any nucleotide. gRNAs may target any region of a target gene which is immediately adjacent (contiguous, adjoining, in 5′ or 3′) to a PAM (e.g., NGG/TTTN or CCN/NAAA for a PAM that would be located on the opposite strand) sequence. In embodiments, the gRNA of the present disclosure has a target sequence that is located in an exon (the gRNA guide sequence consists of the RNA sequence of the target [DNA] sequence which is located in an exon). In embodiments, the gRNA of the present disclosure has a target sequence that is located in an intron (the gRNA guide sequence consists of the RNA sequence of the target [DNA] sequence which is located in an intron). In embodiments, the gRNA may target any region (sequence) which is followed (or preceded, depending on the CRISPR nuclease used) by a PAM in the gene or target polynucleotide of interest.

Although a perfect match between the gRNA guide sequence and the DNA sequence on the targeted gene is preferred, a mismatch between a gRNA guide sequence and target sequence on the gene sequence of interest is also permitted, as long as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. Preferably, the gRNA of the present disclosure comprises 7 mismatches, 6 mismatches, 5 mismatches, 4 mismatches, 3 mismatches, more preferably 2 mismatches, or less, and even more preferably no mismatch, with the corresponding gRNA target gene sequence (less the PAM). Preferably, the gRNA nucleic acid sequence is at least 90%, 91%, 92%, 93%, 94%, 95%, 96%, 97%, 98%, or 99% identical to the gRNA target polynucleotide sequence in the gene of interest. Of course, the smaller the number of nucleotides in the gRNA guide sequence the smaller the number of mismatches tolerated. The binding affinity is thought to depend on the sum of matching gRNA-DNA combinations.

The number of gRNAs administered to or expressed in a target cell in accordance with the methods of the present disclosure may be at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, at least 16 gRNAs, at least 17 gRNAs, or at least 18 gRNAs. The number of gRNAs administered to or expressed in a cell may be between at least 1 gRNA and 15 gRNAs, 1 gRNA and least 10 gRNAs, 1 gRNA and 8 gRNAs, 1 gRNA and 6 gRNAs, 1 gRNA and 4 gRNAs, 1 gRNA and gRNAs, 2 gRNA and 5 gRNAs, or 2 gRNAs and 3 gRNAs.

### CRISPR Nucleases

Recombinant dCas9-FoKI dimeric nucleases (RFNs) have been designed that can recognize extended sequences and edit endogenous genes with high efficiency in human cells. These nucleases comprise a dimerization-dependent wild type FokI nuclease domain fused to a catalytically inactive Cas9 (dCas9) protein. Dimers of the fusion proteins mediate sequence specific DNA cleavage when bound to target sites composed of two half-sites (each bound to a dCas9 monomer domain, i.e., a Cas9 nuclease devoid of nuclease activity) with a spacer sequence between them. The dCas9-FoKI dimeric nucleases require dimerization for efficient genome editing activity and thus, use two gRNAs for introducing a cut into DNA.

The recombinant CRISPR nuclease that may be used in accordance with the present disclosure is i) derived from a naturally occurring Cas; and ii) has a nuclease (or nickase) activity to introduce a DSB (or two SSBs in the case of a nickase) in cellular DNA when in the presence of appropriate gRNA(s). Thus, as used herein, the term “CRISPR nuclease” refers to a recombinant protein which is derived from a naturally occurring Cas nuclease which has nuclease or nickase activity and which functions with the gRNAs of the present disclosure to introduce DSBs (or one or two SSBs) in the targets of interest. In an embodiment, the CRISPR nuclease is St1Cas9. In further embodiments, the CRISPR nuclease is SpCas9 or Cpf1. In another embodiment, the CRISPR nuclease is a Cas9 protein having a nickase activity. As used herein, the term “Cas9 nickase” refers to a recombinant protein which is derived from a naturally occurring Cas9 and which has one of the two nuclease domains inactivated such that it introduces single stranded breaks (SSB) into the DNA. It can be either the RuvC or HNH domain. In a further embodiment, the Cas protein is a dCas9 protein fused with a dimerization-dependant FokI nuclease domain.

Exemplary CRISPR nucleases that may be used in accordance with the present disclosure are provided in Table 1 above.

CRISPR nucleases such as Cas9/nucleases cut 3-4 bases upstream of the PAM sequence. CRISPR nucleases such as Cpf1 on the other hand, generate a 5′ overhang. The cut occurs 19 bases after the PAM on the targeted (+) strand and 23 bases on the opposite strand (Zetsche et al., 2015, PMID 26422227). There can be some off-target DSBs using wildtype Cas9. The degree of off-target effects depends on a number of factors including: how closely homologous the off-target sites are compared to the on-target site, the specific site sequence, and the concentration of nuclease and guide RNA (gRNA). These considerations only matter if the PAM sequence is immediately adjacent to the nearly homologous target sites. The mere presence of additional PAM sequences should not be sufficient to generate off target DSBs; there needs to be extensive homology of the protospacer followed or preceded by PAM.

In embodiments, CRISPR nuclease (Cas or other nuclease/nickase recombinant protein described herein) preferably comprises at least one Nuclear Localization Signal (NLS) to target the protein into the cell nucleus, and the vector further comprises one or more nucleotide sequences encoding the one or more NLSs. Accordingly, as used herein the expression “nuclear localization signal” or “NLS” refers to an amino acid sequence, which ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal, which targets proteins out of the nucleus. Classical NLSs can be further classified as either monopartite or bipartite. The first NLS to be discovered was the sequence PKKKRKV (SEQ ID NO: 61) in the SV40 Large T-antigen (a monopartite NLS). The NLS of nucleoplasmin, KR[PAATKKAGQA]KKKK (SEQ ID NO: 62), is the prototype of the ubiquitous bipartite signal: two clusters of basic amino acids, separated by a spacer of about 10 amino acids. The Cas9 protein exemplified herein is a Cas9 nuclease comprising one or more, preferably two, NLS sequences.

There are many other types of NLS, which are qualified as “non-classical”, such as the acidic M9 domain of hnRNP A1, the sequence KIPIK in yeast transcription repressor Matα2, the complex signals of U snRNPs, as well as a recently identified class of NLSs known as PY-NLSs. Thus, any type of NLS (classical or non-classical) may be used in accordance with the present disclosure, as long as it targets the protein of interest into the nucleus of a target cell. In an embodiment, the NLS is derived from the simian virus 40 large T antigen. In an embodiment, the NLS of the recombinant protein of the present disclosure comprises or consists of the following amino acid sequence SPKKKRKVEAS (SEQ ID NO: 63). In an embodiment the NLS comprises or consists of the sequence KKKRKV (SEQ ID NO: 64). In an embodiment, the NLS comprises or consists of the sequence SPKKKRKVEASPKKKRKV (SEQ ID NO: 65). In another embodiment, the NLS comprises or consists of the sequence KKKRK (SEQ ID NO: 66). In another embodiment, the NLS comprises or consists of the sequence PKKKRKV (SEQ ID NO: 67).

In an embodiment, the CRISPR nuclease comprises a first NLS at its amino terminal end and a second NLS at its carboxy terminal end, and the vector comprises NLS-encoding nucleotide sequences flanking the CRISPR nuclease-encoding nucleotide sequence.

In embodiments, the CRISPR nuclease or Cas nickase may optionally advantageously be coupled to a protein transduction domain entry of the protein into the target cells. Alternatively, the nucleic acid coding for the guide RNA or pegRNA and for the deaminase or reverse transcriptase may be delivered in targeted cells using various viral vectors, virus like particles (VLP), or exosomes.

Protein transduction domains (PTD) may be of various origins and allow intracellular delivery of a given therapeutic by facilitating the translocation of the protein/polypeptide into a cell membrane, organelle membrane, or vesicle membrane. PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle including the mitochondria.

In an embodiment, a PTD is covalently linked to the amino terminus of a recombinant protein of the present disclosure. In another embodiment, a PTD is covalently linked to the carboxyl terminus of a recombinant protein of the present disclosure. Exemplary protein transduction domains include but are not limited to a minimal undecapeptide protein transduction domain (corresponding to residues 47-57 of HIV-1 TAT comprising YGRKKRRQRRR (SEQ ID NO: 49); a polyarginine sequence comprising a number of arginines sufficient to direct entry into a cell (e.g., 3, 4, 5, 6, 7, 8, 9, 10, or 10-50 arginines); a VP22 domain; an Drosophila Antennapedia protein transduction domain; a truncated human calcitonin peptide; RRQRRTSKLMKR (SEQ ID NO: 50); Transportan GWTLNSAGYLLGKINLKALAALAKKIL (SEQ ID NO: 51); KALAWEAKLAKALAKALAKHLAKALAKALKCEA (SEQ ID NO: 52); and RQIKIWFQNRRMKWKK (SEQ ID NO: 53). Further exemplary PTDs include but are not limited to, KKRRQRRR (SEQ ID NO: 54), RKKRRQRRR (SEQ ID NO: 55); or an arginine homopolymer of from 3 arginine residues to 50 arginine residues. In an embodiment, the protein transduction domain is TAT or Pep-1. In an embodiment, the protein transduction domain is TAT and comprises the sequence SGYGRKKRRQRRRC (SEQ ID NO: 56). In another embodiment, the protein transduction domain is TAT and comprises the sequence YGRKKRRQRRR (SEQ ID NO: 57). In another embodiment, the protein transduction domain is TAT and comprises the sequence KKRRQRRR (SEQ ID NO: 58). In another embodiment, the protein transduction domain is Pep-1 and comprises the sequence KETWWETWWTEWSQPKKKRKV (SEQ ID NO: 59).

Other non-limiting examples of PTD include an endosomal escape peptide. Non-limiting examples of such endosomal escape peptides include DT, GALA, PEA, INF-7, LAH4, CM18, HGP, H5WYG, HA2, and EB1.

### Optimization of Codon Degeneracy

Because CRISPR nuclease proteins are (or are derived from) proteins normally expressed in bacteria, it may be advantageous to modify their nucleic acid sequences for optimal expression in eukaryotic cells (e.g., mammalian cells) when designing and preparing CRISPR nuclease recombinant proteins. Similarly, donor or patch nucleic acids of the present disclosure used to introduce specific modifications in the target polynucleotide may use codon degeneracy (e.g., to introduce new restriction sites for enabling easier detection of the targeted modification).

Accordingly, the following codon chart (Table 2) may be used, in a site-directed mutagenic scheme, to produce nucleic acids encoding the same or slightly different amino acid sequences of a given nucleic acid:

TABLE 2
Codons encoding the same amino acid

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

mTOR Inhibitors

In embodiments of the present disclosure, mTOR inhibitors include rapamycin and analogs or derivatives thereof that also act as mTOR inhibitors, commonly referred to as “rapalogs”. Rapamycin (CAS No. 53123-88-9; also referred to as sirolimus), is a macrolide compound produced by Streptomyces hygroscopicus, that inhibits mTOR, and is used as an immunosuppressant, for example to prevent organ transplant rejection, and is also used as an anticancer agent. Rapamycin has the following structure.

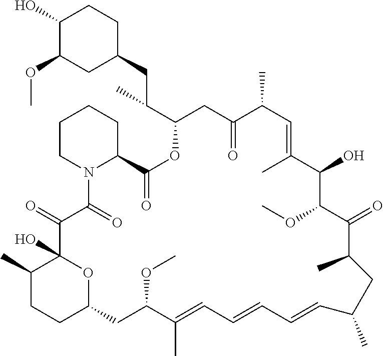 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Rapalogs include for example temsirolimus (CCI-779), everolimus (RAD001), deforolimus (ridaforolimus, AP23573, MK-8669), umirolimus, zotarolimus, and ridaforolimus (AP-23573). In embodiments, any combination or mixture of two or more of rapamycin and rapalogs may be used in the methods of the present disclosure.

In embodiments, the mTOR inhibitor is also an anticancer agent.

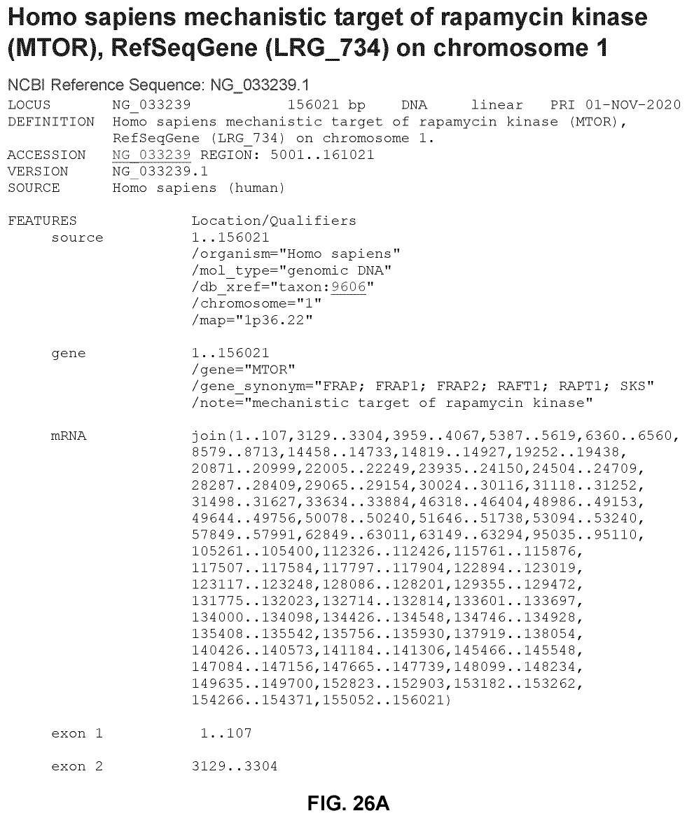

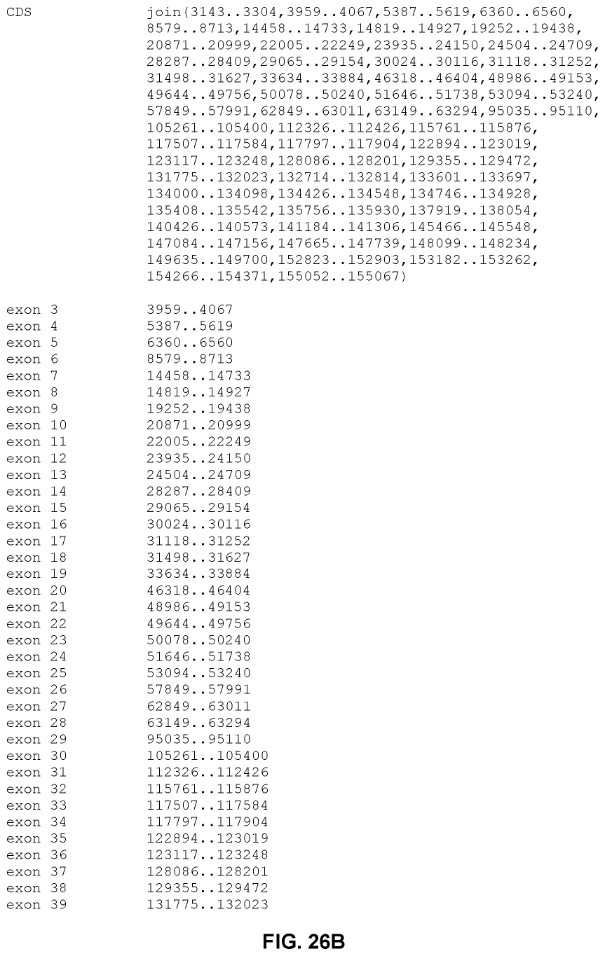

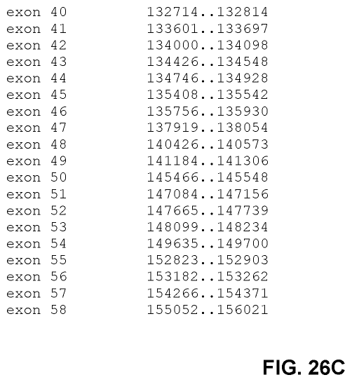

mTOR (mechanistic target of rapamycin; also referred to as FRAP1) is a protein kinase that belongs to the family of phosphatidylinositol-3 kinase (PI3K) related kinases. It functions as a component of two different protein complexes, mTOR complex 1 (mTORC1) and mTOR complex 2 (mTORC2). In mTORC1, it functions as a serine/threonine protein kinase; in mTORC2, it also functions as a tyrosine protein kinase. mTOR is a 2549 amino acid polypeptide containing various functional domains, including: up to 12 HEAT repeats in the N-terminal region, a central FAT domain (located approximately at residues 1513-1910), FRB domain (located at approximately residues 2015-2114), Ser/Thr kinase domains (located at approximately residues 2517-2549), and a C-terminal FATC domain (located at approximately residues 2517-2549). The FRB domain is responsible for the binding or rapamycin and rapalogs. The human mTOR genomic sequence (Accession: NG_033239) is set forth in SEQ ID NO: 1, with details thereof provided in FIG. 26. The human mTOR amino acid sequence is set forth in SEQ ID NO: 2.

In an embodiment, the modification described herein that confers resistance to an mTOR inhibitor is a modification within the mTOR FRB domain, for example in the region of residues 2015-2114. In an embodiment, the modification is at position 2034, which is an alanine in wild type mTOR. In a further embodiment, the modification is at position 2035, which is a serine in wild type mTOR. In a further embodiment, the modification is at position 2108, which is a phenylalanine in wild type mTOR. In embodiments, the substitution is at one or more of positions 2034, 2035, and 2108 of mTOR. In an embodiment, the modification is an amino acid substitution, in a further embodiment a substitution selected from one or more of a F2108L substitution, a F2108I substitution, a F2108M substitution, a F2108V substitution, a F2108K substitution, a F2108G substitution, a S2035T substitution, a S20351 substitution, and a A2034V substitution. In further embodiments, the modification is an amino acid substitution selected from one or more of a F2108L substitution, a F2108I substitution, a F2108M substitution, and a F2108G substitution.

In an embodiment, a nucleic acid, vector, or expression cassette described herein comprises a nucleic acid of interest, and in embodiments, the methods, uses and products herein relate to the use of a nucleic acid of interest. In embodiments, the term “nucleic acid of interest” or “gene of interest” is used to refer to a nucleic acid that encodes a functional peptide or polypeptide (protein) of interest (native or modified peptides/proteins). In an embodiment, the functional peptide or polypeptide is a therapeutic peptide or polypeptide, i.e. a peptide or polypeptide that can be administered to a subject for the purpose of treating or preventing a disease. Any nucleic acid encoding a peptide or polypeptide of interest known to those of ordinary skill in the art is contemplated for inclusion in the synthetic expression cassette. The peptide or polypeptide of interest may be an enzyme, a signaling molecule (e.g., kinase, phosphatase), a receptor, a growth factor (e.g., cytokines), a chemotactic protein (e.g., chemokines), a structural protein (cytoskeletal proteins), a transcription factor, a cell adhesion protein, an antibody or antigen-binding fragment thereof, etc. The peptide or polypeptide may be a naturally-occurring peptide or polypeptide, a fragment or variant thereof, chimeric versions thereof, etc.

### Recombinant Receptors

In an embodiment, the nucleic acid of interest encodes a recombinant receptor, such as a chimeric antigen receptor (CAR). Such CAR typically comprises a ligand-binding domain (e.g. antibody or antibody fragment such as a single-chain variable fragment [scFv]) that provides specificity for a desired antigen (e.g., tumor antigen) linked to an activating intracellular domain portion, such as a T cell or NK cell activating domain, providing a primary activation signal, in some aspects via linkers and/or transmembrane domain(s).

In particular embodiments, the recombinant receptor (e.g., CAR) comprises an intracellular signaling domain, which includes an activating cytoplasmic signaling domain (also interchangeably called an intracellular signaling region), such as an activating cytoplasmic (intracellular) domain capable of inducing a primary activation signal in an immune cell (T cell or NK cell, for example), a cytoplasmic signaling domain of a T cell receptor (TCR) component (e.g. a cytoplasmic signaling domain of a CD3-zeta (CD3 ζ) chain or a functional variant or signaling portion thereof) and/or that comprises an immunoreceptor tyrosine-based activation motif (ITAM).

In some embodiments, the recombinant receptor (e.g., CAR) further comprises an extracellular ligand-binding domain that specifically binds to a ligand (e.g., antigen) antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which is recognized on the cell surface in the context of a major histocompatibility complex (MHC) molecule.

Exemplary recombinant receptors, including CARs and recombinant TCRs, as well as methods for engineering and introducing the receptors into cells, include those described, for example, in international patent application publication numbers WO 2000/14257, WO 2013/126726, WO 2012/129514, WO 2014/031687, WO 2013/166321, WO 2013/071154, WO 2013/123061, US patent application publication numbers US 2002/131960, US 2013/287748, US 2013/0149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain M. et al., Cancer Discov. 3: 388-398, 2013; Davila M. L. et al., PLoS One 8: e61338, 2013; Turtle C. J. et al., Curr. Opin. Immunol. 24: 633-639, 2012; Wu R. et al., Cancer J. 18: 160-175, 2012. In some embodiments, the genetically engineered antigen receptors include a CAR as described in U.S. Pat. No. 7,446,190, and those described in International Patent Application Publication No.: WO 2014/055668.

In some embodiments, the recombinant receptor (e.g. CAR) includes in its extracellular portion an antigen- or ligand-binding domain that binds (specifically binds) to an antigen (or a ligand), such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb). The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies, and antigen binding fragments thereof specifically recognize an antigen of a full-length antibody. In some embodiments, the heavy and light chains of an antibody can be full-length or can be an antigen-binding portion (a Fab, F[ab′]₂, Fv or a single chain Fv fragment [scFv]). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (see, e.g., Kindt T. J. et al. Kuby Immunology, 6^{th} ed., W. H. Freeman and Co., page 91, 2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano S. et al. (J. Immunol. 150: 880-887, 1993) or Clarkson T. et al. (Nature 352: 624-628, 1991).

Single-domain antibodies (sdAbs) are antibody fragments comprising all or a portion of the heavy chain variable domain or all or a portion of the light chain variable domain of an antibody. In certain embodiments, the single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that may or may not be produced by enzyme digestion of a naturally-occurring intact antibody. In some embodiments, the antibody fragment is a scFv.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the CAR comprises an antibody or an antigen-binding fragment (e.g., scFv) that specifically recognizes an antigen, such as an intact antigen, expressed on the surface of a cell.

In some embodiments, the CAR comprises a TCR-like antibody, such as an antibody or an antigen-binding fragment (e.g., scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR comprising an antibody or antigen-binding fragment that exhibits TCR-like specificity directed against peptide-MHC complexes also may be referred to as a TCR-like CAR.

In some embodiments, the recombinant receptors include recombinant T cell receptors (TCRs) and/or TCRs cloned from naturally occurring T cells. In some embodiments, a T cell receptor (TCR) comprises a variable α and β chains (also known as TCRα and TCRβ, respectively) or a variable γ and δ chains (also known as TCRγ and TCRδ, respectively), or a functional fragment thereof such that the molecule is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to MHC molecules. In some embodiments, a TCR also can comprise a constant domain, a transmembrane domain and/or a short cytoplasmic tail (see, e.g., Janeway C. A. Jr et al., Immunobiology: The Immune System in Health and Disease, 3^{rd} ed., Current Biology Publications, p. 4:33, 1997). For example, in some embodiments, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR for a target antigen (e.g., a cancer/tumor antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells, such as from a T cell (e.g., cytotoxic T cell), T cell hybridomas, or other publicly available source. In some embodiments, the T cells can be obtained from in vivo isolated cells. In some embodiments, a high-affinity T cell clone can be isolated from a patient and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst M. R. et al., Clin. Cancer Res. 15: 169-180, 2009; Cohen C. J. et al., J. Immunol. 175: 5799-5808, 2005). In some embodiments, phage display is used to isolate TCRs against a target antigen (see, e.g., Varela-Rohena A. et al., Nat. Med. 14: 1390-1395, 2008 and Li Y. et al., Nat. Biotechnol. 23: 349-354, 2005). In some embodiments, the TCR or antigen-binding portion thereof can be synthetically generated from knowledge of the sequence of the TCR of interest.

In some embodiments, the recombinant receptor (e.g., a CAR such as an antibody or antigen-binding fragment thereof), further includes a spacer, which may be or include at least a portion of an immunoglobulin constant region or variant or modified version thereof, such as a hinge region, e.g., an IgG4 hinge region, and/or a CH1/CL, and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include those having at least about 10 to 220 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. Exemplary spacers include IgG4 hinge alone, IgG4 hinge linked to CH2 and CH3 domains, or IgG4 hinge linked to the CH3 domain. Exemplary spacers include, but are not limited to, those described in Hudecek M. et al. (Clin. Cancer Res. 19: 3153, 2013) or PCT patent publication number WO 2014/031687.

The antigen/ligand recognition domain generally is linked to one or more intracellular signaling components, such as signaling components that mimic activation through an antigen receptor complex, such as a TCR or NK receptor complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region[s] of) the alpha, beta or zeta chain of the TCR, CD28, CD3 epsilon, CD45, CD4, CD5, CD8, CD9, CD16, CD22, CD33, CD37, CD64, CD80, CD86, CD134, CD137, or CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan, and valine will be found at each end of a synthetic transmembrane domain.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one comprising glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 ζ chain. Thus, in some aspects, the CAR is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. In some aspects, the CAR includes a primary cytoplasmic signaling sequence that regulates primary activation of the TCR complex. Primary cytoplasmic signaling sequences that act in a stimulatory manner may comprise signaling motifs which are known as immunoreceptor tyrosine-based activation motifs or ITAMs. Examples of ITAM comprising primary cytoplasmic signaling sequences include those derived from TCR or CD3 ζ, FcR gamma, or FcR beta. In some embodiments, cytoplasmic signaling molecule(s) in the CAR comprise(s) a cytoplasmic signaling domain, portion thereof, or sequence derived from CD3ζ. in some embodiments, to promote full activation, a component for generating a secondary or co-stimulatory signal is also included in the CAR, such as the signaling domain of a costimulatory receptor such as CD28, 4-1BB, OX40, DAP10, and ICOS. In some aspects, an additional CAR is expressed in the same cell and provides the component for generating the secondary or costimulatory signal. In some cases, CARs are referred to as first, second, and/or third generation CARs. In some aspects, a first-generation CAR is one that solely provides an antigen-receptor (e.g., CD3-chain) induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR or other antigen receptor may further include a marker or the cell may further express a marker, such as a surrogate marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. See WO 2014/031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO 2014/031687. For example, the marker can be a truncated EGFR (tEGFR) that is, optionally, linked to a linker sequence, such as a T2A cleavable linker sequence.

Among the antigens that may be targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas (e.g., B cell, T cell, and myeloid leukemias, lymphomas, and multiple myelomas).

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor/cancer or pathogenic cells, as compared to normal or non-targeted cells or tissues. In other embodiments, the antigen is expressed on normal cells and/or is expressed on the engineered cells.

In some embodiments, the antigen (or a ligand) is a tumor antigen or cancer marker. In certain embodiments, the antigen is an integrin (e.g., αᵥβ₆ integrin, αᵥ, β₃ integrin, integrin β₇), B cell maturation antigen (BCMA), B7-H6, carbonic anhydrase 9 (CA9, also known as CAIX or G250), a cancer-testis antigen, cancer/testis antigen IB (CTAG, also known as NY-ESO-1 and LAGE-2), carcinoembryonic antigen (CEA), a cyclin, cyclin A2, C-C motif chemokine ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1CAM), CE7 epitope of L1-CAM, leucine-rich repeat containing 8 family member A (LRRC8A), Lewis Y, melanoma-associated antigen (MAGE)-A1, MAGE-A3, MAGE-A6, mesothelin, c-Met, murine cytomegalovirus (CMV), mucin 1 (MUC1), MUC 16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, trophoblast glycoprotein (TPBG, also known as 5T4), tumor-associated glycoprotein 72 (TAG72), vascular endothelial growth factor receptor (VEGFR), vascular endothelial growth factor receptor 2 (VEGFR2), Wilms tumor 1 (WT-1), galectins (galectin-1, galectin-7) a pathogen-specific antigen, or an antigen associated with a universal tag, and/or biotinylated molecules, and/or molecules expressed by HIV, HCV, HBV or other pathogens such as bacteria and parasites. Antigens targeted by the receptors in some embodiments include antigens associated with a B cell malignancy, such as any of a number of known B cell markers. In some embodiments, the antigen targeted by the receptor is CD20, CD19, CD22, ROR1, CD45, CD21, CD5, CD33, Ig kappa, Ig lambda, CD79a, CD79b, or CD30. In an embodiment, a plurality of recombinant receptors targeting a plurality of antigens are used. In a further embodiment, two recombinant receptors targeting two antigens are used.

In embodiments, the one or more nucleic acids of interest may encode multiple components (monomers) of a complex (oligomer, e.g., a dimer, e.g., a heterodimer), which may in turn be expressed in the target cell (e.g., T cell). In an embodiment, the components may be activated to oligomerize (e.g., dimerize) to form an active complex. In embodiments, such oligomerization or activation may be induced by treating such cells with a suitable inducing agent, such as rapamycin or a rapalog. For example, the one or more nucleic acids of interest may encode the components of a dimerizing agent-regulated immunoreceptor complex (DARIC), which can be induced with rapamycin or a rapalog thereof. In such a case, a non-immunosuppressive concentration of rapamycin or a rapalog may be used to be sufficient induce dimerization while at the same time avoiding or minimizing the inhibition of CAR-T cell activity.

### EXAMPLES

The present disclosure is illustrated in further detail by the following non-limiting examples.

### Example 1: Materials and Methods

### K562 Cell Culture and Transfection

K562 cells were obtained from the ATCC (CCL-243) and NALM6 cells stably transduced to express a FLUC-T2A-RFP-IRES-Puro reporter gene cassette (Biosettia). K562 and NALM6 cells were maintained at 37° C. under 5% CO₂ in RPMI medium supplemented with 10% FBS, penicillin-streptomycin, and GlutaMAX. Cells were routinely tested for the absence of mycoplasma contamination. Rapamycin (Cayman Chemicals, Cat 53123-88-9) was dissolved at 10 mg/mL in DMSO, working dilutions were prepared in water and stored at −20° C. AZD8055 (STEMCELL Technologies) was dissolved at 10 mM in DMSO, working dilutions were prepared in water and stored at −20° C. DMSO alone was diluted in water and used as vehicle control. Ouabain octahydrate (Sigma) was dissolved at δ mg/mL in hot water, working dilutions were prepared in water and stored at −20° C. K562 cells (2×10⁵ cells/transfection) were transfected using a Lonza 4D Nucleofector™ with a SF nucleofection kit (Lonza) following manufacturer's recommendations with the indicated DNA concentrations. Cells were treated with the indicated concentration of rapamycin 3 days post-nucleofection until all non-resistant cells were eliminated. For the isolation of single cell-derived clones, simultaneous coselection and cloning was performed in methylcellulose-based semi-solid RPMI medium supplemented with 100 μM ouabain for 10 days.

### Genome Editing Vectors

Guide RNAs were designed with CRISPOR²⁶ and their sequences are provided in Table 3. When required, DNA sequences for the guides were modified at position 1 to encode a G, owing to the transcription requirement of the human U6 promoter. Guide RNAs were cloned into pX330-U6-Chimeric_BB-CBh-hSpCas9 (Addgene 42230) and their sequences are provided in Table 3. Plasmid donors were cloned into pUC19 with short homology arms (700-800 bp) and all sequences were confirmed by Sanger sequencing. Prime editing (PE) experiments were performed with pCMV-PE2³⁸ (Addgene 132775), pU6-pegRNA-GG-acceptor³⁸ (Addgene 132777), pCMV-PEmax³¹ (Addgene 174820), and pU6-tevopreq1-GG-acceptor⁴⁵ (Addgene 174038). Adenine base editing experiments were performed with pCMV_ABEmax³² (Addgene 112095) and ABE8e³³ (Addgene 138489). To repurpose the mTORC1 signaling reporter mVenus-TOSI previously developed for mouse³⁴, the N-terminal residues (1-82) of the human PDCD4 gene were codon optimized using the GenSmart™ codon optimization tool, synthesized as a gBlocks™, and cloned into ATP1A1_T804N_hPGK1_mScarlet-I_Donor (Addgene 173207) upstream of the mScarlet-I-NLS cassette using AfIII and Ncol.

TABLE 3
gRNAs and pegRNAs used in this study

| | | | |
|---|---|---|---|
| gRNA | Locus | Sequence (5′→3′) | SEQ ID NO: |

gRNA target sequences

| | | | |
|---|---|---|---|
| G38 | MTOR-Exon 45 | CACATGATAATAGAGGTCCC | 3 |
| G45 | MTOR-Exon 45 | GTCGGAACACATGATAATAG | 4 |
| G63 | MTOR-Exon 45 | GCAGCTGCTTTGAGATTCGT | 5 |
| G49 | MTOR-Intron 45 | AGGTAGGATCTTCAGGCTCC | 6 |
| G54 | MTOR-Intron 45 | GGATCTTCAGGCTCCTGGCA | 7 |

pegRNA target sequence

| | | | |
|---|---|---|---|
| G63 | MTOR-Exon 45 | GCAGCTGCTTTGAGATTCGT | 5 |

pegRNA extension sequences

| | | | |
|---|---|---|---|
| F2108L | MTOR-Exon 45 | TGTGTTGCGACGAATCTCAAAGCAG | 8 |
| F2108Saturation | MTOR-Exon 45 | TCATGTGNNKAGACGAATCTCAAAGCAG | 9 |
| F2108L_v2 | MTOR-Exon 45 | TCATGTGTTGAGACGAATCTCAAAGCAG | 10 |
| F2108F | MTOR-Exon 45 | TCATGTGTTTAGACGAATCTCAAAGCAG | 11 |
| F2108I | MTOR-Exon 45 | TCATGTGATCAGACGAATCTCAAAGCAG | 12 |
| F2108M | MTOR-Exon 45 | TCATGTGATGAGACGAATCTCAAAGCAG | 13 |
| F2108V | MTOR-Exon 45 | TCATGTGGTGAGACGAATCTCAAAGCAG | 14 |
| F2108K | MTOR-Exon 45 | TCATGTGAAGAGACGAATCTCAAAGCAG | 15 |
| F2108G | MTOR-Exon 45 | TCATGTGGGCAGACGAATCTCAAAGCAG | 16 |

### Primary Human T Cells Isolation and Culture

Primary CD3⁺ human T cells were isolated from healthy human donors either from fresh whole blood or residual leukoreduction chambers system (HémaQuébec). An EasySep™ Human T cell isolation kit (STEMCELL) was used for positive magnetic selection of CD3⁺ T cells from peripheral blood mononuclear cells (PBMCs). Primary CD3⁺ T cells were cultured with Immunocult-XF T cell expansion medium supplemented with penicillin-streptomycin and 300 U/mL of IL-2. Cells were thawed and activated for 3 days with Immunocult™ human CD3/CD28/CD2 T cell activator (STEMCELL) before transfection.

### Primary Human T Cells Transfection

dsDNA donors were produced as described previously²⁴. The donor sequences were cloned into a cloning vector and PCR amplification (25 cycles) was performed using Kapa-HiFi polymerase. DNA purification was performed by solid-phase reversible immobilization using AMPure XP (Beckman Coulter) magnetic beads. For ribonucleoprotein complex (RNP) formulation, 100 pmols of sgRNAs (IDT) were mixed with 0.8 μL of 100 mg/mL polyglutamic acid (Sigma) and 50 pmols of SpCas9 nuclease (IDT), as previously described²⁷. The RNP mix was incubated at 37° C. for 10 minutes and the dsDNA donors were added to the mix and incubated at room temperature for 5 minutes before transfection. Primary CD3⁺ T cells (1×10⁶ cells/transfection) were transfected using a Lonza 4D Nucleofector™ with a P3 nucleofection kit (Lonza) following manufacturer's recommendations with the indicated DNA concentrations. CD3+ T cells were resuspended in 80 μL media without IL-2 directly after nucleofection and incubated at 37° C. for 15 minutes before transfer into culture media plates. For AAV6 transduction, CD3+ T cells were resuspended in 80 μL media directly after nucleofection and AAV6 vectors were added. Cells were incubated at 37° C. for 8 hours at high cell density before transfer into culture media plates. Cells were treated with 25 nM rapamycin 3 days post-nucleofection.

### Sanger Sequencing Analysis and Out-Out PCRs

Genomic DNA was extracted with QuickExtract DNA extraction solution (EpiCentre) following manufacturer's recommendations. Primers used in this study and the PCR product sizes are provided in Table 4. PCR amplifications were performed with 30 cycles of amplification with Phusion polymerase. Sanger sequencing was performed on PCR amplicons to quantify the percentage of edited alleles using TIDE and TIDER^{28,29}. Kapa-HiFi polymerase was used for out-out PCRs. To detect targeted integration, out-out PCRs were performed with primers that bind outside of the homology regions of the plasmid/dsDNA donors. Wild type K562 or CD3+ T cells genomic DNA was used as a control for all PCRs.

TABLE 4
Primers used in this study

| | | SEQ | PCR |
|---|---|---|---|
| Primer | | ID | product |
| name | Sequence (5′→3′) | NO: | length |
| mTOR- | GCTCCTGAGGAATATCTGCAGTC | 17 | 596 bp |
| In45-F | | | |
| mTOR- | CCTCTGCTTGGATGTGATGACTTG | 18 | |
| In45-R | | | |
| mTOR- | CCATCCTCTGGCATGAGATGTGG | 19 | 1517 bp |
| Out-F | | | |
| mTOR- | AGAAGCCCCCGTTTCAGAGGAG | 20 | |
| Out-R | | | |
| mTOR- | CCTGGAAGAGGCATCTCGTTTG | 21 | 1464 bp |
| LD-F | | | |
| mTOR- | CTAGCACCTTCCCTGCTTGC | 22 | |
| LD-R | | | |
| MTOR- | ACACTCTTTCCCTACACGACGCTCTTC | 23 | 341 BP |
| In45- | CGATCTGCTCCTGAGGAATATCTGCAG | | |
| NGS-F | TC | | |
| (PCR1) | | | |
| MTOR- | CTGACTATAATGACAGTTAACCCTGCC | 24 | |
| In45- | GTGACTGGAGTTCAGACGTGTGCTCTT | | |
| NGS-R | CCGAT | | |
| (PCR1) | | | |

### Flow Cytometry

The percentage of fluorescent cells was quantified using a BD LSRII flow cytometer and 1×10⁵ cells were analyzed for each condition. For antibody staining, K562 cells were incubated with Fc receptor block (eBioscience Cat 16-9161-73) for 20 minutes on ice with PBS and 3% human serum (Sigma). Anti-human CD34 Alexa Fluor 488-conjugated antibody clone QBEnd10 (R and D systems Cat FAB7227G) and anti-human CD52 FITC-conjugated antibody clone HI186 were used for RQR8 and CD52 quantification, respectively. For mTORC1 signaling assays, cells were treated with the indicated concentration of rapamycin or AZD8055 for 24 hours before analysis. FACS sorting was performed using a BD FACS Aria Fusion flow cytometer for the saturation prime editing experiment and 5×10⁵ cells were sorted for each condition. Flow cytometric data visualization and analysis were performed using FlowJo™ software (Tree Star).

### Saturation Prime Editing and High-Throughput DNA Sequencing

The MTOR-F2108 saturation epegRNA (tevopreQ1)⁴⁵ vector was designed to install NNK codons and a silent R2109R PAM mutation at MTOR exon 45, synthesized as a gBlock™ (IDT), and cloned into pU6-tevopreq1-GG-acceptor⁴⁵ (Addgene 174038). K562 cells stably expressing the mSc-TOSI reporter were transfected with PE3max-epegRNA vectors and treated with 0.5 μM rapamycin starting 3 days post-transfection until all non-resistant cells were eliminated. Following selection, cells were treated for 24 hours with 0.5 μM rapamycin and FACS-sorted for low mSc-TOSI fluorescence intensity to enrich cells with functional mTORC1 signaling in the presence of rapamycin. Genomic DNA was harvested after selection and after FACS sorting for low and high mSc-TOSI fluorescence intensity. For amplicons sequencing, primers containing Illumina forward and reverse adapters were used for a first round of PCR using the Kapa-HiFi polymerase. PCR products were purified using AMPure XP magnetic beads and their quality was evaluated by electrophoresis. A second round of barcoding PCR and bead purification was performed, and amplicons were sequenced on an Illumina MiSeq instrument. Alignment of amplicon sequences to the reference MTOR sequence was performed using CRISPResso2⁴⁶, and all possible NNK codons were identified in the vehicle-treated cell pool.

### Cytotoxicity Assay

Luciferase-based cytotoxic assay was performed as previously described^{8,30}. After rapamycin selection, CD19-CAR-T cells were co-cultured in 96-well plates in supplemented RPMI medium with NALM6 cells stably expressing firefly luciferase and RFP at the indicated effector to target ratio (E:T) for 18 hours. Target cells incubated with WT CD3+ T cells were used to determine maximal luciferase expression (maximal relative light units; RLUₘₐₓ). After co-culture, an equal volume of luciferase substrate (Bright-Glo, Promega) was added to each well. Emitted light was detected with a Tecan luminescence plate reader and cell lysis was determined as (1−(RLUₛₐₘₚₗₑ)/(RLUₘₐₓ))×100. FACS-based cytotoxicity assay was performed as previously described³⁵. After rapamycin selection, CD19-CAR-T cells were washed with PBS, and co-cultured in 12-well plates in supplemented RPMI medium with 50:50 mixtures of rapamycin-resistant K562-BFP (CD19-negative) cells and NALM6-RFP cells at the indicated effector to target ratio (E:T) for 72 hours. The relative percentage of NALM6-RFP cells following coculture in the presence or absence of 25 nM rapamycin was used to calculate the percentage of cytotoxicity. Target cells incubated with WT CD3⁺ T cells were used to determine maximal NALM6-RFP growth.

### Example 2: Targeting MTOR with CRISPR-Cas9 Allows the Selection of Rapamycin-Resistant K562 Cells

After an extraordinary 18-month response to everolimus, a rapamycin analog, a patient with metastatic anaplastic thyroid carcinoma relapsed with a resistant tumor harboring the F2108L mutation in MTOR¹⁶. The same mutation was also found in a clone of the MCF-7 breast cancer cell line after 3 months of exposure to rapamycin¹⁷. Of importance, this mutation only confers resistance to first-generation allosteric inhibitors of mTOR, but not to second- and third-generation inhibitors^{16, 17}.

We prepared gRNAs for introducing this mutation into the MTOR locus, to determine if it confers resistance to rapamycin and facilitate the enrichment of CRISPR-engineered cells. We first performed a guide RNA (gRNA) screening in K562 cells to identify highly active gRNAs. SpCas9 target sites surrounding the F2108 codon generated up to 66% indels in K562 cells (FIG. 1). We selected both intron 45 gRNAs, G49 and G54, for further characterization.

We designed plasmid donors harboring a PAM mutation to prevent Cas9 from cleaving edited alleles, and the F2108L mutation to select CRISPR-engineered cells (FIG. 2A). Targeting MTOR with either G49 or G54 gRNA/donor pair allowed the generation of rapamycin-resistant K562 cells with similar efficiencies (FIG. 2). We amplified the locus by PCR and performed Sanger sequencing to analyze the percentage of alleles harboring the F2108L mutation by TIDER. We observed an enrichment of 12% to 57% of alleles harboring the F2108L after rapamycin selection, demonstrating the feasibility of our CRISPR-based strategy to engineer rapamycin-resistant cells.

### Example 3: Targeting Transgene to the MTOR Locus Allows the Selection of Rapamycin-Resistant K562 Cells

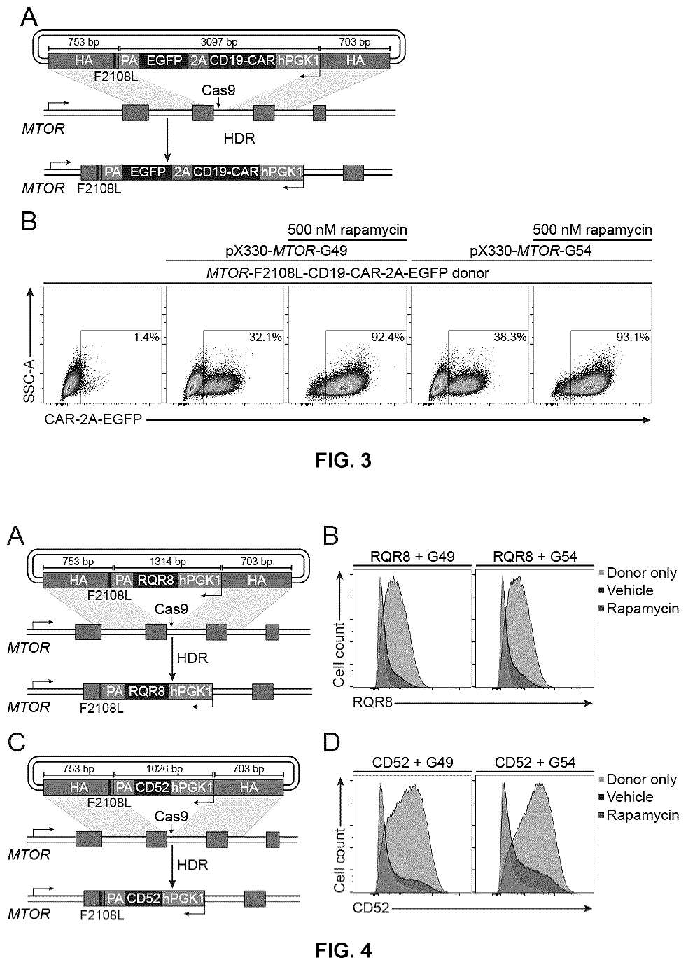

We then designed a donor to simultaneously knock-in a therapeutic transgene and introduce the F2108L mutation to the MTOR locus. To demonstrate our strategy, we used a CD19-targeting chimeric antigen receptor (CD19-CAR) coupled to an EGFP reporter via a 2A self-cleaving peptide to quantify the percentage of edited cells. After gene knock-in, the MTOR-F2108L is expressed on the forward DNA strand while the moderately active human PGK1 promoter drives the expression of our CD19-CAR-2A-EGFP cassette on the reverse DNA strand (FIG. 3A). We observed high knock-in yields without selection (32% to 38%) and enrichment to up to 93% of K562 cells expressing the EGFP reporter (FIG. 3). This two-in-one approach allows the generation of rapamycin-resistant cells expressing a CD19-CAR with a single gene targeting event.

Incorporating a suicide switch to therapeutic cells for in vivo elimination in the event of excessive toxicity is a relevant strategy to engineer safer cell therapies. Inducible caspase 9 safety switch and CD20, the target of the therapeutic antibody rituximab, have been developed as suicide genes for adoptive cell therapies applications¹⁸⁻²⁰. A highly compact 136-amino-acid epitope-based marker/suicide gene named RQR8 has also been developed and used in a CD19-CAR-T cells phase I clinical trial^{21,22}. This marker/suicide gene combines epitopes from both CD34 and CD20, allowing selection with clinically approved systems and selective depletion of transgene-expressing cells using rituximab²¹. Another therapeutic antibody of interest, alemtuzumab, targets CD52. Expressed on normal and malignant lymphocytes, CD52 is a favored target for lymphoma therapy and immunosuppression before bone marrow transplantation²³. However, CD52 has never been used as safety/suicide genes for cell therapy applications and we hypothesized that CD52 could be targeted to the MTOR locus for safer therapies involving CD34+ hematopoietic stem and progenitor cells (HSPCs).

As a first example to demonstrate that therapeutic transgenes other than CARs could also be targeted to MTOR, we targeted RQR8 and CD52 to MTOR and we selected rapamycin-resistant K562 cells expressing the transgenes of interest (FIG. 4). Rapamycin-treated K562 cells showed higher transgene expression (FIGS. 4B and 4D). Overall, different therapeutically relevant transgenes can be targeted to the MTOR locus to generate rapamycin-resistant cells and our genome editing strategy holds potential to engineer safer cell therapies.

### Example 4: mTORC1 Signaling Remains Functional after Targeted Transgene Integration at the MTOR Locus

We then adapted a fluorescent mTORC1 signaling indicator³⁴ (mSc-TOSI) to assess the impact of rapamycin on mTORC1 signaling after targeted transgene integration to the MTOR locus. Active mTORC1 signaling results in rapid phosphorylation of the mSc-TOSI phosphodegron by S6K, ubiquitination, and degradation by the proteasome while its inhibition stabilizes the reporter³⁴ (FIG. 5A). We treated bulk populations of cells harboring the MTOR-F2108L mutation with or without targeted therapeutic transgene integration at the MTOR locus with different concentrations of rapamycin and AZD8055, a second-generation mTOR inhibitor. We observed functional mTORC1 signaling in the presence of 50 nM to 5 000 nM of rapamycin, concentrations that are superior to the immunosuppressive window of 3-22 nM and the maximal blood concentrations observed in clinical settings^{35,37} (FIG. 5B). Of importance, CRISPR-engineered cells harboring the MTOR-F2108L mutation remained sensitive to the second-generation mTOR inhibitor AZD8055 (FIG. 5B), which could be used as a safety switch in case of toxicity. Overall, our observation confirmed that MTOR expression remains functional after targeted transgene integration to the MTOR locus with no marked decrease in mTORC1 signaling compared to the MTOR-F2108L mutation alone in the presence of high concentrations of rapamycin (FIG. 5B). This observation further confirmed that both MTOR-F2108L and a therapeutic transgene of interest can be expressed in opposite directions at the MTOR locus.

While functional in the presence of rapamycin, we still observed a slight mTORC1 signaling decrease upon rapamycin treatment in bulk populations of selected cells (FIG. 5B). We hypothesized that unedited MTOR alleles (WT) were responsible for the slightly higher mTORC1 signaling observed without drug treatment. We then isolated homozygous single cell-derived K562 clones in methylcellulose semi-solid media and performed FACS-based mTORC1 signaling assays. In MTOR-F2108L homozygous clones, mTORC1 signaling remained completely functional upon rapamycin treatment with or without CAR integration (FIG. 6). Our results further demonstrate that targeted integration of a large transgene cassette of 3 kb and expression via the moderately active human PGK1 (hPGK1) promoter did not have a negative functional impact on mTORC1 signaling.

### Example 5: Prime Editing at the MTOR Locus Confers Resistance to Rapamycin

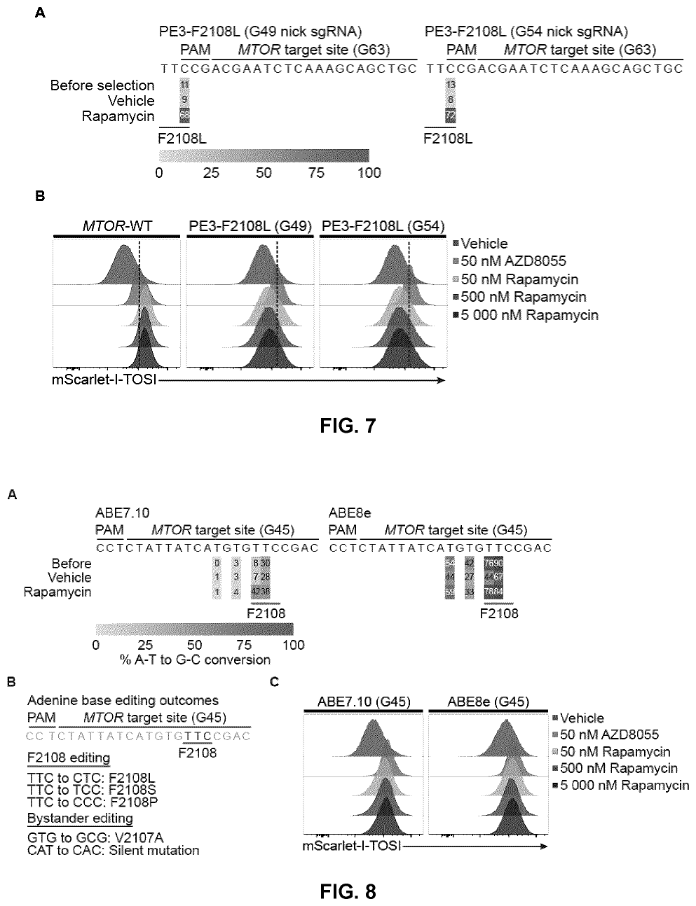

Prime editing is a CRISPR-based genome editing technology that can be used to install nucleotide substitutions, as well as short insertions and deletions without requiring donor DNA or double-strand breaks (DSBs)³⁸⁻⁴⁰. Prime editors are composed of a Cas9 nickase fused to a reverse transcriptase and a programmable prime editing guide RNA (pegRNA) that harbors the modification of interest. We adapted our validated guide RNAs to install the MTOR-F2108L mutations using the prime editing system 3 (PE3)³⁸. We transfected K562 cells stably expressing the mSc-TOSI reporter with PE3 vectors, and we performed rapamycin selection three days post-transfection. The percentage of alleles harboring the MTOR-F2108L mutation markedly increased after rapamycin selection, as determined by BEAT⁴¹ analysis from Sanger sequences (FIG. 7A). We then confirmed that mTORC1 signaling remained functional in the presence of different concentrations of rapamycin after prime editing (FIG. 7B). Therefore, prime editors can be used to generate rapamycin-resistant human cells and we foresee that our strategy could be used to enrich prime editing events at different genes of interest via coselection, as previously described⁴²⁴³.

We then tested whether adenine base editing at MTOR exon 45 could confer resistance to rapamycin in K562 cells. We transfected K562 cells stably expressing the mSc-TOSI reporter with ABE7.10 or ABE8e vectors and performed rapamycin selection. We observed a marked increase in the conversion of the first nucleotide position of the F2108 TTC codon with ABE7.10, suggesting that the CTC codon (encoding F2108L) was enriched upon rapamycin selection (FIG. 8A). In contrast, we did not observe a marked increase in the conversion of the second nucleotide position of the F2108 TTC codon (FIG. 8A), suggesting that the F2108S and F2108P substitutions did not confer resistance to rapamycin (FIG. 8B). We observed highly efficient base editing with ABE8e with up to 90% conversion before selection, but the percentage of modified alleles decreased after multiple cell passages without rapamycin (vehicle-treated cells) (FIG. 8A). This observation suggests that the F2108P modification (FIG. 8B) could have a negative functional impact on mTOR. Interestingly, while ABE8e-targeted K562 cells did not grow robustly in the presence of rapamycin, partial resistance was observed and no marked decrease in the percentage of edited alleles was observed with rapamycin-treated cells (FIG. 8A). These observations suggest that adenine base editing at the MTOR locus only confers partial resistance to rapamycin. To confirm this hypothesis, we performed FACS-based mTORC1 signaling assays and observed very low levels of mTORC1 signaling in the presence of rapamycin (FIG. 8C). Overall, precise editing via HDR or prime editing is a more suitable strategy to engineer rapamycin-resistant cells when using this version of base editor. As such, a base editor variant that would only specify the TTC to CTC (i.e., creation of F2108L) would be expected to confer potent rapamycin resistance.

### Example 6: Identification of Novel Rapamycin Resistance Mutations Via Saturation Prime Editing

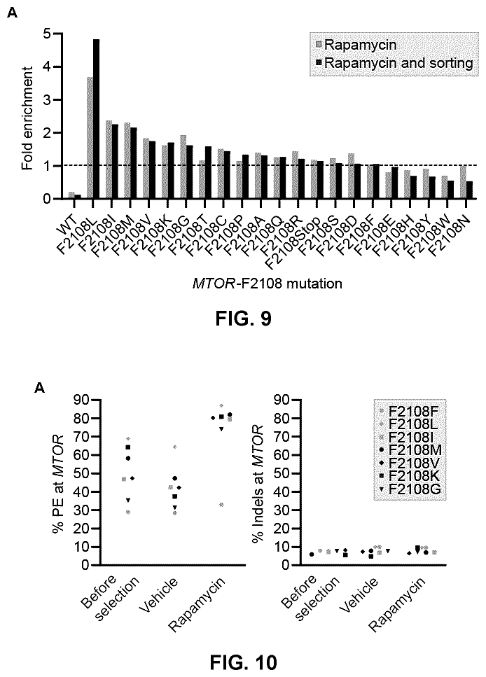

We then took advantage of our prime editing strategy to perform saturation PE⁴⁴ at the MTOR locus to identify new rapamycin resistance mutations. We designed an epegRNA (tevopreQ1)⁴⁵ to install NNK codons at the MTOR-F2108 position and an additional silent R2109R PAM mutation. We transfected K562 cells stably expressing the mSc-TOSI reporter with PE3max-epegRNA vectors and performed rapamycin selection starting 3 days post-transfection until all non-resistant cells were eliminated. We then performed FACS sorting to enrich cells with functional mTORC1 signaling in the presence of rapamycin to identify new MTOR-F2108 mutations that confer resistance. Following rapamycin selection and FACS sorting, we performed high-throughput sequencing and analyzed the fold enrichment of all possible MTOR-F2108 mutations using CRISPResso2⁴⁶ (FIG. 9). All possible NNK codons were detected, confirming that all possible amino acid residues were represented. The fold enrichment from 3 days post-transfection (before selection) was calculated to identify the MTOR-F2108 residues that conferred resistance to rapamycin (FIG. 9). The most enriched mutation identified after rapamycin selection and FACS sorting was F2108L^{16,17} (4.6 fold enrichment), and five additional mutations showed 1.6 to 2.6 fold enrichments, suggesting that these new mutations also conferred resistance to rapamycin.

We then designed epegRNAs to validate the new MTOR-F2108 mutations individually. K562 cells harboring MTOR-F2108 substitutions grew robustly in the presence of 0.5 μM rapamycin and we observed a marked increase in the percentage of alleles harboring the PE-specified modifications after rapamycin selection (FIG. 10). As expected, no resistance and no enrichment were observed with the silent F2108F mutation (FIG. 10). These observations confirmed that the five new mutations identified by saturation prime editing conferred resistance to rapamycin.

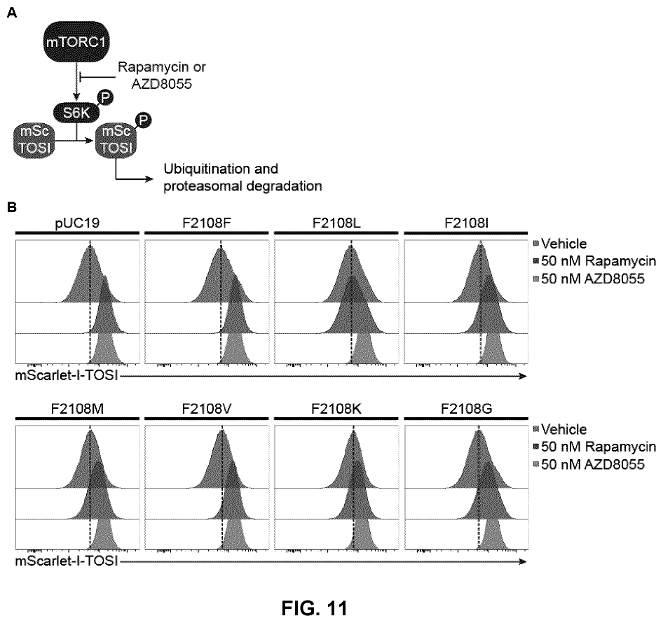

To confirm that mTORC1 signaling remained functional in the presence of rapamycin, we performed a FACS-based assay to monitor the degradation of the mSc-TOSI reporter (FIG. 11A). We observed complete mTORC1 signaling inhibition without epegRNA (pUC19) and with the silent F2108F mutation, and complete rapamycin resistance with the F2108L mutation (FIG. 11B). However, intermediate levels of signaling in the presence of rapamycin were observed with F21081, F2108M, and F2108G while low levels of signaling were observed with F2108V and F2108K (FIG. 11B). These observations suggest that robust K562 cell growth can be maintained with partial mTORC1 signaling. These MTOR-F2108 mutations could potentially be used to pharmacologically fine-tune mTORC1 signaling while maintaining robust cellular growth. mTOR plays key roles in hematopoietic stem cell maintenance and self renewal^{47,48} as well as in T cell fate decisions and differentiation^{49,50}. Reducing mTORC1 signaling has been shown to trigger the formation of stem cell-like memory T cells and also enhance CAR-T cell antitumor activity^{51,52}. In addition, Lamarthbée et al.⁵³ have shown that transient mTOR inhibition with rapamycin rescues 4-1BB CAR-Tregs from tonic signal-induced dysfunction. Considering that mTORC1 signaling acts as a metabolic rheostat⁵⁴, fine-tuning this pathway could potentially have a positive impact on CAR-T cells persistence, and consequently, on CAR-T cells antitumor activity. Our results demonstrate that engineering the MTOR locus with CRISPR may permit the marker-free selection of cells with different levels of mTORC1 signaling in the presence of rapamycin.

### Example 7: Targeting MTOR Allows the Selection of Rapamycin-Resistant Primary Human CD3+ T Cells

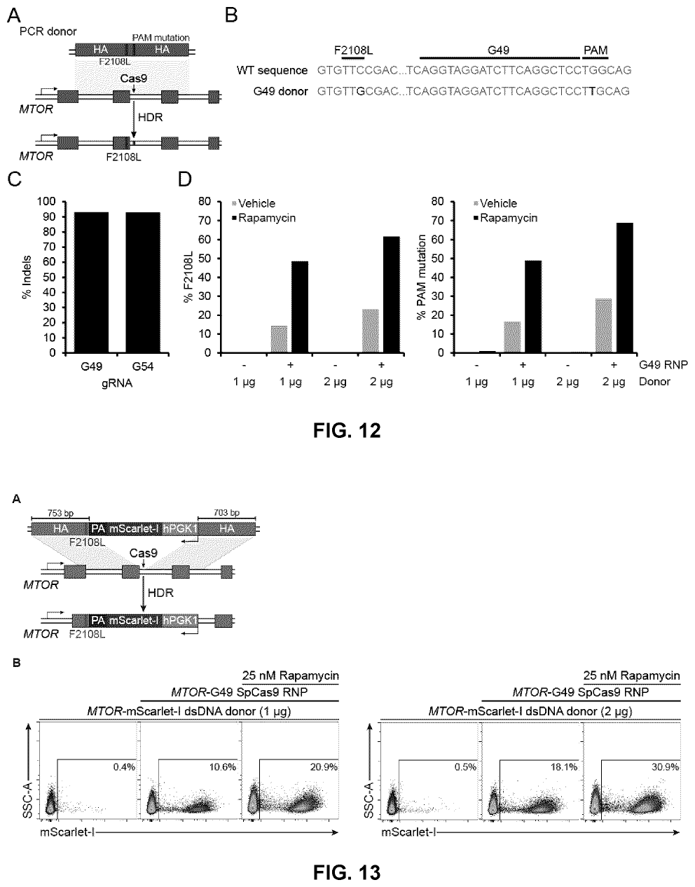

To adapt our genome editing strategies to primary CD3⁺ T cells, we first electroporated SpCas9 ribonucleoprotein complex (RNP) to assess the activity of our MTOR intron 45 gRNAs. Our highly active gRNAs generated up to 93% indels in primary T cells (FIG. 12), and we selected gRNA 49 for further experiments for its closer proximity to the F2108 codon. Non-viral T cell reprogramming with CRISPR-Cas9 and linear double-stranded DNA (dsDNA) donors is an efficient way to perform small gene knock-in⁴⁵. We used this method to introduce the F2108L mutation to the MTOR locus by co-transfecting the G49 RNP and donor pair. Three days post-transfection, we treated cells with rapamycin for 15 days before genotyping. Using 2 μg of dsDNA donor, up to 63% and 69% of the alleles harbored the F2108L and the PAM mutations after selection, respectively (FIG. 12D). Our strategy allows efficient generation of rapamycin-resistant CD3*T cells by non-viral targeting.

We then performed non-viral targeting to introduce the fluorescent mScarlet-I reporter to the MTOR locus (FIG. 13A). We transfected CD3⁺ T cells with MTOR-G49 RNP and 1 μg or 2 μg of dsDNA donor and performed rapamycin selection for 8 days before FACS-based quantification. We observed a marked increase in the percentage of CD3⁺ T cells expressing the fluorescent reporter after rapamycin selection from 11% to 21% and 18% to 31% with 1 μg or 2 μg of dsDNA donor, respectively (FIG. 13B). Hence, targeting a reporter transgene to the MTOR locus allows the enrichment of rapamycin-resistant CD3*T cells.

### Example 8: Combinatorial Tumor Targeting with Rapamycin and CAR-T Cells

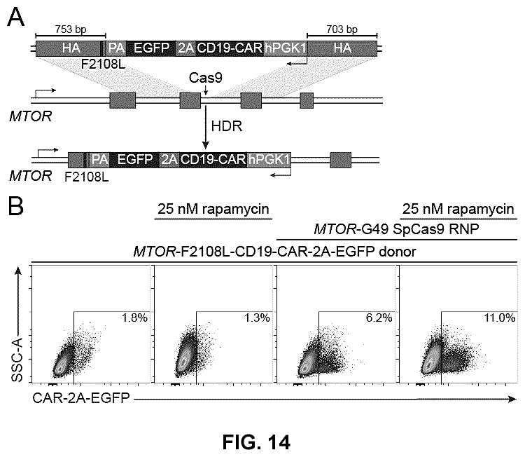

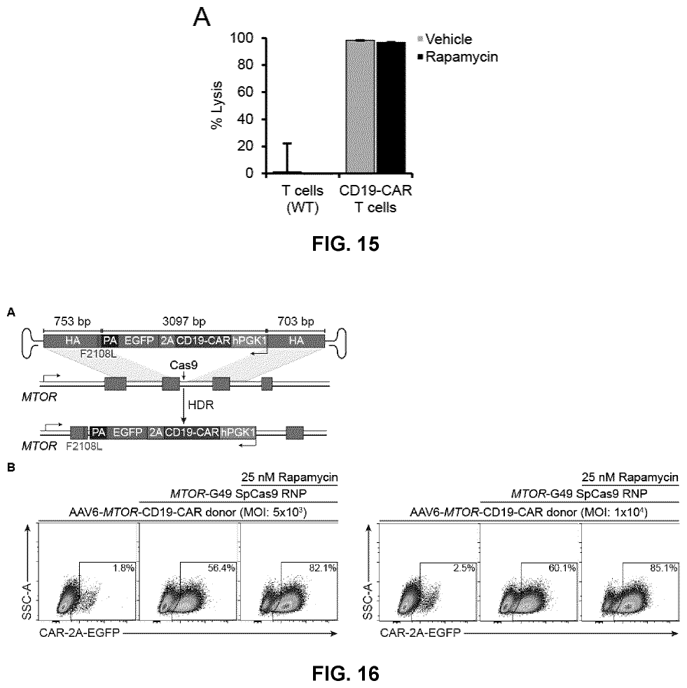

We then performed non-viral targeting to introduce the CD19-CAR-2A-EGFP gene cassette to the MTOR locus using a linear dsDNA donor (FIG. 14A). While the largest dsDNA donor reported in the literature is 2.1 kb long and only generated 12.3% of modified cells²⁴, we used a 4.6 kb dsDNA donor to generate rapamycin-resistant CAR T cells. We treated cells with rapamycin for 8 days and we achieved 6% and 11% knock-in without and with selection, respectively (FIG. 14B). We thus show that a large gene cassette can be targeted to the MTOR locus to generate rapamycin-resistant CAR-T cells.

We then used our rapamycin-resistant CD19-CAR-T cells to assess their antitumor activity against a well characterized acute lymphoblastic leukemia model. We used NALM6 cells stably expressing a FLUC-2A-RFP-IRES-Puro reporter to perform a luciferase-based cytotoxicity assay. We co-cultured CD19-CAR T cells and NALM6 cells with an effector to target ratio of 10:1 for 18 hours and we measured the luminescence emitted by viable NALM6 cells after incubation. Taking advantage of the short half-life of firefly luciferase in culture medium (≈30 min), the cytotoxicity of effector cells can be measured by the decrease in the luminescence signal from lysed target cells²⁵. We observed up to 97% and 98% of cell lysis with and without rapamycin, respectively, demonstrating that targeting a CD19-CAR to the MTOR locus allows the generation of rapamycin-resistant CAR-T cells that remains functional in presence of the immunosuppressant. Overall, targeting MTOR allows a two-in-one strategy to enrich CRISPR-engineered cells and target cancer cells in combination with rapamycin.

### Example 9: Highly Efficient CAR Targeting at MTOR with AAV6 Delivery

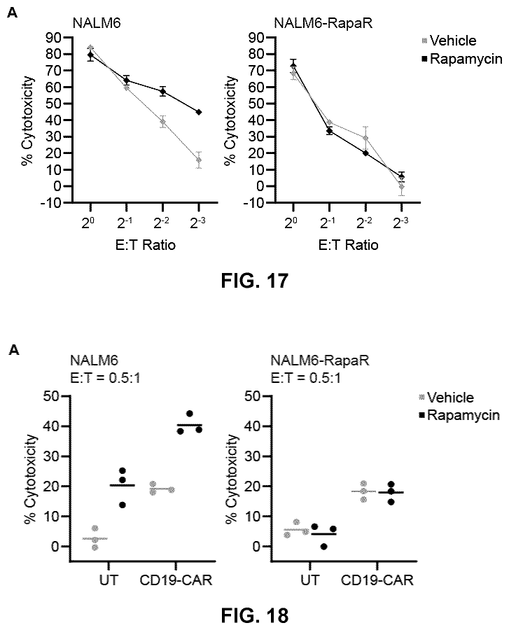

We then targeted the CD19-CAR-2A-EGFP cassette to the MTOR locus using an adeno-associated virus 6 (AAV6) vector (FIG. 16A). Directly after Cas9 RNP nucleofection, CD3⁺ T cells were transduced with different multiplicity of infection (MOI) with the AAV6 vector in a small volume for 8 hours and then transferred to the culture medium for three days before rapamycin treatment. Cells were reactivated four days post-transfection and expanded before the FACS-based quantification of transgene-expressing cells. We observed a marked increase in the percentage of CAR-2A-EGFP⁺ cells after 8 days of rapamycin selection, allowing the enrichment of 82% and 85% of CAR-T cells with MOIs of 5×10³ and 1×10⁴, respectively (FIG. 16B). Hence, Cas9 nucleofection and AAV6-mediated delivery of the DNA repair template followed by rapamycin selection is a highly efficient strategy to engineer CAR-T cells. Using these enriched CAR-T cells, we readily targeted NALM6 cells with and without rapamycin (FIG. 17). Of importance, we observed higher levels of cytotoxicity with low effector to target ratios, confirming that rapamycin can be used in combination with CAR-T cells to target cancer cells (FIG. 17).

We then adapted a coculture cytotoxicity assay based on FACS to measure the combinatorial impact of rapamycin and CAR-T cells on growth inhibition and cytotoxicity, respectively. We used NALM6-FLUC-RFP as target cells and K562-EBFP-RapaR-CD19-cells as control cells to measure growth inhibition and cytotoxicity. Using a low effector to target ratio of 0.5:1, we observed a combinatorial impact of rapamycin and CAR-T cells on growth inhibition and cytotoxicity (FIG. 18). This combinatorial effect was not observed with NALM6-RapaR cells that were engineered to be resistant to rapamycin (MTOR-F2108L), further confirming that rapamycin and CAR-T cells can be used in combination to increase anti-tumor activity (FIG. 18). We foresee that our strategy could both facilitate the enrichment of CRISPR-engineered CAR T cells and improve tumor eradication. All in all, MTOR can be used a selectable genomic harbor for targeted integration of therapeutic transgenes, providing a versatile platform for multiple cell therapy applications.

### Example 10: Highly Efficient Dimerization Agent-Regulated Immunoreceptor Complex (DARIC) Targeting at MTOR with AAV6 Delivery

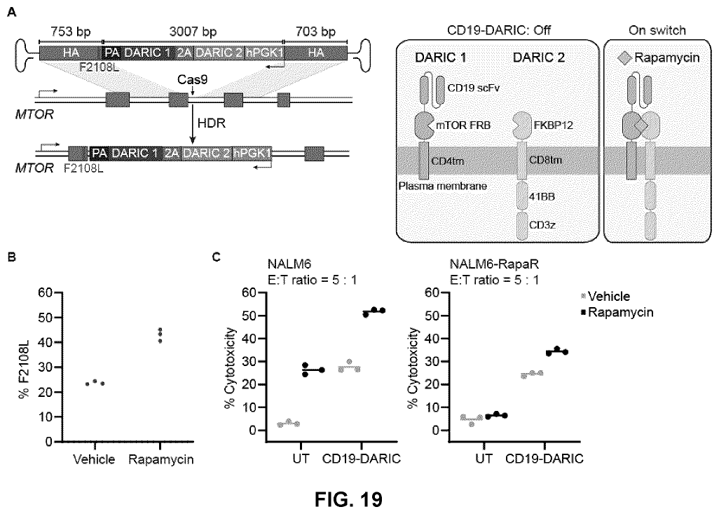

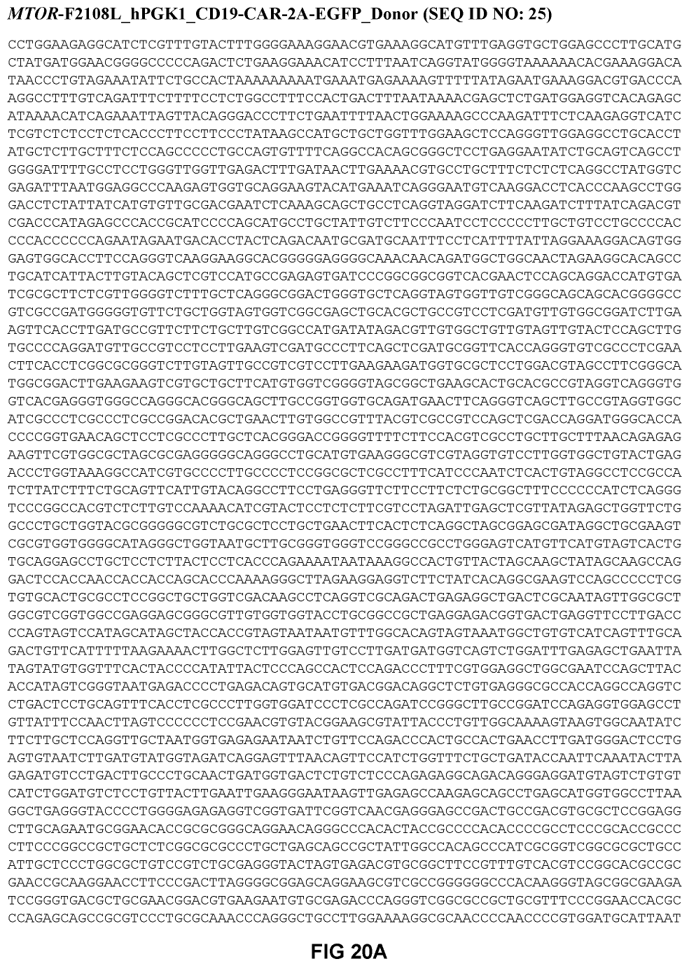

A dimerizing agent-regulated immunoreceptor complex (DARIC) that can be induced with rapamycin has previously been reported (FIG. 19A)³⁵. Using this strategy allows sensitive and adaptable pharmacological control of therapeutic CAR-T cells through extracellular receptor dimerization with rapamycin (FIG. 19A). However, this technology relies on nonimmunosuppressive concentrations of rapamycin to prevent the inhibition of CAR-T cells activity. We then tested whether we could target a CD19-DARIC transgene to the MTOR locus to pharmacologically control DARIC-T cells with rapamycin. We transfected CD3e T cells with MTOR-G49 RNP followed by AAV6 transduction. Three days post-transfection, we treated cells with rapamycin for 8 days before genotyping and observed a marked increase in the percentage of alleles harboring the F2108L mutation (FIG. 19B). We then performed a FACS-based cytotoxicity assay to target NALM6-FLUC-RFP cells with CD19-DARIC-T cells. To prevent receptor dimerization in the absence of rapamycin, we used vehicle-treated CD19-DARIC-T cells. Although we observed receptor activity in the absence of rapamycin, combinatorial targeting markedly increased the percentage of cytotoxicity (FIG. 19C). Overall, our strategy could be used to generate rapamycin-resistant CAR-T cells that can be pharmacologically controlled via extracellular receptor dimerization using rapamycin.

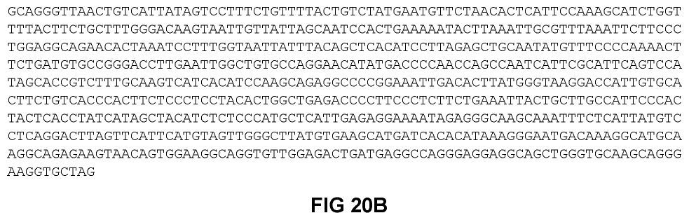

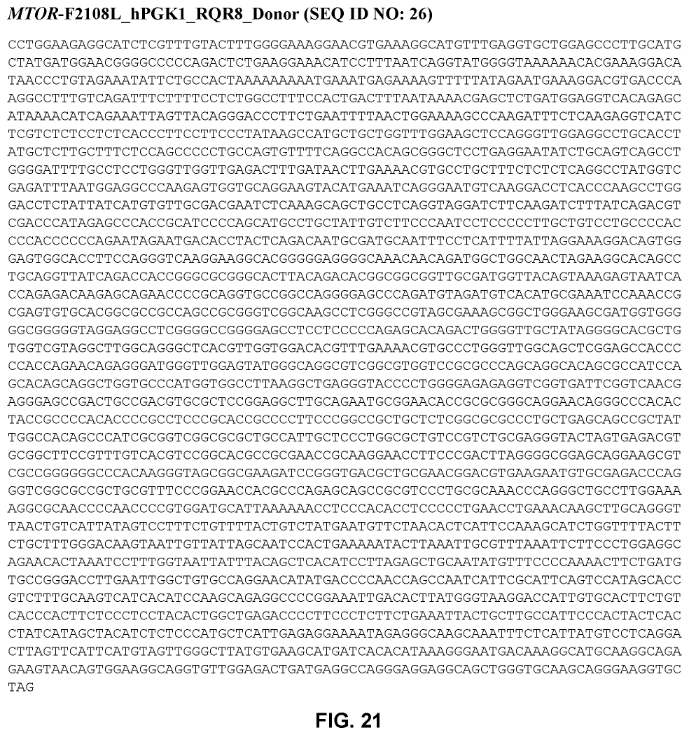

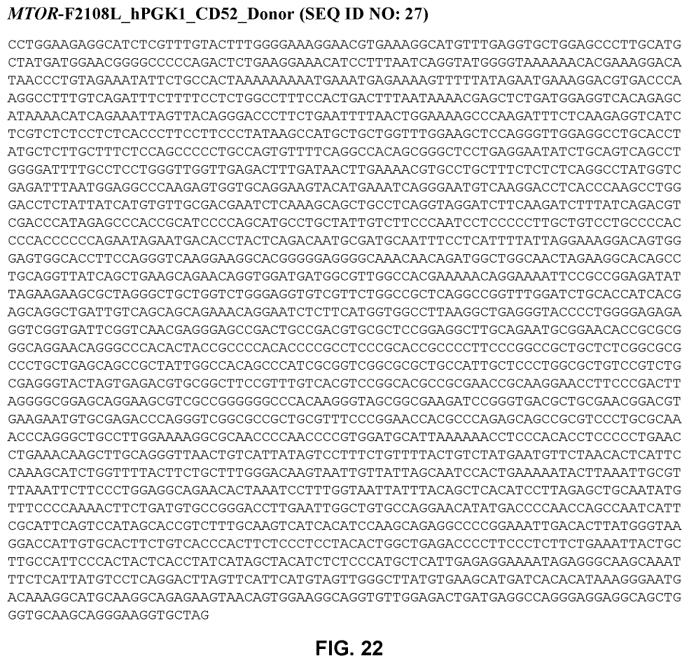

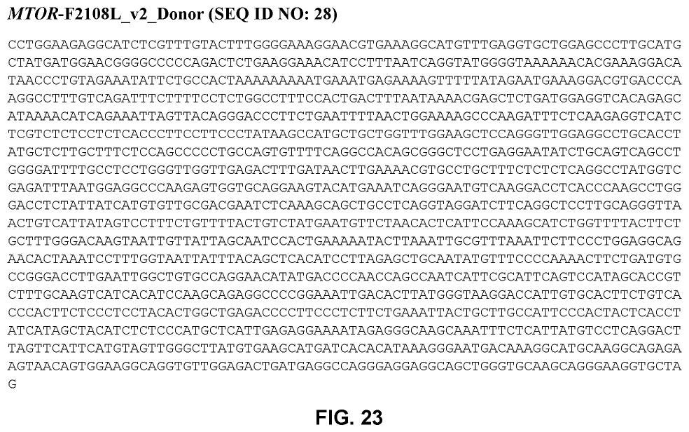

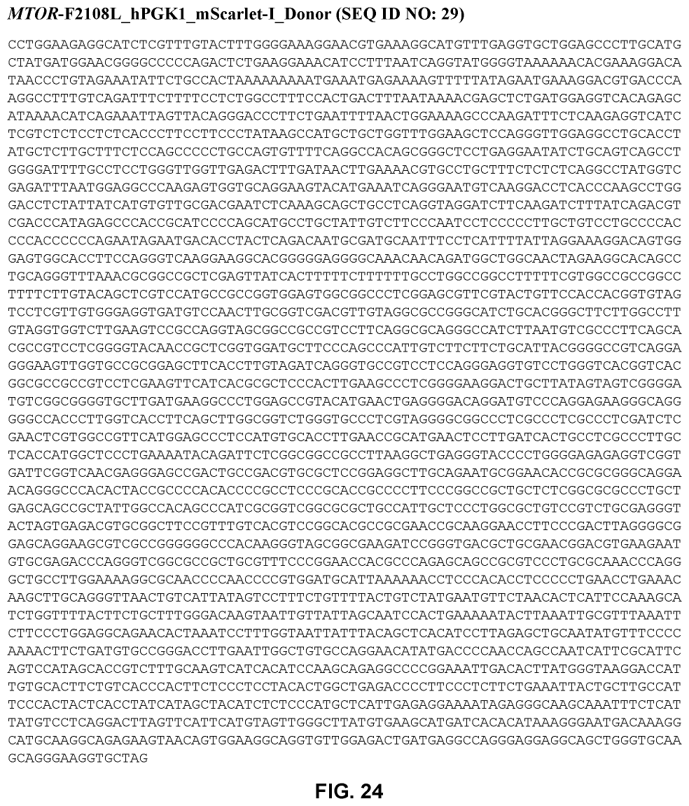

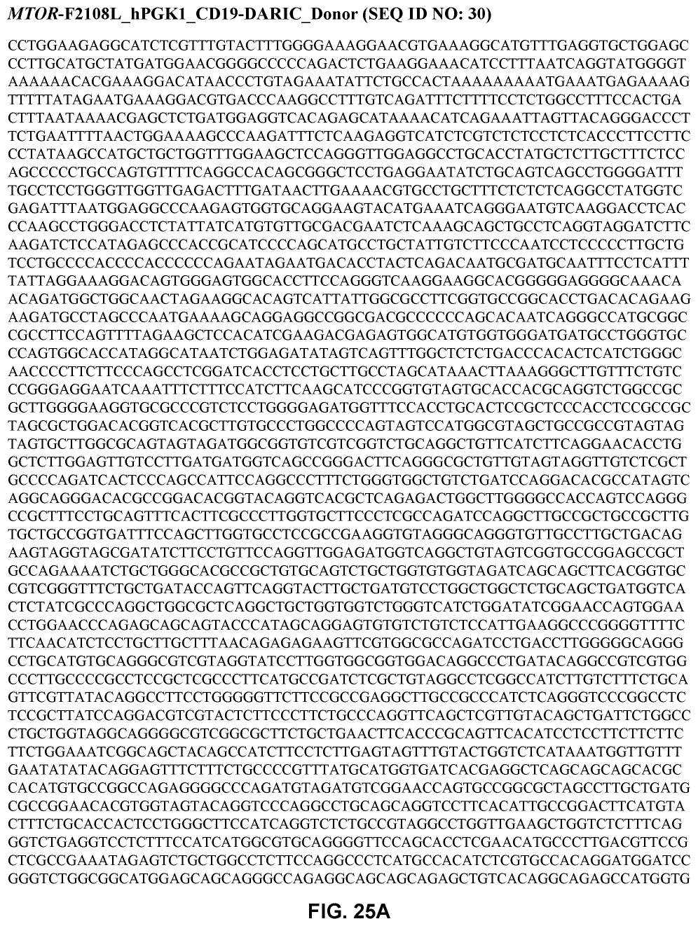

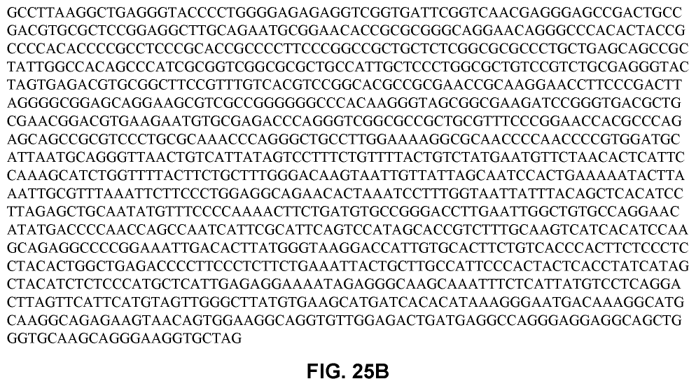

TABLE 5
Sequences described herein

| SEQ ID | |
|---|---|
| NO(s): | Description |
| 1-2 | human MTOR nucleotide (SEQ ID NO: 1) and protein |
| | (SEQ ID NO: 2) sequences (see FIGS. 26A-26C) |
| 3-7 | gRNA target sequences (Table 3) |
| 8-16 | pegRNA target and extension sequences (Table 3) |
| 17-24 | primer sequences (Table 4) |
| 25-30 | donor sequences (see FIGS. 20-25) |
| 31-33 | target sites (see FIG. 1) |
| 34-39 | target sites/donor sequences (see FIG. 2) |
| 40-41 | target sites (see FIG. 7A) |
| 42-44 | target sites (see FIGS. 8A-8B) |
| 45-48 | target sites/donor sequences (see FIG. 12B) |
| 49-59 | protein transduction domain (PTD) sequences |
| 60 | CRISPR nuclease recognition sequence |
| 61-67 | nuclear localization signals (NLSs) |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “α”, “an” and “the” include corresponding plural references unless the context clearly dictates otherwise.

### REFERENCES

1. Bailey, S. R. & Maus, M. V. Gene editing for immune cell therapies. Nat. Biotechnol. 37, 1425-1434 (2019).
2. Ellis, G. I., Sheppard, N. C. & Riley, J. L. Genetic engineering of T cells for immunotherapy. Nat. Rev. Genet. 16, 103-107 (2021).
3. Hong, M., Clubb, J. D. & Chen, Y. Y. Engineering CAR-T cells for next-generation cancer therapy. Cancer Cell 38, 473-488 (2020).
4. Labanieh, L., Majzner, R. G. & Mackall, C. L. Programming CAR-T cells to kill cancer. Nat. Biomed. Eng. 2, 377-391 (2018).
5. Larson, R. C. & Maus, M. V. Recent advances and discoveries in the mechanisms and functions of CAR T cells. Nat. Rev. Cancer 21, 145-161 (2021).
6. Bozza, M. et al. A non viral, non-integrating, DNA Nanovector platform for the safe, rapide, and persistent manufacture of recombinant T-cells. Sci. Adv. 7, eabf1333 (2021).
7. Cromer, M. K. et al. Gene replacement of α-globin with β-globin restores hemoglobin balance in β-thalassemia-derived hematopoietic stem and progenitor cells. Nat. Med. 27, 677-687 (2021).
8. Eyquem, J. et al. Targeting a CAR to the TRAC locus with CRISPR/Cas9 enhances tumour rejection. Nature 543, 113-117 (2017).
9. Von Kalle, C., Deichmann, A. & Schmidt, M. Vector integration and tumorigenesis. Hum. Gene Ther. 25, 475-481 (2014).
10. Hacein-Bey-Abina, S. et al. Insertional oncogenesis in 4 patients after retrovirus-mediated gene therapy of SCID-X1. J. Clin. Invest. 118, 3132-3142 (2008).
11. Nobles, C. L. et al. CD19-targeting CAR T cell immunotherapy outcomes correlate with genomic modification by vector integration. J. Clin. Invest. 130, 673-685 (2019).
12. Chang, C. H. et al. Metabolic competition in the tumor microenvironment is a driver of cancer progression. Cell 162, 1229-1241 (2015).
13. McGuirk, S., Audet-Delage, Y. & St-Pierre, J. Metabolic fitness and plasticity in cancer progression. Trends in Cancer 6, 49-61 (2020).
14. Saxton, R. A. & Sabatini, D. M. mTOR signaling in growth, metabolism, and disease. Cell 168, 960-976 (2017).
15. Mossmann, D., Park, S. & Hall, M. N. mTOR signalling and cellular metabolism are mutual determinants in cancer. Nat. Rev. Cancer 18, 744-757 (2018).
16. Wagle, N. et al. Response and acquired resistance to everolimus in anaplastic thyroid cancer. N. Engl. J. Med. 371, 1426-1433 (2014).
17. Rodrik-Outmezguine, V. S. et al. Overcoming mTOR resistance mutations with a new-generation mTOR inhibitor. Nature 534, 272-276 (2016).
18. Di Stasi, A. et al. Inducible apoptosis as a safety switch for adoptive cell therapy. N. Engl. J. Med. 365, 1673-1683 (2011).
19. Straathof, K. C. et al. An inducible caspase 9 safety switch for T-cell therapy. Blood 105, 4247-4254 (2005).
20. Griffioen, M. et al. Retroviral transfer of human CD20 as a suicide gene for adoptive T-cell therapy. Haematologica 94, 1316-1320 (2009).
21. Philip, B. et al. A highly compact epitope-based marker/suicide gene for easier and safer T-cell therapy. Blood 124, 1277-1287 (2014).
22. Benjamin, R. et al. Genome-edited, donor-derived allogeneic anti-CD19 chimeric antigen receptor T cells in paediatric and adult B-cell acute lymphoblastic leukaemia: results of two phase 1 studies. Lancet 396, 1885-1894 (2020).
23. Saito, Y. et al. CD52 as a molecular target for immunotherapy to treat acute myeloid leukemia with high EVI1 expression. Leukemia 25, 921-931 (2011).
24. Roth, T. L. et al. Reprogramming human T cell function and specificity with non-viral genome targeting. Nature 559, 405-409 (2018).
25. Kiesgen, S., Messinger, J. C., Chintala, N. K., Adusumilli, P. S. & Tano, Z. Comparative analysis of assays to measure CAR T-cell-mediated cytotoxicity. Nat. Protoc. (2021) doi:10.1038/s41596-020-00467-0.
26. Concordet, J. P. & Haeussler, M. CRISPOR: Intuitive guide selection for CRISPR/Cas9 genome editing experiments and screens. Nucleic Acids Res. 46, 242-245 (2018).
27. Nguyen, D. N. et al. Polymer-stabilized Cas9 nanoparticles and modified repair templates increase genome editing efficiency. Nat. Biotechnol. 38, 44.49 (2019).
28. Brinkman, E. K., Chen, T., Amendola, M. & Van Steensel, B. Easy quantitative assessment of genome editing by sequence trace decomposition. Nucleic Acids Res. 42, e168 (2014).
29. Brinkman, E. K. et al. Easy quantification of template-directed CRISPR/Cas9 editing. Nucleic Acids Res. 46, e58-e58 (2018).
30. Hamieh, M. et al. CAR T cell trogocytosis and cooperative killing regulate tumour antigen escape. Nature 568, 112-116 (2019).
31. Chen, P. J. et al. Enhanced prime editing systems by manipulating cellular determinants of editing outcomes. Cell 184, 5635-5652 (2021).
32. Koblan, L. W. et al. Improving cytidine and adenine base editors by expression optimization and ancestral reconstruction. Nat. Biotechnol. 36, 843-848 (2018).
33. Richter, M. F. et al. Phage-assisted evolution of an adenine base editor with improved Cas domain compatibility and activity. Nat. Biotechnol. 38, 883-891 (2020).
34. Oki, T. et al. Imaging dynamic mTORC1 pathway activity in vivo reveals marked shifts that support time-specific inhibitor therapy in AML. Nat. Commun. 12, 245 (2021).
35. Leung, W.-H. et al. Sensitive and adaptable pharmacological control of CAR T cells through extracellular receptor dimerization. JCI Insight 4, e124430 (2019).
36. Clement, K. et al. CRISPResso2 provides accurate and rapid genome editing sequence analysis. Nat. Biotechnol. 37, 215-226 (2019).
37. Mahalati, K. & Kahan, B. D. Clinical pharmacokinetics of sirolimus. Clin. Pharmacokinet. 40, 573-585 (2001).
38. Anzalone, A. V. et al. Search-and-replace genome editing without double-strand breaks or donor DNA. Nature 576, 149-157 (2019).
39. Yan, J., Cirincione, A. & Adamson, B. Prime editing: Precision genome editing by reverse transcription. Mol. Cell 77, 210-212 (2020).
40. Anzalone, A. V, Koblan, L. W. & Liu, D. R. Genome editing with CRISPR-Cas nucleases, base editors, transposases and prime editors. Nat. Biotechnol. 38, 824-844 (2020).
41. Xu, L., Liu, Y. & Han, R. BEAT: A Python program to quantify base editing from Sanger sequencing. CRISPR J. 2, 223-229 (2019).
42. Agudelo, D. et al. Marker-free coselection for CRISPR-driven genome editing in human cells. Nat. Methods 14, 615-620 (2017).
43. Levesque, S. et al. Marker-free coselection for successive rounds of prime editing in human cells. BioRxiv https://doi. org/10.1101/2021.11.02.464583 (2021).
44. Anzalone, A. V et al. Saturation variant interpretation using CRISPR prime editing. Nat. Biotechnol. 40, 885-895. (2022) doi:10.1038/s41587-021-01201-1.
45. Nelson, J. W. et al. Engineered pegRNAs improve prime editing efficiency. Nat. Biotechnol. 40, 402-410 (2021).
46. Clement, K. et al. CRISPResso2 provides accurate and rapid genome editing sequence analysis. Nat. Biotechnol. 37, 215-226 (2019).
47. Huang, J., Nguyen-Mccarty, M., Hexner, E. O., Danet-Desnoyers, G. & Klein, P. S. Maintenance of hematopoietic stem cells through regulation of Wnt and mTOR pathways. Nat. Med. 18, 1778-1785 (2012).
48. Liu, L. et al. E R-associated degradation preserves hematopoietic stem cell quiescence and self-renewal by restricting mTOR activity. Blood 136, 2975-2986 (2020).
49. Chi, H. Regulation and function of mTOR signalling in T cell fate decisions. Nat. Rev. Immunol. 12, 325-338 (2012).
50. Araki, K. et al. mTOR regulates memory CD8 T-cell differentiation. Nature 460, 108-112 (2009).
51. Dormond, O., Romero, P., Zhang, L. & Romero, P. Modulation of mTOR signalling triggers the formation of stem cell-like memory T cells. EBioMedicine 4, 50-61 (2016).
52. Alizadeh, D. et al. I15 enhances CAR-T cell antitumor activity by reducing mTORC1 activity and preserving their stem cell memory phenotype. Cancer Immunol. Res. 7, 759-772 (2019).
53. Lamarthée, B. et al. Transient mTOR inhibition rescues 4-1BB CAR-Tregs from tonic signal-induced dysfunction. Nat. Commun. 12, 6446 (2021).
54. Valvezan, A. J. & Manning, B. D. Molecular logic of mTORC1 signalling as a metabolic rheostat. Nat. Metab. 1, 321-333 (2019).

1. A method for nuclease-based modification of a target polynucleotide at an MTOR locus in a target cell, the method comprising:
(i) introducing a first modification at the MTOR locus, wherein the first modification confers resistance to an mTOR inhibitor;
(ii) introducing a second modification at the MTOR locus, wherein the second modification comprises introduction of a nucleic acid of interest at the MTOR locus.

2. The method of claim 1, wherein said nuclease-based modification in the target polynucleotide is introduced by homology-directed repair (HDR) and the method comprises introducing into the cell one or more donor nucleic acids comprising the first and second modifications

3. (canceled)

4. The method of claim 1, further comprising:
(iii) contacting the target cell with the mTOR inhibitor;
(iv) selecting a cell comprising the first and second modifications by virtue of increased tolerance to the mTOR inhibitor, thereby enriching for the cell comprising the first and second modifications.

5-6. (canceled)

7. The method of claim 1, wherein the method comprises providing the target cell with a CRISPR nuclease and one or more gRNAs comprising one or more guide sequences having one or more target sequences in the target polynucleotide, such that the one or more gRNAs direct the cleavage of the target polynucleotide at the MTOR locus to introduce the first and second modifications, wherein the one or more target sequences are each contiguous to a protospacer adjacent motif recognized by the CRISPR nuclease.

8. The method of claim 1, wherein the method comprises providing said cell with:
a. a1) at least one guide RNA comprising one or more guide sequences having one or more target sequences in the target polynucleotide, such that the one or more gRNAs direct the cleavage of the target polynucleotide at the MTOR locus to introduce the first and second modifications, wherein the one or more target sequences are each contiguous to a protospacer adjacent motif recognized by a Cas9 nickase; or
a2) a vector comprising a nucleic acid sequence corresponding to the guide RNA for expressing the guide RNA; and
b. b1) a Cas nickase-deaminase fusion protein comprising (i) a Cas nickase polypeptide domain and (ii) a cytidine deaminase polypeptide domain or an adenosine deaminase polypeptide domain; or
b2) a vector comprising a nucleic acid sequence encoding the Cas nickase-deaminase fusion protein, for expression of the Cas nickase-deaminase fusion protein; and
wherein at least one modification is introduced into the target polynucleotide, wherein the at least one modification comprises modification of a cytidine into a thymidine resulting in a C to T substitution or an adenosine into an inosine resulting in an A to G substitution, and wherein the at least one modification results in the introduction of the first modification.

9-11. (canceled)

12. The method of claim 1, wherein the method comprises providing said cell with:
a. a1) at least one pegRNA comprising one or more guide sequences having one or more target sequences in the target polynucleotide, such that the one or more pegRNAs direct the cleavage of the target polynucleotide at the MTOR locus to introduce the first and second modifications, wherein the one or more target sequences are each contiguous to a protospacer adjacent motif recognized by a Cas9 nickase; or
a2) a vector comprising a nucleic acid sequence corresponding to the pegRNA for expressing the pegRNA; and
b. b1) a Cas nickase-reverse transcriptase fusion protein comprising (i) a Cas nickase polypeptide domain, and (ii) a reverse transcriptase polypeptide domain; or
b2) a vector comprising a nucleic acid sequence encoding the Cas nickase-reverse transcriptase fusion protein, for expression of the Cas nickase-reverse transcriptase fusion protein; and
wherein at least one modification is introduced into the target polynucleotide, wherein the at least one modification results in the introduction of the first modification.

13-41. (canceled)

42. The method of claim 1, further comprising modification of one or more further target polynucleotides at one or more further loci other than the MTOR locus in the target cell, the method further comprising introducing one or more further modifications at the one or more further loci, wherein the one or more further modifications comprise introduction of one or more further nucleic acids of interest at the one or more further loci.

43. The method of claim 42, wherein the method comprises providing the target cell with a CRISPR nuclease and one or more gRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by the CRISPR nuclease.

44. The method of claim 42, wherein the method comprises providing said cell with:
a. a1) one or more gRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by the CRISPR nuclease; or
a2) one or more vectors comprising one or more nucleic acid sequences corresponding to the one or more gRNAs for expressing the one or more gRNAs; and
b. b1) a Cas nickase-deaminase fusion protein comprising (i) a Cas nickase polypeptide domain and (ii) a cytidine deaminase polypeptide domain or an adenosine deaminase polypeptide domain; or
b2) a vector comprising a nucleic acid sequence encoding the Cas nickase-deaminase fusion protein, for expression of the Cas nickase-deaminase fusion protein; and
wherein at least one of the one or more further modifications comprises modification of a cytidine into a thymidine resulting in a C to T substitution or an adenosine into an inosine resulting in an A to G substitution.

45. The method of claim 42, wherein the method comprises providing said cell with:
a. a1) one or more pegRNAs comprising one or more guide sequences having one or more target sequences in the one or more further target polynucleotides, such that the one or more gRNAs direct the cleavage of the one or more further target polynucleotides at the one or more further loci to introduce the one or more further modifications, wherein the one or more further target sequences are each contiguous to a protospacer adjacent motif recognized by a Cas9 nickase; or
a2) one or more vectors comprising one or more nucleic acid sequences corresponding to the one or more pegRNAs for expressing the one or more pegRNAs; and
b. b1) a Cas nickase-reverse transcriptase fusion protein comprising (i) a Cas nickase polypeptide domain and (ii) a reverse transcriptase polypeptide domain; or
b2) a vector comprising a nucleic acid sequence encoding the Cas nickase-reverse transcriptase fusion protein, for expression of the Cas nickase-reverse transcriptase fusion protein.

46. One or more gRNAs as defined in claim 7.

47-48. (canceled)

49. One or more vectors comprising nucleic acid sequence(s) encoding the one or more gRNAs of claim 46.

50. (canceled)

51. A cell comprising the modified polynucleotide comprising the first and second modifications as defined in claim 1.

52. A cell comprising nucleic acid sequence(s) encoding the one or more gRNAs of claim 46.

53-63. (canceled)

64. A method for treating a disease, condition or disorder in a subject, the method comprising administering an effective amount of the one or more gRNAs of claim 46, to said subject.

65. The method of claim 64, wherein the disease, condition, or disorder is associated with expression of an antigen, and wherein the nucleic acid of interest encodes a recombinant receptor that specifically binds to the antigen.

66-88. (canceled)

89. One or more pegRNAs as defined in claim 12.

90. One or more vectors comprising nucleic acid sequence(s) encoding the one or more pegRNAs of claim 89.

91. A cell comprising nucleic acid sequence(s) encoding the one or more pegRNAs of claim 89.

92. A method for treating a disease, condition or disorder in a subject, the method comprising administering an effective amount of the one or more pegRNAs of claim 89 to said subject.
